(19) United States
(12) Patent Application Publication
(10) Pub. No.: US 2018/0318311 A1
(43) Pub. Date: Nov. 8, 2018

(54) TRICYCLIC 2-QUINOLINONES AS ANTIBACTERIALS

(71) Applicant: Novartis AG, Basel (CH)

(72) Inventors: Guillaume LAPOINTE, San Francisco, CA (US); Wosenu MERGO, Oakland, CA (US); Heinz Ernst MOSER, San Mateo, CA (US); Alexey RIVKIN, Emeryville, CA (US); Colin Keith SKEPPER, Alameda, CA (US); Sarah Louise Williams, Livermore, CA (US)

(21) Appl. No.: 15/971,156

(22) Filed: May 4, 2018

Related U.S. Application Data
(60) Provisional application No. 62/501,990, filed on May 5, 2017.

(51) Int. Cl. A61K 31/5383; A61P 31/04

(52) U.S. Cl. CPC A61K 31/5383; A61K 31/473; A61P 31/04

(57) ABSTRACT

This invention is in the field of medicinal chemistry and relates to compounds, and pharmaceutical compositions thereof that are useful as antibacterial agents. The compounds are useful as inhibitors of bacterial gyrase activity and of bacterial infections, and have the structure of Formula (I):

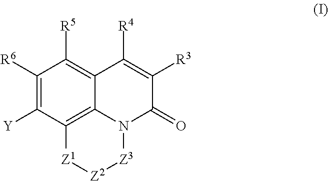

as further described herein. The invention further provides pharmaceutical compositions comprising a compound of Formula (I) and methods of using the compounds and compositions to treat bacterial infections.

### CROSS REFERENCE TO RELATED APPLICATION

This application claims the benefit of priority to U.S. Ser. No. 62/501,990, filed the May 5, 2017, the contents of which are incorporated herein by reference.

### FIELD OF THE INVENTION

This invention is in the field of medicinal chemistry and relates to compounds, and pharmaceutical compositions thereof, that exhibit antibacterial activity. The compounds are inhibitors of bacterial DNA gyrase activity, as data herein demonstrates. The invention also relates to methods for treating bacterial infections in mammals and to methods for decreasing bacterial quantity in a biological sample using these compounds.

### BACKGROUND OF THE INVENTION

Some known antimicrobial agents inhibit bacterial DNA synthesis by acting on DNA gyrase and topoisomerase. DNA gyrase and topoisomerase IV are both type II topoisomerases, consisting of two protein subunits that act as A₂B₂ heterotetramers. The ATPase domain resides on one polypeptide of the dimer (GyrB in DNA gyrase, ParE in topoisomerase IV), while the DNA cleavage core lies on a second polypeptide (GyrA in DNA gyrase, ParC in topoisomerase IV).

Some antibacterial inhibitors of gyrase including aminocoumarins such as novobiocin, function as competitive inhibitors of energy transduction of DNA gyrase by binding to the ATPase active site in GyrB. In contrast, the quinolone antibiotics such as nalidixic acid, ciprofloxacin and moxifloxacin, preferentially bind these enzymes at the cleavage core (GyrA and ParC) and prevent DNA replication and thus halt cell division in both Gram positive and Gram negative bacteria. Although first site resistance mutations generally occur in gyrA, mutations in gyrB also have been shown to reduce susceptibility to these known quinolones.

Bacterial DNA synthesis inhibitors (e.g. fluoroquinolones) have been used to treat primarily Gram-negative infections and have historically achieved good clinical outcomes. A wealth of knowledge exists for the quinolone class of compounds, including bioavailability, tissue distribution, PK/PD relationships and photoxicity. Most of the known fluoroquinolones possess a keto-acid functionality, either a carboxylic acid (ciprofloxacin and moxifloxacin, levofloxacin, the monocyclic and bicyclic 2-pyridone and 4-pyridones), hydroxylamine (quinazolinediones and tricyclic isoquinolones), or a hydrazine (quinazolinediones) group, which relate to DNA gyrase and topoisomerase activity and presumably bind to a divalent cation in the activated complex. Most inhibitors also possess an amine functional group attached to the core heterocycle, making these compounds zwitterionic in nature. Monocyclic 2-pyridone and 4-pyridone (e.g., Ro-13-5478) inhibitors possess this amine functionality attached to a phenyl group. The zwitterionic nature of these inhibitors relate to the permeation of these compounds into the Gram-negative cell using porin channels.

Quinolone antibiotics have been highly effective, but wide-scale deployment of the current drugs, including usage of the effective second generation quinolones that have become generic drugs (e.g., ciprofloxacin), threatens their future long-term utility. Quinolone resistance is already rising in both hospitals and the community at large. See Tessier and Nicolau, Antimicrob. Agents Chemother. 54(6), 2887-89 (2013). To combat such resistant strains, new gyrase inhibitors that are active against bacteria resistant to current quinolones, especially antibiotics targeting multi-drug resistant (MDR) pathogens that retain efficacy against bacteria that are resistant to known quinolones, would address an important unmet medical need.

The present invention relates to antibacterial compounds having activity against both wild-type and quinolone-resistant bacteria. It relates particularly to compounds having activity against quinolone-resistant bacteria, including multi-drug resistant (MDR) strains of e.g. Pseudomonas aeruginosa, as well as antibacterial activity against wild-type and quinolone-resistant Gram-positive pathogens, including methicillin-resistant Staphylococcus aureus (MRSA). The present invention also relates to compounds with selectivity between bacterial topoisomerase IV and DNA gyrase enzyme inhibition compared to human topoisomerase II enzyme inhibition, providing a therapeutic index consistent with in vivo use to treat bacterial infections in humans.

### SUMMARY OF THE INVENTION

The compounds of this invention and pharmaceutical compositions thereof are useful as antibacterials; without being bound by theory, it is believed they act as gyrase inhibitors. The compounds of the invention are useful for the treatment of bacterial infection in subjects in need thereof, especially in humans and other mammals. These compounds include compounds of formula (I):

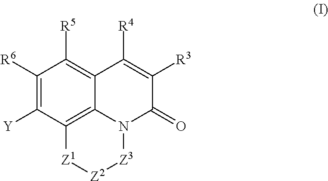

wherein:

Z¹ is selected from the group consisting of O, S, NR¹, and C(R¹)₂;

Z² is selected from C(R¹)₂, O, —C(R¹)₂—C(R¹)₂—, and a bond connecting Z¹ to Z³, provided that when Z² is O, Z¹ is C(R¹)₂;

Z³ is C(R¹)₂;

wherein R¹ is independently selected at each occurrence from H and C₁-C₃ alkyl that is optionally substituted with up to three groups selected from halo, hydroxyl, C₁-C₃-alkoxy, and CN;

R³ is selected from the group consisting of H, -L¹-OR², -L¹-CN, -L¹-N(R²)₂, -L¹-COOR², -L¹-CON(R²)₂, -L¹-N(R²)C(O)R², -L¹-N(R²)C(O)OR, -L¹-SO₂R, -L¹-N(R²)—SO₂—R, and -L¹-SO₂—N(R²)₂; wherein each L¹ is a bond, or a C₁-C₄ straight or branched chain alkylene linker;

each R is independently C₁-C₄ alkyl optionally substituted with one to three groups selected from halogen, —OH, alkoxy, CN, —NH₂, —NH(C₁-C₄ alkyl), —N(C₁-C₄ alkyl)₂, —SO₂(C₁-C₄ alkyl), and oxo;

each R² is independently H or C₁-C₄ alkyl optionally substituted with up to three groups selected from halogen, —OH, alkoxy, CN, —NR¹²R¹³, —SO₂R and oxo;

or two R² on the same nitrogen can be taken together to form a 4-6 membered heterocyclic ring optionally containing an additional heteroatom selected from N, O and S as a ring member and optionally substituted with up to three groups selected from halogen, —OH, C₁-C₄ alkyl, C₁-C₄ alkoxy, CN, —NR¹²R¹³, and oxo;

R⁴ is selected from the group consisting of H, halo, C₁-C₆ alkyl, C₁-C₄ haloalkyl, -L²-OR², -L²-CN, -L²-N(R²)₂, and -L²-NR²C(O)—R²;

each L² is independently selected from a bond and a divalent straight chain or branched C₁-C₆ alkyl;

R⁵ is selected from the group consisting of H, halo, amino, CN, C₁-C₄ alkyl, C₁-C₄ alkoxy, and C₁-C₄ haloalkyl;

R⁶ is selected from the group consisting of H, halo, CN, C₁-C₄ alkyl, C₁-C₄ alkoxy, and C₁-C₄ haloalkyl;

Y is a group of the formula —NR^{7A}R^{7B},

wherein R^{7A} is selected from the group consisting of H, —C(O)R², —C(O)OR², and C₁-C₆ alkyl optionally substituted with up to two groups independently selected from halogen, —OH, C₁-C₄ haloalkyl, C₁-C₄ alkoxy, oxo, ═N—OR², —N(R²)₂, C₃-C₇ cycloalkyl, —COOR², —C(O)N(R²)₂, —NR²C(O)R², —NR²C(O)OR, and a 4-6 membered heteroaryl or heterocyclyl group that contains up to two heteroatoms selected from N, O and S as ring members and is optionally substituted with up to two groups selected from hydroxy, amino, halogen, C₁-C₄ alkyl, C₁-C₄ haloalkyl, and C₁-C₄ alkoxy;

R^{7B} is -L³-Q³ or C₁-C₆ alkyl optionally substituted with up to two groups independently selected from halogen, —OH, C₁-C₄ haloalkyl, C₁-C₄ alkoxy, oxo, —N(R²)₂, C₃-C₇ cycloalkyl, —COOR², —C(O)N(R²)₂, —NR²C(O)R², —NR²C(O)OR, and a 4-6 membered heteroaryl or heterocyclyl group that contains up to two heteroatoms selected from N, O and S as ring members and is optionally substituted with up to two groups selected from hydroxy, amino, halogen, C₁-C₄ alkyl, C₁-C₄ haloalkyl, and C₁-C₄ alkoxy,

wherein L³ is a bond or a straight or branched chain C₁-C₆ alkyl linker, and Q³ is selected from pyridinyl and a 4-7 membered heterocyclyl containing one or two heteroatoms selected from N, O and S as ring members, and wherein Q³ is optionally substituted with up to three groups selected from halogen, CN, —OH, C₁-C₄ alkyl, C₁-C₄ haloalkyl, C₁-C₄ alkoxy, oxo, ═N—OR², —N(R²)₂, —COOR², —C(O)N(R²)₂, —NR²C(O)R², —NR²C(O)OR;

or R^{7A} and R^{7B} together with the nitrogen atom to which they are attached form a 4- to 7-membered monocyclic group optionally including one additional heteroatom selected from N, O and S as a ring member, or a 6-10 membered bicyclic heterocyclic group optionally including one or two additional heteroatoms selected from N, O and S as ring members, wherein the monocyclic or bicyclic heterocyclic group formed by R^{7A} and R^{7B} together with the nitrogen atom to which they are attached is optionally substituted by up to four groups selected from halogen, —CN, hydroxy, phenyl, oxo, —OR⁹, —N(R⁹)₂, —COOR⁹, —C(O)N(R⁹)₂, C₁-C₄ alkyl, ═C(R⁸)₂, C₁-C₄ haloalkyl, C₁-C₄ alkoxy, oxo, C₃-C₆ cycloalkyl, and a 4-6 membered heteroaryl or heterocyclyl group that contains up to two heteroatoms selected from N, O and S as ring members,

wherein the C₁-C₄ alkyl, C₃-C₆ cycloalkyl, phenyl, and 4-6 membered heteroaryl or heterocyclyl are each optionally substituted by up to three groups independently selected from halogen, —CN, hydroxy, oxo, —OR¹⁰, ═N—OR¹⁰, —N(R¹⁰)₂, —COOR¹⁰, —N(R¹⁰)—C(O)—O—(C₁-C₄ alkyl), —C(O)N(R¹⁰)₂, C₁-C₄ alkyl, C₁-C₄ haloalkyl, and C₁-C₄ alkoxy;

R⁸ is selected independently at each occurrence from the group consisting of H, halo, CN, C₁-C₄ alkoxy, C₁-C₄ haloalkyl, and C₁-C₄ alkyl optionally substituted with hydroxy or amino;

R⁹ and R¹⁰ are each independently selected from H and C₁-C₄ alkyl optionally substituted with up to three groups selected from halogen, —OH, C₁-C₄ alkoxy, CN, —NR¹²R¹³, —SO₂R and oxo;

or two R⁹ or two R¹⁰ on the same nitrogen can be taken together to form a 4-6 membered heterocyclic ring optionally containing an additional heteroatom selected from N, O and S as a ring member and optionally substituted with up to three groups selected from halogen, —OH, C₁-C₄ alkyl, C₁-C₄ alkoxy, CN, —NR¹²R¹³, and oxo;

each R¹¹ is independently hydrogen or C₁-C₄ alkyl optionally substituted with one or two groups selected from halogen, —OH, C₁-C₄ alkoxy, CN, —NH₂, —NH(C₁-C₄ alkyl), —N(C₁-C₄alkyl)₂, —SO₂(C₁-C₄ alkyl), and oxo;

each R¹² and R¹³ is independently hydrogen or C₁-C₄ alkyl optionally substituted with one or two groups selected from halogen, —OH, C₁-C₄ alkoxy, CN, —NH₂, —NH(C₁-C₄ alkyl), —N(C₁-C₄ alkyl)₂, —SO₂(C₁-C₄ alkyl), and oxo;

or each R¹² and R¹³ together with the nitrogen atom to which they are both attached can form a 4- to 6-membered heterocyclyl optionally including an additional heteroatom selected from N, O and S as a ring member and optionally substituted by one to three substituents selected from OH, halogen, oxo, ═N—OR¹¹, C₁-C₆ alkyl optionally substituted by one to three halogen atoms or NH₂, C₁-C₆ alkoxy optionally substituted by one or more OH or C₁-C₆ alkoxy groups, and —C(O)O—C₁-C₆ alkyl;

including pharmaceutically acceptable salts of such compounds. Various additional embodiments of the compounds of the invention are described below.

These compounds, and pharmaceutical compositions containing them, are useful for treating or lessening the severity of bacterial infections. In particular, the compounds of the present invention are useful in treating or lessening the severity of upper respiratory infections, lower respiratory infections, ear infections, pleuropulmonary and bronchial infections, urinary tract infections, intra-abdominal infections, cardiovascular infections, a blood stream infection, sepsis, CNS infections, skin and soft tissue infections, GI infections, bone and joint infections, genital infections, eye infections, or granulomatous infections. The compounds are effective against a range of bacteria, including both Gram-positive and Gram-negative bacteria.

### DETAILED DESCRIPTION OF THE INVENTION

For purposes of interpreting this specification, the following definitions will apply unless specified otherwise and whenever appropriate, terms used in the singular will also include the plural and vice versa.

### Definitions

The term “a,” “an,” “the” and similar terms used in the context of the present invention (especially in the context of the claims) are to be construed to cover both the singular and plural unless otherwise indicated herein or clearly contradicted by the context.

As used herein, the term “halogen “(or halo) refers to fluorine, bromine, chlorine or iodine, in particular fluorine or chlorine. Halogen-substituted groups and moieties, such as alkyl substituted by halogen (haloalkyl) can be mono-, poly- or per-halogenated.

As used herein, unless otherwise specified, the term “heteroatom” refers to nitrogen (N), oxygen (O) or sulfur (S).

As used herein, the term “alkyl” refers to a fully saturated branched or unbranched hydrocarbon moiety having up to 10 carbon atoms. Unless otherwise provided, alkyl refers to hydrocarbon moieties having 1 to 6 carbon atoms (which may be written as C₁-C₆, or C₁-C₆ alkyl), or alternatively 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. A substituted alkyl is an alkyl group containing one or more substituents in place of a hydrogen atom of the corresponding unsubstituted alkyl group, such as one, two or three substituents, up to the number of Hydrogens on the unsubstituted alkyl group. Suitable substituents for alkyl groups, if not otherwise specified, may be selected from halogen, CN, oxo, hydroxy, amino, and C₁-C₄alkoxy groups.

As used herein, the term “alkylene” refers to a divalent alkyl group having 1 to 10 carbon atoms, and two open valences to attach to other components. Unless otherwise provided, alkylene refers to moieties having typically 1 to 6 carbon atoms, or alternatively 1 to 4 carbon atoms. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene, n-decylene and the like. A substituted alkylene is an alkylene group containing one or more, such as one, two or three substituents; unless otherwise specified, suitable substituents are selected from the substituents listed above for alkyl groups.

As used herein, the term “haloalkyl” refers to an alkyl as defined herein, which is substituted by one or more halogen atoms as defined herein. The haloalkyl can be monohaloalkyl, dihaloalkyl, trihaloalkyl, or polyhaloalkyl including perhaloalkyl. A monohaloalkyl can have one iodo, bromo, chloro or fluoro within the alkyl group. Chloro and fluoro are preferred on alkyl or cycloalkyl groups; fluoro, chloro and bromo are often preferred on aryl or heteroaryl groups. Dihaloalkyl and polyhaloalkyl groups can have two or more of the same halo atoms or a combination of different halo groups within the alkyl. Typically the polyhaloalkyl contains up to 12, or 10, or 8, or 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-alkyl refers to an alkyl having all hydrogen atoms replaced with halo atoms, e.g, trifluoromethyl.

As used herein, the term “alkoxy” refers to alkyl-O—, wherein alkyl is defined above. Representative examples of alkoxy include, but are not limited to, methoxy, ethoxy, propoxy, 2-propoxy, butoxy, tert-butoxy, pentyloxy, hexyloxy, and the like. Typically, alkoxy groups have 1-6 carbons, more commonly 1-4 carbon atoms.

A substituted alkoxy is an alkoxy group containing one or more, such as one, two or three substituents on the alkyl portion of the alkoxy. Unless otherwise specified, suitable substituents are selected from the substituents listed above for alkyl groups.

Similarly, each alkyl part of other groups like “alkoxyalkyl”, “alkoxycarbonyl”, “alkoxy-carbonylalkyl”, “alkylsulfonyl”, “alkylsulfoxyl”, “alkylamino”, or “haloalkyl” shall have the same meaning as described in the above-mentioned definition of “alkyl”. When used in this way, unless otherwise indicated, the alkyl group is often a 1-4 carbon alkyl and is not further substituted by groups other than the components named. When such alkyl groups are substituted, suitable substituents are those named above for alkyl groups unless otherwise specified.

As used herein, the term “haloalkoxy” refers to haloalkyl-O—, wherein haloalkyl is defined above. Representative examples of haloalkoxy include, but are not limited to, fluoromethoxy, difluoromethoxy, trifluoromethoxy, trichloromethoxy, 2-chloroethoxy, 2,2,2-trifluoroethoxy, 1,1,1,3,3,3-hexafluoro-2-propoxy, and the like.

As used herein, the term “cycloalkyl” refers to saturated or unsaturated non-aromatic monocyclic, bicyclic, tricyclic or spirocyclic hydrocarbon groups having 3-12 carbon atoms: the cycloalkyl group may be unsaturated, and may be fused to another ring that can be saturated, unsaturated or aromatic, provided the ring atom of the cycloalkyl group that is connected to the molecular formula of interest is in a non-aromatic ring. Unless otherwise provided, cycloalkyl refers to cyclic hydrocarbon groups having between 3 and 12 ring carbon atoms or between 3 and 8 ring carbon atoms. Frequently, cycloalkyl groups are saturated monocyclic rings having 3-7 ring atoms unless otherwise specified.

A substituted cycloalkyl is a cycloalkyl group substituted by one, or two, or three or more substituents, up to the number of hydrogens on the unsubstituted group. Typically, a substituted cycloalkyl will have 1-4 or 1-2 substituents. Suitable substituents, unless otherwise specified, are independently selected from the group consisting of halogen, hydroxyl, thiol, cyano, nitro, oxo, C₁-C₄-alkylimino, C₁-C₄-alkoximino, hydroxyimino, C₁-C₄-alkyl, C₂-C₄-alkenyl, C₂-C₄-alkynyl, C₁-C₄-alkoxy, C₁-C₄-thioalkyl, C₂-C₄-alkenyloxy, C₂-C₄-alkynyloxy, C₁-C₄-alkylcarbonyl, carboxy, C₁-C₄-alkoxycarbonyl, amino, C₁-C₄-alkylamino, di-C₁-C₄-alkylamino, C₁-C₄-alkylaminocarbonyl, di-C₁-C₄-alkylaminocarbonyl, C₁-C₄-alkylcarbonylamino, C₁-C₄-alkylcarbonyl(C₁-C₄-alkyl)amino, C₁-C₄-alkylsulfonyl, C₁-C₄-alkylsulfamoyl, and C₁-C₄-alkylaminosulfonyl, where each of the aforementioned hydrocarbon groups (e.g., alkyl, alkenyl, alkynyl, alkoxy residues) may be further substituted by one or more groups independently selected at each occurrence from the list of substituents for ‘alkyl’ groups provided above. Preferred substituents for cycloalkyl groups include C₁-C₄alkyl and the substituent groups listed above for alkyl groups.

Exemplary monocyclic hydrocarbon groups include, but are not limited to, cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like. Exemplary tricyclic hydrocarbon groups include adamantyl and the like.

Similarly, each cycloalkyl part of other groups like “cycloalkyloxy”, “cycloalkylalkyl”, or “halocycloalkyl” shall have the same meaning as described in the above-mentioned definition of “cycloalkyl”. When used in these terms, the cycloalkyl is typically a monocyclic 3-7 carbon ring, that is unsubstituted or substituted with 1-2 groups. When optionally substituted, the substituents are typically selected from C₁-C₄ alkyl and those set forth above as suitable for alkyl groups.

As used herein, the term “aryl” refers to an aromatic hydrocarbon group having 6-10 carbon atoms in the ring portion. Typically, aryl is monocyclic, bicyclic or tricyclic aryl having 6-10 carbon atoms, e.g., phenyl or naphthyl. Furthermore, the term “aryl” as used herein, refers to an aromatic substituent which can be a single aromatic ring, or multiple aromatic rings that are fused together. Non-limiting examples include phenyl, naphthyl and tetrahydronaphthyl, provided the tetrahydronaphthyl is connected to the formula of interest through a carbon of the aromatic ring of the tetrahydronaphthyl group.

A substituted aryl is an aryl group substituted by 1-5 (such as one, or two, or three) substituents independently selected from the group consisting of hydroxyl, thiol, cyano, nitro, C₁-C₄-alkyl, C₂-C₄-alkenyl, C₂-C₄-alkynyl, C₁-C₄-alkoxy, C₁-C₄-thioalkyl, C₂-C₄-alkenyloxy, C₂-C₄-alkynyloxy, halogen, C₁-C₄-alkylcarbonyl, carboxy, C₁-C₄-alkoxycarbonyl, amino, C₁-C₄-alkylamino, di-C₁-C₄-alkylamino, C₁-C₄-alkylaminocarbonyl, di-C₁-C₄-alkylaminocarbonyl, C₁-C₄-alkylcarbonylamino, C₁-C₄-alkylcarbonyl(C₁-C₄-alkyl)amino, C₁-C₄-alkyl sulfonyl, sulfamoyl, C₁-C₄-alkylsulfamoyl, and C₁-C₄-alkylaminosulfonyl where each of the afore-mentioned hydrocarbon groups (e.g., alkyl, alkenyl, alkynyl, alkoxy residues) may be further substituted by one or more groups independently selected at each occurrence from the groups listed above as suitable substituents for alkyl groups.

Similarly, the term aryl when used as part of other groups like “aryloxy” or “arylalkyl” shall have the same meaning as described in the above-mentioned definition of “aryl”.

As used herein, the term “heterocyclyl” or “heterocyclic” or “heterocycle” refers to a heterocyclic group that is saturated or partially saturated but not aromatic, and is preferably a monocyclic or a polycyclic ring (in case of a polycyclic ring particularly a bicyclic, tricyclic or spirocyclic ring); and has 3 to 12, more typically 3 to 8 and most often 5 or 6 ring atoms; wherein one or more, preferably one to four, especially one or two ring atoms are heteroatoms independently selected from 0, S and N (the remaining ring atoms therefore being carbon). Preferably, a heterocyclyl group has one or two such heteroatoms as ring atoms, and commonly the heteroatoms are not directly connected to each other. The bonding ring (i.e. the ring connecting to the Formula of interest) preferably has 4 to 12, especially 5 to 7 ring atoms. The heterocyclic group can be fused to an aromatic ring, provided it is attached to the Formula of interest at an atom of the heterocyclic group that is not aromatic. The heterocyclic group can be attached to the Formula of interest via a heteroatom (typically nitrogen) or a carbon atom of the heterocyclic group. The heterocyclyl can include fused or bridged rings as well as spirocyclic rings, and only one ring of a polycyclic heterocyclic group needs to contain a heteroatom as a ring. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, and the like.

A substituted heterocyclyl is a heterocyclyl group independently substituted by 1-5 (such as one, or two, or three) substituents selected from the substituents described above for a cycloalkyl group.

Similarly, the term heterocyclyl used as part of other groups like “heterocyclylalkyl” shall have the same meaning as described in the above definition of “heterocyclyl”.

As used herein, the term “heteroaryl” refers to a 5-14 membered monocyclic- or bicyclic- or tricyclic-aromatic ring system, having 1 to 8 heteroatoms as ring members; the heteroatoms are selected from N, O and S unless otherwise specified. Typically, the heteroaryl in a compound of the invention is a 5-10 membered ring system or a 5-7 membered ring system(e.g., 5-7 membered monocyclic or an 8-10 membered bicyclic group). Typical heteroaryl groups include 2- or 3-thienyl, 2- or 3-furyl, 1-, 2- or 3-pyrrolyl, 1-, 2-, 4-, or 5-imidazolyl, 1-, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-(1,2,4)-triazolyl, 4- or 5-(1,2,3)-triazolyl, 1- or 2-tetrazolyl, 2-, 3-, or 4-pyridyl, 3- or 4-pyridazinyl, 2-pyrazinyl, and 2-, 4-, or 5-pyrimidinyl.

The term “heteroaryl” also refers to a group in which a heteroaromatic ring is fused to one or more aryl, cycloalkyl, or heterocyclyl rings, where the radical or point of attachment to the Formula of interest is on a heteroaromatic ring. Typical fused heteroaryl groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, and 2-, 4-, 5-, 6-, or 7-benzothiazolyl.

A substituted heteroaryl is a heteroaryl group containing one or more substituents selected from the substituents described above as suitable for an aryl group, unless otherwise specified.

The term “hydroxy” or “hydroxyl” refers to the group —OH.

The term “spiro” as used herein includes 3- to 6-cycloalkyl or 4- to 6-atom heterocyclic rings having one or two heteroatoms selected from N, O and S as ring members, which can optionally be substituted as defined, wherein the spiro ring is fused onto a single carbon atom of a non-aromatic ring, making the carbon atom shared by both rings a spirocyclic center. Q is a suitable substituent for attachment to the spirocyclic ring, e.g. H or C₁-C₄ alkyl.

Illustrative examples of spiro groups are:

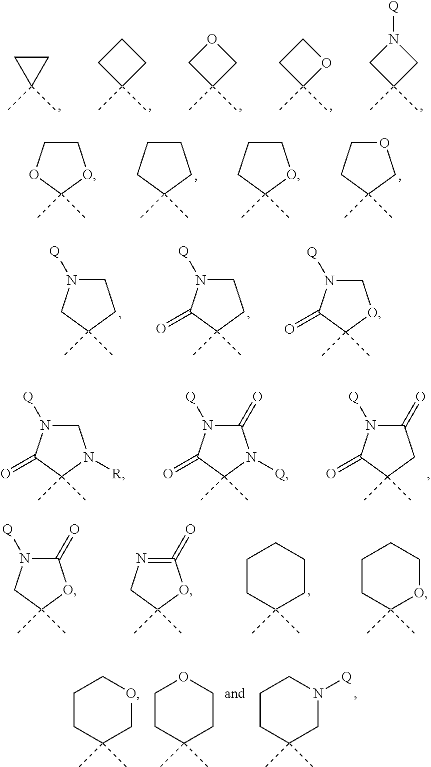

where the dashed bonds in each structure represent bonds of a non-aromatic ring with which the spirocyclic group shares one atom.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, suitable for use in a pharmaceutical composition, as would be known to those skilled in the art (see, for example, Remington's Pharmaceutical Sciences, 18th Ed., Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease by reducing or inhibiting the activity of gyrase; or reduce or inhibit the expression of gyrase. In another non-limiting embodiment, the term “a therapeutically effective amount” refers to the amount of the compound of the present invention that, when administered to a subject treats or ameliorates a bacterial infection in said subject.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In some embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

Various embodiments of the invention are described herein. It will be recognized that features specified in each embodiment may be combined with other specified features to provide further embodiments. The following enumerated embodiments are representative:

1. A compound of formula (I):

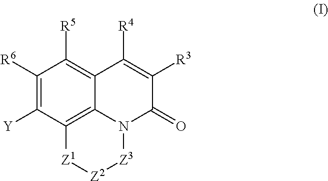

wherein:

wherein:

Z¹ is selected from the group consisting of O, S, NR¹, and C(R¹)₂;

Z² is selected from C(R¹)₂, O, —C(R¹)₂—C(R¹)₂—, and a bond connecting Z¹ to Z³, provided that when Z² is O, Z¹ is C(R¹)₂;

Z³ is C(R¹)₂;

wherein R¹ is independently selected at each occurrence from H and C₁-C₃ alkyl that is optionally substituted with up to three groups selected from halo, hydroxyl, C₁-C₃-alkoxy, and CN;

R³ is selected from the group consisting of H, -L¹-OR², -L¹-CN, -L¹-N(R²)₂, -L¹-COOR², -L¹-CON(R²)₂, -L¹-N(R²)C(O)R², -L¹-N(R²)C(O)OR, -L¹-SO₂R, -L¹-N(R²)-SO₂-R, and -L¹-SO₂-N(R²)₂; wherein each L¹ is a bond, or a C₁-C₄ straight or branched chain alkylene linker;

each R is independently C₁-C₄ alkyl optionally substituted with one to three groups selected from halogen, —OH, C₁-C₄ alkoxy, CN, —NH₂, —NH(C₁-C₄ alkyl), —N(C₁-C₄ alkyl)₂, —SO₂(C₁-C₄ alkyl), and oxo;

each R² is independently H or C₁-C₄ alkyl optionally substituted with up to three groups selected from halogen, —OH, C₁-C₄ alkoxy, CN, —NR¹²R¹³, —SO₂R and oxo;

or two R² on the same nitrogen can be taken together to form a 4-6 membered heterocyclic ring optionally containing an additional heteroatom selected from N, O and S as a ring member and optionally substituted with up to three groups selected from halogen, —OH, C₁-C₄ alkyl, C₁-C₄ alkoxy, CN, —NR¹²R¹³, and oxo;

R⁴ is selected from the group consisting of H, halo, C₁-C₆ alkyl, C₁-C₄ haloalkyl, -L²-OR², OR², -L²-CN, -L²-N(R²)₂, and -L²-NR²C(O)—R²;

each L² is independently selected from a bond and a divalent straight chain or branched C₁-C₆ alkyl;

R⁵ is selected from the group consisting of H, halo, amino, CN, C₁-C₄ alkyl, C₁-C₄ alkoxy, and C₁-C₄ haloalkyl;

R⁶ is selected from the group consisting of H, halo, CN, C₁-C₄ alkyl, C₁-C₄ alkoxy, and C₁-C₄ haloalkyl;

Y is a group of the formula —NR^{7A}R^{7B},

wherein R^{7A} is selected from the group consisting of H, —C(O)R², —C(O)OR², and C₁-C₆ alkyl optionally substituted with up to two groups independently selected from halogen, —OH, C₁-C₄ haloalkyl, C₁-C₄ alkoxy, oxo, ═N—OR², —N(R²)₂, C₃-C₇ cycloalkyl, —COOR², —C(O)N(R²)₂, —NR²C(O)R², —NR²C(O)OR, and a 4-6 membered heteroaryl or heterocyclyl group that contains up to two heteroatoms selected from N, O and S as ring members and is optionally substituted with up to two groups selected from hydroxy, amino, halogen, C₁-C₄ alkyl, C₁-C₄ haloalkyl, and C₁-C₄ alkoxy;

R^{7B} is -L³-Q³ or C₁-C₆ alkyl optionally substituted with up to two groups independently selected from halogen, —OH, C₁-C₄ haloalkyl, C₁-C₄ alkoxy, oxo, —N(R²)₂, C₃-C₇ cycloalkyl, —COOR², —C(O)N(R²)₂, —NR²C(O)R², —NR²C(O)OR, and a 4-6 membered heteroaryl or heterocyclyl group that contains up to two heteroatoms selected from N, O and S as ring members and is optionally substituted with up to two groups selected from hydroxy, amino, halogen, C₁-C₄ alkyl, C₁-C₄ haloalkyl, and C₁-C₄ alkoxy,

wherein L³ is a bond or a straight or branched chain C₁-C₆ alkyl linker, and Q³ is selected from pyridinyl and a 4-7 membered heterocyclyl containing one or two heteroatoms selected from N, O and S as ring members, and wherein Q³ is optionally substituted with up to three groups selected from halogen, CN, —OH, C₁-C₄ alkyl, C₁-C₄ haloalkyl, C₁-C₄ alkoxy, oxo, ═N—OR², —N(R²)₂, —COOR², —C(O)N(R²)₂, —NR²C(O)R², —NR²C(O)OR;

or R^{7A} and R^{7B} together with the nitrogen atom to which they are attached form a 4- to 7-membered monocyclic group optionally including one additional heteroatom selected from N, O and S as a ring member, or a 6-10 membered bicyclic heterocyclic group optionally including one or two additional heteroatoms selected from N, O and S as ring members, wherein the monocyclic or bicyclic heterocyclic group formed by R^{7A} and R^{7B} together with the nitrogen atom to which they are attached is optionally substituted by up to four groups selected from halogen, —CN, hydroxy, phenyl, oxo, —OR⁹, —N(R⁹)₂, —COOR⁹, —C(O)N(R⁹)₂, C₁-C₄ alkyl, ═C(R⁸)₂, C₁-C₄ haloalkyl, C₁-C₄ alkoxy, oxo, C₃-C₆ cycloalkyl, and a 4-6 membered heteroaryl or heterocyclyl group that contains up to two heteroatoms selected from N, O and S as ring members,

wherein the C₁-C₄ alkyl, C₃-C₆ cycloalkyl, phenyl, and 4-6 membered heteroaryl or heterocyclyl are each optionally substituted by up to three groups independently selected from halogen, —CN, hydroxy, oxo, —OR¹⁰, ═N—OR¹⁰, —N(R¹⁰)₂, —COOR¹⁰, —N(R¹⁰)—C(O)—O—(C₁-C₄ alkyl), —C(O)N(R¹⁰)₂, C₁-C₄ alkyl, C₁-C₄ haloalkyl, and C₁-C₄ alkoxy;

R⁸ is selected independently at each occurrence from the group consisting of H, halo, CN, C₁-C₄ alkoxy, C₁-C₄ haloalkyl, and C₁-C₄ alkyl optionally substituted with hydroxy or amino;

R⁹ and R¹⁰ are each independently selected from H and C₁-C₄ alkyl optionally substituted with up to three groups selected from halogen, —OH, C₁-C₄ alkoxy, CN, —NR¹²R¹³, —SO₂R and oxo; or two R⁹ or two R¹⁰ on the same nitrogen can be taken together to form a 4-6 membered heterocyclic ring optionally containing an additional heteroatom selected from N, O and S as a ring member and optionally substituted with up to three groups selected from halogen, —OH, C₁-C₄ alkyl, C₁-C₄ alkoxy, CN, —NR¹²R¹³, and oxo;

each R¹¹ is independently hydrogen or C₁-C₄ alkyl optionally substituted with one or two groups selected from halogen, —OH, C₁-C₄ alkoxy, CN, —NH₂, —NH(C₁-C₄ alkyl), —N(C₁-C₄alkyl)₂, —SO₂(C₁-C₄ alkyl), and oxo;

each R¹² and R¹³ is independently hydrogen or C₁-C₄ alkyl optionally substituted with one or two groups selected from halogen, —OH, C₁-C₄ alkoxy, CN, —NH₂, —NH(C₁-C₄ alkyl), —N(C₁-C₄ alkyl)₂, —SO₂(C₁-C₄ alkyl), and oxo;

or each R¹² and R¹³ together with the nitrogen atom to which they are both attached can form a 4- to 6-membered heterocyclyl optionally including an additional heteroatom selected from N, O and S as a ring member and optionally substituted by one to three substituents selected from OH, halogen, oxo, ═N—OR¹¹, C₁-C₆ alkyl optionally substituted by one to three halogen atoms or NH₂, C₁-C₆ alkoxy optionally substituted by one or more OH or C₁-C₆ alkoxy groups, and —C(O)O—C₁-C₆ alkyl;

or a pharmaceutically acceptable salt thereof.

Each compound of the Examples herein is a specific embodiment of the compounds of the invention.

2. The compound of embodiment 1, wherein R³ is H or COOR².

3. The compound of embodiment 1 or 2, or a pharmaceutically acceptable salt thereof, wherein R⁵ is H or halogen. In certain of these embodiments, R⁵ is H.

4. The compound of any one of the preceding embodiments, wherein R⁶ is H or F; or a pharmaceutically acceptable salt thereof. In certain of these embodiments, R⁶ is F.

5. The compound of any one of the preceding embodiments, wherein each R¹ is independently selected from H and methyl; or a pharmaceutically acceptable salt thereof.

6. The compound of any one of the preceding embodiments, wherein R³ is H; or a pharmaceutically acceptable salt thereof.

7. The compound of any one of embodiments 1-5, wherein R³ is —COOH; or a pharmaceutically acceptable salt thereof.

8. The compound of any one of the preceding embodiments, wherein R⁴ is H; or a pharmaceutically acceptable salt thereof.

9. The compound of any one of embodiments 1-7, wherein R⁴ is —CH₂—N(R²)₂; or a pharmaceutically acceptable salt thereof. In certain of these embodiments, R⁴ is —CH₂NH₂.

10. The compound of any one of the preceding embodiments, which is of the formula (II):

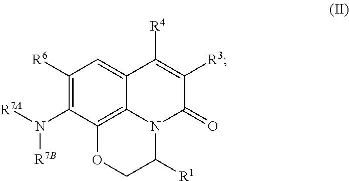

or a pharmaceutically acceptable salt thereof.

In certain of these embodiments, R¹ is methyl. In certain of these embodiments, R⁶ is F.

11. The compound according to embodiment 10, wherein R^{7A} is H; or a pharmaceutically acceptable salt thereof.

12. The compound according to embodiment 10, wherein R^{7A} and R^{7B} together with the nitrogen atom to which they are attached form a 4- to 7-membered monocyclic heterocyclic group optionally including one additional heteroatom selected from N, O and S as a ring member,

or a 6-10 membered bicyclic heterocyclic group optionally including one or two additional heteroatoms selected from N, O and S as ring members,

wherein the monocyclic or bicyclic heterocyclic group formed by R^{7A} and R^{7B} together with the nitrogen atom to which they are attached is optionally substituted by up to three groups selected from halogen, —CN, hydroxy, phenyl, oxo, —OR⁹, —N(R⁹)₂, —COOR⁹, —C(O)N(R⁹)₂, C₁-C₄ alkyl, C₁-C₄ haloalkyl, C₁-C₄alkoxy, oxo, C₃-C₆ cycloalkyl, and a 4-6 membered heteroaryl or heterocyclyl group that contains up to two heteroatoms selected from N, O and S as ring members,

wherein the C₁-C₄ alkyl, C₃-C₆ cycloalkyl, phenyl, and 4-6 membered heteroaryl or heterocyclyl are each optionally substituted by up to three groups independently selected from halogen, —CN, hydroxy, oxo, —OR¹⁰, ═N—OR¹⁰, —N(R¹⁰)₂, —COOR¹⁰, —C(O)N(R¹⁰)₂, C₁-C₄ alkyl, C₁-C₄ haloalkyl, and C₁-C₄alkoxy,

or a pharmaceutically acceptable salt thereof.

13. The compound according to embodiment 1, or a pharmaceutically acceptable salt thereof, wherein the compound has formula (IV):

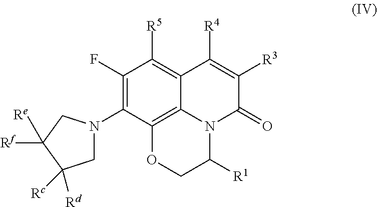

wherein,

R¹ is independently at each occurrence hydrogen or methyl;

R³ is hydrogen, halo , C₁₋₂ alkyl, or C₁₋₂ haloalkyl;

R⁴ is H or —CH₂NH₂,

R⁵ is H, Me or halo;

R^{c} and R^{f} are independently selected from hydrogen and halo, or R^{c} and R^{f} taken together with the atoms to which they are attached form a cyclopropyl ring;

R^{d} and R^{e} are each independently selected from the group consisting of H, —NH₂, —CH₂NH₂, —CH₂NHCH₃, OH, CH₂OH,

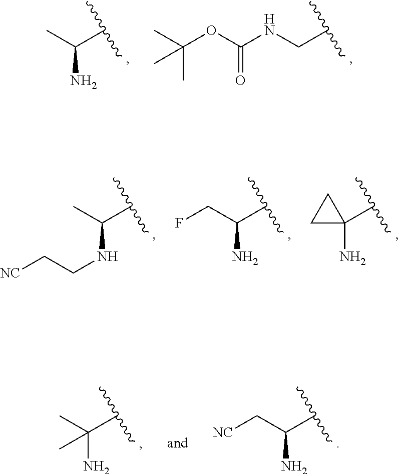

14. The compound according to embodiment 1, or a pharmaceutically acceptable salt thereof, wherein:

R³ is hydrogen, C₁₂ alkyl, C₁₋₂ haloalkyl, CN, —C(O)OH, C(O)—O—(C₁-C₄ alkyl) or —S(O)₂—(C₁-C₄ alkyl);

Z¹ is O or CH₂;

Z³ is CHR¹;

each R¹ is independently H or methyl;

Y is selected from:

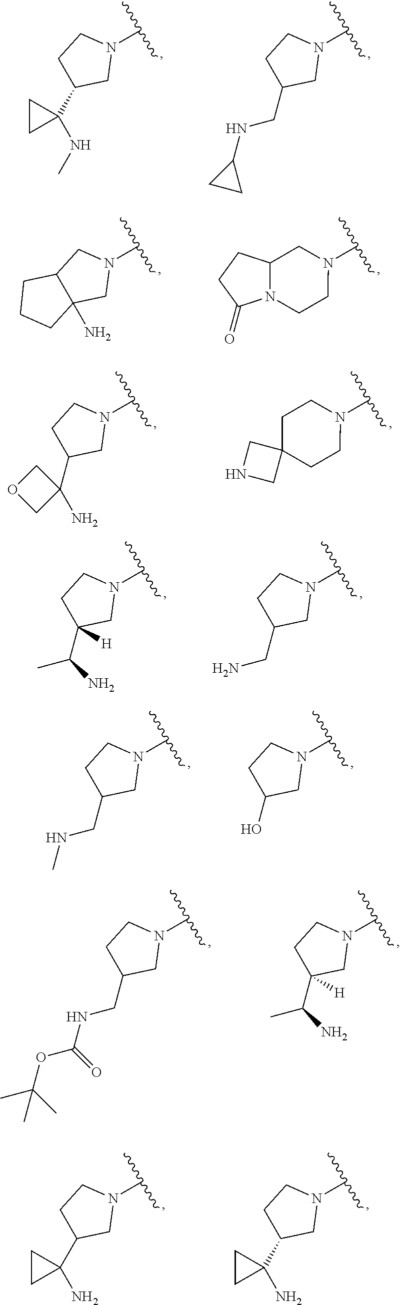

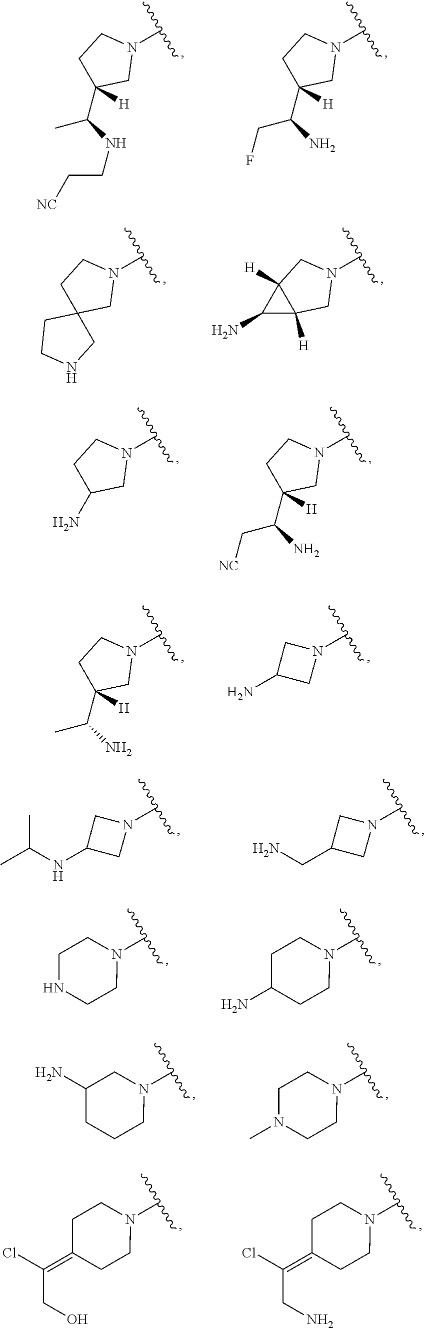

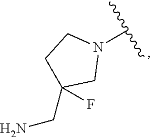 [SMILES: *N1CCC(F)(CN)C1]

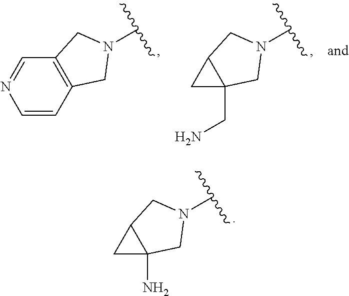 [SMILES: *N1Cc2ccncc2C1 ; *N1CC2CC2(CN)C1 ; *N1CC2CC2(N)C1]

15. The compound of embodiment 1, which is a compound of formula (VI):

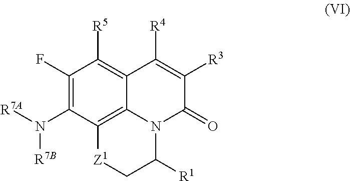

wherein,

R¹ is H, methyl, CH₂F, CH₂OH, or CH₂OMe;

R³ is hydrogen or —COOR²;

R² is H or C₁-C₄ alkyl;

R⁴ is hydrogen or —CH₂NH₂;

Z¹ is O or CH₂;

R⁵ is hydrogen, Me or halo; and

R^{7A} and R^{7B} together with the nitrogen atom to which they are attached form a 5- to 6-membered monocyclic heterocyclic group optionally including one additional heteroatom selected from N, O and S as a ring member, or a 6-10 membered bicyclic heterocyclic group optionally including one additional heteroatom selected from N, O and S as a ring member,

wherein the monocyclic or bicyclic heterocyclic group formed by R^{7A} and R^{7B} together with the nitrogen atom to which they are attached is optionally substituted by up to four groups selected from halogen, —CN, hydroxy, phenyl, oxo, —OR⁹, —N(R⁹)₂, —COOR⁹, —C(O)N(R⁹)₂, C₁-C₄ alkyl, C₁-C₄ haloalkyl, C₃-C₆ cycloalkyl, and a 4-6 membered heteroaryl or heterocyclyl group that contains up to two heteroatoms selected from N, O and S as ring members,

wherein the C₁-C₄ alkyl, C₃-C₆ cycloalkyl, phenyl, and 4-6 membered heteroaryl or heterocyclyl are each optionally substituted by up to three groups independently selected from halogen, —CN, hydroxy, oxo, —OR¹⁰, ═N—OR¹⁰, —N(R¹⁰)₂, —COOR¹⁰, —N(R¹⁰)-C(O)—O—(C₁-C₄ alkyl), —C(O)N(R¹⁰)₂, C₁-C₄ alkyl, C₁-C₄ haloalkyl, and C₁-C₄ alkoxy;

or a pharmaceutically acceptable salt thereof.

16. The compound of embodiment 15, wherein the group represented by —NR^{7A}R^{7B is} selected from:

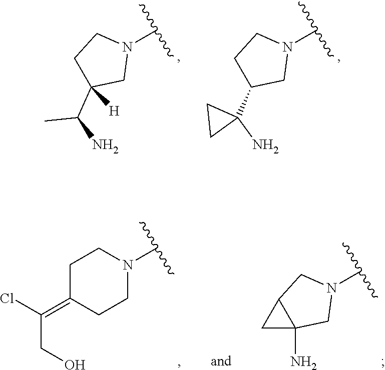

or a pharmaceutically acceptable salt thereof.

17. A pharmaceutical composition, comprising:

the compound according to any one of embodiments 1-16, and a pharmaceutically acceptable carrier, adjuvant or vehicle. In certain of these embodiments, the compound is selected from the Examples herein.

18. The pharmaceutical composition according to embodiments 7, further comprising an additional therapeutic agent with antibacterial activity.

19. A method for treating a subject having a bacterial infection, comprising:

administering to the subject in need thereof an antibacterially effective amount of the compound according to any one of embodiments 1-16.

20. The method of embodiment 19, wherein the bacterial infection is an infection comprising at least one bacterium selected from the group consisting of Pseudomonas aeruginosa and other Pseudomonas species, Stenotrophomonas maltophilia, Burkholderia cepacia and other Burkholderia species, Acinetobacter baumannii and other Acinetobacter species, Achromobacter xylosoxidans, Alcaligenes denitrificans and other Achromobacteraceae, Citrobacter freundii and other Citrobacter species, Campylobacter jejuni, Klebsiella pneumoniae, Klebsiella oxytoca and other Klebsiella species, Enterobacter cloacae, Enterobacter aerogenes and other Enterobacter species, Escherichia coli, Salmonella enterica and other Salmonella species, Yersinia pestis, Proteus vulgaris and other Proteus species, Serratia marscens and other Serratia species, Morganella morganii and other members of the Enterobacteriaceae family, Neisseria meningitidis, Haemophilus influenzae, Moraxella cattharallis, Bacteroides fragilis, Bacteroides thetaiotaomicron and other Bacteriodes species, Pasteurella multicoda and other Pasteurella species, Fransicella tularensis, Shigella dysenteriae and other Shigella species, Vibrio cholera and other Vibrio species, Bordetella pertussis and other Bordetella species, Helicobactor pylori and other Helicobacter species, Legionella pneumophila and Campylobactor jejuni, Staphylococcus aureus, Staphylococcus epidermidis and other Staphylococcus species, Enterococcus faecalis, Enterococcus faecium and other Enterococcus species, Streptococcus pneumoniae, Streptococcus pyogenes, Streptococcus agalactiae and other Streptococcus species, Bacillus anthracis and other Bacillus species, Peptostreptococcus magnus and other Peptostreptococcus species, Clostridium difficile and other Clostridium species, Listeria monocytogenes and other Listeria species, and Corynebacterium diptheriae and other Corynebacterium species.

The compounds as defined in the embodiments may be synthesized by the general synthetic routes below, specific examples of which are described in more detail in the Examples section. Reaction schemes in the Examples illustrate methods used to make selected compounds of the invention, and can be adapted for synthesis of additional compounds of the invention using standard methods and available starting materials. The following general methods can be used. The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

Within the scope of this text, a readily removable group that is not a constituent of the particular desired end product of the compounds of the present invention is designated a “protecting group”, a term that is well understood by those of skill in the art. A characteristic of protecting groups is that they can be removed readily (i.e. without the occurrence of undesired secondary reactions) for example by solvolysis, reduction, photolysis or alternatively under physiological conditions (e.g. by enzymatic cleavage). The protection of functional groups by such protecting groups, the protecting groups themselves, and their cleavage reactions are well known in the art and are described in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der Organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosauren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974.

Salts of compounds of the present invention having at least one salt-forming group may be prepared in a manner known to those skilled in the art. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralization of salts, such as acid addition salts, to the isoelectric point, e.g. with weak bases, or by treatment with ion exchangers.

Salts can be converted into the free compounds in accordance with methods known to those skilled in the art. Metal and ammonium salts can be converted, for example, by treatment with suitable acids, and acid addition salts, for example, by treatment with a suitable basic agent.

Mixtures of isomers obtainable according to the invention can be separated in a manner known to those skilled in the art into the individual isomers; diastereoisomers can be separated, for example, by partitioning between polyphasic solvent mixtures, recrystallization and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallization, or by chromatography over optically active column materials.

Intermediates and final products can be worked up and/or purified according to standard methods, e.g. using chromatographic methods, distribution methods, (re-) crystallization, and the like.

The following applies in general to all processes mentioned herein before and hereinafter.

All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds of the present invention, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

All starting materials, building blocks, reagents, acids, bases, dehydrating agents, solvents and catalysts utilized to synthesize the compounds of the present invention are either commercially available or can be produced by organic synthesis methods known to one of ordinary skill in the art (Houben-Weyl 4^{th} Ed. 1952, Methods of Organic Synthesis, Thieme, Volume 21).

The term “an optical isomer” or “a stereoisomer” refers to any of the various stereoisomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-lngold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

As used herein, the terms “salt” or “salts” refers to an acid addition or base addition salt of a compound of the present invention. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns Ito XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds of the present invention. Isotopically labeled compounds have structures depicted by the formulas given herein wherein one or more atoms of the structure is enriched in or represents an isotope having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds of the present invention, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound of the present invention may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagent in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present invention. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the present invention that contain groups capable of acting as donors and/or acceptors for hydrogen bonds may be capable of forming co-crystals with suitable co-crystal formers. These co-crystals may be prepared from compounds of the present invention by known co-crystal forming procedures. Such procedures include grinding, heating, co-subliming, co-melting, or contacting in solution compounds of the present invention with the co-crystal former under crystallization conditions and isolating co-crystals thereby formed. Suitable co-crystal formers include those described in WO 2004/078163. Hence the invention further provides co-crystals comprising a compound of the present invention.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures; and/or

e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules where the active ingredient is mixed with water or an oil medium, such as peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurized container, pump, spray, atomizer or nebulizer, with or without the use of a suitable propellant.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a product comprising a compound according to any one of Formulae (I) to (V), or a pharmaceutically acceptable salt thereof, and at least one other therapeutic agent as a combined preparation for simultaneous, separate or sequential use in therapy. In one embodiment, the therapy is the prevention or treatment of a disease or condition mediated by gyrase activity. Products provided as a combined preparation include a composition comprising the compound according to any one of Formulae (I) to (V), or a pharmaceutically acceptable salt thereof and the other therapeutic agent(s) together in the same pharmaceutical composition, or the compound according to any one of Formulae (I) to (V), or a pharmaceutically acceptable salt thereof and the other therapeutic agent(s) in separate form, e.g. in the form of a kit.

In one embodiment, the invention provides a pharmaceutical composition comprising a compound according to any one of Formulae (I) to (V), or a pharmaceutically acceptable salt thereof, and another therapeutic agent(s). Optionally, the pharmaceutical composition may comprise a pharmaceutically acceptable excipient, as described above.

In one embodiment, the invention provides a kit comprising two or more separate pharmaceutical compositions, at least one of which contains a compound according to anyone of Formulae (I) to (V), or a pharmaceutically acceptable salt thereof. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Accordingly, the invention provides the use of a compound of formulae (I) to (V) for preventing and/or treating a disease or condition mediated by gyrase activity, wherein the medicament is prepared for administration with another therapeutic agent. The invention also provides the use of another therapeutic agent for preventing and/or treating a disease or condition mediated by gyrase activity, wherein the medicament is administered with a compound according to any one of Formulae (I) to (V), or a pharmaceutically acceptable salt thereof.

The invention also provides a compound of formulae (I) to (V) for use in a method of prevention and/or treating a disease or condition mediated by gyrase activity, wherein the compound according to any one of Formulae (I) to (V), or a pharmaceutically acceptable salt thereof, is prepared for administration with another therapeutic agent. The invention also provides another therapeutic agent for use in a method of preventing and/or treating a disease or condition mediated by gyrase activity wherein the other therapeutic agent is prepared for administration with a compound according to any one of Formulae (I) to (V), or a pharmaceutically acceptable salt thereof. The invention also provides a compound according to any one of Formulae (I) to (V), or a pharmaceutically acceptable salt thereof, for use in a method of preventing and/or treating a disease or condition mediated by gyrase activity wherein the compound according to any one of Formulae (I) to (V), or a pharmaceutically acceptable salt thereof, is administered with another therapeutic agent. The invention also provides another therapeutic agent for use in a method of preventing and/or treating a disease or condition mediated by gyrase activity wherein the other therapeutic agent is administered with a compound according to any one of Formulae (I) to (V), or a pharmaceutically acceptable salt thereof.

The compounds and compositions described herein can be used or administered in combination with one or more therapeutic agents that act as immunomodulators, e.g., an activator of a costimulatory molecule, or an inhibitor of an immune-inhibitory molecule, or a vaccine. The Programmed Death 1 (PD-1) protein is an inhibitory member of the extended CD28/CTLA4 family of T cell regulators (Okazaki et al. (2002) Curr Opin Immunol 14: 391779-82; Bennett et al. (2003) J. Immunol. 170:711-8). PD-1 is expressed on activated B cells, T cells, and monocytes. PD-1 is an immune-inhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745), and is up-regulated in chronic infections. The interaction between PD-1 and PD-L1 can act as an immune checkpoint, which can lead to, e.g., a decrease in infiltrating lymphocytes, a decrease in T-cell receptor mediated proliferation, and/or immune evasion by cancerous or infected cells (Dong et al. (2003) J Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J Immunol. 170:1257-66). Immunomodulation can be achieved by binding to either the immune-inhibitory protein (e.g., PD-1) or to binding proteins that modulate the inhibitory protein (e.g., PD-L1, PD-L2).

In one embodiment, the combination therapies of the invention include an immunomodulator that is an inhibitor or antagonist of an inhibitory molecule of an immune checkpoint molecule. In another embodiment the immunomodulator binds to a protein that naturally inhibits the immuno-inhibitory checkpoint molecule. When used in combination with antibacterial compounds, these immunomodulators can enhance the antimicrobial response, and thus enhance efficacy relative to treatment with the antibacterial compound alone.

The term “immune checkpoints” refers to a group of molecules on the cell surface of CD4 and CD8 T cells. These molecules can effectively serve as “brakes” to down-modulate or inhibit an adaptive immune response. Immune checkpoint molecules include, but are not limited to, Programmed Death 1 (PD-1), Cytotoxic T-Lymphocyte Antigen 4 (CTLA-4), B7H1, B7H4, OX-40, CD137, CD40, and LAG3, which directly inhibit immune cells. Immunotherapeutic agents which can act as immune checkpoint inhibitors useful in the methods of the present invention, include, but are not limited to, inhibitors of PD-L1, PD-L2, CTLA4, TIM3, LAG3, VISTA, BTLA, TIGIT, LAIR1, CD160, 2B4 and/or TGFR beta. Inhibition of an inhibitory molecule can be performed by inhibition at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is a polypeptide, e.g., a soluble ligand, or an antibody or antigen-binding fragment thereof, that binds to the inhibitory molecule.

By “in combination with,” it is not intended to imply that the therapy or the therapeutic agents must be administered at the same time and/or formulated for delivery together, although these methods of delivery are within the scope described herein. The immunomodulator can be administered concurrently with, prior to, or subsequent to, one or more compounds of the invention, and optionally one or more additional therapies or therapeutic agents. The therapeutic agents in the combination can be administered in any order. In some embodiments, the levels utilized in combination will be lower than those utilized individually.

In certain embodiments, the antibacterial compounds described herein, e.g., compounds of Formulas (I)-(V) as described herein including those of embodiments 1-17, are administered in combination with one or more immunomodulators that are inhibitors of PD-1, PD-L1 and/or PD-L2. Each such inhibitor may be an antibody, an antigen binding fragment thereof, an immunoadhesin, a fusion protein, or an oligopeptide. Examples of such immunomodulators are known in the art.

In some embodiments, the immunomodulator is an anti-PD-1 antibody chosen from MDX-1106, Merck 3475 or CT-011.

In some embodiments, the immunomodulator is an immunoadhesin (e.g., an immunoadhesin comprising an extracellular or PD-1 binding portion of PD-L1 or PD-L2 fused to a constant region (e.g., an Fc region of an immunoglobulin sequence).

In some embodiments, the immunomodulator is a PD-1 inhibitor such as AMP-224.

In some embodiments, the immunomodulator is a PD-L1 inhibitor such as anti-PD-L1 antibody.

In some embodiments, the immunomodulator is an anti-PD-L1 binding antagonist chosen from YW243.55.S70, MPDL3280A, MEDI-4736, MSB-0010718C, or MDX-1105. MDX-1105, also known as BMS-936559, is an anti-PD-L1 antibody described in WO2007/005874. Antibody YW243.55.S70 is an anti-PD-L1 described in WO 2010/077634.

In some embodiments, the immunomodulator is nivolumab (CAS Registry Number: 946414-94-4). Alternative names for nivolumab include MDX-1106, MDX-1106-04, ONO-4538, or BMS-936558. Nivolumab is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449, EP2161336 and WO2006/121168.

In some embodiments, the immunomodulator is an anti-PD-1 antibody Pembrolizumab. Pembrolizumab (also referred to as Lambrolizumab, MK-3475, MK03475, SCH-900475 or KEYTRUDA®; Merck) is a humanized IgG4 monoclonal antibody that binds to PD-1. Pembrolizumab and other humanized anti-PD-1 antibodies are disclosed in Hamid, 0. et al. (2013) New England Journal of Medicine 369 (2): 134-44, U.S. Pat. No. 8,354,509, WO2009/114335, and WO2013/079174.

In some embodiments, the immunomodulator is Pidilizumab (CT-011; Cure Tech), a humanized IgGlk monoclonal antibody that binds to PD1. Pidilizumab and other humanized anti-PD-1 monoclonal antibodies are disclosed in WO2009/101611.

Other anti-PD1 antibodies useful as immunomodulators for use in the methods disclosed herein include AMP 514 (Amplimmune), and anti-PD1 antibodies disclosed in U.S. Pat. No. 8,609,089, US 2010028330, and/or US 20120114649. In some embodiments, the anti-PD-L1 antibody is MSB0010718C. MSB0010718C (also referred to as A09-246-2; Merck Serono) is a monoclonal antibody that binds to PD-L1.

In some embodiments, the immunomodulator is MDPL3280A (Genentech/Roche), a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. Other anti-PD-L1 binding agents useful as immunomodulators for methods of the invention include YW243.55.S70 (see WO2010/077634), MDX-1105 (also referred to as BMS-936559), and anti-PD-L1 binding agents disclosed in WO2007/005874.

In some embodiments, the immunomodulator is AMP-224 (B7-DCIg; Amplimmune; e.g., disclosed in WO2010/027827 and WO2011/066342), is a PD-L2 Fc fusion soluble receptor that blocks the interaction between PD1 and B7-H1.

In some embodiments, the immunomodulator is an anti-LAG-3 antibody such as BMS-986016. BMS-986016 (also referred to as BMS986016) is a monoclonal antibody that binds to LAG-3. BMS-986016 and other humanized anti-LAG-3 antibodies are disclosed in US 2011/0150892, WO2010/019570, and WO2014/008218

In certain embodiments, the combination therapies disclosed herein include a modulator of a costimulatory molecule or an inhibitory molecule, e.g., a co-inhibitory ligand or receptor. In one embodiment, the costimulatory modulator, e.g., agonist, of a costimulatory molecule is chosen from an agonist (e.g., an agonistic antibody or antigen-binding fragment thereof, or soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

In another embodiment, the combination therapies disclosed herein include an immunomodulator that is a costimulatory molecule, e.g., an agonist associated with a positive signal that includes a costimulatory domain of CD28, CD27, ICOS and/or GITR.

Exemplary GITR agonists include, e.g., GITR fusion proteins and anti-GITR antibodies (e.g., bivalent anti-GITR antibodies), such as, a GITR fusion protein described in U.S. Pat. No. 6,111,090, European Patent No.: 090505B1, U.S Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. No. 7,812,135, U.S. Pat. No. 8,388,967, U.S. Pat. No. 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, the immunomodulator used is a soluble ligand (e.g., a CTLA-4-Ig), or an antibody or antibody fragment that binds to PD-L1, PD-L2 or CTLA4. For example, the anti-PD-1 antibody molecule can be administered in combination with an anti-CTLA-4 antibody, e.g., ipilimumab, for example. Exemplary anti-CTLA4 antibodies include Tremelimumab (IgG2 monoclonal antibody available from Pfizer, formerly known as ticilimumab, CP-675,206); and Ipilimumab (CTLA-4 antibody, also known as MDX-010, CAS No. 477202-00-9).

In one embodiment, an anti-PD-1 antibody molecule is administered after treatment with a compound of the invention as described herein.

In another embodiment, an anti-PD-1 or PD-L1 antibody molecule is administered in combination with an anti-LAG-3 antibody or an antigen-binding fragment thereof. In another embodiment, the anti-PD-1 or PD-L1 antibody molecule is administered in combination with an anti-TIM-3 antibody or antigen-binding fragment thereof. In yet other embodiments, the anti-PD-1 or PD-L1 antibody molecule is administered in combination with an anti-LAG-3 antibody and an anti-TIM-3 antibody, or antigen-binding fragments thereof. The combination of antibodies recited herein can be administered separately, e.g., as separate antibodies, or linked, e.g., as a bispecific or trispecific antibody molecule. In one embodiment, a bispecific antibody that includes an anti-PD-1 or PD-L1 antibody molecule and an anti-TIM-3 or anti-LAG-3 antibody, or antigen-binding fragment thereof, is administered. In certain embodiments, the combination of antibodies recited herein is used to treat a cancer as described herein (e.g., a solid tumor). The efficacy of the aforesaid combinations can be tested in animal models known in the art. For example, the animal models to test the synergistic effect of anti-PD-1 and anti-LAG-3 are described, e.g., in Woo et al. (2012) Cancer Res. 72(4):917-27).

Exemplary immunomodulators that can be used in the combination therapies include, but are not limited to, e.g., afutuzumab (available from Roche®); pegfilgrastim (Neulasta®); lenalidomide (CC-5013, Revlimid®); thalidomide (Thalomid®), actimid (CC4047); and cytokines, e.g., IL-21 or IRX-2 (mixture of human cytokines including interleukin 1, interleukin 2, and interferon y, CAS 951209-71-5, available from IRX Therapeutics).

Exemplary doses of such immunomodulators that can be used in combination with the antibacterial compounds of the invention include a dose of anti-PD-1 antibody molecule of about 1 to 10 mg/kg, e.g., 3 mg/kg, and a dose of an anti-CTLA-4 antibody, e.g., ipilimumab, of about 3 mg/kg.

Examples of embodiments of the methods of using the antibacterial compounds of the invention in combination with an immunomodulator include these:

i. A method to treat a bacterial infection in a subject, comprising administering to the subject a compound of Formula (I) including any of embodiments 1-17 as described herein, and an immunomodulator.

ii. The method of embodiment i, wherein the immunomodulator is an activator of a costimulatory molecule or an inhibitor of an immune checkpoint molecule.

iii. The method of either of embodiments i and ii, wherein the activator of the costimulatory molecule is an agonist of one or more of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 and CD83 ligand.

iv. The method of any of embodiments i-iii above, wherein the inhibitor of the immune checkpoint molecule is chosen from PD-1, PD-L1, PD-L2, CTLA4, TIM3, LAG3, VISTA, BTLA, TIGIT, LAIR1, CD160, 2B4 and TGFR beta.

v. The method of any of any of embodiments i-iii, wherein the inhibitor of the immune checkpoint molecule is chosen from an inhibitor of PD-1, PD-L1, LAG-3, TIM-3 or CTLA4, or any combination thereof.

vi. The method of any of embodiments i-v, wherein the inhibitor of the immune checkpoint molecule is a soluble ligand or an antibody or antigen-binding fragment thereof, that binds to the immune checkpoint molecule.

vii. The method of any of embodiments i-vi, wherein the antibody or antigen-binding fragment thereof is from an IgG1 or IgG4 (e.g., human IgG1 or IgG4).

viii. The method of any of embodiments i-vii, wherein the antibody or antigen-binding fragment thereof is altered, e.g., mutated, to increase or decrease one or more of: Fc receptor binding, antibody glycosylation, the number of cysteine residues, effector cell function, or complement function.

ix. The method of any of embodiments i-viii, wherein the antibody molecule is a bispecific or multispecific antibody molecule that has a first binding specificity to PD-1 or PD-L1 and a second binding specifity to TIM-3, LAG-3, or PD-L2.

x. The method of any of embodiments i-ix, wherein the immunomodulator is an anti-PD-1 antibody chosen from Nivolumab, Pembrolizumab or Pidilizumab.

xi. The method of any of embodiments i-x, wherein the immunomodulator is an anti-PD-L1 antibody chosen from YW243.55.570, MPDL3280A, MEDI-4736, MSB-0010718C, or MDX-1105.

xii. The method of any of embodiments i-x, wherein the immunomodulator is an anti-LAG-3 antibody molecule.

xiii. The method of embodiment xii, wherein the anti-LAG-3 antibody molecule is BMS-986016.

xiv. The method of any of embodiments i-x, wherein the immunomodulator is an anti-PD-1 antibody molecule administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg., e.g., once a week to once every 2, 3, or 4 weeks.

xv. The method of embodiment xiv, wherein the anti-PD-1 antibody molecule is administered at a dose from about 10 to 20 mg/kg every other week.

xvi. The method of embodiment xv, wherein the anti-PD-1 antibody molecule, e.g., nivolumab, is administered intravenously at a dose from about 1 mg/kg to 3 mg/kg, e.g., about 1 mg/kg, 2 mg/kg or 3 mg/kg, every two weeks.

xvii. The method of embodiment xv, wherein the anti-PD-1 antibody molecule, e.g., nivolumab, is administered intravenously at a dose of about 2 mg/kg at 3-week intervals.

### Methods of Use

The compounds according to the any of Formulae I to V in free form or in pharmaceutically acceptable salt forms, exhibit valuable pharmacological properties including inhibiting DNA gyrase activity in bacteria as well as acting as antibacterials.

According to one embodiment, the present invention provides a method of inhibiting bacterial DNA gyrase activity in a subject, administering to said subject a compound of formulae I-V or a composition comprising a compound of formula I-V and a pharmaceutically acceptable carrier, adjuvant, or vehicle.

According to another embodiment, the present invention provides a method of decreasing bacterial quantity in a subject, comprising administering to said

subject a compound of formula I-V or a composition comprising a compound of formula I-V and a pharmaceutically acceptable carrier, adjuvant, or vehicle.

According to another embodiment, the present invention provides a method of preventing, treating, or lessening the severity of a bacterial infection in a subject, comprising administering to said subject a compound of formula I-V or a composition comprising a compound of formula I-VI and a pharmaceutically acceptable carrier, adjuvant, or vehicle.

According to another embodiment, the methods of the present invention are useful to treat patients in the veterinary field including, but not limited to, zoo, laboratory, and farm animals, including primates, rodents, and birds. Examples of said animals include, but are not limited to guinea pigs, hamsters, gerbils, rat, mice, rabbits, dogs, cats, horses, pigs, sheep, cows, goats, deer, rhesus monkeys, monkeys, tamarinds, apes, baboons, gorillas, chimpanzees, orangutans, gibbons, ostriches, chickens, turkeys, ducks, and geese.

In another embodiment, the present invention provides a method wherein the bacterial infection to be treated or prevented is characterized by the presence of one or more fermentative or non-fermentative Gram-negative bacteria selected from the group consisting of Pseudomonas aeruginosa and other Pseudomonas species, Stenotrophomonas maltophilia, Burkholderia cepacia and other Burkholderia species, Acinetobacter baumannii and other Acinetobacter species, Achromobacter xylosoxidans, Alcaligenes denitrificans and other Achromobacteraceae, Citrobacter freundii and other Citrobacter species, Campylobacter jejuni, Klebsiella pneumoniae, Klebsiella oxytoca and other Klebsiella species, Enterobacter cloacae, Enterobacter aerogenes and other Enterobacter species, Escherichia coli, Salmonella enterica and other Salmonella species, Yersinia pestis, Proteus vulgaris and other Proteus species, Serratia marscens and other Serratia species, Morganella morganii and other members of the Enterobacteriaceae family, Neisseria meningitidis, Haemophilus influenzae, Moraxella cattharallis, Bacteroides fragilis, Bacteroides thetaiotaomicron and other Bacteriodes species, Pasteurella multicoda and other Pasteurella species, Fransicella tularensis, Shigella dysenteriae and other Shigella species, Vibrio cholera and other Vibrio species, Bordetella pertussis and other Bordetella species, Helicobactor pylori and other Helicobacter species, Legionella pneumophila and Campylobactor jejuni.

In another embodiment, the present invention provides a method wherein the bacterial infection to be treated or prevented is characterized by the presence of one or more fermentative or non-fermentative Gram-positive bacteria selected from the group consisting of Staphylococcus aureus, Staphylococcus epidermidis and other Staphylococcus species, Enterococcus faecalis, Enterococcus faecium and other Enterococcus species, Streptococcus pneumoniae, Streptococcus pyogenes, Streptococcus agalactiae and other Streptococcus species, Bacillus anthracis and other Bacillus species, Peptostreptococcus magnus and other Peptostreptococcus species, Clostridium difficile and other Clostridium species, Listeria monocytogenes and other Listeria species, Corynebacterium diptheriae and other Corynebacterium species.

According to another embodiment, the present invention comprises administering to the subject one or more additional therapeutic antibacterial agents other than a compound of the present invention.

According to another embodiment, the invention comprises administering to said subject one or more additional therapeutic agents either as part of a multiple dosage form together with said compound or as a separate dosage form, wherein said one or more additional therapeutic agents include an antibiotic selected from a natural penicillin, a penicillinase-resistant penicillin, an antipseudomonal penicillin, an aminopenicillin, a first generation cephalosporin, a second generation cephalosporin, a third generation cephalosporin, a fourth generation cephalosporin, a carbapenem, a cephamycin, a monobactam, a quinolone, a fluoroquinolone, an aminoglycoside, a macrolide, a ketolide, a tetracycline, a glycopeptide, a streptogramin, an oxazolidinone, a rifamycin, or other antibiotics. The subject for these methods may be a human.

According to another embodiment, the invention comprises administering to said subject one or more additional therapeutic agents either as part of a multiple dosage form together with said compound or as a separate dosage form wherein said one or more additional therapeutic agents are selected from a natural penicillin including Benzathine penicillin G, Penicillin G and Penicillin V, from a penicillinase-resistant penicillin including Cloxacillin, Dicloxacillin, Nafcillin and Oxacillin, from a antipseudomonal penicillin including Carbenicillin, Mezlocillin, Piperacillin, Piperacillin/tazobactam, Ticaricillin and Ticaricillin/Clavulanate, from an aminopenicillin including Amoxicillin, Ampicillin and Ampicillin/Sulbactam, from a first generation cephalosporin including Cefazolin, Cefadroxil, Cephalexin and Cephadrine, from a second generation cephalosporin including Cefaclor, Cefaclor-CD, Cefamandole, Cefonacid, Cefprozil, Loracarbef and Cefuroxime, from a third generation cephalosporin including Cefdinir, Cefixime, Cefoperazone, Cefotaxime, Cefpodoxime, Ceftazidime, Ceftibuten, Ceftizoxme and Ceftriaxone, from a fourth generation cephalosporin including Cefepime, Ceftaroline and Ceftolozane, from a Cephamycin including Cefotetan and Cefoxitin, from a carbapenem including Imipenem, Doripenem and Meropenem, from a monobactam including Aztreonam and Carumonam, from a quinolone including Cinoxacin, Nalidixic acid, Oxolinincacid and Pipemidic acid, from a fluoroquinolone including Cirpofloxacin, Enoxacin, Gatifloxacin, Grepafloxacin, Levofloxacin, Lomefloxacin, Moxifloxacin, Norfloxacin, Ofloxacin and Sparfloxacin, from an aminoglycoside including Amikacin, Gentamicin, Kanamycin, Neomycin, Netilmicin, Spectinomycin, Streptomycin, Plazomycin and Tobramycin, from a macrolide including Azithromycin, Clarithromycin and Erythromycin, from a ketolide including Telithromycin, from a Tetracycline including Chlortetracycline, Demeclocycline, Doxycycline, Minocycline, Tigecycline and Tetracycline, from a glycopeptide including Oritavancin, Teicoplanin, Dalbavancin, Telavancin and Vancomycin, from a streptogramin including Dalfopristin/quinupristin, from an oxazolidone including Linezolid, from a Rifamycin including Rifabutin and Rifampin and from other antibiotics including bactitracin, chloramphenicol, clindamycin, isoniazid, metronidazole, polymyxin B, pyrazinamide, and trimethoprim/sulfamethoxazole.

According to another embodiment, the present invention provides a method of preventing, treating, or lessening the severity of a bacterial infection in a subject wherein the bacterial infection to be treated or prevented is selected from one or more of the following: upper respiratory infections, lower respiratory infections, ear infections, pleuropulmonary and bronchial infections, urinary tract infections, intra-abdominal infections, complicated urinary tract infections, complicated intra-abdominal infections, cardiovascular infections, a blood stream infection, sepsis, CNS infections, skin and soft tissue infections, GI infections, bone and joint infections, genital infections, eye infections, or granulomatous infections.

In another embodiment, the bacterial infection to be treated is selected from one or more of the following: pharyngitis, sinusitis, otitis externa, otitis media, bronchitis, empyema, pneumonia, cystitis and pyelonephritis, renal calculi, prostatitis, peritonitis, dialysis-associated peritonitis, visceral abscesses, endocarditis, myocarditis, pericarditis, transfusion-associated sepsis, meningitis, encephalitis, brain abscess, osteomyelitis, arthritis, genital ulcers, urethritis, vaginitis, cervicitis, gingivitis, conjunctivitis, keratitis, endophthalmitisa, or an infection of febrile neutropenic subjects.

According to another embodiment, the invention provides a method for treating or preventing a susceptible bacterial organism in a subject wherein said method further comprises the step of administering to said patient an additional therapeutic agent either as part of a multiple dosage form together with said compound or as a separate dosage form.

According to another embodiment, the invention provides a method for treating or preventing a susceptible bacterial organism in a subject wherein said method further comprises the step of administering to said subject an agent that increases the susceptibility of bacterial organisms to antibiotics.

According to another embodiment of the present invention, the methods further comprise the step of administering to a subject one or more additional therapeutic agents that increase the susceptibility of the bacterial organisms to antibiotics. For example, where a compound of the invention is administered with a beta-lactam such as a monobactam, penicillin, carbapenem, cephamycin or cephalosporin.

According to another embodiment of the present invention, the methods further comprise the step of administering to a subject one or more additional therapeutic agents that increase the susceptibility of bacterial organisms to antibiotics including a biofilm inhibitor.

In another embodiment, the pharmaceutical compositions and methods of this invention will be useful generally for controlling bacterial infections in vivo caused by the following organisms: Pseudomonas aeruginosa and other Pseudomonas species, Stenotrophomonas maltophilia, Burkholderia cepacia and other Burkholderia species, Acinetobacter baumannii and other Acinetobacter species, Achromobacter xylosoxidans, Alcaligenes denitrificans and other Achromobacteraceae, Citrobacter freundii and other Citrobacter species, Campylobacter jejuni, Klebsiella pneumoniae, Klebsiella oxytoca and other Klebsiella species, Enterobacter cloacae, Enterobacter aerogenes and other Enterobacter species, Escherichia coli, Salmonella enterica and other Salmonella species, Yersinia pestis, Proteus vulgaris and other Proteus species, Serratia marscens and other Serratia species, Morganella morganii and other members of the Enterobacteriaceae family, Neisseria meningitidis, Haemophilus influenzae, Moraxella cattharallis, Bacteroides fragilis, Bacteroides thetaiotaomicron and other Bacteriodes species, Pasteurella multicoda and other Pasteurella species, Fransicella tularensis, Shigella dysenteriae and other Shigella species, Vibrio cholera and other Vibrio species, Bordetella pertussis and other Bordetella species, Helicobactor pylori and other Helicobacter species, Legionella pneumophila, Campylobactor jejuni, Staphylococcus aureus, Staphylococcus epidermidis and other Staphylococcus species, Enterococcus faecalis, Enterococcus faecium and other Enterococcus species, Streptococcus pneumoniae, Streptococcus pyogenes, Streptococcus agalactiae and other Streptococcus species, Bacillus anthracis and other Bacillus species, Peptostreptococcus magnus and other Peptostreptococcus species, Clostridium difficile and other Clostridium species, Listeria monocytogenes and other Listeria species, Corynebacterium diptheriae and other Corynebacterium species.

In another embodiment, the pharmaceutical compositions and methods of this invention will be useful generally for controlling bacterial infections in vivo caused by the following organisms: Streptococcus pneumoniae, Streptococcus pyogenes, Enterococcus faecalis, Enterococcus faecium, Staphylococcus aureus, Bacillus anthracis, Mycobacterium tuberculosis, and coagulase-negative staphylococci such as Staphylococcus epidermidis and Staphylococcus saprophyticus.

The compositions and methods will therefore be useful for controlling, treating or reducing the advancement, severity or effects of nosocomial or non-nosocomial infections.

Examples of nosocomial and non-nosocomial infections include but are not limited to upper respiratory infections, lower respiratory infections, ear infections, pleuropulmonary and bronchial infections, urinary tract infections, intra-abdominal infections, cardiovascular infections, cardiovascular infections, blood stream infection, sepsis, CNS infections, skin and soft tissue infections, GI infections, bone and joint infections, genital infections, or granulomatous infections. Examples of specific bacterial infections include but are not limited to pharyngitis, sinusitis, otitis externa, otitis media, bronchitis, empyema, pneumonia, cystitis and pyelonephritis, renal calculi, prostatitis, peritonitis, dialysis-associated peritonitis, visceral abscesses, endocarditis, myocarditis, pericarditis, transfusion-associated sepsis, meningitis, encephalitis, brain abscess, osteomyelitis, arthritis, genital ulcers, urethritis, vaginitis, cervicitis, gingivitis, conjunctivitis, keratitis, endophthalmitisa, or an infection of febrile neutropenic subjects.

Most preferably, the pharmaceutically acceptable compositions of this invention are formulated for oral administration.

Dosage levels of between about 0.01 and about 100mg/kg body weight per day, preferably between 0.5 and about 75 mg/kg body weight per day and most preferably between about 1 and 50 mg/kg body weight per day of the active ingredient compound are useful in a monotherapy for the prevention and treatment of bacterial infections caused by bacteria such as Streptococcus pneumoniae, Streptococcus pyogenes, Enterococcus faecalis, Enterococcus faecium, Klebsiella pneumoniae, Enterobacter sps., Proteus sps., Pseudomonas aeruginosa, E. coli, Serratia marcesens, Staphylococcus aureus, Haemophilus inftuenzae, Bacillus anthracis, Mycoplasma pneumoniae, Moraxella catarrhalis, Chlamydia pneumoniae, Legionella pneumophila, Mycobacterium tuberculosis, Helicobacter pylori and coagulase-negative staphylococci such as Staphylococcus epidermidis.

Dosage levels of between about 0.01 and about 100mg/kg body weight per day, preferably between 0.5 and about 75 mg/kg body weight per day and most preferably between about 1 and 50 mg/kg body weight per day of the active ingredient compound are useful in a monotherapy for the prevention and treatment of resistant bacterial infections caused by bacteria such as methicillin-resistant Staphylococcus aureus, fluoroquinolone resistant Staphylococcus aureus, vancomycin intermediate resistant Staphylococcus aureus, linezolid-resistant Staphylococcus aureus, penicillin-resistant Streptococcus pneumoniae, macrolide-resistant Streptococcus pneumoniae, fluoroquinolone-resistant Streptococcus pneumoniae, vancomycin-resistant Enterococcus faecalis, linezolid-resistant Enterococcus faecalis, fluoroquinolone-resistant Enterococcus faecalis, vancomycin-resistant Enterococcus faecium, linezolid-resistant Enterococcus faecium, fluoroquinolone-resistant Enterococcus faecium, ampicillin-resistant Enterococcus faecium, macrolide-resistant Haemophilus influenzae, β-Iactam-resistant Haemophilus influenzae, fluoroquinolone-resistant Haemophilus inftuenzae, β-Iactam-resistant Moraxella catarrhalis, methicillin-resistant Staphylococcus epidermidis, fluoroquinolone-resistant Staphylococcus epidermidis, macrolide-resistant Mycoplasma pneumoniae, isoniazid-resistant Mycobacterium tuberculosis, rifampin-resistant Mycobacterium tuberculosis, methicillin-resistant coagulase-negative staphylcocci, fluoroquinolone-resistant coagulase-negative staphylcocci, glycopeptide-intermediate resistant Staphylococcus aureus, vancomycin-resistant Staphylococcus aureus, hetero vancomycin-intermediate resistant Staphylococcus aureus, hetero vancomycin-resistant Staphylococcus aureus, macrolide-lincosamide-streptogramin-resistant staphylococci, β-Iactam-resistant Enterococcus faecalis, β-Iactam-resistant Enterococcus faecium, ketolide-resistant Streptococcus pneumoniae, ketolide-resistant Streptococcus pyogenes, macrolide-resistant Streptococcus pyogenes, or vancomycin-resistant Staphylococcus epidermidis.

The pharmaceutical compositions of this invention will be administered from about 1 to 5 times per day or alternatively, as a continuous infusion. Or, alternatively, the compositions of the present invention may be administered in a pulsatile method. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

In an embodiment that comprises a combination of a compound of formula I-VI and one or more additional therapeutic or prophylactic agents, both the compound and the additional agent should be present at dosage levels of between about 10% to 80% of the dosage normally administered in a monotherapy regime.

The skilled artisan will appreciate, lower or higher doses than those recited above may be required. Specific dosage and treatment regimens for any particular subject will depend upon the judgment of the treating physician.

The compounds as defined in the embodiments may be synthesized by the general synthetic routes shown below, with specific examples described in more detail in the examples section. Reaction schemes in the examples section illustrate methods used to make selected compounds of the invention, and can be adapted for synthesis of additional compounds of the invention using standard methods and available starting materials. The following general methods can be used.

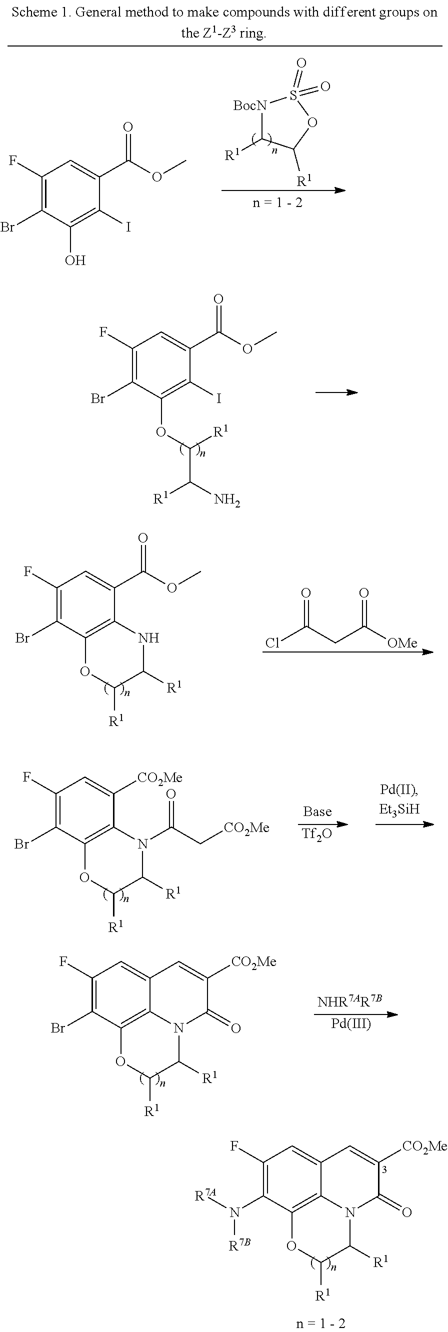

A variety of rings of different size and substituents can be introduced at the Z¹, Z² and Z³ positions via the route shown in Scheme 1. In addition, the ester group at the C3 position can be modified by conventional methods to introduce a variety of substituents at that position. The ester can also be hydrolyzed and removed by decarboxylation. The bromide allows easy introduction of amines at C7 through metal-catalyzed coupling reaction.

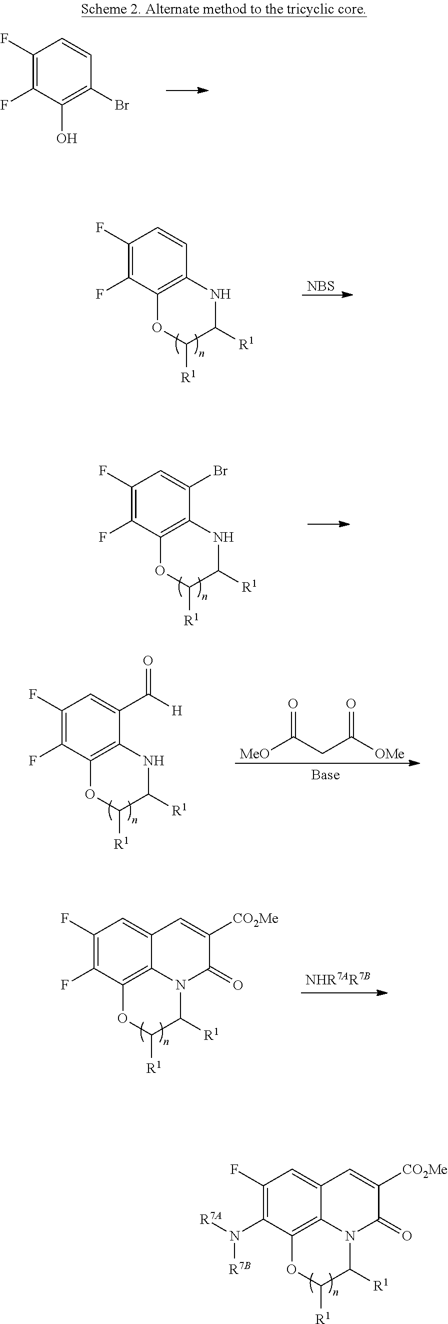

A second, alternate approach relies on the condensation of malonate with aldehyde to form the tricyclic core. This approach avoids the need to adjust the oxidation state at C4 after the condensation. Amines at C7 could be introduced through a S_{N}Ar on the aryl fluoride, complementing the coupling approach used in scheme 1.

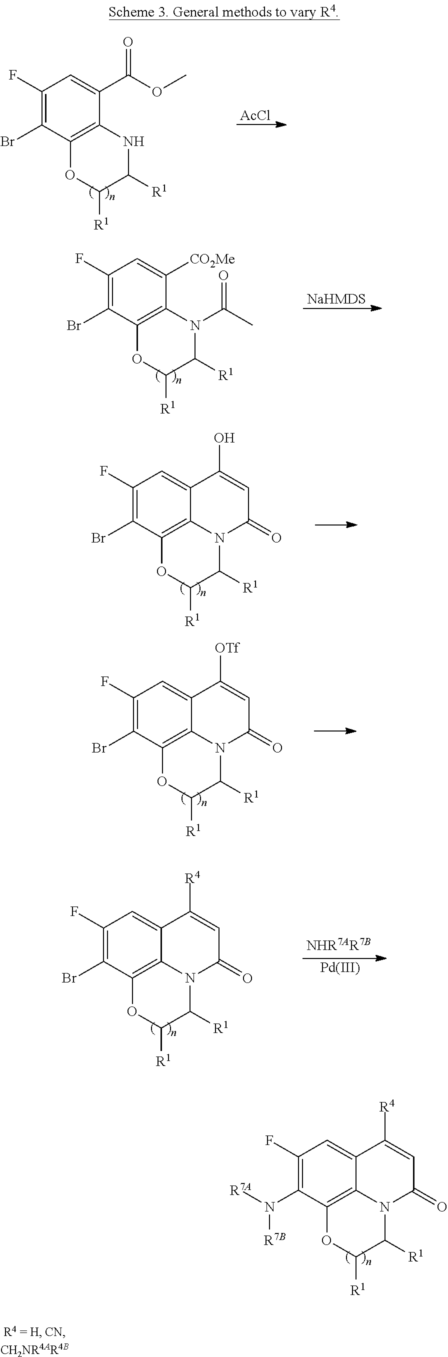

Using this method, a triflate can be introduced at the C4-position and be used as an handle to introduce various groups by metal-catalyzed coupling reactions. The functional groups introduced at the C4 position can then be further modified by known methods—examples of such modifications are included in the examples below.

### EXAMPLES

### General Conditions:

The analytical HPLC conditions are as follows:

(Method A) The compounds and/or intermediates were characterized by high performance liquid chromatography (HPLC) using a UPLC Waters instrument (Milford, Mass.). HPLC solvent A was 100% Water with 0.1% trifluoroacetic acid (TFA) and solvent B was 100% acetonitrile with 0.1% TFA from EMD Chemicals Inc. The instrument was a Waters ACQUITY UPLC system with 1.2 mL/min flow rate; column Kinetex-C18, 2.6 um, 2.1×50 mm from Phenomenex, column temperature: 50° C.; gradient: 2-88% solvent B over 1.29 min or 9.79 min period; compounds were detected by ultraviolet light (UV) absorption at either 220 or 254 nm.

(Method B) The compounds and/or intermediates were characterized by high performance liquid chromatography (HPLC) on a Waters ACQUITY H class UPLC system with 0.55 mL/min flow rate; column BEH-C18, 1.7 um, 2.1×50 mm from Waters, column temperature: ambient; gradient (solvent A is 2 mM Ammonium Acetate and 0.1% Formic Acid in Water, solvent B is 0.1% Formic Acid in acetonitrile): 5% solvent B hold for 0.4 min, 5-40% solvent B over 0.6 min, 40-60% solvent B over 1.2 min, 60-100% solvent B over 2.3 min then 100% solvent B over 3 min; compounds were detected by ultraviolet light (UV) absorption at 236 nm.

(Method C) The compounds and/or intermediates were characterized by high performance liquid chromatography (HPLC) on a Agilent 1290 infinity RRLC system with 1 mL/min flow rate; column ZORBAX SB C8, 5 um, 250×4.6 mm from Agilent, column temperature: ambient; gradient (solvent A is 0.1% Formic Acid in Water, solvent B is 0.1% Formic Acid in acetonitrile): 10-30% solvent B over 25 min, 30-100% solvent B over 5 min, then 100% solvent B over 5 min; compounds were detected by ultraviolet light (UV) absorption at 238 nm.

HPLC/Mass spectrometric analysis (LC/MS) was performed on Waters ACQUITY UPLC system and equipped with a ZQ 2000 or SQD MS system; Column: Kinetex by Phenomenex, 2.6 um, 2.1×50mm, column temperature: 50° C.; gradient: 2-88% (or 0-45%, or 65-95%) solvent B over a 1.29 min period; flow rate 1.2 mL/min. Compounds were detected by a Waters Photodiode Array Detector. All masses were reported as those of the protonated parent ions, molecular weight range 150-850; cone voltage 20V.

NMR spectra were run on open access Varian 400 NMR, Bruker 400 MHz and Bruker 500 MHz nmr spectrometers. Spectra were measured at 298K and were referenced using the solvent peak unless otherwise specified.

Preparative separations are carried out using a Combiflash Rf system (Teledyne Isco, Lincoln, Nebr.) with RediSep silica gel cartridges (Teledyne Isco, Lincoln, Nebr.) or SiliaSep silica gel cartridges (Silicycle Inc., Quebec City, Canada) or by flash column chromatography using silica gel (230-400 mesh) packing material, or by HPLC using a Waters 2767 Sample Manager, C-18 reverse phase Sunfire column, 30×50 mm, flow 75 mL/min. Typical solvents employed for the Combiflash Rf system and flash column chromatography are dichloromethane, methanol, ethyl acetate, hexane, heptane, acetone, aqueous ammonia (or ammonium hydroxide), and triethyl amine. Typical solvents employed for the reverse phase HPLC are varying concentrations of acetonitrile and water with 0.1% trifluoroacetic acid or 0.1% formic acid.

The following examples are intended to illustrate the invention and are not to be construed as being limitations thereon. Temperatures are given in degrees centigrade. If not mentioned otherwise, all evaporations are performed under reduced pressure. The structure of final products, intermediates and starting materials is confirmed by standard analytical methods, e.g., MS, and NMR. Abbreviations used are those conventional in the art. If not defined, the terms have their generally accepted meanings.

### Abbreviations:

BiPy 2,2′-bipyridine
br broad
d doublet
DCM dichloromethane
DCE Dichloroethane
DMF N,N-dimethylformamide
DMAP 4-Dimethylaminopyridine
DMSO dimethylsulfoxide
EDCI 1-ethyl-3-(3′-dimethylaminopropyl) carbodiimide
EtOAc ethyl acetate
HPLC high pressure liquid chromatography
LCMS liquid chromatography and mass spectrometry
MeOH methanol
MS mass spectrometry
m multiplet
min minutes
ml milliliter(s)
NMR nuclear magnetic resonance
ppm parts per million
Rt retention time
RT or rt room temperature
s singlet
t triplet
THF tetrahydrofuran
UPLC Ultra Performance Liquid Chromatography

Referring to the examples that follow, compounds of the invention were synthesized using the methods described herein, and other methods that are known in the art.

The various starting materials, intermediates, and compounds of the preferred embodiments may be isolated and purified, where appropriate, using conventional techniques such as precipitation, filtration, crystallization, evaporation, distillation, and chromatography. Unless otherwise stated, all starting materials are obtained from commercial suppliers and used without further purification. Salts may be prepared from compounds by known salt-forming procedures.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

### Example 1.1

### (S)-10-((R)-3-(1-aminocyclopropyl)pyrrolidin-1-yl)-9-fluoro-3-methyl-5-oxo-2,3-dihydro-5H-[1,4]oxazino[2,3,4-ij]quinoline-6-carboxylic acid hydrochloride salt

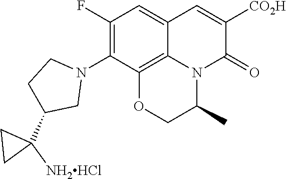

The title compound was prepared in accordance with the following scheme(Procedure A):

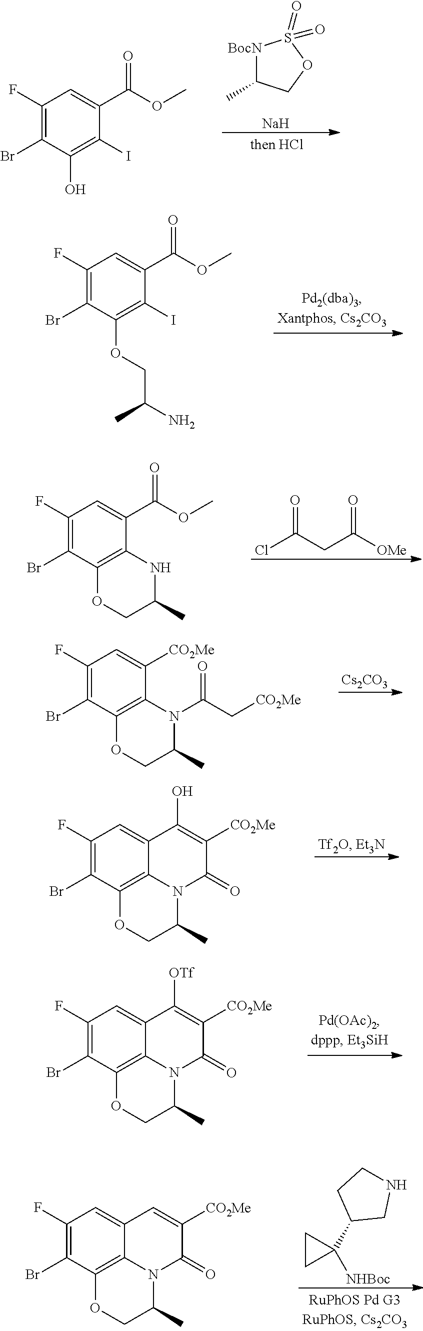

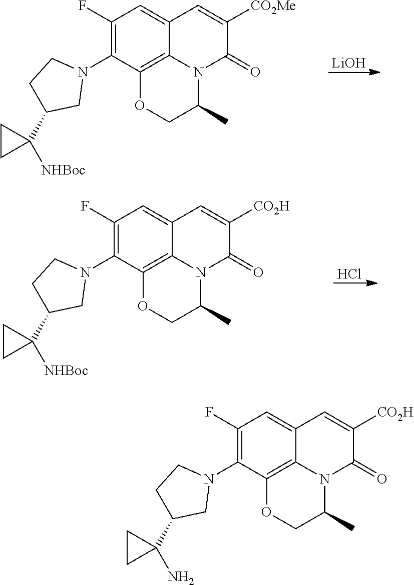 [SMILES: COC(=O)c1cc2cc(F)c(N3CC[C@@H](C4(NC(=O)OC(C)(C)C)CC4)C3)c3c2n(c1=O)[C@@H](C)CO3 ; "LiOH" ; C[C@H]1COc2c(N3CC[C@@H](C4(NC(=O)OC(C)(C)C)CC4)C3)c(F)cc3cc(C(=O)O)c(=O)n1c23 ; Cl ; C[C@H]1COc2c(N3CC[C@@H](C4(N)CC4)C3)c(F)cc3cc(C(=O)O)c(=O)n1c23]

### (i). Methyl (S)-3-(2-aminopropoxy)-4-bromo-5-fluoro-2-iodobenzoate hydrochloride

To a solution of methyl 4-bromo-5-fluoro-3-hydroxy-2-iodobenzoate (3.50 g, 9.34 mmol) in anhydrous DMF (10 mL) was added sodium hydride (0.41 g, 10.3 mmol) and the resulting mixture was stirred at rt for 5 minutes. tert-Butyl (S)-4-methyl-1,2,3-oxathiazolidine-3-carboxylate 2,2-dioxide (2.44 g, 10.3 mmol) in DMF (5 mL) was added and the mixture was stirred at rt for 2 h. EtOAc was added, followed by HCl 0.2N. The phase were separated and the aqueous layer was further extracted with EtOAc. The combined organic layer was washed with water and brine. The organic phase was dried on Na₂SO₄, filtered, concentrated. 1,4-Dioxane (10 ml) and water (1.2 mL) were added, followed by concentrated sulfuric acid (3.6 mL). The solution was stirred at rt for 30 minutes. The mixture was quenched with saturated aq. NaHCO₃ and extracted with DCM. The combined organic extract was concentrated to remove the DCM and 5mL of 4N HCl in dioxane was added. The solvent was evaporated to give a beige solid (4.33 g, 99% yield) corresponding to the product. ¹H NMR (400 MHz, DMSO) δ 8.39 (s, 2H), 7.61 (d, J=8.4 Hz, 1H), 4.15-4.05 (m, 2H), 3.89 (s, 3H), 3.69 (s, 1H), 1.43-1.42 (d, J=6.8 Hz, 3H). LCMS (m/z): 434.2 [M+2].

### (ii). Methyl (S)-8-bromo-7-fluoro-3-methyl-3,4-dihydro-2H-benzo[b][1, 4]oxazine-5-carboxylate

To a solution of methyl (S)-3-(2-aminopropoxy)-4-bromo-5-fluoro-2-iodobenzoate hydrochloride (4.70 g, 10.0 mmol) in anhydrous dioxane (45 ml) were added Pd₂(dba)₃ (0.92 g, 1.00 mmol), xantphos (1.74 g, 3.01 mmol) and cesium carbonate (9.81 g, 30.1 mmol). The mixture was stirred at 80° C. overnight. Water was added to the mixture and the slurry was extracted with DCM. The combined organic extract was dried on Na₂SO₄, filtered and concentrated in vacuo. The crude material was purified by silica gel chromatography (100% heptane to 100% EtOAc), affording a yellow solid (1.82 g, 60% yield), which contained a small amount of dba and des-bromo product as impurities. LCMS (m/z): 304.3 [M].

### (iii). Methyl (S)-8-bromo-7-fluoro-4-(3-methoxy-3-oxopropanoyl)-3-methyl-3,4-dihydro-2H-benzo[b][1, 4]oxazine-5-carboxylate

To a solution of methyl (S)-8-bromo-7-fluoro-3-methyl-3,4-dihydro-2H-benzo[b][1,4]oxazine-5-carboxylate (1.82 g, 5.97 mmol) in anhydrous toluene (30 mL) was added methyl 3-chloro-3-oxo-propionate (3.26 g, 2.56 mL, 23.9 mmol). The solution was stirred at 60° C. for 1 h. The solvent was evaporated and the crude material was purified by silica gel chromatogrpahy (0-100% EtOAc/heptane) to provide an orange oil (1.75 g, 73% yield). LCMS (m/z): 406.3 [M+2].

### (iv). Methyl (S)-10-bromo-9-fluoro-7-hydroxy-3-methyl-5-oxo-2,3-dihydro-5H-[1,4]oxazino[2,3,4-ij]quinoline-6-carboxylate

To a solution of methyl (S)-8-bromo-7-fluoro-4-(3-methoxy-3-oxopropanoyl)-3-methyl-3,4-dihydro-2H-benzo[b][1,4]oxazine-5-carboxylate (1.75 g, 4.33 mmol) in anhydrous acetonitrile (40 mL) was added cesium carbonate (4.23 g, 13.0 mmol). The slurry was stirred at 60° C. for 3 h. The reaction mixture was diluted with water and acidified with 1N HCl solution to pH 2-3. EtOAc was added, and the phases were separated. The aqueous layer was extracted with EtOAc, and the organic phase was dried on Na₂SO₄, filtered and concentrated. The crude material was purified by silica gel chromatography using 100% heptane to 80% EtOAc in heptane, affording a light yellow solid (1.16 g, 72% yield). ¹H NMR (400 MHz, DMSO) δ 12.93 (s, 1H), 7.54 (d, J=9.2 Hz, 1H), 4.93-4.89 (m, 1H), 4.64-4.60 (m, 1H), 4.25-4.22 (m, 1H), 3.88 (s, 3H), 1.21 (d, J=8.8 Hz, 3H). LCMS (m/z): 372.3 [M].

### (v). Methyl (S)-10-bromo-9-fluoro-3-methyl-5-oxo-7-(((trifluoromethyl)sulfonyl)oxy)-2,3-dihydro-5H-[1,4]oxazino[2,3,4-ij]quinoline-6-carboxylate

To a solution of methyl (S)-10-bromo-9-fluoro-7-hydroxy-3-methyl-5-oxo-2,3-dihydro-5H-[1,4]oxazino[2,3,4-ij]quinoline-6-carboxylate (1.16 g, 3.13 mmol) in anhydrous DCM (25 mL) was added triethylamine (949 mg, 1.31 mL, 9.38 mmol) and triflic anhydride (2.65 g, 1.58 mL, 9.38 mmol) at 0° C. The reaction was complete after 15 minutes. The solvent was evaporated and the crude material was carried on to the next step without further purification. Give a dark brown oil corresponding to the product. LCMS (m/z): 503.9 [M].

### (vi). Methyl (S)-10-bromo-9-fluoro-3-methyl-5-oxo-2,3-dihydro-5H-[1,4]oxazino[2,3,4-ij]quinoline-6-carboxylate

Crude methyl (S)-10-bromo-9-fluoro-3-methyl-5-oxo-7-(((trifluoromethyl)sulfonyl)oxy)-2,3-dihydro-5H-[1,4]oxazino[2,3,4-ij]quinoline-6-carboxylate was dissolved in dry DMF (20 mL). DPPP (0.39 g, 0.94 mmol), palladium(II) acetate (0.11 g, 0.47 mmol) and triethylsilane (908 mg, 1.25 mL, 7.81 mmol) were added sequentially under N₂ purging. The reaction mixture was stirred at 60° C. for 4 h. The crude mixture was diluted with EtOAc and extracted with water. The organic layer was washed with water, dried on Na₂SO₄ and filtered. The solvent was evaporated, and the crude residue was purified by silica gel chromatography (0-60% EtOAc/Heptane) to afford a yellow solid as the desired product (797 mg, 72% yield) with some amount (ca. 10%) of des-bromo product present. ¹H NMR (400 MHz, CDCl₃) δ 8.40 (s, 1H), 7.10 (d, J=7.6 Hz, 1H), 5.21-5.19 (m, 1H), 4.62-4.59 (m, 1H), 4.23-4.19 (m, 1H), 3.99 (s, 3H), 1.45 (d, J=6.8 Hz, 3H). LCMS (m/z): 358.2 [M+2].

### (vii). Methyl (S)-10-((R)-3-(1-((tert-butoxycarbonyl)amino)cyclopropyl)pyrrolidin-1-yl)-9-fluoro-3-methyl-5-oxo-2,3-dihydro-5H-[1,4]oxazino[2,3,4-ij]quinoline-6-carboxylate

A microwave vial was charged with methyl (S)-10-bromo-9-fluoro-3-methyl-5-oxo-2,3-dihydro-5H-[1,4]oxazino[2,3,4-ij]quinoline-6-carboxylate (300 mg, 0.84 mmol) and toluene (10 mL). tert-Butyl (S)-(1-(pyrrolidin-3-yl)cyclopropyl)carbamate (286 mg, 1.26 mmol), RuPhos (79 mg, 0.17 mmol), RuPhos Pd G3 (141 mg, 0.17 mmol) and cesium carbonate (823 mg, 2.53 mmol) were added. The mixture was heated to 90° C. for 4 h. The crude mixture was filtered through a disposable filter funnel and volatiles were evaporated under reduced pressure. Silica gel chromatography (0-100% EtOAc/heptane) provided the product (221 mg, 52% yield) as a yellow solid. ¹H NMR (400 MHz, DMSO-d₆) δ 8.36 (s, 1H), 7.38-7.13 (m, 1H), 4.90 (d, J=6.2 Hz, 1H), 4.40 (d, J=11.1 Hz, 1H), 3.98 (d, J=9.5 Hz, 1H), 3.87-3.74 (m, 4H), 3.73-3.63 (m, 1H), 3.55 (q, J=8.7 Hz, 2H), 2.17-2.03 (m, 1H), 1.85 (s, 1H), 1.64 (quin, J=9.8 Hz, 1H), 1.45-1.33 (m, 9H), 1.25 (d, J=6.6 Hz, 3H), 0.64 (d, J=3.1 Hz, 4H). LCMS (m/z): 502.5 [M+1].

### (viii). (S)-10-((R)-3-(1-((tert-butoxycarbonyl)amino)cyclopropyl)pyrrolidin-1-yl)-9-fluoro-3-methyl-5-oxo-2,3-dihydro-5H-[1,4]oxazino[2,3,4-ij]quinoline-6-carboxylic acid

Methyl (S)-10-((R)-3-(1-((tert-butoxycarbonyl)amino)cyclopropyl)pyrrolidin-1-yl)-9-fluoro-3-methyl-5-oxo-2,3-dihydro-5H-[1,4]oxazino[2,3,4-ij]quinoline-6-carboxylate (566 mg, 1.13 mmol) was dissolved in MeOH (12 mL) and water (3 mL). Lithium hydroxide hydrate (189 mg, 4.51 mmol) was added at rt. After stirring overnight the mixture was diluted with water, and treated with 1 M HCl. The resulting mixture was extracted with EtOAc, and the combined organic extract was washed with brine, dried over Na₂SO₄, filtered and concentrated under reduced pressure. The product was used directly in the next step without further purification. LCMS (m/z): 488.5 [M+1].

### (ix). (S)-10-((R)-3-(1-aminocyclopropyl)pyrrolidin-1-yl)-9-fluoro-3-methyl-5-oxo-2,3-dihydro-5H-[1,4]oxazino[2,3,4-ij]quinoline-6-carboxylic acid hydrochloride salt

Crude (S)-10-((R)-3-(1-((tert-butoxycarbonyl)amino)cyclopropyl)pyrrolidin-1-yl)-9-fluoro-3-methyl-5-oxo-2,3-dihydro-5H-[1,4]oxazino[2,3,4-ij]quinoline-6-carboxylic acid was dissolved in DCM (5 mL) and HCl 4N in dioxane (5 mL) was added. The solution was stirred at rt for 2 h. Et₂O (5 mL) was added, the precipitate was filtered and washed with more Et₂O to afford the product (434 mg, 90% yield) as a yellow solid. 1H NMR (400 MHz, Methanol-d4) δ 8.70 (s, 1H), 7.31 (d, J=13.5 Hz, 1H), 5.13 (q, J=6.1 Hz, 1H), 4.53 (d, J=11.3 Hz, 1H), 4.08 (d, J=9.6 Hz, 1H), 3.99 (dt, J=9.9, 6.9 Hz, 1H), 3.77 (dq, J=18.2, 10.0, 8.8 Hz, 4H), 2.59 (dt, J=16.7, 7.6 Hz, 1H), 2.17-2.06 (m, 1H), 1.70 (dt, J=20.2, 10.1 Hz, 1H), 1.43 (d, J=6.7 Hz, 3H), 1.04-0.93 (m, 4H). LCMS: tR=2.87 min, m/z=388.17 [M+1] (10 minute run, method A).

Alternatively, the Boc group could be removed using this procedure: (S)-10-((R)-3-(1-aminocyclopropyl)pyrrolidin-1-yl)-9-fluoro-3-methyl-5-oxo-2,3-dihydro-5H-[1,4]oxazino[2,3,4-ij]quinoline-6-carboxylic acid trifluoroacetate salt: (S)-10-((R)-3-(1-((tert-butoxycarbonyl)amino)cyclopropyl)pyrrolidin-1-yl)-9-fluoro-3-methyl-5-oxo-2,3-dihydro-5H-[1,4]oxazino[2,3,4-ij]quinoline-6-carboxylic acid (214 mg, 0.44 mmol) was dissolved in DCM (5 mL) and TFA (5 mL) was added. The solution was stirred at rt for 2 h. The solvent was evaporated and the crude material purified on reverse phase HPLC, affording the product (153 mg, 68% yield) as a yellow solid. LCMS: tR=2.87 min, m/z=388.17 [M+1] (10 minute run, method A).

Alternatively, the title compound could be prepared using the route depicted below (Procedure B):

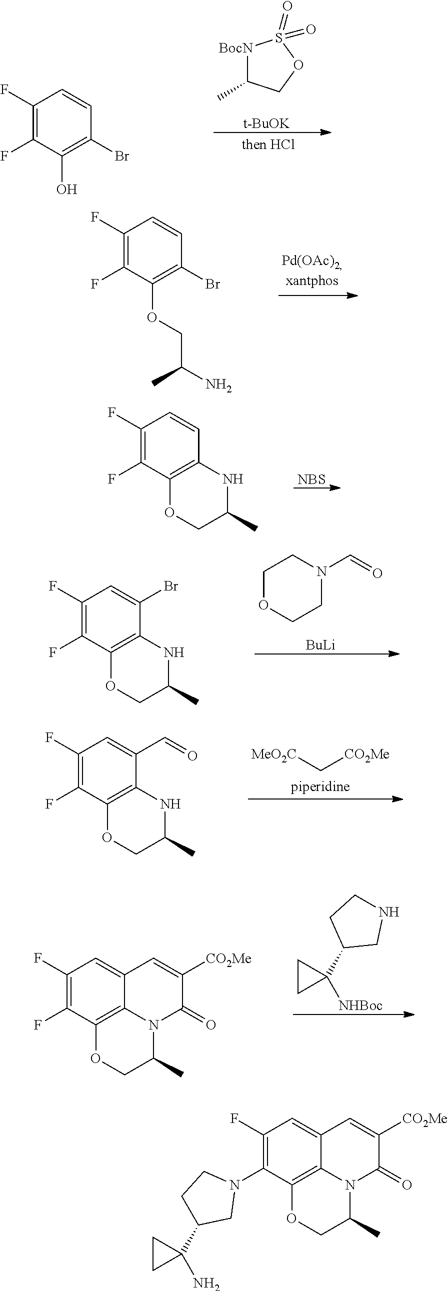

### (i). (S)-1-(6-bromo-2,3-difluorophenoxy)propan-2-amine

To a solution of 6-bromo-2,3-difluoro-phenol (19.0 g, 81.8 mmol) in DMF (120 mL) was added t-BuOK (12.1 g, 107 mmol) and the resulting mixture was stirred at rt for 15 minutes. tert-Butyl (S)-4-methyl-1,2,3-oxathiazolidine-3-carboxylate 2,2-dioxide (17.0 g, 71.6 mmol) was added and the mixture was stirred at 40° C. for 4 h. After completion the mixture was diluted with TBME, and 7.5% citric acid solution was added into the mixture. The organic layer was separated and washed with brine. The organic layer was dried over Na₂SO₄, filtered and concentrated under reduced pressure to give a yellow oil. The crude material was diluted in EtOAc (200 mL) and 4N HCl/IPA(100 mL) was added at rt. The mixture was stirred at rt for 16 h. After completion the mixture was filtered and the solid was dissolved in H₂O (200 mL). The solution was washed with EtOAc. The aqueous phase was basified with aqueous NaHCO₃ and extracted with EtOAc. The organic phase was dried over Na₂SO₄ and concentrated in vacuo to give (S)-1-(6-bromo-2,3-difluorophenoxy)propan-2-amine (12.0 g, 64% yield) as a yellow oil. ¹H NMR (400 MHz, Chloroform-d) δ 7.28-7.19 (m, 1H), 6.81 (q, J=9.1 Hz, 1H), 4.11 (dd, J=9.0, 2.7 Hz, 1H), 3.83 (t, J=8.4 Hz, 1H), 3.43-3.30 (m, 1H), 1.15 (d, J=6.6 Hz, 3H).

### (ii). (S)-7,8-difluoro-3-methyl-3,4-dihydro-2H-benzo[b][1, 4]oxazine

A mixture of (S)-1-(6-bromo-2,3-difluorophenoxy)propan-2-amine (12.0 g, 45.1 mmol), Pd(OAc)₂ (607 mg, 2.70 mmol), xantphos (1.57 g, 2.70 mmol), t-BuONa (8.89 g, 92.4 mmol) in toluene (120 mL) was stirred at 100° C. for 18 h. After completion, the reaction was cooled to rt and water and EtOAc were added. The phases were separated, and the aqueous layer was extracted with EtOAc. The organic phase was dried over Na₂SO₄, filtered and concentrated to give the crude product as a brown oil. The material was purified by silica gel chromatography using 10% EtOAc in heptane as eluent to give (S)-7,8-difluoro-3-methyl-3,4-dihydro-2H-benzo[b][1,4]oxazine (5.20 g, 62% yield) as a yellow oil. ¹H NMR (400 MHz, Chloroform-d) δ 6.54 (ddd, J=10.0, 9.1, 7.9 Hz, 1H), 6.25 (ddd, J=9.0, 4.8, 2.3 Hz, 1H), 4.27 (dd, J=10.5, 2.8 Hz, 1H), 3.78 (dd, J=10.5, 8.2 Hz, 1H), 3.49 (tdd, J=10.9, 5.5, 2.8 Hz, 1H), 1.19 (d, J=6.4 Hz, 3H).

(iii). (S)-5-bromo-7,8-difluoro-3-methyl-3,4-dihydro-2H-benzo[b][1,4]oxazine: To a mixture of (S)-7,8-difluoro-3-methyl-3,4-dihydro-2H-benzo[b][1,4]oxazine (1.00 g, 5.40 mmol) in MeCN (10 mL) was added NBS (0.96 g, 5.40 mmol) followed by stirring at rt for 12 h. After completion the reaction was diluted with EtOAc and Na₂CO₃ saturated aqueous solution was added. The organic layer was separated and washed with Na₂CO₃ saturated aqueous solution and brine. The organic layer was concentrated and the crude material was purified by silica gel chromatography using as eluent 5% EtOAc in heptane, affording (S)-5-bromo-7,8-difluoro-3-methyl-3,4-dihydro-2H-benzo[b][1,4]oxazine (639 mg, 45% yield). ¹H NMR (400 MHz, Chloroform-d) δ 6.89 (dd, J=9.6, 7.4 Hz, 1H), 4.28 (dd, J=10.5, 2.8 Hz, 1H), 3.79 (dd, J=10.5, 8.1 Hz, 1H), 3.56 (dtt, J=9.2, 6.5, 2.8 Hz, 1H), 1.26 (d, J=6.4 Hz, 3H).

### (iv). (S)-7,8-difluoro-3-methyl-3,4-dihydro-2H-benzo[b][1,4]oxazine-5-carbaldehyde

To a solution of (S)-5-bromo-7,8-difluoro-3-methyl-3,4-dihydro-2H-benzo[b][1,4]oxazine (1.50 g, 5.68 mmol) in THF (15 mL) was added n-BuLi (7.95 mL, 19.9 mmol) at −78° C. The mixture was warmed to −40° C. and stirred at this temperature for 1 h. Morpholine-4-carbaldehyde (2.74 g, 23.9 mmol) was then added dropwise. The reaction mixture was allowed to warm to rt followed by stirring for 30 minutes. After completion, 4 mL 1M HCl was added and the mixture was stirred at rt for another 30 minutes. The mixture was then poured into EtOAc and aqueous NH₄Cl. The phases were separated, and the organic layer was washed by brine, dried over anhydrous sodium sulfate, and filtered. Removal of the solvent provided a residue which was purified by silica gel chromatography (5% EtOAc in heptane) to afford the product (420 mg, 35% yield) as light yellow solid. ¹H NMR (400 MHz, Chloroform-d) δ 9.68 (s, 1H), 7.97 (br s, 1H), 6.91 (dd, J=9.9, 7.8 Hz, 1H), 4.31 (dd, J=10.5, 3.1 Hz, 1H), 3.81 (dd, J=10.5, 7.6 Hz, 1H), 3.69 (qt, J=6.7, 3.1 Hz, 1H), 1.28 (d, J=6.5 Hz, 3H).

### (v). Methyl (S)-9,10-difluoro-3-methyl-5-oxo-2,3-dihydro-5H-[1,4]oxazino[2,3,4-ij]quinoline-6-carboxylate

(S)-7,8-difluoro-3-methyl-3,4-dihydro-2H-benzo[b][1,4]oxazine-5-carbaldehyde (5.00 g, 23.5 mmol) was dissolved in toluene (240 mL). Dimethyl malonate (9.30 g, 8.05 mL, 70.4 mmol), piperidine (4.00 g, 4.64 mL, 47.0 mmol) and acetic acid (2.82 g, 2.69 mL, 46.9 mmol) were added and the mixture was heated to 110° C. for 5 h. After completion the solution was cooled to rt and saturated NaHCO₃ aqueous solution was added. The phases were separated, and the aqueous layer was extracted with EtOAc. The organic layer was washed with brine, and the solvent was removed under reduced pressure. The crude residue was tritured in TBME (20 mL) for 30 minutes, filtered and 10 mL of EtOAc was added. The suspension was stirred at 60° C. for 2 h and overnight at rt. The solid was collected to afford methyl (S)-9,10-difluoro-3-methyl-5-oxo-2,3-dihydro-5H-[1,4]oxazino[2,3,4-ij]quinoline-6-carboxylate (4.00 g, 58% yield) as a yellow solid.

### (vi). Methyl (S)-10-((R)-3-(1-((tert-butoxycarbonyl)amino)cyclopropyl)pyrrolidin-1-yl)-9-fluoro-3-methyl-5-oxo-2,3-dihydro-5H-[1,4]oxazino[2,3,4-ij]quinoline-6-carboxylate

To a solution of (R)-tert-butyl (1-(pyrrolidin-3-yl)cyclopropyl)carbamate (620 mg, 2.63 mmol) in DMSO (9 mL) were added methyl (S)-9,10-difluoro-3-methyl-5-oxo-2,3-dihydro-5H-[1,4]oxazino[2,3,4-ij]quinoline-6-carboxylate (620 mg, 2.07 mmol) and DIPEA (1.07 g, 1.44 mL, 8.27 mmol). The solution was then heated and stirred at 90° C. for 4 h. The solution was cooled to rt after which point a yellow solid precipitated. Water (18 mL) was slowly added and the solution was stirred at rt for 1 h. The solid was filtered and washed with water (5 mL) to afford the product (1.03 g) as a wet solid. LCMS: m/z=502.21 [M+1].

Using the procedures described for Example 1.1 the following compounds were prepared:

| | | | | HPLC | |
|---|---|---|---|---|---|
| Ex | | | | method, | LCMS |
| # | Structure | Chemical Name | Procedure | t_{R} (min) | [M + 1] |
| 1.2 | 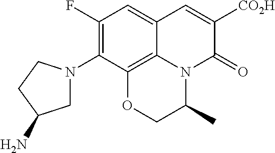 [SMILES: C[C@H]1COc2c(N3CC[C@H](N)C3)c(F)cc3cc(C(=O)O)c(=O)n1c23] | (S)-10-((S)-3- aminopyrrolidin-1-yl)-9- fluoro-3-methyl-5-oxo-2,3- dihydro-5H- [1,4]oxazino[2,3,4- ij]quinoline-6-carboxylic acid | A | A, 1.28 | 348.2 |
| 1.3 | 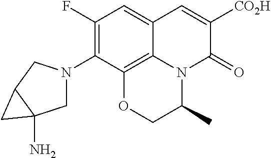 [SMILES: C[C@H]1COc2c(N3CC4CC4(N)C3)c(F)cc3cc(C(=O)O)c(=O)n1c23] | (3S)-10-(1-amino-3- azabicyclo[3.1.0]hexan-3- yl)-9-fluoro-3-methyl-5- oxo-2,3-dihydro-5H- [1,4]oxazino[2,3,4- ij]quinoline-6-carboxylic acid | A | A, 1.46 | 360.3 |
| 1.4 | 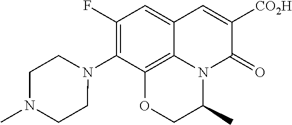 [SMILES: C[C@H]1COc2c(N3CCN(C)CC3)c(F)cc3cc(C(=O)O)c(=O)n1c23] | (S)-9-fluoro-3-methyl-10- (4-methylpiperazin-1-yl)-5- oxo-2,3-dihydro-5H- [1,4]oxazino[2,3,4- ij]quinoline-6-carboxylic acid (TFA salt) | A | A, 1.08 | 362.3 |
| 1.5 | 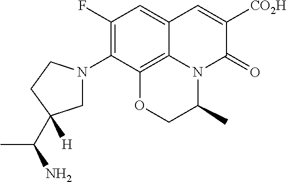 [SMILES: C[C@H](N)[C@@H]1CCN(c2c(F)cc3cc(C(=O)O)c(=O)n4c3c2OC[C@@H]4C)C1] | (S)-10-((R)-3-((S)-1- aminoethyl)pyrrolidin-1- yl)-9-fluoro-3-methyl-5- oxo-2,3-dihydro-5H- [1,4]oxazino[2,3,4- ij]quinoline-6-carboxylic acid (HCl salt) | B | A, 2.52 | 376.3 |
| 1.6² | 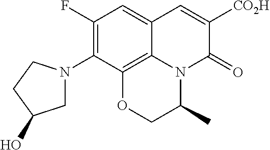 [SMILES: C[C@H]1COc2c(N3CC[C@H](O)C3)c(F)cc3cc(C(=O)O)c(=O)n1c23] | (S)-9-fluoro-10-((S)-3- hydroxypyrrolidin-1-yl)-3- methyl-5-oxo-2,3-dihydro- 5H-[1,4]oxazino[2,3,4- ij]quinoline-6-carboxylic acid | A | A, 2.93 | 349.2 |
| 1.7¹ | 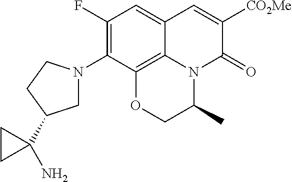 [SMILES: COC(=O)c1cc2cc(F)c(N3CC[C@@H](C4(N)CC4)C3)c3c2n(c1=O)[C@@H](C)CO3] | Methyl (S)-10-((R)-3-(1- aminocyclopropyl)pyrrolidin- 1-yl)-9-fluoro-3-methyl- 5-oxo-2,3-dihydro-5H- [1,4]oxazino[2,3,4- ij]quinoline-6-carboxylate (TFA salt) | A | A, 6.21 | 402.3 |
| 1.8² | 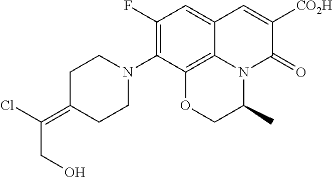 [SMILES: C[C@H]1COc2c(N3CCC(=C(Cl)CO)CC3)c(F)cc3cc(C(=O)O)c(=O)n1c23] | (S)-10-(4-(1-chloro-2- hydroxyethylidene)piperidin- 1-yl)-9-fluoro-3-methyl- 5-oxo-2,3-dihydro-5H- [1,4]oxazino[2,3,4- ij]quinoline-6-carboxylic acid | A | B, 1.90 | 423.24 |
| 1.9 | 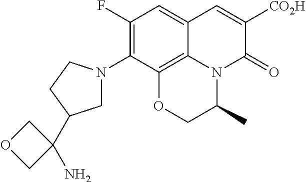 [SMILES: C[C@H]1COc2c(N3CCC(C4(N)COC4)C3)c(F)cc3cc(C(=O)O)c(=O)n1c23] | (3S)-10-(3-(3-aminooxetan- 3-yl)pyrrolidin-1-yl)-9- fluoro-3-methyl-5-oxo-2,3- dihydro-5H- [1,4]oxazino[2,3,4- ij]quinoline-6-carboxylic acid (HCl salt) | A | B, 1.39 | 404.44 |
| 1.10 | 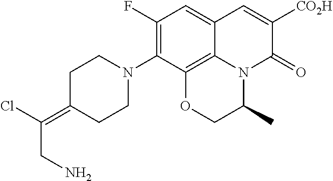 [SMILES: C[C@H]1COc2c(N3CCC(=C(Cl)CN)CC3)c(F)cc3cc(C(=O)O)c(=O)n1c23] | (S)-10-(4-(2-amino-1- chloroethylidene)piperidin- 1-yl)-9-fluoro-3-methyl-5- oxo-2,3-dihydro-5H- [1,4]oxazino[2,3,4- ij]quinoline-6-carboxylic acid (formate salt) | A | B, 1.48 | 422.3 |
| 1.11 | 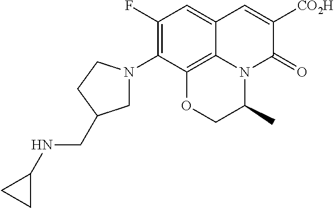 [SMILES: C[C@H]1COc2c(N3CCC(CNC4CC4)C3)c(F)cc3cc(C(=O)O)c(=O)n1c23] | (3S)-10-(3- ((cyclopropylamino)methyl) pyrrolidin-1-yl)-9-fluoro- 3-methyl-5-oxo-2,3- dihydro-5H- [1,4]oxazino[2,3,4- ij]quinoline-6-carboxylic acid (formate salt) | A | B, 1.44 | 402.39 |
| 1.12 | 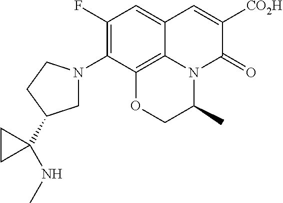 [SMILES: CNC1([C@@H]2CCN(c3c(F)cc4cc(C(=O)O)c(=O)n5c4c3OC[C@@H]5C)C2)CC1] | (S)-9-fluoro-3-methyl-10- ((R)-3-(1- (methylamino)cyclopropyl) pyrrolidin-1-yl)-5-oxo-2,3- dihydro-5H- [1,4]oxazino[2,3,4- ij]quinoline-6-carboxylic acid (HCl salt) | A | B, 1.45 | 402.44 |
| 1.13 | 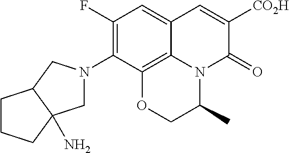 [SMILES: C[C@H]1COc2c(N3CC4CCCC4(N)C3)c(F)cc3cc(C(=O)O)c(=O)n1c23] | (3S)-10-(3a- aminohexahydrocyclopenta [c]pyrrol-2(1H)-yl)-9- fluoro-3-methyl-5-oxo-2,3- dihydro-5H- [1,4]oxazino[2,3,4- ij]quinoline-6-carboxylic acid (HCl salt) | A | B, 2.00 | 388.43 |
| 1.14² | 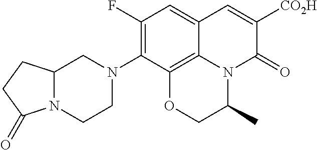 [SMILES: C[C@H]1COc2c(N3CCN4C(=O)CCC4C3)c(F)cc3cc(C(=O)O)c(=O)n1c23] | (3S)-9-fluoro-3-methyl-5- oxo-10-(6- oxohexahydropyrrolo[1,2- a]pyrazin-2(1H)-yl)-2,3- dihydro-5H- [1,4]oxazino[2,3,4- ij]quinoline-6-carboxylic acid | A | B, 1.65 | 402.3 |
| 1.15² | 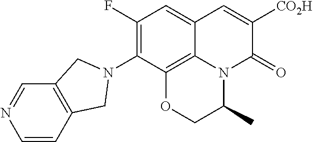 [SMILES: C[C@H]1COc2c(N3Cc4ccncc4C3)c(F)cc3cc(C(=O)O)c(=O)n1c23] | (S)-10-(1,3-dihydro-2H- pyrrolo[3,4-c]pyridin-2-yl)- 9-fluoro-3-methyl-5-oxo- 2,3-dihydro-5H- [1,4]oxazino[2,3,4- ij]quinoline-6-carboxylic acid | A | B, 1.43 | 382.33 |
| 1.16 | 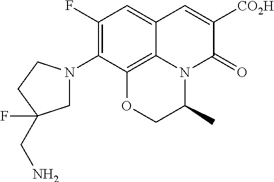 [SMILES: C[C@H]1COc2c(N3CCC(F)(CN)C3)c(F)cc3cc(C(=O)O)c(=O)n1c23] | (3S)-10-(3-(aminomethyl)- 3-fluoropyrrolidin-1-yl)-9- fluoro-3-methyl-5-oxo-2,3- dihydro-5H- [1,4]oxazino[2,3,4- ij]quinoline-6-carboxylic acid (HCl salt) | A | B, 1.39 | 380.33 |
| 1.17 | 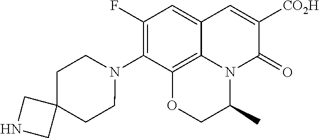 [SMILES: C[C@H]1COc2c(N3CCC4(CC3)CNC4)c(F)cc3cc(C(=O)O)c(=O)n1c23] | (S)-9-fluoro-3-methyl-5- oxo-10-(2,7- diazaspiro[3,5]nonan-7-yl)- 2,3-dihydro-5H- [1,4]oxazino[2,3,4- ij]quinoline-6-carboxylic acid (HCl salt) | A | B, 1.31 | 388.41 |
| 1.18 | 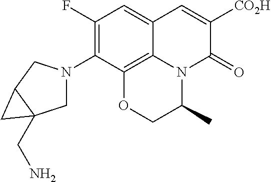 [SMILES: C[C@H]1COc2c(N3CC4CC4(CN)C3)c(F)cc3cc(C(=O)O)c(=O)n1c23] | (3S)-10-(1-(aminomethyl)- 3-azabicyclo[3.10]hexan- 3-yl)-9-fluoro-3-methyl-5- oxo-2,3-dihydro-5H- [1,4]oxazino[2,3,4- ijlquinoline-6-carboxylic acid (HCl salt) | A | B, 1.31 | 374.25 |
| 1.19 | 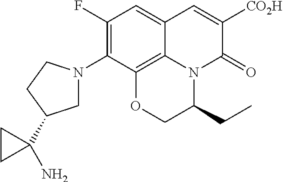 [SMILES: CC[C@H]1COc2c(N3CC[C@@H](C4(N)CC4)C3)c(F)cc3cc(C(=O)O)c(=O)n1c23] | (S)-10-((R)-3-(1- aminocyclopropyl)pyrrolidin- 1-yl)-3-ethyl-9-fluoro-5- oxo-2,3-dihydro-5H- [1,4]oxazino[2,3,4- ij]quinoline-6-carboxylic acid (HCl salt) | A | B, 1.50 | 402.4 |
| 1.20 | 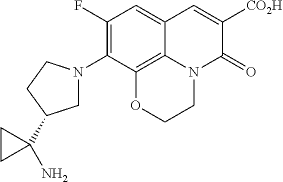 [SMILES: NC1([C@@H]2CCN(c3c(F)cc4cc(C(=O)O)c(=O)n5c4c3OCC5)C2)CC1] | (R)-10-(3-(1- aminocyclopropyl)pyrrolidin- 1-yl)-9-fluoro-5-oxo-2,3- dihydro-5H- [1,4]oxazino[2,3,4- ij]quinoline-6-carboxylic acid (HCl salt) | A | B, 1.43 | 374.23 |
| 1.21 | 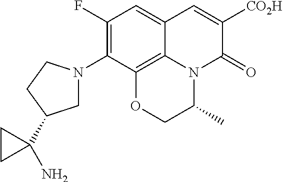 [SMILES: C[C@@H]1COc2c(N3CC[C@@H](C4(N)CC4)C3)c(F)cc3cc(C(=O)O)c(=O)n1c23] | (R)-10-((R)-3-(1- aminocyclopropyl)pyrrolidin- 1-yl)-9-fluoro-3-methyl- 5-oxo-2,3-dihydro-5H- [1,4]oxazino[2,3,4- ij]quinoline-6-carboxylic acid | A | B, 1.50 | 388.3 |
| 1.22 | 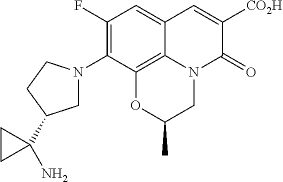 [SMILES: C[C@@H]1Cn2c(=O)c(C(=O)O)cc3cc(F)c(N4CC[C@@H](C5(N)CC5)C4)c(c32)O1] | (R)-10-((R)-3-(1- aminocyclopropyl)pyrrolidin- 1-yl)-9-fluoro-2-methyl- 5-oxo-2,3-dihydro-5H- [1,4]oxazino[2,3,4- ij]quinoline-6-carboxylic acid (HCl salt) | A | B, 1.50 | 388.3 |
| 1.23 | 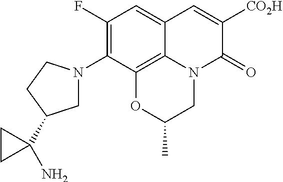 [SMILES: C[C@H]1Cn2c(=O)c(C(=O)O)cc3cc(F)c(N4CC[C@@H](C5(N)CC5)C4)c(c32)O1] | (S)-10-((R)-3-(1- aminocyclopropyl)pyrrolidin- 1-yl)-9-fluoro-2-methyl- 5-oxo-2,3-dihydro-5H- [1,4]oxazino[2,3,4- ij]quinoline-6-carboxylic acid | A | B, 1.41 | 388.32 |
| 1.24 | 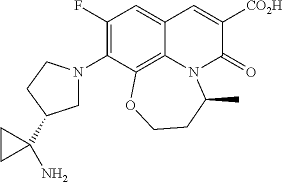 [SMILES: C[C@H]1CCOc2c(N3CC[C@@H](C4(N)CC4)C3)c(F)cc3cc(C(=O)O)c(=O)n1c23] | (S)-11-((R)-3-(1- aminocyclopropyl)pyrrolidin- 1-yl)-10-fluoro-4-methyl- 6-oxo-3,4-dihydro-2H,6H- [1,4]oxazepino[2,3,4- ij]quinoline-7-carboxylic acid (HCl salt) | A | B, 1.39 | 402.42 |
| 1.25² | 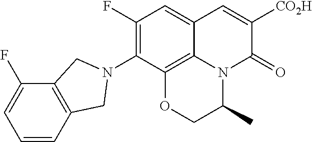 [SMILES: C[C@H]1COc2c(N3Cc4cccc(F)c4C3)c(F)cc3cc(C(=O)O)c(=O)n1c23] | (S)-9-fluoro-10-(4- fluoroisoindolin-2-yl)-3- methyl-5-oxo-2,3-dihydro- 5H-[1,4]oxazino[2,3,4- ij]quinoline-6-carboxylic acid | A | B, 1.98 | 399.2 |
| 1.26 | 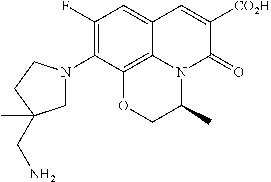 [SMILES: C[C@H]1COc2c(N3CCC(C)(CN)C3)c(F)cc3cc(C(=O)O)c(=O)n1c23] | (3S)-10-(3-(aminomethyl)- 3-methylpyrrolidin-1-yl)-9- fluoro-3-methyl-5-oxo-2,3- dihydro-5H- [1,4]oxazino[2,3,4- ij]quinoline-6-carboxylic acid (HCl salt) | A | B, 1.33 | 376.3 |
| 1.27 | 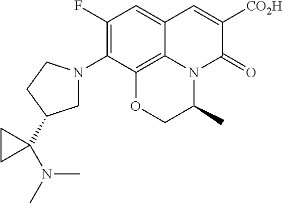 [SMILES: C[C@H]1COc2c(N3CC[C@@H](C4(N(C)C)CC4)C3)c(F)cc3cc(C(=O)O)c(=O)n1c23] | (S)-10-((R)-3-(1- (dimethylamino)cyclopropyl) pyrrolidin-1-yl)-9-fluoro- 3-methyl-5-oxo-2,3- dihydro-5H- [1,4]oxazino[2,3,4- ij]quinoline-6-carboxylic acid | A | B, 1.41 | 416.4 |
| 1.28³ | 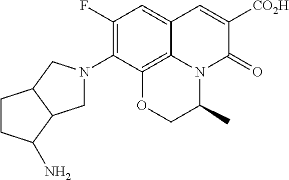 [SMILES: C[C@H]1COc2c(N3CC4CCC(N)C4C3)c(F)cc3cc(C(=O)O)c(=O)n1c23] | (3S)-10-(4- aminohexahydrocyclopenta [c]pyrrol-2(1H)-yl)-9- fluoro-3-methyl-5-oxo-2,3- dihydro-5H- [1,4]oxazino[2,3,4- ij]quinoline-6-carboxylic acid (TFA salt) | A | B, 1.37 | 388.4 |
| 1.29³ | 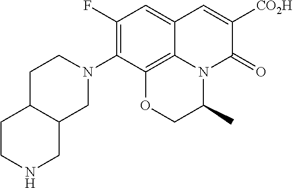 [SMILES: C[C@H]1COc2c(N3CCC4CCNCC4C3)c(F)cc3cc(C(=O)O)c(=O)n1c23] | (3S)-9-fluoro-3-methyl-10- (octahydro-2,7- naphthyridin-2(1H)-yl)-5- oxo-2,3-dihydro-5H- [1,4]oxazino[2,3,4- ij]quinoline-6-carboxylic acid (HCl salt) | A | B, 1.38 | 402.4 |
| 1.30 | 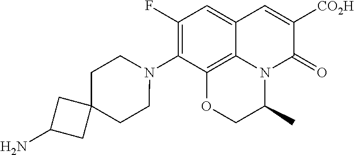 [SMILES: C[C@H]1COc2c(N3CCC4(CC3)CC(N)C4)c(F)cc3cc(C(=O)O)c(=O)n1c23] | (S)-10-(2-amino-7- azaspiro[3.5]nonan-7-yl)-9- fluoro-3-methyl-5-oxo-2,3- dihydro-5H- [1,4]oxazino[2,3,4- ij]quinoline-6-carboxylic acid (HCl salt) | A | B, 1.35 | 402.6 |
| 1.31² | 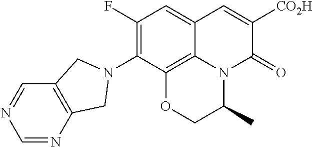 [SMILES: C[C@H]1COc2c(N3Cc4cncnc4C3)c(F)cc3cc(C(=O)O)c(=O)n1c23] | (S)-10-(5,7-dihydro-6H- pyrrolo[3,4-d]pyrimidin-6- yl)-9-fluoro-3-methyl-5- oxo-2,3-dihydro-5H- [1,4]oxazino[2,3,4- ij]quinoline-6-carboxylic acid | A | B, 1.46 | 383.3 |
| 1.32³ | 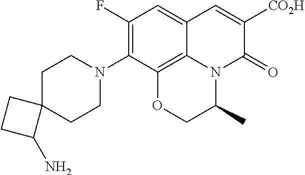 [SMILES: C[C@H]1COc2c(N3CCC4(CCC4N)CC3)c(F)cc3cc(C(=O)O)c(=O)n1c23] | (3S)-10-(1-amino-7- azaspiro[3.5]nonan-7-yl)-9- fluoro-3-methyl-5-oxo-2,3- dihydro-5H- [1,4]oxazino[2,3,4- ij]quinoline-6-carboxylic acid (formate salt) | A | B, 1.35 | 402.4 |
| 1.33³ | 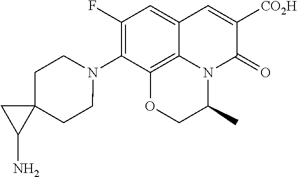 [SMILES: C[C@H]1COc2c(N3CCC4(CC3)CC4N)c(F)cc3cc(C(=O)O)c(=O)n1c23] | (3S)-10-(1-amino-6- azaspiro[2.5]octan-6-yl)-9- fluoro-3-methyl-5-oxo-2,3- dihydro-5H- [1,4]oxazino[2,3,4- ij]quinoline-6-carboxylic acid (formate salt) | A | B, 1.37 | 388.3 |
| 1.34² | 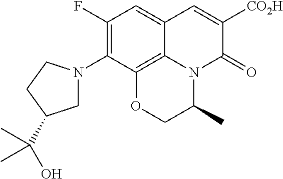 [SMILES: C[C@H]1COc2c(N3CC[C@@H](C(C)(C)O)C3)c(F)cc3cc(C(=O)O)c(=O)n1c23] | (S)-9-fluoro-10-((R)-3-(2- hydroxypropan-2- yl)pyrrolidin-1-yl)-3- methyl-5-oxo-2,3-dihydro- 5H-[1,4]oxazino[2,3,4- ij]quinoline-6-carboxylic acid | A | B, 1.60 | 391.48 |
| 1.35 | 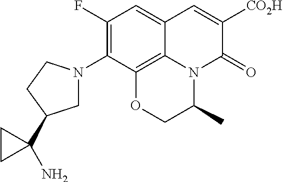 [SMILES: C[C@H]1COc2c(N3CC[C@H](C4(N)CC4)C3)c(F)cc3cc(C(=O)O)c(=O)n1c23] | (S)-10-((S)-3-(1- aminocyclopropyl)pyrrolidin- 1-yl)-9-fluoro-3-methyl- 5-oxo-2,3-dihydro-5H- [1,4]oxazino[2,3,4- ij]quinoline-6-carboxylic acid (HCl salt) | A | B, 1.36 | 388.7 |
| 1.36 | 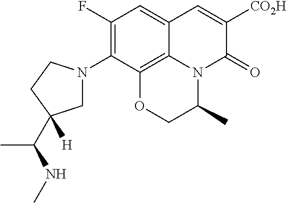 [SMILES: CN[C@@H](C)[C@@H]1CCN(c2c(F)cc3cc(C(=O)O)c(=O)n4c3c2OC[C@@H]4C)C1] | (S)-9-fluoro-3-methyl-10- ((R)-3-((S)-1- (methylamino)ethyl) pyrrolidin-1-yl)-5-oxo-2,3- dihydro-5H- [1,4]oxazino[2,3,4- ij]quinoline-6-carboxylic acid (HCl salt) | A | B, 1.36 | 390.6 |
| 1.37 | 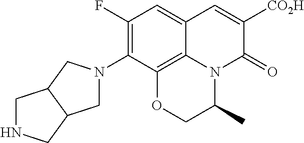 [SMILES: C[C@H]1COc2c(N3CC4CNCC4C3)c(F)cc3cc(C(=O)O)c(=O)n1c23] | (3S)-9-fluoro-10- (hexahydropyrrolo[3,4- c]pyrrol-2(1H)-yl)-3- methyl-5-oxo-2,3-dihydro- 5H-[1,4]oxazino[2,3,4- ij]quinoline-6-carboxylic acid (HCl salt) | A | B, 1.34 | 374.6 |
| 1.38⁵ | 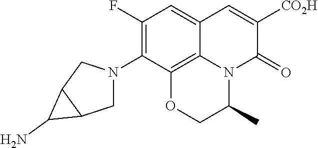 [SMILES: C[C@H]1COc2c(N3CC4C(N)C4C3)c(F)cc3cc(C(=O)O)c(=O)n1c23] | (3S)-10-(6-amino-3- azabicyclo[3.1.0]hexan-3- yl)-9-fluoro-3-methyl-5- oxo-2,3-dihydro-5H- [1,4]oxazino[2,3,4- ij]quinoline-6-carboxylic acid (HCl salt) | A | B, 1.30 | 360.4 |
| 1.39³ | 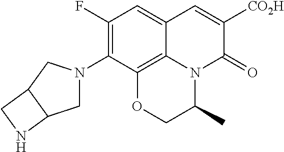 [SMILES: C[C@H]1COc2c(N3CC4CNC4C3)c(F)cc3cc(C(=O)O)c(=O)n1c23] | (3S)-10-(3,6- diazabicyclo[3.2.0]heptan- 3-yl)-9-fluoro-3-methyl-5- oxo-2,3-dihydro-5H- [1,4]oxazino[2,3,4- ij]quinoline-6-carboxylic acid (formate salt) | A | B, 1.32 | 360.5 |
| 1.40 | 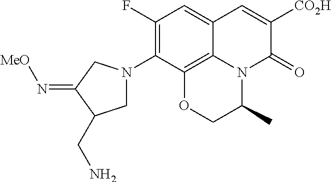 [SMILES: CO/N=C1\CN(c2c(F)cc3cc(C(=O)O)c(=O)n4c3c2OC[C@@H]4C)CC1CN] | (3S)-10-((Z)-3- (aminomethyl)-4- (methoxyimino)pyrrolidin- 1-yl)-9-fluoro-3-methyl-5- oxo-2,3-dihydro-5H- [1,4]oxazino[2,3,4- ij]quinoline-6-carboxylic acid | A | B, 1.33 | 405.5 |
| 1.41 | 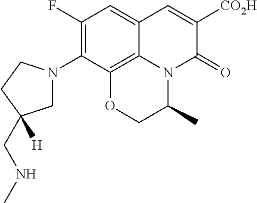 [SMILES: CNC[C@@H]1CCN(c2c(F)cc3cc(C(=O)O)c(=O)n4c3c2OC[C@@H]4C)C1] | (S)-9-fluoro-3-methyl-10- ((S)-3- ((methylamino)methyl) pyrrolidin-1-yl)-5-oxo-2,3- dihydro-5H- [1,4]oxazino[2,3,4- ij]quinoline-6-carboxylic acid (HCl salt) | A | B, 1.35 | 376.4 |
| 1.42³ | 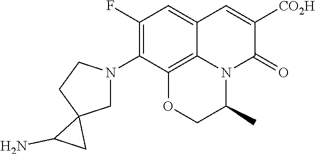 [SMILES: C[C@H]1COc2c(N3CCC4(CC4N)C3)c(F)cc3cc(C(=O)O)c(=O)n1c23] | (3S)-10-(1-amino-5- azaspiro[2.4]heptan-5-yl)- 9-fluoro-3-methyl-5-oxo- 2,3-dihydro-5H- [1,4]oxazino[2,3,4- ij]quinoline-6-carboxylic acid (formate salt) | A | B, 1.31 | 374.5 |
| 1.43³ | 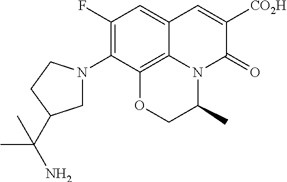 [SMILES: C[C@H]1COc2c(N3CCC(C(C)(C)N)C3)c(F)cc3cc(C(=O)O)c(=O)n1c23] | (3S)-10-(3-(2- aminopropan-2- yl)pyrrolidin-1-yl)-9- fluoro-3-methyl-5-oxo-2,3- dihydro-5H- [1,4]oxazino[2,3,4- ij]quinoline-6-carboxylic acid (HCl salt) | A | B, 1.33 | 390.5 |
| 1.44² | 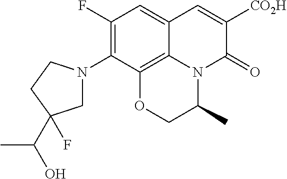 [SMILES: CC(O)C1(F)CCN(c2c(F)cc3cc(C(=O)O)c(=O)n4c3c2OC[C@@H]4C)C1] | (3S)-9-fluoro-10-(3-fluoro- 3-(1- hydroxyethyl)pyrrolidin-1- yl)-3-methyl-5-oxo-2,3- dihydro-5H- [1,4]oxazino[2,3,4- ij]quinoline-6-carboxylic acid | A | B, 1.56 | 395.4 |
| 1.45³ | 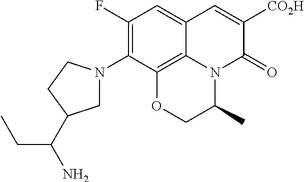 [SMILES: CCC(N)C1CCN(c2c(F)cc3cc(C(=O)O)c(=O)n4c3c2OC[C@@H]4C)C1] | (3S)-10-(3-(1- aminopropyl)pyrrolidin-1- yl)-9-fluoro-3-methyl-5- oxo-2,3-dihydro-5H- [1,4]oxazino[2,3,4- ij]quinoline-6-carboxylic acid (HCl salt) | A | B, 1.37 | 390.6 |
| 1.46³ | 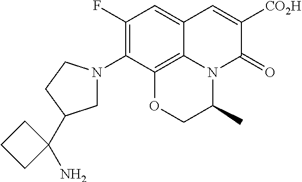 [SMILES: C[C@H]1COc2c(N3CCC(C4(N)CCC4)C3)c(F)cc3cc(C(=O)O)c(=O)n1c23] | (3S)-10-(3-(1- aminocyclobutyl)pyrrolidin- 1-yl)-9-fluoro-3-methyl-5- oxo-2,3-dihydro-5H- [1,4]oxazino[2,3,4- ij]quinoline-6-carboxylic acid (HCl salt) | A | B, 1.37 | 402.5 |
| 1.47³ | 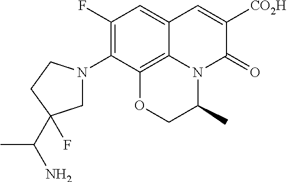 [SMILES: CC(N)C1(F)CCN(c2c(F)cc3cc(C(=O)O)c(=O)n4c3c2OC[C@@H]4C)C1] | (3S)-10-(3-(1-aminoethyl)- 3-fluoropyrrolidin-1-yl)-9- fluoro-3-methyl-5-oxo-2,3- dihydro-5H- [1,4]oxazino[2,3,4- ij]quinoline-6-carboxylic acid (TFA salt) | A | C, 23.61 | 394.2 |
| 1.48³ | 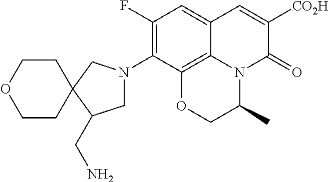 [SMILES: C[C@H]1COc2c(N3CC(CN)C4(CCOCC4)C3)c(F)cc3cc(C(=O)O)c(=O)n1c23] | (3S)-10-(4-(aminomethyl)- 8-oxa-2- azaspiro[4.5]decan-2-yl)-9- fluoro-3-methyl-5-oxo-2,3- dihydro-5H- [1,4]oxazino[2,3,4- ij]quinoline-6-carboxylic acid (HCl salt) | A | B, 1.34 | 432.6 |
| 1.49³ | 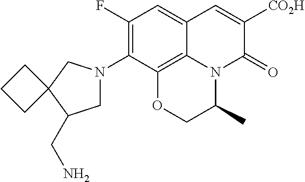 [SMILES: C[C@H]1COc2c(N3CC(CN)C4(CCC4)C3)c(F)cc3cc(C(=O)O)c(=O)n1c23] | (3S)-10-(8-(aminomethyl)- 6-azaspiro[3.4]octan-6-yl)- 9-fluoro-3-methyl-5-oxo- 2,3-dihydro-5H- [1,4]oxazino[2,3,4- ij]quinoline-6-carboxylic acid (HCl salt) | A | B, 1.42 | 402.6 |
| 1.50³ | 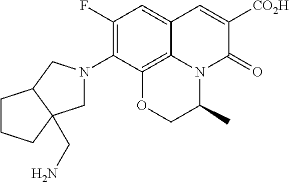 [SMILES: C[C@H]1COc2c(N3CC4CCCC4(CN)C3)c(F)cc3cc(C(=O)O)c(=O)n1c23] | (3S)-10-(3a- (aminomethyl)hexahydro- cyclopenta[c]pyrrol-2(1H)- yl)-9-fluoro-3-methyl-5- oxo-2,3-dihydro-5H- [1,4]oxazino[2,3,4- ij]quinoline-6-carboxylic acid (HCl salt) | A | B, 1.46 | 402.6 |
| 1.51 | 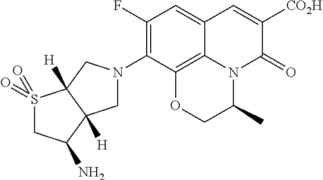 [SMILES: C[C@H]1COc2c(N3C[C@H]4[C@@H](C3)S(=O)(=O)C[C@@H]4N)c(F)cc3cc(C(=O)O)c(=O)n1c23] | (S)-10-((3R,3aR,6aS)-3- amino-1,1- dioxidohexahydro-5H- thieno[2,3-c]pyrrol-5-yl)-9- fluoro-3-methyl-5-oxo-2,3- dihydro-5H- [1,4]oxazino[2,3,4- ij]quinoline-6-carboxylic acid (formate salt) | B | B, 1.31 | 438.2 |
| 1.52⁴ | 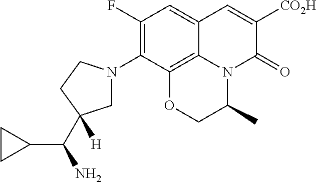 [SMILES: C[C@H]1COc2c(N3CC[C@@H]([C@@H](N)C4CC4)C3)c(F)cc3cc(C(=O)O)c(=O)n1c23] | (S)-10-((R)-3-((S)- amino(cyclopropyl)methyl) pyrrolidin-1-yl)-9-fluoro-3- methyl-5-oxo-2,3-dihydro- 5H-[1,4]oxazino[2,3,4- ij]quinoline-6-carboxylic acid | A | B, 1.34 | 402.47 |
| 1.53⁴ | 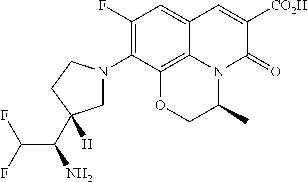 [SMILES: C[C@H]1COc2c(N3CC[C@@H]([C@@H](N)C(F)F)C3)c(F)cc3cc(C(=O)O)c(=O)n1c23] | (S)-10-((R)-3-((R)-1- amino-2,2- difluoroethyl)pyrrolidin-1- yl)-9-fluoro-3-methyl-5- oxo-2,3-dihydro-5H- [1,4]oxazino[2,3,4- ij]quinoline-6-carboxylic acid | A | B, 1.32 | 412.5 |
| 1.54 | 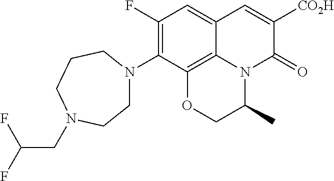 [SMILES: C[C@H]1COc2c(N3CCCN(CC(F)F)CC3)c(F)cc3cc(C(=O)O)c(=O)n1c23] | (S)-10-(4-(2,2- difluoroethyl)-1,4- diazepan-1-yl)-9-fluoro-3- methyl-5-oxo-2,3-dihydro- 5H-[1,4]oxazino[2,3,4- ij]quinoline-6-carboxylic acid | B | B, 1.36 | 426.39 |
| 1.55 | 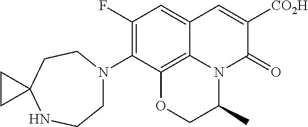 [SMILES: C[C@H]1COc2c(N3CCNC4(CC3)CC4)c(F)cc3cc(C(=O)O)c(=O)n1c23] | (S)-9-fluoro-3-methyl-5- oxo-10-(4,7- diazaspiro[2.6]nonan-7-yl)- 2,3-dihydro-5H- [1,4]oxazino[2,3,4- ij]quinoline-6-carboxylic acid (formate salt) | B | B, 1.35 | 388.3 |
| 1.56⁴ | 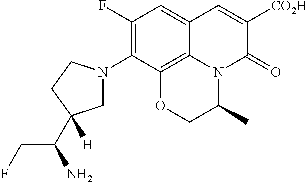 [SMILES: C[C@H]1COc2c(N3CC[C@@H]([C@@H](N)CF)C3)c(F)cc3cc(C(=O)O)c(=O)n1c23] | (S)-10-((R)-3-((R)-1- amino-2- fluoroethyl)pyrrolidin-1- yl)-9-fluoro-3-methyl-5- oxo-2,3-dihydro-5H- [1,4]oxazino[2,3,4- ij]quinoline-6-carboxylic acid (formate salt) | A | B, 1.32 | 394.43 |
| 1.57 | 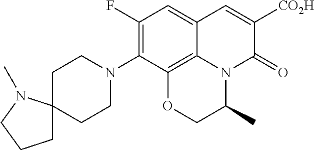 [SMILES: C[C@H]1COc2c(N3CCC4(CCCN4C)CC3)c(F)cc3cc(C(=O)O)c(=O)n1c23] | (S)-9-fluoro-3-methyl-10- (1-methyl-1,8- diazaspiro[4.5]decan-8-yl)- 5-oxo-2,3-dihydro-5H- [1,4]oxazino[2,3,4- ij]quinoline-6-carboxylic acid | B | B, 1.34 | 416.57 |
| 1.58 | 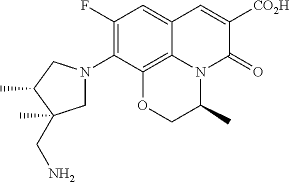 [SMILES: C[C@@H]1CN(c2c(F)cc3cc(C(=O)O)c(=O)n4c3c2OC[C@@H]4C)C[C@@]1(C)CN] | (S)-10-((3R,4S)-3- (aminomethyl)-3,4- dimethylpyrrolidin-1-yl)-9- fluoro-3-methyl-5-oxo-2,3- dihydro-5H- [1,4]oxazino[2,3,4- ij]quinoline-6-carboxylic acid | B | B, 1.40 | 390.47 |
| 1.59 | 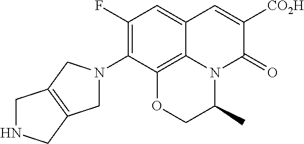 [SMILES: C[C@H]1COc2c(N3CC4=C(CNC4)C3)c(F)cc3cc(C(=O)O)c(=O)n1c23] | (S)-9-fluoro-3-methyl-5- oxo-10-(3,4,5,6- tetrahydropyrrolo[3,4- c]pyrrol-2(1H)-yl)-2,3- dihydro-5H- [1,4]oxazino[2,3,4- ij]quinoline-6-carboxylic acid | B | B, 1.31 | 372.3 |
| 1.60 | 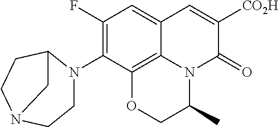 [SMILES: C[C@H]1COc2c(N3CCN4CCC3C4)c(F)cc3cc(C(=O)O)c(=O)n1c23] | (3S)-10-(1,4- diazabicyclo[3.2.1]octan-4- yl)-9-fluoro-3-methyl-5- oxo-2,3-dihydro-5H- [1,4]oxazino[2,3,4- ij]quinoline-6-carboxylic acid | A | B, 1.30 | 374.4 |
| 1.61 | 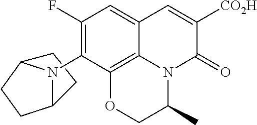 [SMILES: C[C@H]1COc2c(N3C4CCC3CC4)c(F)cc3cc(C(=O)O)c(=O)n1c23] | (S)-10-(7- azabicyclo[2.2.1]heptan-7- yl)-9-fluoro-3-methyl-5- oxo-2,3-dihydro-5H- [1,4]oxazino[2,3,4- ij]quinoline-6-carboxylic acid | B | B, 1.99 | 359.43 |
| 1.62³ | 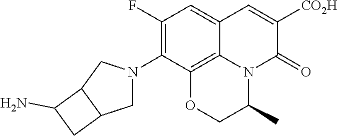 [SMILES: C[C@H]1COc2c(N3CC4CC(N)C4C3)c(F)cc3cc(C(=O)O)c(=O)n1c23] | (3S)-10-(6-amino-3- azabicyclo[3.2.0]heptan-3- yl)-9-fluoro-3-methyl-5- oxo-2,3-dihydro-5H- [1,4]oxazino[2,3,4- ij]quinoline-6-carboxylic acid | A | B, 1.38 | 374.37 |
| 1.63⁵ | 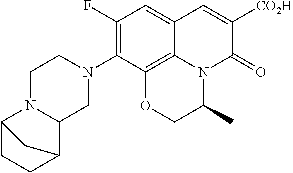 [SMILES: C[C@H]1COc2c(N3CCN4C5CCC(C5)C4C3)c(F)cc3cc(C(=O)O)c(=O)n1c23] | (3S)-9-fluoro-3-methyl-10- (octahydro-2H-6,9- methanopyrido[1,2- a]pyrazin-2-yl)-5-oxo-2,3- dihydro-5H- [1,4]oxazino[2,3,4- ij]quinoline-6-carboxylic acid | B | B, 1.48 | 414.52 |
| 1.64⁵ | 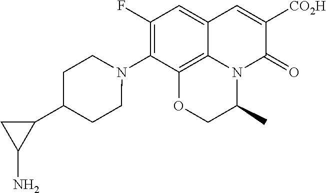 [SMILES: C[C@H]1COc2c(N3CCC(C4CC4N)CC3)c(F)cc3cc(C(=O)O)c(=O)n1c23] | (3S)-10-(4-(2- aminocyclopropyl)piperidin- 1-yl)-9-fluoro-3-methyl-5- oxo-2,3-dihydro-5H- [1,4]oxazino[2,3,4- ij]quinoline-6-carboxylic acid | B | B, 1.39 | 402.52 |
| 1.65⁵ | 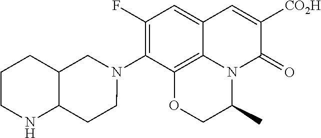 [SMILES: C[C@H]1COc2c(N3CCC4NCCCC4C3)c(F)cc3cc(C(=O)O)c(=O)n1c23] | (3S)-9-fluoro-3-methyl-10- (octahydro-1,6- naphthyridin-6(2H)-yl)-5- oxo-2,3-dihydro-5H- [1,4]oxazino[2,3,4- ij]quinoline-6-carboxylic acid | B | B, 1.32 | 402.5 |
| 1.66 | 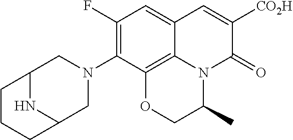 [SMILES: C[C@H]1COc2c(N3CC4CCCC(C3)N4)c(F)cc3cc(C(=O)O)c(=O)n1c23] | (3S)-10-(3,9- diazabicyclo[3.3.1]nonan- 3-yl)-9-fluoro-3-methyl-5- oxo-2,3-dihydro-5H- [1,4]oxazino[2,3,4- ij]quinoline-6-carboxylic acid (HCl salt) | B | B, 1.33 | 388.43 |
| 1.67 | 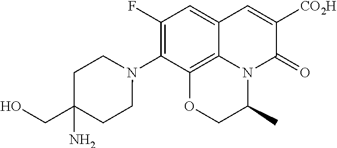 [SMILES: C[C@H]1COc2c(N3CCC(N)(CO)CC3)c(F)cc3cc(C(=O)O)c(=O)n1c23] | (S)-10-(4-amino-4- (hydroxymethyl)piperidin- 1-yl)-9-fluoro-3-methyl-5- oxo-2,3-dihydro-5H- [1,4]oxazino[2,3,4- ij]quinoline-6-carboxylic acid (TFA salt) | B | B, 1.32 | 392.47 |
| 1.68 | 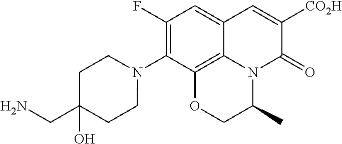 [SMILES: C[C@H]1COc2c(N3CCC(O)(CN)CC3)c(F)cc3cc(C(=O)O)c(=O)n1c23] | (S)-10-(4-(aminomethyl)-4- hydroxypiperidin-1-yl)-9- fluoro-3-methyl-5-oxo-2,3- dihydro-5H- [1,4]oxazino[2,3,4- ij]quinoline-6-carboxylic acid (HCl salt) | B | B, 1.43 | 392.62 |
| 1.69⁵ | 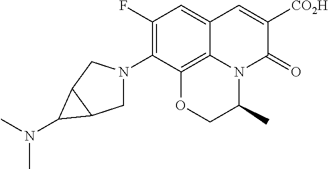 [SMILES: C[C@H]1COc2c(N3CC4C(C3)C4N(C)C)c(F)cc3cc(C(=O)O)c(=O)n1c23] | (3S)-10-(6- (dimethylamino)-3- azabicyclo[3.1.0]hexan-3- yl)-9-fluoro-3-methyl-5- oxo-2,3-dihydro-5H- [1,4]oxazino[2,3,4- ij]quinoline-6-carboxylic acid | B | B, 1.40 | 388.42 |
| 1.70⁵ | 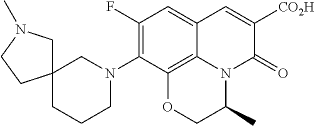 [SMILES: C[C@H]1COc2c(N3CCCC4(CCN(C)C4)C3)c(F)cc3cc(C(=O)O)c(=O)n1c23] | (3S)-9-fluoro-3-methyl-10- (2-methyl-2,7- diazaspiro[4.5]decan-7-yl)- 5-oxo-2,3-dihydro-5H- [1,4]oxazino[2,3,4- ij]quinoline-6-carboxylic acid (formate salt) | B | B, 1.38 | 416.52 |
| 1.71⁵ | 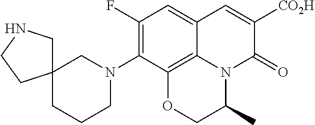 [SMILES: C[C@H]1COc2c(N3CCCC4(CCNC4)C3)c(F)cc3cc(C(=O)O)c(=O)n1c23] | (3S)-9-fluoro-3-methyl-5- oxo-10-(2,7- diazaspiro[4.5]decan-7-yl)- 2,3-dihydro-5H- [1,4]oxazino[2,3,4- ij]quinoline-6-carboxylic acid (formate salt) | B | B, 1.38 | 402.52 |
| 1.72 | 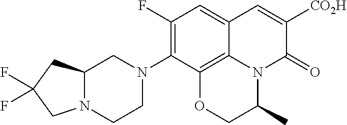 [SMILES: C[C@H]1COc2c(N3CCN4CC(F)(F)C[C@H]4C3)c(F)cc3cc(C(=O)O)c(=O)n1c23] | (S)-10-((S)-7,7- difluorohexahydropyrrolo [1,2-a]pyrazin-2(1H)-yl)-9- fluoro-3-methyl-5-oxo-2,3- dihydro-5H- [1,4]oxazino[2,3,4- ij]quinoline-6-carboxylic acid | B | B, 1.84 | 424.52 |
| 1.73 | 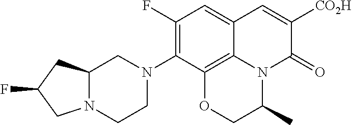 [SMILES: C[C@H]1COc2c(N3CCN4C[C@@H](F)C[C@H]4C3)c(F)cc3cc(C(=O)O)c(=O)n1c23] | (S)-9-fluoro-10-((7S,8aS)- 7- fluorohexahydropyrrolo[1,2- a]pyrazin-2(1H)-yl)-3- methyl-5-oxo-2,3-dihydro- 5H-[1,4]oxazino[2,3,4- ij]quinoline-6-carboxylic acid | B | B, 1.32 | 406.44 |
| 1.74 | 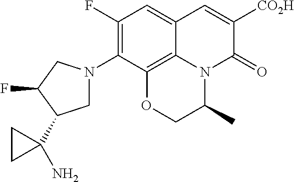 [SMILES: C[C@H]1COc2c(N3C[C@H](F)[C@@H](C4(N)CC4)C3)c(F)cc3cc(C(=O)O)c(=O)n1c23] | (S)-10-((3R,4R)-3-(1- aminocyclopropyl)-4- fluoropyrrolidin-1-yl)-9- fluoro-3-methyl-5-oxo-2,3- dihydro-5H- [1,4]oxazino[2,3,4- ij]quinoline-6-carboxylic acid (HCl salt) | A | B, 1.35 | 406.40 |
| 1.75 | 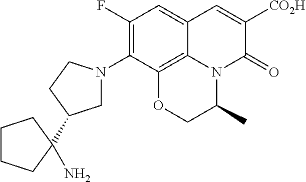 [SMILES: C[C@H]1COc2c(N3CC[C@@H](C4(N)CCCC4)C3)c(F)cc3cc(C(=O)O)c(=O)n1c23] | (S)-10-((R)-3-(1- aminocyclopentyl)pyrrolidin- 1-yl)-9-fluoro-3-methyl- 5-oxo-2,3-dihydro-5H- [1,4]oxazino[2,3,4- ij]quinoline-6-carboxylic acid (HCl salt) | B | B, 1.40 | 416.5 |
| 1.76 | 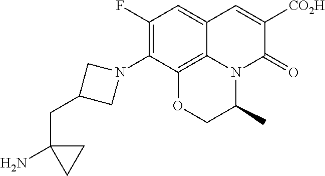 [SMILES: C[C@H]1COc2c(N3CC(CC4(N)CC4)C3)c(F)cc3cc(C(=O)O)c(=O)n1c23] | (S)-10-(3-((1- aminocyclopropyl)methyl) azetidin-1-yl)-9-fluoro-3- methyl-5-oxo-2,3-dihydro- 5H-[1,4]oxazino[2,3,4- ij]quinoline-6-carboxylic acid (formate salt) | B | B, 1.43 | 388.5 |
| 1.77³ | 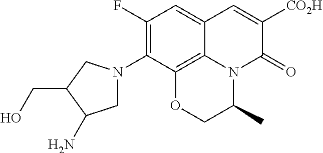 [SMILES: C[C@H]1COc2c(N3CC(N)C(CO)C3)c(F)cc3cc(C(=O)O)c(=O)n1c23] | (3S)-10-(3-amino-4- (hydroxymethyl)pyrrolidin- 1-yl)-9-fluoro-3-methyl-5- oxo-2,3-dihydro-5H- [1,4]oxazino[2,3,4- ij]quinoline-6-carboxylic acid (TFA salt) | B | B, 1.31 | 378.21 |
| 1.78³ | 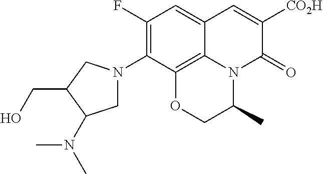 [SMILES: C[C@H]1COc2c(N3CC(CO)C(N(C)C)C3)c(F)cc3cc(C(=O)O)c(=O)n1c23] | (3S)-10-(3-(dimethylamino)-4- (hydroxymethyl)pyrrolidin-1- yl)-9-fluoro-3-methyl-5-oxo-2,3- dihydro-5H-[1,4]oxazino[2,3,4- ij]quinoline-6-carboxylic acid (TFA salt) | B | B, 1.33 | 406.2 |
| 1.79 | 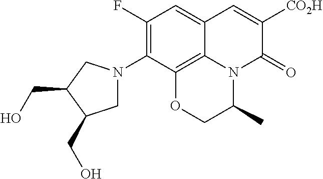 [SMILES: C[C@H]1COc2c(N3C[C@@H](CO)[C@@H](CO)C3)c(F)cc3cc(C(=O)O)c(=O)n1c23] | (S)-10-((3R,4S)-3,4- bis(hydroxymethyl)pyrrolidin- 1-yl)-9-fluoro-3-methyl- 5-oxo-2,3-dihydro-5H- [1,4]oxazino[2,3,4- ij]quinoline-6-carboxylic acid | B | A, 1.62 | 393.2 |

¹Hydrolysis (step viii) not performed
²Amine deprotection (step ix) not performed
³Single compound, absolute stereochemistry unknown.
⁴Stereochemistry assigned by analogy to exemple 1.5.
⁵Mixture of diastereoisomers.

HPLC method A: Characterized by high performance liquid chromatography (HPLC) on a Waters ACQUITY UPLC system with 1.2 mL/min flow rate; column Kinetex-C18, 2.6 um, 2.1×50 mm from Phenomenex, column temperature: 50° C.; gradient: 2-88% MeCN in water with 0.1% TFA over a 9.29 min period (unless indicated otherwise); compounds were detected by ultraviolet light (UV) absorption at 220 nm.

HPLC method B: Characterized by high performance liquid chromatography (HPLC) on a Waters ACQUITY H class UPLC system with 0.55 mL/min flow rate; column BEH-C18, 1.7 um, 2.1×50 mm from Waters, column temperature: ambiant; gradient (solvent A is 2 mM Ammonium Acetate and 0.1% Formic Acid in Water, solvent B is 0.1% Formic Acid in acetonitrile) : 5% solvent B hold for 0.4 min, 5-40% solvent B over 0.6 min, 40-60% solvent B over 1.2 min, 60-100% solvent B over 2.3 min then 100% solvent B over 3 min; compounds were detected by ultraviolet light (UV) absorption at 236 nm.

HPLC method C: Characterized by high performance liquid chromatography (HPLC) on a Agilent 1290 infinity RRLC system with 1 mL/min flow rate; column ZORBAX SB C8, 5 um, 250×4.6 mm from Agilent, column temperature: ambient; gradient (solvent A is 0.1% Formic Acid in Water, solvent B is 0.1% Formic Acid in acetonitrile): 10-30% solvent B over 25 min, 30-100% solvent B over 5 min, then 100% solvent B over 5 min; compounds were detected by ultraviolet light (UV) absorption at 238 nm.

### Example 2

### (S)-10-((R)-3-(1-aminocyclopropyl)pyrrolidin-1-yl)-9-fluoro-3-(hydroxymethyl)-5-oxo-2,3-dihydro-5H-[1,4]oxazino[2,3,4-ij]quinoline-6-carboxylic acid formate salt

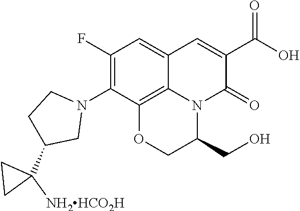

The title compound was prepared in accordance to the following scheme:

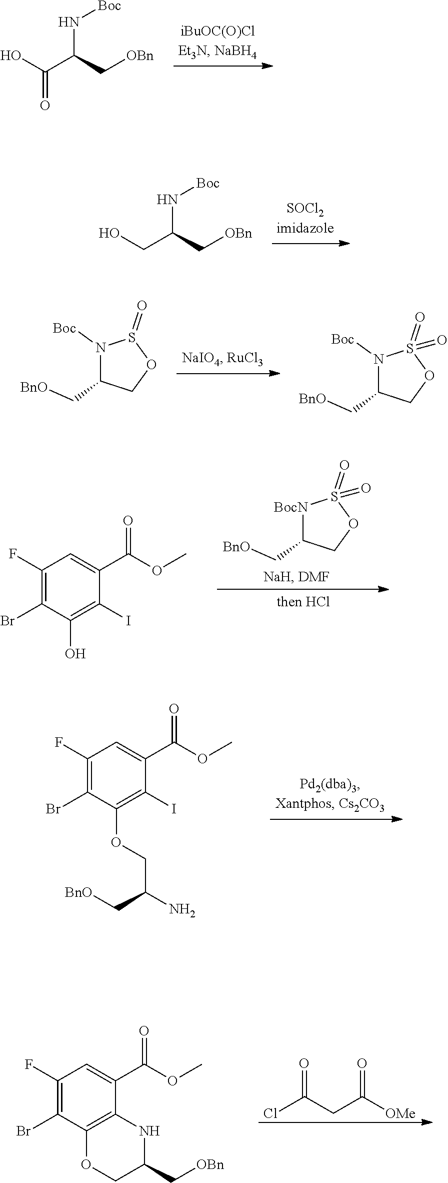

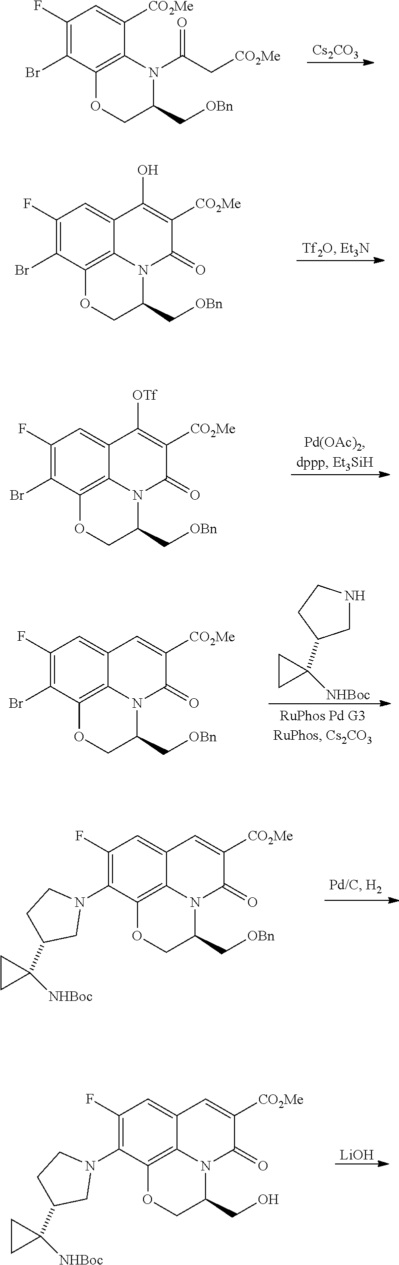

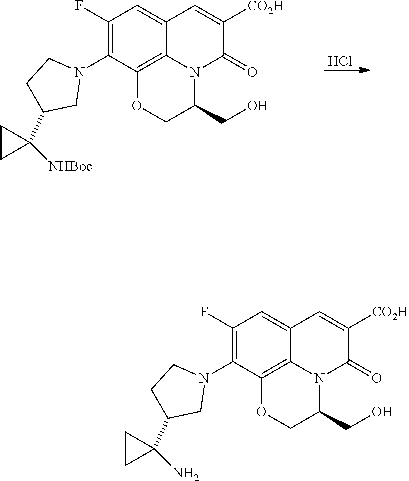 [SMILES: CC(C)(C)OC(=O)NC1([C@@H]2CCN(c3c(F)cc4cc(C(=O)O)c(=O)n5c4c3OC[C@@H]5CO)C2)CC1 ; Cl ; NC1([C@@H]2CCN(c3c(F)cc4cc(C(=O)O)c(=O)n5c4c3OC[C@@H]5CO)C2)CC1]

(i). tert-Butyl (S)-(1-(benzyloxy)-3-hydroxypropan-2-yl)carbamate: O-Benzyl-N-(tert-butoxycarbonyl)-L-serine (22.0 g, 74.6 mmol) was dissolved in dry THF (500 mL) and cooled to 0° C. Et₃N (22.6 g, 31.2 mL, 224 mmol) and isobutylchloroformate (15.4 g, 112 mmol) were added at 0° C. and the reaction mixture was stirred at rt for 1 h. In a separate flask sodium borohydride (14.0 g, 373 mmol) was added to water (500 mL) at 0-5° C. The first reaction mixture was slowly added to the ice-cold flask containing NaBH₄ in water and the reaction mixture was stirred at rt for 16 h. The reaction mixture was quenched with cold water and extracted with EtOAc. The organic layer was washed with water, brine, dried over sodium sulfate and concentrated under vacuum to afford a crude residue which was purified by silica gel column chromatography (3-5% ethyl acetate in hexane), affording the desired product (9.00 g, 43% yield) as a yellow solid. ¹H NMR (400 MHz, MeOD) δ 7.29 (m, 5H), 4.61 (s, 2H), 3.75 (m, 1H), 3.6 (m, 2H), 3.5 (m, 2H), 1.45 (s, 9H).

(ii). tert-Butyl (4S)-4-((benzyloxy)methyl)-1,2,3-oxathiazolidine-3-carboxylate 2-oxide: Imidazole (12.3 g, 182 mmol) was dissolved in DCM (250 mL) and the solution was cooled to 0° C. Thionyl chloride (6.47 g, 54.5 mmol) in DCM (38 mL) was added dropwise and the resulting suspension was stirred at rt for 1 h. The reaction mixture was cooled to −78° C., and a solution of tert-butyl (S)-(1-(benzyloxy)-3-hydroxypropan-2-yl)carbamate (8.50 g, 30.3 mmol) in DCM (97 mL) was added over a period of 1 h. The resulting mixture was stirred at rt for 16 h. The mixture was filtered through celite and washed with DCM. The organic layer was washed with water, brine, dried over sodium sulfate and concentrated under vacuum affording the title compound (9.50 g, 95% yield) as a yellow gum which was directly used in the next step without any further purification. LCMS (m/z): 328.4 [M+2].

(iii). tert-Butyl (S)-4-((benzyloxy)methyl)-1,2,3-oxathiazolidine-3-carboxylate 2,2-dioxide:

tert-Butyl (4S)-4-((benzyloxy)methyl)-1,2,3-oxathiazolidine-3-carboxylate 2-oxide (9.50 g, 29.0 mmol) was dissolved in MeCN (257 mL) and cooled to 0° C. Sodium periodate (31.5 g, 145 mmol), RuCl₃ (0.06 g, 0.29 mmol) were added followed by slow addition of water (176 mL). The reaction mixture was stirred at 0° C. for 30 minutes. The mixture was diluted with water and extracted with EtOAc. The organic layer was washed with water, brine, dried over sodium sulfate and concentrated under vacuum to afford a crude residue which was dissolved in Et₂O and filtered through a pad of silica to afford the desired product (8.00 g, 80% yield) as a white solid. ¹H NMR (400 MHz, CDCl₃) δ 7.4 (m, 5H), 4.62-4.57 (m, 4H), 4.43 (m, 1H), 4.2 (m, 1H), 3.77-3.64 (m, 2H), 1.62 (s, 9H).

(iv). Methyl (S)-3-(3-(benzyloxy)-2-((tert-butoxycarbonyl)amino)propoxy)-4-bromo-5-fluoro-2-iodobenzoate: To a solution of methyl 4-bromo-5-fluoro-3-hydroxy-2-iodobenzoate (9.20 g, 24.7 mmol) in anhydrous DMF (552 mL) was added sodium hydride (1.18 g, 29.6 mmol) portionwise at −10° C. After the addition, the resulting slurry was further stirred at rt for 15 minutes. tert-Butyl (S)-4-((benzyloxy)methyl)-1,2,3-oxathiazolidine-3-carboxylate 2,2-dioxide (7.12 g, 24.7 mmol) in DMF (92 mL) was added dropwise at 0° C. and the mixture was stirred at rt for 2 h. The reaction mixture was quenched with cold water, acidified with 1N HCl and extracted with EtOAc. The organic layer was washed with water, brine, dried over sodium sulfate and concentrated under vacuum. The residue was dissolved in DCM (60 mL) and cooled to 0° C. 4N HCl in dioxane (40 mL) was added and the reaction mixture was stirred at rt for 2 h. The reaction mixture was concentrated under vacuum to afford a crude residue which was purified by silica gel chromatography (0-5% MeOH in DCM), affording the desired product (3.80 g, 46% yield). ¹H NMR (400 MHz, MeOD) δ 7.40 (m, 5H), 4.60 (s, 2H), 4.18-4.09 (m, 2H), 3.80-3.60 (m, 2H), 3.55 (m, 1H). LCMS (m/z): 540.20 [M+2].

(v).Methyl (S)-3-((benzyloxy)methyl)-8-bromo-7-fluoro-3,4-dihydro-2H-benzo [b][1,4]oxazine-5-carboxylate: To a solution of methyl (S)-3-(3-(benzyloxy)-2-((tert-butoxycarbonyl)amino)propoxy)-4-bromo-5-fluoro-2-iodobenzoate (3.80 g, 7.06 mmol) in anhydrous dioxane (50 mL) was added cesium carbonate (6.88 g, 21.2 mmol). Pd₂(dba)₃ (0.32 g, 0.35 mmol), XantPhos (0.61 g, 1.06 mmol) were added and reaction mixture was stirred at 60° C. for 16 h. The crude reaction mixture was filtered through a celite pad and washed with excess of EtOAc. The filtrate was concentrated under vaccum to afford a crude residue which was purified by silica gel column chromatography (0-2% ethyl acetate in hexane), affording the desired product (2.20 g, 76% yield) as a yellow solid. LCMS (m/z): 412.24 [M+2].

(vi). Methyl (S)-3-((benzyloxy)methyl)-8-bromo-7-fluoro-4-(3-methoxy-3-oxopropanoyl)-3,4-dihydro-2H-benzo[b][1,4]oxazine-5-carboxylate: To a solution of methyl (S)-3-((benzyloxy)methyl)-8-bromo-7-fluoro-3,4-dihydro-2H-benzo [b][1,4]oxazine-5-carboxylate (2.10 g, 5.12 mmol) in anhydrous toluene (40 mL) was added methyl 3-chloro-3-oxo-propionate (2.78 g, 20.5 mmol). After addition, the mixture was stirred at 60° C. for 6 h. The reaction mixture was quenched with water and extracted with EtOAc. The organic layer was washed with saturated NaHCO₃, brine, dried over sodium sulfate and concentrated under vacuum to afford the desired product (2.40 g, 92% yield) which was directly used in the next step without any further purification. LCMS (m/z): 512.24 [M+2].

(vii). Methyl (S)-3-((benzyloxy)methyl)-10-bromo-9-fluoro-7-hydroxy-5-oxo-2,3-dihydro-5H-[1,4]oxazino[2,3,4-ij]quinoline-6-carboxylate: To a solution of methyl (S)-3-((benzyloxy)methyl)-8-bromo-7-fluoro-4-(3-methoxy-3-oxopropanoyl)-3,4-dihydro-2H-benzo[b][1,4]oxazine-5-carboxylate (2.40 g, 4.70 mmol) in anhydrous MeCN (40 mL) was added cesium carbonate (4.60 g, 14.1 mmol). After addition, the mixture was stirred at 60° C. for 3 h. The reaction mixture was quenched with cold water, acidified with 1N HCl to pH 3 and extracted with EtOAc. The organic layer was dried over sodium sulfate and concentrated under vacuum to afford a crude residue which was purified by trituration with n-pentane, affording the desired product (2.20 g, 98% yield) as a beige solid. LCMS (m/z): 480.2 [M+2].

(viii). Methyl (S)-3-((benzyloxy)methyl)-10-bromo-9-fluoro-5-oxo-7-(((trifluoro methyl)sulfonyl)oxy)-2,3-dihydro-5H-[1,4]oxazino[2,3,4-ij]quinoline-6-carboxylate: To a solution of methyl (S)-3-((benzyloxy)methyl)-10-bromo-9-fluoro-7-hydroxy-5-oxo-2,3-dihydro-5H-[1,4]oxazino[2,3,4-ij]quinoline-6-carboxylate (2.00 g, 4.18 mmol) in DCM (40 mL) was added triethylamine (3.81 g, 5.20 mL, 37.7 mmol). After addition, the solution was cooled to −78° C. Trifluoromethanesulfonic anhydride (5.30 g, 18.8 mmol) was added dropwise and the reaction mixture was stirred for 10 minutes at −78° C. The reaction mixture was concentrated under vacuum to afford a brown gum (2.50 g) which was directly used in the next step without any further purification. LCMS (m/z): 612.2 [M+2].

(ix). Methyl (S)-3-((benzyloxy)methyl)-10-bromo-9-fluoro-5-oxo-2,3-dihydro-5H-[1,4]oxazino[2,3,4-ij]quinoline-6-carboxylate: Methyl (S)-3-((benzyloxy)methyl)-10-bromo-9-fluoro-5-oxo-7-(((trifluoromethyl)sulfonyl)oxy)-2,3-dihydro-5H-[1,4]oxazino[2,3,4-ij]quinoline-6-carboxylate (2.50 g, 4.09 mmol) was dissolved in dry DMF (100 mL) and degassed with nitrogen for 5 minutes. DPPP (0.50 g, 1.23 mmol), Pd(II)OAc (0.14 g, 0.61 mmol) were added and the reaction mixture was further degassed for 5 minutes. The reaction mixture was cooled to 0° C. and triethylsilane (1.18 g, 10.2 mmol) was added dropwise. The reaction mixture was stirred at 60° C. for 6 h. The reaction mixture was quenched with cold water and extracted with EtOAc. The organic layer was washed with cold water, brine, dried over sodium sulfate and concentrated under vacuum to afford a crude residue which was purified by silica gel column chromatography (0-30% EtOAc in hexane), affording the desired product (1.20 g, 64% yield) as a yellow solid. ¹H NMR (400 MHz, MeOD) δ 8.6 (s, 1H), 7.36 (d, J=8.4, 1H), 7.33-7.26 (m, 5H), 5.20 (m, 1H), 5.00 (m, 1H), 4.58 (m, 2H), 4.30 (m, 1H), 3.93 (s, 3H), 3.73 (m, 2H). LCMS (m/z): 464.2 [M+2].

(x). Methyl (S)-3-((benzyloxy)methyl)-10-((R)-3-(1-((tert-butoxycarbonyl) amino)cyclopropyl)pyrrolidin-1-yl)-9-fluoro-5-oxo-2,3-dihydro-5H-[1,4]oxazino[2,3,4-ij]quinoline-6-carboxylate: To a solution of methyl (S)-3-((benzyloxy)methyl)-10-bromo-9-fluoro-5-oxo-2,3-dihydro-5H-[1,4]oxazino[2,3,4-ij]quinoline-6-carboxylate (0.50 g, 1.08 mmol) in toluene (10 mL) was added tert-butyl (R)-(1-(pyrrolidin-3-yl)cyclopropyl)carbamate (0.29 g, 1.29 mmol) and cesium carbonate (1.00 g, 3.25 mmol), followed by RuPhos (0.14 g, 0.16 mmol) and RuPhos.PdG3 (0.08 g, 0.16 mmol). After the addition the reaction mixture was heated to 90° C. for 5 h. The crude reaction mixture was filtered through celite and washed with excess of EtOAc. The filtrate was concentrated under vacuum to afford a crude residue which was purified by silica gel flash chromatography (30-35% EtOAc in hexane), affording the desired product (0.75 g) as an orange gum. LCMS (m/z): 608.26 [M+1].

(xi). Methyl (S)-10-((R)-3-(1-((tert-butoxycarbonyl)amino)cyclopropyl)pyrrolidin-1-yl)-9-fluoro-3-(hydroxymethyl)-5-oxo-2,3-dihydro-5H-[1,4]oxazino[2,3,4-ij]quinoline-6-carboxylate: 10% Palladium on carbon (50% in water, 0.70 g) was added to a solution of methyl (S)-3-((benzyloxy)methyl)-10-((R)-3-(1-((tert-butoxycarbonyl) amino)cyclopropyl)pyrrolidin-1-yl)-9-fluoro-5-oxo-2,3-dihydro-5H-[1,4]oxazino[2,3,4-ij]quinoline-6-carboxylate (0.70 g, 1.15 mmol) in methanol (10 mL). The slurry was stirred at rt under a H₂ atmosphere (1 atm) for 3 h. The reaction mixture was filtered through celite and concentrated under vacuum to afford the desired product (0.45 g, 75% yield). LCMS (m/z): 518.2 [M+1].

(xii). (S)-10-((R)-3-(1-((tert-butoxycarbonyl)amino)cyclopropyl)pyrrolidin-1-yl)-9-fluoro-3-(hydroxymethyl)-5-oxo-2,3-dihydro-5H-[1,4]oxazino[2,3,4-ij]quinoline-6-carboxylic acid: Methyl (S)-10-((R)-3-(1-((tert-butoxycarbonyl)amino)cyclopropyl)pyrrolidin-1-yl)-9-fluoro-3-(hydroxymethyl)-5-oxo-2,3-dihydro-5H-[1,4]oxazino[2,3,4-ij]quinoline-6-carboxylate (0.14 g, 0.27 mmol) was dissolved in MeOH (2 mL) and water (2 mL). 1M Lithium hydroxide in water (0.80 mL, 0.81 mmol) was added and the reaction mixture was stirred at rt for 2 h. The mixture was diluted with cold water, acidified with 1N HCl to pH 4 and extracted with EtOAc. The organic layer was dried over sodium sulfate and concentrated under vacuum to afford the desired product (0.13 g, 95% yield) as a yellow solid which was directly used in the next step without any further purification. LCMS (m/z): 504.4 [M+1].

(xiii). (S)-10-((R)-3-(1-aminocyclopropyl)pyrrolidin-1-yl)-9-fluoro-3-(hydroxymethyl)-5-oxo-2,3-dihydro-5H-[1,4]oxazino[2,3,4-ij]quinoline-6-carboxylic acid compound formic acid salt: (S)-10-((R)-3-(1-((tert-butoxycarbonyl)amino)cyclopropyl)pyrrolidin-1-yl)-9-fluoro-3-(hydroxymethyl)-5-oxo-2,3-dihydro-5H-[1,4]oxazino[2,3,4-ij]quinoline-6-carboxylic acid (0.13 g, 0.25 mmol) was dissolved in DCM (2 mL) and cooled to 0° C. 4N HCl in dioxane (1 mL) was added and the reaction mixture was stirred at rt for 4 h. The reaction mixture was concentrated under vacuum to afford a crude residue which was purified by preparative HPLC (formic acid buffer) to obtain the desired product (26.0 mg, 25% yield) as a yellow solid. ¹H NMR (400 MHz, DMSO) δ 8.25 (s, 1H), 7.52 (d, J=12 Hz, 1H), 4.83-4.78 (m, 2H), 4.06-4.03 (m, 1H), 4.00-3.90 (m, 1H), 3.80-3.70 (m, 1H), 3.63-3.50 (m, 3H), 3.48-3.40 (m, 2H), 2.09-2.00 (m, 1H), 1.87-1.85 (m, 1H), 1.74-1.69 (m, 1H), 0.53-0.50 (m, 4H). LCMS: t_{R}=1.36 min, m/z=404.34 [M+1] (3.5 minute run, method B).

### Example 3

### (S)-10-((R)-3-(1-aminocyclopropyl)pyrrolidin-1-yl)-9-fluoro-3-(methoxymethyl)-5-oxo-2,3-dihydro-5H-[1,4]oxazino[2,3,4-ij]quinoline-6-carboxylic acid formate salt

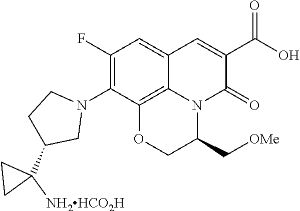

The title compound was prepared in accordance to the following scheme:

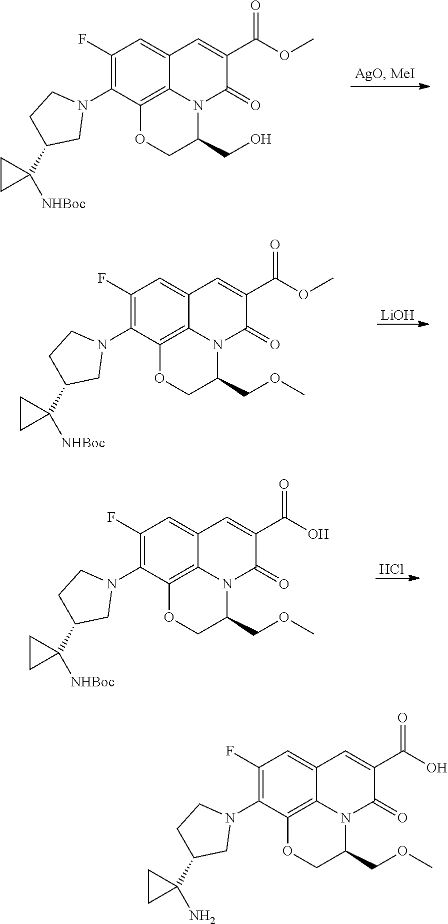 [SMILES: COC(=O)c1cc2cc(F)c(N3CC[C@@H](C4(NC(=O)OC(C)(C)C)CC4)C3)c3c2n(c1=O)[C@@H](CO)CO3 ; COC[C@H]1COc2c(N3CC[C@@H](C4(NC(=O)OC(C)(C)C)CC4)C3)c(F)cc3cc(C(=O)OC)c(=O)n1c23 ; "LiOH" ; COC[C@H]1COc2c(N3CC[C@@H](C4(NC(=O)OC(C)(C)C)CC4)C3)c(F)cc3cc(C(=O)O)c(=O)n1c23 ; Cl ; COC[C@H]1COc2c(N3CC[C@@H](C4(N)CC4)C3)c(F)cc3cc(C(=O)O)c(=O)n1c23]

(i). Methyl (S)-10-((R)-3-(1-((tert-butoxycarbonyl)amino)cyclopropyl)pyrrolidin-1-yl)-9-fluoro-3-(methoxymethyl)-5-oxo-2,3-dihydro-5H-[1,4]oxazino[2,3,4-ij]quinoline-6-carboxylate: Methyl (S)-10-((R)-3-(1-((tert-butoxycarbonyl)amino)cyclopropyl)pyrrolidin-1-yl)-9-fluoro-3-(hydroxymethyl)-5-oxo-2,3-dihydro-5H-[1,4]oxazino[2,3,4-ij]quinoline-6-carboxylate (0.48 g, 0.95 mmol) was dissolved in toluene (10 mL) and silver oxide (1.00 g, 4.64 mmol), methyl iodide (0.65 g, 4.64 mmol) were added. After addition, the reaction mixture was heated to 40° C. for 16 h. The crude reaction mixture was filtered through celite and washed with excess EtOAc. The filtrate was concentrated to afford a crude residue which was used in the next reaction without further purification. LCMS (m/z): 532.26 [M+1].

(ii). (S)-10-((R)-3-(1-((tert-butoxycarbonyl)amino)cyclopropyl)pyrrolidin-1-yl)-9-fluoro-3-(methoxymethyl)-5-oxo-2,3-dihydro-5H-[1,4]oxazino[2,3,4-ij]quinoline-6-carboxylic acid: Methyl (S)-10-((R)-3-(1-((tert-butoxycarbonyl)amino)cyclopropyl)pyrrolidin-1-yl)-9-fluoro-3-(methoxymethyl)-5-oxo-2,3-dihydro-5H-[1,4]oxazino[2,3,4-ij]quinoline-6-carboxylate (0.40 g, 0.75 mmol) was dissolved in MeOH (5 mL) and water (5 mL). 1M Lithium hydroxide in water (2.25 mL, 2.25 mmol) was added and the reaction mixture was stirred at rt for 2 h. The mixture was diluted with cold water, acidified with 1N HCl to pH 4 and extracted with EtOAc. The organic layer was dried over sodium sulfate and concentrated under vacuum to afford the desired product (0.20 g, 51% yield) as yellow solid which was directly used in the next step without any further purification. LCMS (m/z): 518.4 [M+1].

(iii). (S)-10-((R)-3-(1-aminocyclopropyl)pyrrolidin-1-yl)-9-fluoro-3-(methoxymethyl)-5-oxo-2,3-dihydro-5H-[1,4]oxazino[2,3,4-ij]quinoline-6-carboxylic acid: ((S)-10-((R)-3-(1-((tert-butoxycarbonyl)amino)cyclopropyl)pyrrolidin-1-yl)-9-fluoro-3-(methoxymethyl)-5-oxo-2,3-dihydro-5H-[1,4]oxazino[2,3,4-ij]quinoline-6-carboxylic acid (0.20 g, 0.39 mmol) was dissolved in DCM (1 mL) and cooled to 0° C. 4N HCl in dioxane (2 mL) was added and the reaction mixture was stirred at rt for 4 h. The reaction mixture was concentrated under vacuum to afford a crude residue which was purified by prep HPLC purification to obtain the desired product (58.0 mg, 36% yield) as a yellow solid. ¹H NMR (400 MHz, MeOD) δ 8.53 (s, 1H), 7.26 (d, J=13.2 Hz, 1H), 5.16-5.14 (m, 1H), 4.85-4.82 (m, 1H), 4.06-4.03 (m, 2H), 3.95-3.91 (m, 3H), 3.65-3.62 (m, 2H), 3.45 (s, 3H), 2.75-2.71 (m, 1H), 2.14-2.10 (m, 1H), 1.78-1.74 (m, 1H), 0.98-0.90 (m, 4H). LCMS: t_{R}=1.49 min, m/z=418.4 [M+1] (3.5 minute run, method B).

### Example 4

### (R)-10-((R)-3-(1-aminocyclopropyl)pyrrolidin-1-yl)-9-fluoro-3-(fluoromethyl)-5-oxo-2,3-dihydro-5H-[1,4]oxazino[2,3,4-ij]quinoline-6-carboxylic acid

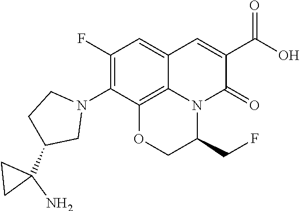 [SMILES: NC1([C@@H]2CCN(c3c(F)cc4cc(C(=O)O)c(=O)n5c4c3OC[C@@H]5CF)C2)CC1]

The title compound was prepared in accordance to the following scheme:

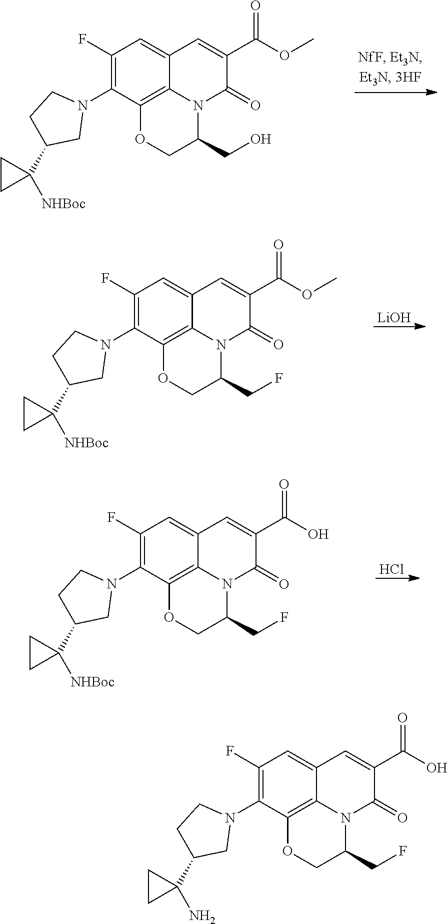 [SMILES: COC(=O)c1cc2cc(F)c(N3CC[C@@H](C4(NC(=O)OC(C)(C)C)CC4)C3)c3c2n(c1=O)[C@@H](CO)CO3 ; COC(=O)c1cc2cc(F)c(N3CC[C@@H](C4(NC(=O)OC(C)(C)C)CC4)C3)c3c2n(c1=O)[C@@H](CF)CO3 ; "LiOH" ; CC(C)(C)OC(=O)NC1([C@@H]2CCN(c3c(F)cc4cc(C(=O)O)c(=O)n5c4c3OC[C@@H]5CF)C2)CC1 ; Cl ; NC1([C@@H]2CCN(c3c(F)cc4cc(C(=O)O)c(=O)n5c4c3OC[C@@H]5CF)C2)CC1]

(i). Methyl (R)-10-((R)-3-(1-((tert-butoxycarbonyl)amino)cyclopropyl)pyrrolidin-1-yl)-9-fluoro-3-(fluoromethyl)-5-oxo-2,3-dihydro-5H-[1,4]oxazino[2,3,4-ij]quinoline-6-carboxylate: Methyl (S)-10-((R)-3-(1-((tert-butoxycarbonyl)amino)cyclopropyl)pyrrolidin-1-yl)-9-fluoro-3-(hydroxymethyl)-5-oxo-2,3-dihydro-5H-[1,4]oxazino[2,3,4-ij]quinoline-6-carboxylate (0.20 g, 0.39 mmol) was dissolved in MeCN (15 mL) and cooled to -40° C. Perfluoro-1-butanesulfonyl fluoride (0.23 g, 0.77 mmol), Et₃N (0.16 g, 0.21 mL, 1.55 mmol) and Et₃N.3HF (124 mg, 0.13 mL, 0.77 mmol) were added and the reaction mixture was stirred overnight at rt. The reaction mixture was diluted with ethyl acetate and washed with saturated NaHCO₃ solution, dried over Na₂SO₄ and concentrated under vacuum. The crude residue was purified by silica gel chromatography (30-35% EtOAc in hexane) affording the desired product (0.16 g, 80% yield) as a yellow solid. LCMS (m/z): 520.55 [M+1].

(ii). ((R)-10-((R)-3-(1-((tert-butoxycarbonyl)amino)cyclopropyl)pyrrolidin-1-yl)-9-fluoro-3-(fluoromethyl)-5-oxo-2,3-dihydro-5H-[1,4]oxazino[2,3,4-ij]quinoline-6-carboxylic acid: Methyl (R)-10-((R)-3-(1-((tert-butoxycarbonyl)amino)cyclopropyl)pyrrolidin-1-yl)-9-fluoro-3-(fluoromethyl)-5-oxo-2,3-dihydro-5H-[1,4]oxazino[2,3,4-ij]quinoline-6-carboxylate (0.16 g, 0.31 mmol) was dissolved in MeOH (3 mL) and water (1 mL). 1M Lithium hydroxide in water (0.92 mL, 0.92 mmol) was added and the reaction mixture was stirred at rt for 2 h. The mixture was diluted with cold water, acidified with 1N HCl to pH 4 and extracted with EtOAc. The combined organic layer was dried over sodium sulfate and concentrated under vacuum to afford the desired product (146 mg, 94% yield) as a yellow solid which was directly used in the next step without any further purification. LCMS (m/z): 506.52 [M+1].

(iii). (R)-10-((R)-3-(1-aminocyclopropyl)pyrrolidin-1-yl)-9-fluoro-3-(fluoromethyl)-5-oxo-2,3-dihydro-5H-[1,4]oxazino[2,3,4-ij]quinoline-6-carboxylic acid: ((R)-10-((R)-3-(1-((tert-butoxycarbonyl)amino)cyclopropyl)pyrrolidin-1-yl)-9-fluoro-3-(fluoromethyl)-5-oxo-2,3-dihydro-5H-[1,4]oxazino[2,3,4-ij]quinoline-6-carboxylic acid (146 mg, 0.29 mmol) was dissolved in DCM (4 mL) and cooled to 0° C. 4N HCl in dioxane (2 mL) was added and the reaction mixture was stirred at rt for 4 h. The reaction mixture was concentrated under vacuum to afford a crude residue which was purified by prep HPLC purification, affording the desired product (24.0 mg, 20% yield) as a yellow solid. ¹H NMR (400 MHz, MeOD) δ 8.54 (s, 1H), 7.25 (m, 1H), 5.24-5.00 (m, 2H), 4.83-4.78 (m, 2H), 4.63 (m, 1H), 4.10-4.06 (m, 1H), 4.00-3.90 (m, 1H), 3.91-3.57 (m, 3H), 2.44 (m, 1H), 2.18 (m, 1H), 1.77 (m, 1H), 0.89 (m, 4H). LCMS: t_{R}=1.50 min, m/z=406.34 [M+1] (3.5 minute run, method B).

### Example 5

### (S)-10-((R)-3-(1-aminocyclopropyl)pyrrolidin-1-yl)-9-fluoro-3-methyl-5-oxo-2,3-dihydro-5H-[1,4]oxazino[2,3,4-ij]quinoline-6-carbonitrile trifluoroacetate salt

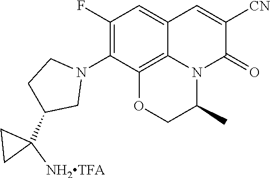

The title compound was prepared in accordance with the following scheme:

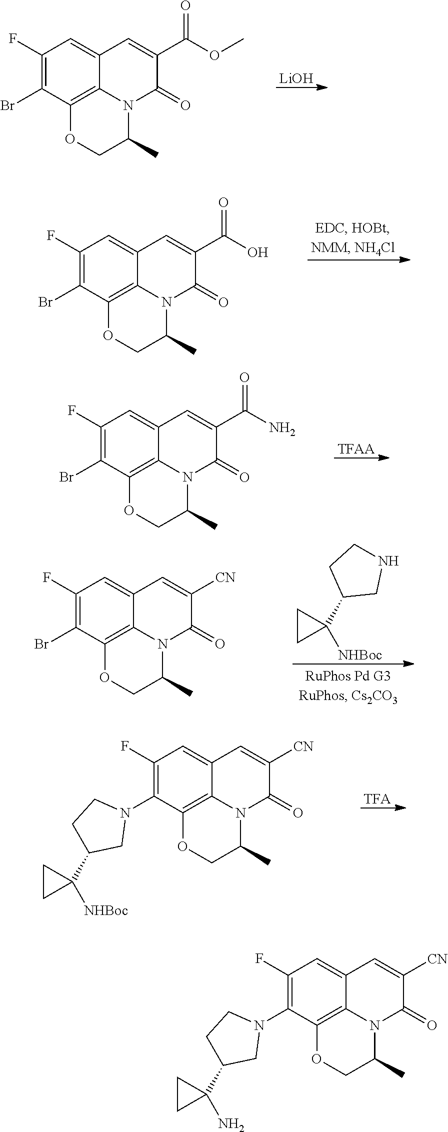

(i). (S)-10-bromo-9-fluoro-3-methyl-5-oxo-2,3-dihydro-5H-[1,4]oxazino[2,3,4-ij]quinoline-6-carboxylic acid: To a solution of methyl (S)-10-bromo-9-fluoro-3-methyl-5-oxo-2,3-dihydro-5H-[1,4]oxazino[2,3,4-ij]quinoline-6-carboxylate (0.40 g, 1.12 mmol) in MeOH:H₂O (3:1, 8 mL) was added LiOH.H₂O (1M in water, 6.75 mL, 6.75 mmol). The mixture was stirred at rt for 3 h. After completion, the mixture was concentrated under reduced pressure and diluted with cold water, acidified with HCl 1N to pH 2-3 and extracted with EtOAc. The organic layer was washed with brine, dried over sodium sulfate and concentrated under vacuum to afford the desired product (0.36 g, 72% yield) which was directly used in the next step without any further purification. ¹H NMR (400 MHz, DMSO) δ 14.18 (s, 1H), 8.90 (s, 1H), 7.78 (d, J=8.4 Hz, 1H), 5.09-5.06 (m, 1H), 4.72-4.69 (m, 1H), 4.33-4.30 (m, 1H), 1.33 (d, J=6.4 Hz, 3H). LCMS (m/z): 344.16 [M+2].

(ii). (S)-10-bromo-9-fluoro-3-methyl-5-oxo-2,3-dihydro-5H-[1,4]oxazino[2,3,4-ij]quinoline-6-carboxamide: (S)-10-bromo-9-fluoro-3-methyl-5-oxo-2,3-dihydro-5H-[1,4]oxazino[2,3,4-ij]quinoline-6-carboxylic acid (0.36 g, 1.07 mmol), EDC.HCl (0.25 g, 1.28 mmol) and HOBt (0.22 g, 1.61 mmol) were dissolved in DMF (8 mL) under nitrogen and allowed to stir at 0-5° C. for 15 minutes. N-methylmorpholine (0.54 g, 5.35 mmol) and NH₄Cl (0.12 g, 2.14 mmol) were added and the resulting mixture was stirred at rt for 2 h. After completion, the reaction mixture was quenched with ice cold water and extracted with EtOAc. The combined organic layer was washed with cold water, brine, dried over sodium sulfate and concentrated under vacuum to afford the desired product (0.30 g, 77% yield) which was directly used in the next step without any further purification. LCMS (m/z): 343.21 [M+2].

(iii). (S)-10-bromo-9-fluoro-3-methyl-5-oxo-2,3-dihydro-5H-[1,4]oxazino[2,3,4-ij]quinoline-6-carbonitrile: (S)-10-bromo-9-fluoro-3-methyl-5-oxo-2,3-dihydro-5H-[1,4]oxazino[2,3,4-ij]quinoline-6-carboxamide (0.30 g, 0.88 mmol), was dissolved in pyridine (3 mL) at 0° C. Trifluoroacetic anhydride (0.92 g, 4.41 mmol) was added slowly to the above solution and the mixture was stirred for 1 h at 0° C. The reaction mixture was quenched with 1N HCl and extracted by EtOAc. The combined organic layer was concentrated under vacuum to afford a crude residue which was purified by silica gel chromatography (100% hexane to 100% EtOAc) affording the desired product (0.20 g, 74% yield). ¹H NMR (400 MHz, DMSO) δ 8.20 (s, 1H), 7.12 (d, J=7.6 Hz, 1H), 5.18 (m, 1H), 4.65-4.62 (m, 1H), 4.27-4.23 (m, 1H), 1.49-1.47 (m, 3H). LCMS (m/z): 325.02 [M+2].

(iv). tert-Butyl (1-((R)-1-((S)-6-cyano-9-fluoro-3-methyl-5-oxo-2,3-dihydro-5H-[1,4]oxazino[2,3,4-ij]quinolin-10-yl)pyrrolidin-3-yl)cyclopropyl)carbamate: To (S)-10-bromo-9-fluoro-3-methyl-5-oxo-2,3-dihydro-5H-[1,4]oxazino[2,3,4-ij]quinoline-6-carbonitrile (0.20 g, 0.62 mmol), tert-butyl (R)-(1-(pyrrolidin-3-yl)cyclopropyl)carbamate (0.17 g, 0.74 mmol) and Cs₂CO₃ (0.51 g, 1.55 mmol) in toluene (8 mL) were added RuPhos (43.0 mg, 0.09 mmol) and RuPhos.PdG₃ (26.0 mg, 0.03 mmol). The reaction mixture was then heated to 80° C. for 5 h. After completion, the reaction slurry was filtered through celite and washed with excess of EtOAc. The organic layer was concentrated under vacuum to afford a crude residue which was purified using silica gel chromatography (25-75% EtOAc in Hexane) affording the desired product (0.07 g, 24% yield) as a pale yellow gum. LCMS (m/z): 469.47 [M+1].

(v). (S)-10-((R)-3-(1-aminocyclopropyl)pyrrolidin-1-yl)-9-fluoro-3-methyl-5-oxo-2,3-dihydro-5H-[1,4]oxazino[2,3,4-ij]quinoline-6-carbonitrile: tert-butyl (1-((R)-1-((S)-6-cyano-9-fluoro-3-methyl-5-oxo-2,3-dihydro-5H-[1,4]oxazino[2,3,4-ij]quinolin-10-yl)pyrrolidin-3-yl)cyclopropyl)carbamate (0.05 g, 0.10 mmol) was dissolved in DCM (5 mL) and cooled to 0° C. TFA (0.5 mL) was added and the reaction mixture was stirred at rt for 2 h. The reaction mixture was concentrated under vacuum to afford a crude residue which was dissolved in water and lyophilized, affording the desired product (14.0 mg, 35% yield). ¹H NMR (400 MHz, Methanol-d₄) δ 8.34 (s, 1H), 7.17 (d, J=13.6 Hz, 1H), 5.02-4.93 (m, 1H), 4.51-4.48 (m, 1H), 4.06-3.95 (m, 2H), 3.79-3.70 (m, 3H), 2.61-2.56 (m, 1H), 2.15-2.09 (m, 1H), 1.73-1.68 (m, 1H), 1.40-1.37 (m, 1H), 1.00-0.97 (m, 4H). LCMS: t_{R}=1.43 min, m/z=369.42 [M+1] (3.5 minute run, method B).

### Example 6.1

### (S)-9-fluoro-3-methyl-10-((R)-3-(1-((2,2,2-trifluoroacetyl)-14-azanyl)cyclopropyl)pyrrolidin-1-yl)-2,3-dihydro-5H-[1,4]oxazino[2,3,4-ij]quinolin-5-one trifluoroacetate salt

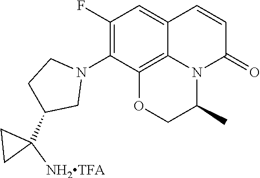

The title compound was prepared in accordance with the following scheme:

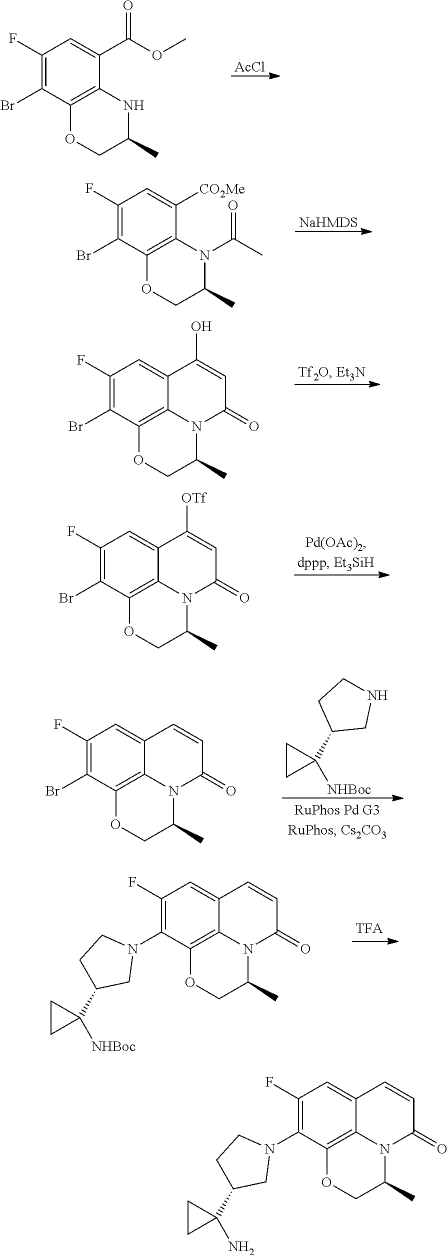

(i). Methyl (S)-4-acetyl-8-bromo-7-fluoro-3-methyl-3,4-dihydro-2H-benzo[b][1,4]oxazine-5-carboxylate: Methyl (S)-8-bromo-7-fluoro-3-methyl-3,4-dihydro-2H-benzo[b][1,4]oxazine-5-carboxylate (2.00 g, 6.57 mmol) was dissolved in toluene (20 mL). Acetyl chloride (1.50 g, 19.7 mmol) was added dropwise at rt and the reaction mixture was stirred at 60° C. for 4 h. The reaction mixture was quenched with water, neutralized with NaHCO₃ solution and extracted with EtOAc. The organic layer was washed with brine, dried over sodium sulfate and concentrated under vacuum to afford a crude residue which was purified by silica gel chromatography (0-50% EtOAc in Hexane) affording the desired product (1.70 g, 75% yield). ¹H NMR (400 MHz, CDCl₃) (2 rotamers presents) δ 7.41 (d, J=8.6 Hz, 1H), 7.21 (d, J=8.4 Hz, 1H), 5.35-5.33 (m, 2H), 4.46-4.31 (m, 4H), 3.90 (d, J=18.7 Hz, 6H), 2.27 (d, J=6.0 Hz, 3H), 1.99 (s, 3H), 1.39 (d, J=6.8 Hz, 3H), 1.17 (d, J=7.1 Hz, 3H). LCMS (m/z): 348.2 [M+2].

(ii). (S)-10-bromo-9-fluoro-7-hydroxy-3-methyl-2,3-dihydro-5H-[1,4]oxazino[2,3,4-ij]quinolin-5-one: Methyl (S)-4-acetyl-8-bromo-7-fluoro-3-methyl-3,4-dihydro-2H-benzo[b][1,4]oxazine-5-carboxylate (1.90 g, 5.54 mmol) was dissolved in dry THF (80 mL) and the reaction mixture was cooled to -78° C. NaHMDS (1.0 M solution in THF, 11.0 mL, 11.0 mmol) was added dropwise and the reaction mixture was stirred at -78° C. for 1 h. The reaction mixture was quenched with water at −78° C. and poured into water at rt. The aqueous layer was acidified with diluted HCl, the white precipitate was filtered, washed with cool water and dried under vacuum to afford the desired product (1.40 g, 81% yield). LCMS (m/z): 316.2 [M+2].

(iii). (S)-10-bromo-9-fluoro-3-methyl-5-oxo-2,3-dihydro-5H-[1,4]oxazino[2,3,4-ij]quinolin-7-yl trifluoromethanesulfonate: To (S)-10-bromo-9-fluoro-7-hydroxy-3-methyl-2,3-dihydro-5H-[1,4]oxazino[2,3,4-ij]quinolin-5one (1.40 g, 4.45 mmol) in DCM (15 mL) was added Et₃N (2.70 g, 3.72 mL, 26.8 mmol) followed by cooling to −78° C. Trifluoromethanesulfonic anhydride (5.00 g, 17.8 mmol) was added dropwise and the reaction mixture was stirred at −78° C. for 30 minutes. The reaction mixture was diluted with EtOAc and washed with cool water, brine, dried over sodium sulfate and concentrated under vacuum to afford a crude residue which was purified by silica gel chromatography (0-30% EtOAc in Hexane), affording the desired product (1.40 g, 70% yield). ¹H NMR (400 MHz, CDCl₃) δ 7.23 (d, J=8.1 Hz, 1H), 6.87-6.79 (m, 1H), 5.16 (d, J=5.3 Hz, 1H), 4.62 (s, 1H), 4.25 (d, J=9.5 Hz, 1H), 1.48 (d, J=6.7 Hz, 3H). LCMS (m/z): 448.2 [M+2].

(iv). (S)-10-bromo-9-fluoro-3-methyl-2,3-dihydro-5H-[1,4]oxazino[2,3,4-ij]quinolin-5-one: (S)-10-bromo-9-fluoro-3-methyl-5-oxo-2,3-dihydro-5H-[1,4]oxazino[2,3,4-ij]quinolin-7-yl trifluoromethanesulfonate (0.70 g, 1.56 mmol) was dissolved in dry DMF (10 mL). DPPP (0.19 g, 0.47 mmol), Pd(II)OAc (0.05 g, 0.23 mmol) were added and the reaction mixture was degassed with nitrogen for 5 minutes. Triethylsilane (0.40 g, 3.92 mmol) was added and the reaction mixture was stirred at 60° C. for 1 h. The reaction mixture was quenched with cool water and extracted with EtOAc. The organic layer was washed with water, brine, dried over sodium sulfate and concentrated under vacuum to afford a crude residue which was purified by silica gel column chromatography (0-35% EtOAc in Hexane), affording the desired product (0.30 g, 64% yield). ¹H NMR (400 MHz, CDCl₃) δ 7.69-7.62 (m, 1H), 7.04 (d, J=8.1 Hz, 1H), 6.79 (t, J=8.5 Hz, 1H), 5.18-5.11 (m, 1H), 4.60 (d, J=11.4 Hz, 1H), 4.29-4.19 (m, 1H), 1.46 (d, J=6.7 Hz, 3H). LCMS (m/z): 300.2 [M+2].

(v). tert-Butyl (1-((R)-1-((S)-9-fluoro-3-methyl-5-oxo-2,3-dihydro-5H-[1,4]oxazino[2,3,4-ij]quinolin-10-yl)pyrrolidin-3-yl)cyclopropyl)carbamate: A microwave vial was charged with (S)-10-bromo-9-fluoro-3-methyl-2,3-dihydro-5H-[1,4]oxazino[2,3,4-ij]quinolin-5-one (73.0 mg, 0.24 mmol) and toluene (2 mL). tert-Butyl (S)-(1-(pyrrolidin-3-yl)cyclopropyl)carbamate (83.0 mg, 0.37 mmol), RuPhos (23.0 mg, 0.05 mmol), RuPhos Pd G3 (41.0 mg, 0.05 mmol) and cesium carbonate (239 mg, 0.74 mmol) were added. The mixture was heated to 90° C. for 4 h. The crude mixture was filtered through a disposable filter funnel and volatiles were evaporated under reduced pressure. Silica gel chromatography (0-100% EtOAc/heptane) provided the product (20.0 mg, 18% yield) as a yellow solid. LCMS (m/z): 444.5 [M+1].

(vi). (S)-10-((R)-3-(1-aminocyclopropyl)pyrrolidin-1-yl)-9-fluoro-3-methyl-5-oxo-2,3-dihydro-5H-[1,4]oxazino[2,3,4-ij]quinoline-6-carboxylic acid trifluoroacetate salt: tert-Butyl (1-((R)-1-((S)-9-fluoro-3-methyl-5-oxo-2,3-dihydro-5H-[1,4]oxazino[2,3,4-ij]quinolin-10-yl)pyrrolidin-3-yl)cyclopropyl)carbamate (20.0 mg, 0.04 mmol) was dissolved in DCM (0.5 mL) and TFA (0.5 mL) was added, followed by stirring at rt for 1 h. The solvent was evaporated and the crude material was purified on reverse phase HPLC, affording the product (8.00 mg, 27% yield) as a yellow solid. ¹H NMR (400 MHz, Methanol-d₄) δ 7.76 (d, J=9.5 Hz, 1H), 7.05 (d, J=12.8 Hz, 1H), 6.51 (d, J=9.4 Hz, 1H), 5.00 (q, J=6.5 Hz, 1H), 4.48 (d, J=11.3 Hz, 1H), 4.07 (dd, J=11.3, 2.0 Hz, 1H), 3.79-3.49 (m, 4H), 2.48 (p, J=7.4 Hz, 1H), 2.23-2.08 (m, 1H), 1.78 (dq, J=12.3, 8.6 Hz, 1H), 1.41-1.29 (m, 3H), 1.04-0.90 (m, 4H). LCMS: t_{R}=1.70 min, m/z=344.4 [M+1] (10 minute run, method A).

Using the procedures described for Example 6.1 the following compound was prepared:

| Ex | | | HPLC, | LCMS |
|---|---|---|---|---|
| # | Structure | Chemical Name | t_{R} (min) | [M + 1] |
| 6.2 | 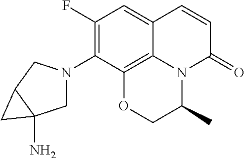 [SMILES: C[C@H]1COc2c(N3CC4CC4(N)C3)c(F)cc3ccc(=O)n1c23] | (3S)-10-(1-amino-3- azabicyclo[3.1.0]hexan-3-yl)-9- fluoro-3-methyl-2,3-dihydro-5H- [1,4]oxazino[2,3,4-ij]quinolin-5- one hydrochloride salt (HCl salt) | 1.38 | 316.21 |

HPLC method: Characterized by high performance liquid chromatography (HPLC) on a Waters ACQUITY H class UPLC system with 0.55 mL/min flow rate; column BEH-C18, 1.7 um, 2.1×50 mm from Waters, column temperature: ambiant; gradient (solvent A is 2 mM Ammonium Acetate and 0.1% Formic Acid in Water, solvent B is 0.1% Formic Acid in acetonitrile) : 5% solvent B hold for 0.4 min, 5-40% solvent B over 0.6 min, 40-60% solvent B over 1.2 min, 60-100% solvent B over 2.3 min then 100% solvent B over 3 min; compounds were detected by ultraviolet light (UV) absorption at 236 nm.

### Example 7.1

### (3S)-7-(aminomethyl)-10-(3-(aminomethyl)-3-fluoropyrrolidin-1-yl)-9-fluoro-3-methyl-2,3-dihydro-5H-[1,4]oxazino[2,3,4-ij]quinolin-5-one trifluoroacetate salt

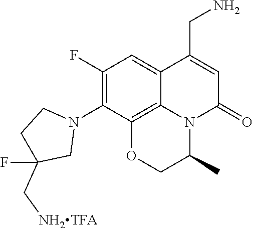

The title compound was prepared in accordance with the following scheme:

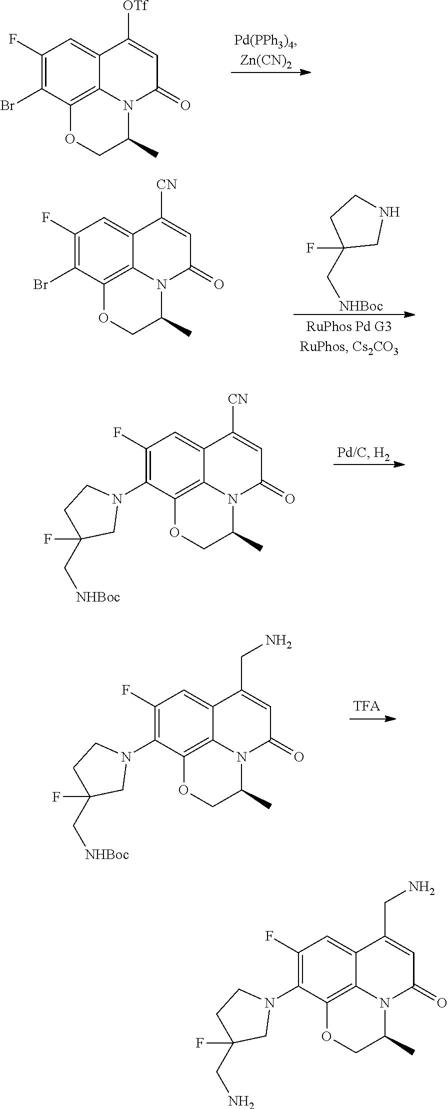

(i). (S)-10-bromo-9-fluoro-3-methyl-5-oxo-2,3-dihydro-5H-[1,4]oxazino[2,3,4-ij]quinoline-7-carbonitrile: A solution of (S)-10-bromo-9-fluoro-3-methyl-5-oxo-2,3-dihydro-5H-[1,4]oxazino[2,3,4-ij]quinolin-7-yl trifluoromethanesulfonate (255 mg, 0.57 mmol) in DMF (5.5 mL) was treated with Zinc(II) cyanide (34.9 mg, 0.30 mmol) and palladium tetrakistriphenylphosphine (66.0 mg, 0.06 mmol). The mixture was heated to 80° C. overnight. After completion of the reaction, the slurry was quenched with aqueous NaHCO₃ and extracted with EtOAc. The organic extract was washed with saturated aqueous NaHCO₃, brine, dried over Na₂SO₄, filtered and concentrated under reduced pressure. Silica gel chromatography (0-60% EtOAc/heptane) provided the product (132 mg, 72% yield) as a yellow solid. ¹H NMR (400 MHz, Chloroform-d) δ 7.35 (d, J=7.9 Hz, 1H), 7.18 (s, 1H), 5.16-5.07 (m, 1H), 4.65-4.55 (m, 1H), 4.20 (dd, J=11.5, 2.3 Hz, 1H), 1.43 (d, J=6.7 Hz, 3H). LCMS (m/z): 325.0 [M+2].

(ii).tert-Butyl ((1-((S)-7-cyano-9-fluoro-3-methyl-5-oxo-2,3-dihydro-5H-[1,4]oxazino[2,3,4-ij]quinolin-10-yl)-3-fluoropyrrolidin-3-yl)methyl)carbamate: A microwave vial was charged with (S)-10-bromo-9-fluoro-3-methyl-5-oxo-2,3-dihydro-5H-[1,4]oxazino[2,3,4-ij]quinoline-7-carbonitrile (70.0 mg, 0.22 mmol) and toluene (2 mL). tert-Butyl ((3-fluoropyrrolidin-3-yl)methyl)carbamate (110 mg, 0.50 mmol), RuPhos (20.0 mg, 0.04 mmol), RuPhos Pd G3 (36.0 mg, 0.04 mmol) and cesium carbonate (212 mg, 0.65 mmol) were added. The mixture was heated at 90° C. overnight. The crude mixture was filtered through a disposable filter funnel and volatiles were evaporated under reduced pressure. Silica gel chromatogrpahy (0-100% EtOAc/heptane) provided the product (87.0 mg, 87% yield) as a yellow solid. LCMS (m/z): 461.2 [M+1].

(iii). tert-Butyl ((1-((S)-7-(aminomethyl)-9-fluoro-3-methyl-5-oxo-2,3-dihydro-5H-[1,4]oxazino[2,3,4-ij]quinolin-10-yl)-3-fluoropyrrolidin-3-yl)methyl)carbamate: A mixture of tert-butyl ((1-((S)-7-cyano-9-fluoro-3-methyl-5-oxo-2,3-dihydro-5H-[1,4]oxazino[2,3,4-ij]quinolin-10-yl)-3-fluoropyrrolidin-3-yl)methyl)carbamate (85.0 mg, 0.18 mmol) in 2M NH₃ in MeOH (2.5 mL) was treated with Pd/C (10% dry wt, 50% water, 118 mg, 0.06 mmol). The flask was partially evacuated and back-filled with H₂ 5 times, then stirred under a balloon of hydrogen for 1 h. After completion, the reaction mixture was passed through a syringe filter and concentrated under reduced pressure to give the product as a yellow oil. LCMS (m/z): 465.3 [M+1].

(iv). (3 S)-7-(aminomethyl)-10-(3-(aminomethyl)-3-fluoropyrrolidin-1-yl)-9-fluoro-3-methyl-2,3-dihydro-5H-[1,4]oxazino[2,3,4-ij]quinolin-5-one trifluoroacetate salt: tert-butyl ((1-((S)-7-(aminomethyl)-9-fluoro-3-methyl-5-oxo-2,3-dihydro-5H-[1,4]oxazino[2,3,4-ij]quinolin-10-yl)-3-fluoropyrrolidin-3-yl)methyl)carbamate (86.0 mg, 0.18 mmol) was dissolved in DCM (1 mL) and TFA (1 mL) was added. The mixture was then stirred at rt for 30 minutes. The solvent was evaporated and the crude material purified on reverse phase HPLC, affording the product (46.0 mg, 42% yield) as a yellow solid. ¹H NMR (400 MHz, Methanol-d₄) δ 7.27 (d, J=14.1 Hz, 1H), 6.61 (s, 1H), 5.13-5.00 (m, 1H), 4.55 (d, J=11.3 Hz, 1H), 4.41 (s, 2H), 4.25-4.02 (m, 3H), 3.87-3.65 (m, 2H), 3.51 (ddd, J=20.5, 8.1, 1.5 Hz, 2H), 2.47-2.07 (m, 2H), 1.40 (d, J=5.8 Hz, 3H). LCMS: t_{R}=1.02 min, m/z=365.5 [M+1] (10 minute run, method A).

Using the procedures described for Example 7.1 the following compounds were prepared:

| | | | Method, | |
|---|---|---|---|---|
| Ex | | | LCMS | LCMS |
| # | Structure | Chemical Name | t_{R} (min) | [M + 1] |
| 7.2 | 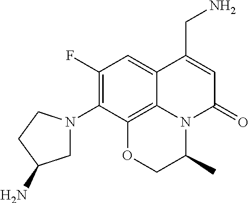 [SMILES: C[C@H]1COc2c(N3CC[C@H](N)C3)c(F)cc3c(CN)cc(=O)n1c23] | (S)-7-(aminomethyl)-10-((S)-3- aminopyrrolidin-1-yl)-9-fluoro- 3-methyl-2,3-dihydro-5H- [1,4]oxazino[2,3,4-ij]quinolin-5- one (TFA salt) | A, 0.78 | 333.2 |
| 7.3 | 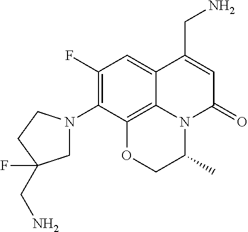 [SMILES: C[C@@H]1COc2c(N3CCC(F)(CN)C3)c(F)cc3c(CN)cc(=O)n1c23] | (3R)-7-(aminomethyl)-10-(3- (aminomethyl)-3- fluoropyrrolidin-1-yl)-9-fluoro-3- methyl-2,3-dihydro-5H- [1,4]oxazino[2,3,4-ij]quinolin-5- one (TFA salt) | A, 0.37 | 365.4 |
| 7.4 | 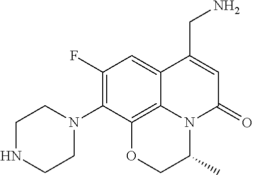 [SMILES: C[C@@H]1COc2c(N3CCNCC3)c(F)cc3c(CN)cc(=O)n1c23] | (R)-7-(aminomethyl)-9-fluoro-3- methyl-10-(piperazin-1-yl)-2,3- dihydro-5H-[1,4]oxazino[2,3,4- ij]quinolin-5-one (TFA salt) | A, 0.21 | 333.3 |
| 7.5 | 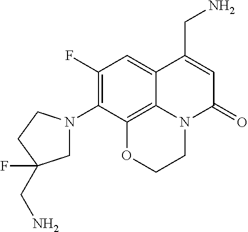 [SMILES: NCc1cc(=O)n2c3c(c(N4CCC(F)(CN)C4)c(F)cc13)OCC2] | 7-(aminomethyl)-10-(3- (aminomethyl)-3- fluoropyrrolidin-1-yl)-9-fluoro- 2,3-dihydro-5H- [1,4]oxazino[2,3,4-ij]quinolin-5- one (TFA salt) | A, 0.24 | 351.3 |
| 7.6¹ | 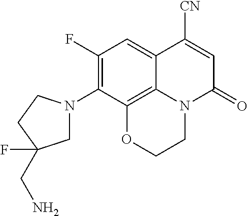 [SMILES: N#Cc1cc(=O)n2c3c(c(N4CCC(F)(CN)C4)c(F)cc13)OCC2] | 10-(3-(aminomethyl)-3- fluoropyrrolidin-1-yl)-9-fluoro-5- oxo-2,3-dihydro-5H- [1,4]oxazino[2,3,4-ij]quinoline- 7-carbonitrile (TFA salt) | — | 347.3 |
| 7.7 | 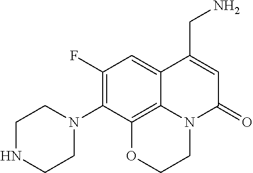 [SMILES: NCc1cc(=O)n2c3c(c(N4CCNCC4)c(F)cc13)OCC2] | 7-(aminomethyl)-9-fluoro-10- (piperazin-1-yl)-2,3-dihydro-5H- [1,4]oxazino[2,3,4-ij]quinolin-5- one (TFA salt) | A, 0.19 | 319.3 |
| 7.8¹ | 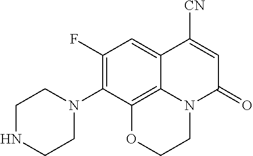 [SMILES: N#Cc1cc(=O)n2c3c(c(N4CCNCC4)c(F)cc13)OCC2] | 9-fluoro-5-oxo-10-(piperazin-1- yl)-2,3-dihydro-5H- [1,4]oxazino[2,3,4-ij]quinoline- 7-carbonitrile (TFA salt) | — | 315.3 |
| 7.9 | 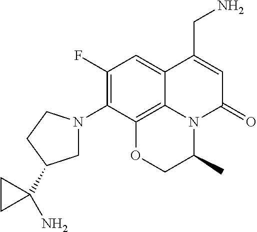 [SMILES: C[C@H]1COc2c(N3CC[C@@H](C4(N)CC4)C3)c(F)cc3c(CN)cc(=O)n1c23] | (S)-10-((R)-3-(1- aminocyclopropyl)pyrrolidin-1- yl)-7-(aminomethyl)-9-fluoro-3- methyl-2,3-dihydro-5H- [1,4]oxazino[2,3,4-ij]quinolin-5- one (formate salt) | B, 1.24 | 373.33 |
| 7.10 | 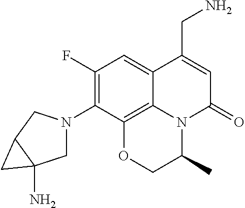 [SMILES: C[C@H]1COc2c(N3CC4CC4(N)C3)c(F)cc3c(CN)cc(=O)n1c23] | (3S)-10-(1-amino-3- azabicyclo[3.1.0]hexan-3-yl)-7- (aminomethyl)-9-fluoro-3- methyl-2,3-dihydro-5H- [1,4]oxazino[2,3,4-ij]quinolin-5- one (TFA salt) | B, 2.33 | 345.3 |

¹Step (iii) not performed

HPLC method A: Characterized by high performance liquid chromatography (HPLC) on a Waters ACQUITY UPLC system with 1.2 mL/min flow rate; column Kinetex-C18, 2.6 um, 2.1×50 mm from Phenomenex, column temperature: 50° C.; gradient: 2-88% MeCN in water with 0.1% TFA over a 9.29 min period (unless indicated otherwise); compounds were detected by ultraviolet light (UV) absorption at 220 nm.

HPLC method B: Characterized by high performance liquid chromatography (HPLC) on a Waters ACQUITY H class UPLC system with 0.55 mL/min flow rate; column BEH-C18, 1.7 um, 2.1×50 mm from Waters, column temperature: ambiant; gradient (solvent A is 2 mM Ammonium Acetate and 0.1% Formic Acid in Water, solvent B is 0.1% Formic Acid in acetonitrile): 5% solvent B hold for 0.4 min, 5-40% solvent B over 0.6 min, 40-60% solvent B over 1.2 min, 60-100% solvent B over 2.3 min then 100% solvent B over 3 min; compounds were detected by ultraviolet light (UV) absorption at 236 nm.

### Example 8

### 10-((R)-3-(1-aminocyclopropyl)pyrrolidin-1-yl)-9-fluoro-3-methyl-5-oxo-2,3-dihydro-1H,5H-pyrido[3,2,1-ij]quinoline-6-carboxylic acid trifluoroacetate salt

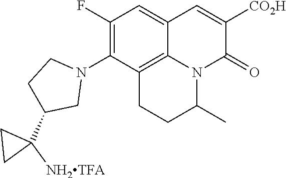

The title compound was prepared in accordance with the following scheme:

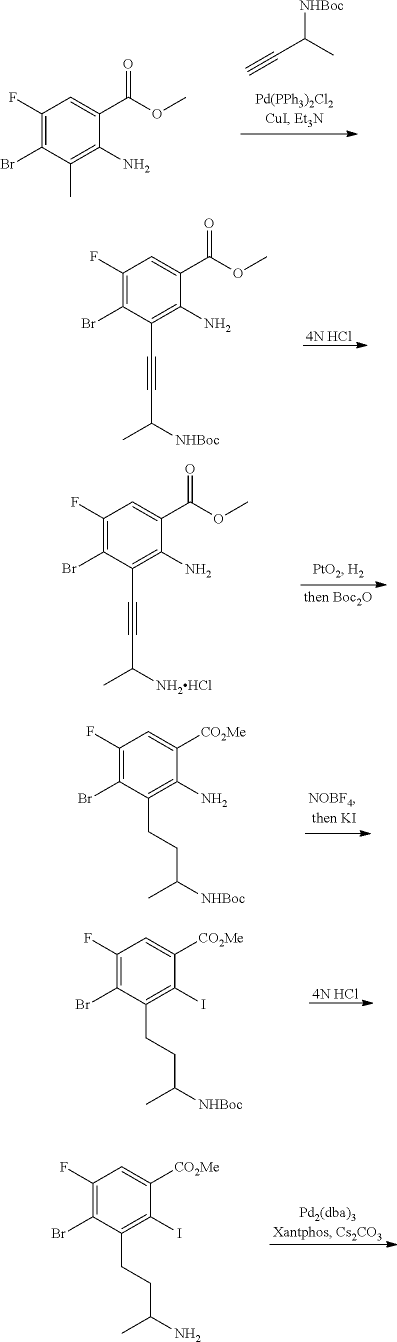

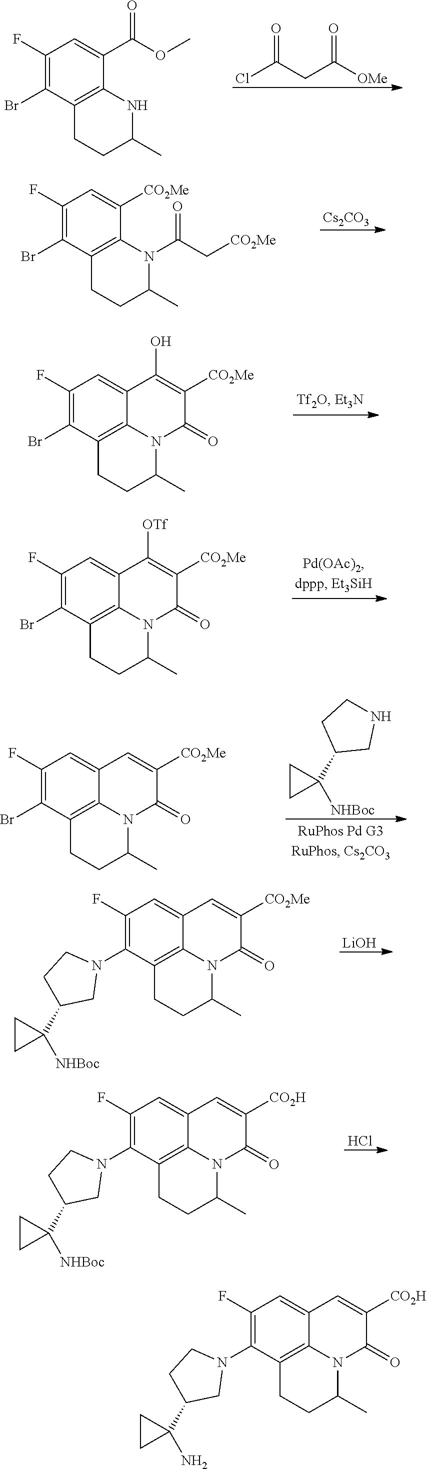

(i). Methyl 2-amino-4-bromo-3-(3-((tert-butoxycarbonyl)amino)but-1-yn-1-yl)-5-fluorobenzoate: A mixture of methyl 2-amino-4-bromo-5-fluoro-3-iodobenzoate (3.30 g, 8.82 mmol), tert-butyl but-3-yn-2-ylcarbamate (1.87 g, 11.0 mmol), copper(I) iodide (0.08 g, 0.44 mmol) and bis(triphenylphosphine)palladium(II) chloride (0.62 g, 0.88 mmol) in Et₃N (30 mL) was stirred at 65° C. for 2 h. The mixture was then cooled down, diluted with ethyl acetate, filtered, concentrated and purified by silica gel chromatography (100% heptane to 100% ethyl acetate), yielding the desired product (2.95 g, 85% yield). LCMS (m/z): 417.4 [M+2].

(ii). Methyl 2-amino-3-(3-aminobut-1-yn-1-yl)-4-bromo-5-fluorobenzoate: To methyl 2-amino-4-bromo-3-(3-((tert-butoxycarbonyl)amino)but-1-yn-1-yl)-5-fluorobenzoate (2.90 g, 6.98 mmol) in MeOH (20 mL) was added 4N HCl in dioxane (17.5 mL, 69.8 mmol) and the resulting solution was stirred at rt for 3 h. The solvent was evaporated, and the resulting crude product was used directly in the subsequent step without further purification. LCMS (m/z): 318.3 [M+2].

(iii). Methyl 2-amino-4-bromo-3-(3-((tert-butoxycarbonyl)amino)butyl)-5-fluorobenzoate: To a degassed solution of methyl 2-amino-3-(3-aminobut-1-yn-1-yl)-4-bromo-5-fluorobenzoate (2.46 g, 6.98 mmol) in EtOH (20 mL) was added Platinum(IV) oxide (0.32 g, 1.40 mmol). The reaction vessel was stirred under an hydrogen atmosphere for 1.5 h. After completion, the solvent was evaporated and THF (20 mL) was added, followed by Et₃N (3.18 g, 4.38 mL, 31.4 mmol) and Boc anhydride (2.75 g, 12.6 mmol). The resulting suspension was stirred at rt for 1 h. The slurry was then filtered, the solvent evaporated and the crude material purified by silica gel chromatography (100% heptane to 100% ethyl acetate) affording the desired product (1.72 g, 65% yield). LCMS (m/z): 421.3 [M+2].

(iv). Methyl 4-bromo-3-(3-((tert-butoxycarbonyl)amino)butyl)-5-fluoro-2-iodobenzoate: Methyl 2-amino-4-bromo-3-(3-((tert-butoxycarbonyl)amino)butyl)-5-fluorobenzoate (500 mg, 1.19 mmol) in DCM (6 mL) was added to a stirred suspension of nitrosonium tetrafluoroborate (279 mg, 2.39 mmol) at −20° C. The mixture was stirred at this temperature for 1 h. The DCM was evaporated and the resulting diazonium salt was immediately dissolved in acetonitrile (12 mL). To this solution was added dibenzo-18-crown-6 (43.0 mg, 0.12 mmol) and potassium iodide (990 mg, 5.96 mmol). The suspension was stirred at rt for 30 minutes and then at 65° C. for 14 h. The reaction mixture was filtered and the solvent removed. The crude mixture was purified by silica gel chromatography (100% heptane to 100% ethyl acetate) affording the desired product (228 mg, 36% yield). LCMS (m/z): 532.3 [M+2].

(v). Methyl 3-(3-aminobutyl)-4-bromo-5-fluoro-2-iodobenzoate hydrochloride: Methyl 4-bromo-3-(3-((tert-butoxycarbonyl)amino)butyl)-5-fluoro-2-iodobenzoate (300 mg, 0.57 mmol) was dissolved in DCM/MeOH (4/4 mL) and 4N HCl in dioxane (1.42 mL, 5.66 mmol) was added. The resulting solution was stirred at rt for 2 h. The solvent was evaporated and the product was used in the next step without further purification. LCMS (m/z): 432.2 [M+2].

(vi). Methyl 5-bromo-6-fluoro-2-methyl-1,2,3,4-tetrahydroquinoline-8-carboxylate: To a solution of methyl 3-(3-aminobutyl)-4-bromo-5-fluoro-2-iodobenzoate hydrochloride (251 mg, 0.54 mmol) in anhydrous dioxane (2 ml) was added Pd₂(dba)₃ (49.0 mg, 0.05 mmol), xantphos (93.0 mg, 0.16 mmol) and cesium carbonate (701 mg, 2.15 mmol). The mixture was stirred at 90° C. overnight. The suspension was filtered and concentrated in vacuo. The crude material was purified by silica gel chromatography (100% heptane to 40% EtOAc in heptane), affording the product (130 mg, 80% yield). LCMS (m/z): 304.3 [M].

(vii). Methyl 5-bromo-6-fluoro-1-(3-methoxy-3-oxopropanoyl)-2-methyl-1,2,3,4-tetrahydroquinoline-8-carboxylate: To a solution of methyl 5-bromo-6-fluoro-2-methyl-1,2,3,4-tetrahydroquinoline-8-carboxylate (300 mg, 0.99 mmol) in anhydrous toluene (4 mL) was added methyl 3-chloro-3-oxo-propionate (542 mg, 3.97 mmol), followed by stirring at 60° C. for 1 h. The solvent was evaporated and the crude material was purified by silica gel chromatography (0-100% EtOAc/heptane), providing the desired product (399 mg, 99% yield). LCMS (m/z): 404.0 [M+2].

(viii). Methyl 10-bromo-9-fluoro-7-hydroxy-3-methyl-5-oxo-2,3-dihydro-1H,5H-pyrido[3,2,1-ij]quinoline-6-carboxylate: To a solution of methyl 5-bromo-6-fluoro-1-(3-methoxy-3-oxopropanoyl)-2-methyl-1,2,3,4-tetrahydroquinoline-8-carboxylate (310 mg, 0.77 mmol) in acetonitrile (6 mL) was added cesium carbonate (753 mg, 2.31 mmol). The mixture was then stirred at 60° C. for 1 h. The reaction mixture was quenched with water and acidified with 1N HCl solution to adjust the pH to 2-3. EtOAc was added, and the phases were separated. The aqueous layer was extracted with EtOAc, and the organic layer was dried on Na₂SO₄, filtered and concentrated. The crude material was purified by silica gel chromatography (100% heptane to 100% EtOAc), affording the desired product (285 mg, 99% yield). ¹H NMR (400 MHz, Methanol-d₄) δ 7.79 (d, J=8.6 Hz, 1H), 5.28-5.19 (m, 1H), 3.97 (s, 3H), 3.36-3.29 (m, 1H), 3.15 (dd, J=17.8, 4.9 Hz, 1H), 2.98 (ddd, J=18.1, 13.8, 5.8 Hz, 1H), 2.24-2.14 (m, 1H), 1.96 (tt, J=13.8, 5.0 Hz, 1H), 1.23 (d, J=6.7 Hz, 3H). LCMS (m/z): 372.2 [M+2].

(ix). Methyl 10-bromo-9-fluoro-3-methyl-5-oxo-7-(((trifluoromethyl)sulfonyl)oxy)-2,3-dihydro-1H,5H-pyrido[3,2,1-ij]quinoline-6-carboxylate: To a solution of methyl 10-bromo-9-fluoro-7-hydroxy-3-methyl-5-oxo-2,3-dihydro-1H,5H-pyrido[3,2,1-ij]quinoline-6-carboxylate (300 mg, 0.81 mmol) in DCM (6 mL) was added triethylamine (246 mg, 0.34 mL, 2.43 mmol) and triflic anhydride (686 mg, 0.41 mL, 2.43 mmol) at 0° C. The reaction was completed after 15 minutes. The solvent was evaporated, affording the product as a dark brown oil. LCMS (m/z): 502.2 [M].

(x).Methyl 10-bromo-9-fluoro-3-methyl-5-oxo-2,3-dihydro-1H,5H-pyrido[3,2,1-ij]quinoline-6-carboxylate: Crude methyl 10-bromo-9-fluoro-3-methyl-5-oxo-7-(((trifluoromethyl)sulfonyl)oxy)-2,3-dihydro-1H,5H-pyrido[3,2,1-ij]quinoline-6-carboxylate (400 mg, 0.80 mmol) was dissolved in dry DMF (4 mL). DPPP (99.0 mg, 0.24 mmol), palladium(II) acetate (27.0 mg, 0.12 mmol) and triethylsilane (232 mg, 0.32 mL, 1.99 mmol) were added sequentially under N₂ purging. The reaction mixture was stirred at 60° C. for 4 h. The crude mixture was diluted with EtOAc and extracted with water. The organic layer was washed with water, dried on Na₂SO₄ and filtered. The solvent was evaporated, and the crude residue was purified by silica gel chromatography (0-100% EtOAc/Hexane) to afford a yellow solid as the desired product (141 mg, 50% yield, 2 steps) with some amount of des-bromo product present. ¹H NMR (500 MHz, Methanol-d₄) δ 8.52 (s, 1H), 7.59 (d, J=8.1 Hz, 1H), 5.40-5.23 (m, 1H), 3.93 (s, 3H), 3.21 (dd, J=17.8, 4.9 Hz, 1H), 3.04 (ddd, J=18.2, 13.8, 5.7 Hz, 1H), 2.30-2.23 (m, 1H), 2.11-1.97 (m, 1H), 1.30 (d, J=6.7 Hz, 3H). LCMS (m/z): 356.2 [M+2].

(xi). Methyl 10-((R)-3-(1-((tert-butoxycarbonyl)amino)cyclopropyl)pyrrolidin-1-yl)-9-fluoro-3-methyl-5-oxo-2,3-dihydro-1H,5H-pyrido[3,2,1-ij]quinoline-6-carboxylate: A microwave vial was charged with methyl 10-bromo-9-fluoro-3-methyl-5-oxo-2,3-dihydro-1H,5H-pyrido[3,2,1-ij]quinoline-6-carboxylate (138 mg, 0.39 mmol) and toluene (4 mL). tert-Butyl (S)-(1-(pyrrolidin-3-yl)cyclopropyl)carbamate (176 mg, 0.78 mmol), RuPhos (18.0 mg, 0.04 mmol), RuPhos Pd G3 (33.0 mg, 0.04 mmol) and cesium carbonate (381 mg, 1.17 mmol) were added. The mixture was heated to 100° C. for 1.5 h. The crude mixture was filtered through a disposable filter funnel and volatiles were evaporated under reduced pressure. Silica gel chromatography (0-100% EtOAc/heptane) provided the diastereomeric mixture (117 mg) as a yellow solid. The mixture was further separated by SFC (Column: AS 21×250 mm, CO₂/EtOH=85/15, flow rate 100 ml/min) to afford the desired diastereoisomer (relative stereochemistry not determined, 55 mg, 28% yield). LCMS (m/z): 500.3 [M+1].

(xii). 10-((R)-3-(1-((tert-butoxycarbonyl)amino)cyclopropyl)pyrrolidin-1-yl)-9-fluoro-3-methyl-5-oxo-2,3-dihydro-1H,5H-pyrido[3,2,1-ij]quinoline-6-carboxylic acid: Methyl 10-((R)-3-(1-((tert-butoxycarbonyl)amino)cyclopropyl)pyrrolidin-1-yl)-9-fluoro-3-methyl-5-oxo-2,3-dihydro-1H,5H-pyrido[3,2,1-ij]quinoline-6-carboxylate (55.0 mg, 0.11 mmol) was dissolved in MeOH (1 mL) and water (1 mL). Lithium hydroxide hydrate (18.5 mg, 0.44 mmol) was added at rt. After stirring for 3 h the mixture was diluted with water, and treated with 1 M HCl. The aqueous layer was extracted with EtOAc, and the organic extract was washed with brine, dried over Na₂SO₄, filtered and concentrated under reduced pressure to afford the desired product. LCMS (m/z): 486.3 [M+1].

(xi). (S)-10-((R)-3-(1-aminocyclopropyl)pyrrolidin-1-yl)-9-fluoro-3-methyl-5-oxo-2,3-dihydro-5H-[1,4]oxazino[2,3,4-ij]quinoline-6-carboxylic acid hydrochloride salt: Crude (S)-10-((R)-3-(1-((tert-butoxycarbonyl)amino)cyclopropyl)pyrrolidin-1-yl)-9-fluoro-3-methyl-5-oxo-2,3-dihydro-5H-[1,4]oxazino[2,3,4-ij]quinoline-6-carboxylic acid (12.0 mg, 0.03 mmol) was dissolved in DCM (1 mL) and HCl 4N in dioxane (1 mL) was added. The resulting mixture was stirred at rt for 2 h. The solvent was evaporated, and the crude residue was purified by Prep HPLC purification (TFA buffer) affording the desired product (4.50 mg, 36% yield). ¹H NMR (400 MHz, Methanol-d₄) δ 8.78 (s, 1H), 7.58 (d, J=12.5 Hz, 1H), 5.36-5.20 (m, 1H), 3.91-3.84 (m, 1H), 3.76 (td, J=8.8, 3.6 Hz, 1H), 3.19 (q, J=7.7 Hz, 1H), 3.09-2.94 (m, 3H), 2.84-2.72 (m, 1H), 2.27-2.15 (m, 2H), 1.99-1.82 (m, 2H), 1.37 (d, J=6.7 Hz, 3H), 0.97 (dd, J=11.2, 4.3 Hz, 4H). LCMS: t_{R}=2.19 min, m/z=386.6 [M+1] (10 minute run, method A).

### Example 9.1

### 9-((R)-3-(1-aminocyclopropyl)pyrrolidin-1-yl)-8-fluoro-2-methyl-4-oxo-1,2-dihydro-4H-pyrrolo[3,2,1-ij]quinoline-5-carboxylic acid hydrochloride salt

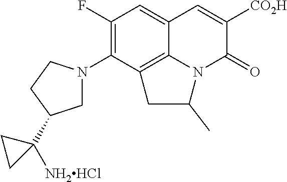

The title compound was prepared in accordance with the following scheme:

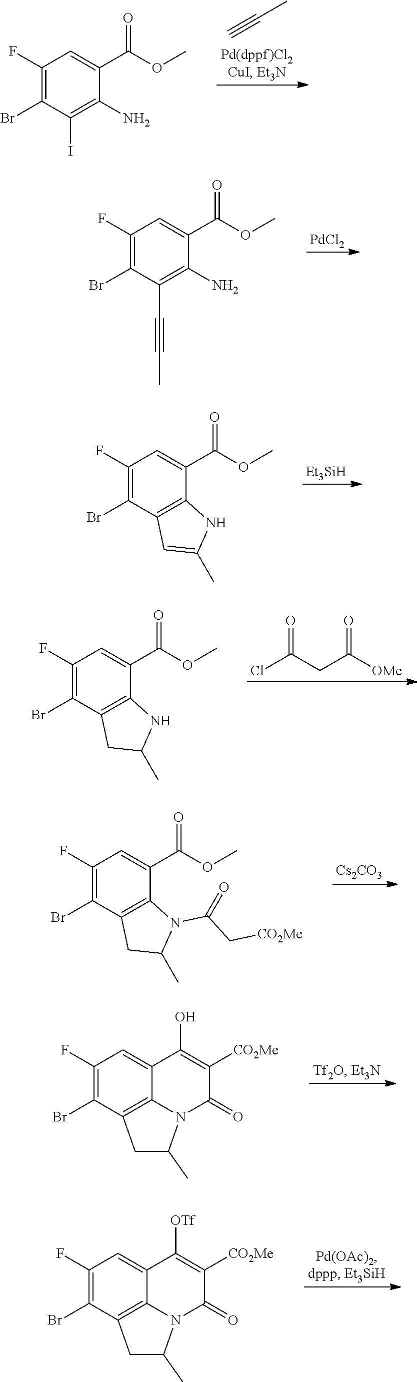

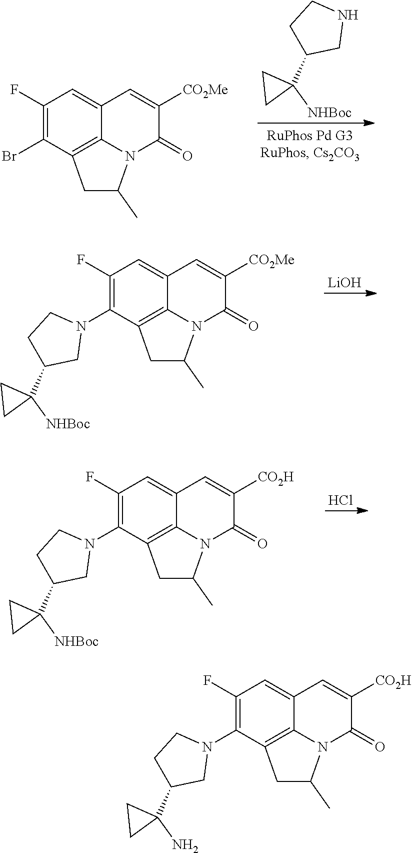

(i). Methyl 2-amino-4-bromo-5-fluoro-3-(prop-1-yn-1-yl)benzoate: To a mixture of methyl 2-amino-4-bromo-5-fluoro-3-iodobenzoate (4.00 g, 10.7 mmol), copper(I) iodide (0.21 g, 1.07 mmol), (dppf)palladium(II) chloride dichloromethane adduct (0.44 g, 0.54 mmol) in Et₃N (30 mL) and DMF (20 mL) was bubbled propyne gas (excess, not quantified) for 10 minutes at 0° C. The reaction was tightly sealed and stirred at 70° C. for 24 h. The mixture was then cooled down to rt, filtered through celite, concentrated and purified by silica gel chromatography (100% heptane to 30% ethyl acetate in heptane), yielding the desired product (2.00 g, 65% yield). ¹H NMR (400 MHz, Chloroform-d) δ 7.56 (d, J=9.4 Hz, 1H), 6.36 (br s, 2H), 3.87 (s, 3H), 2.22 (s, 3H). LCMS (m/z): 288.2 [M+2].

(ii). Methyl 4-bromo-5-fluoro-2-methyl-1H-indole-7-carboxylate: To methyl 2-amino-4-bromo-5-fluoro-3-(prop-1-yn-1-yl)benzoate (2.50 g, 8.74 mmol) in acetonitrile (20 mL) was added palladium(II) chloride (0.04 g, 0.22 mmol) and the resulting mixture was stirred at reflux for 3 h. The reaction mixture was cooled down to rt, the solvent was evaporated, and the resulting crude product was purified by silica gel chromatography (100% heptane to 100% ethyl acetate), yielding the desired product (2.35 g, 94% yield). ¹H NMR (500 MHz, DMSO-d₆) δ 11.33 (s, 1H), 7.51 (d, J=9.7 Hz, 1H), 6.30 (s, 1H), 3.95 (s, 3H), 2.47 (s, 3H). LCMS (m/z): 288.1 [M+2].

(iii). Methyl 4-bromo-5-fluoro-2-methylindoline-7-carboxylate: To a suspension of methyl 4-bromo-5-fluoro-2-methyl-1H-indole-7-carboxylate (1.10 g, 3.84 mmol) in TFA (8 mL) was added triethylsilane (4.47 g, 6.14 mL, 38.4 mmol). The resulting mixture was stirred at 65° C. for 1 h. After completion the mixture was cooled down to rt, the solvent was evaporated, and the resulting crude product was diluted with EtOAc and extracted with NaHCO₃ saturated solution. The organic layer was further washed with water, dried on MgSO₄ and filtered. The solvent was evaporated, delivering the desired product (1.10 g, 99% yield). The product was used in the next step without further purification. LCMS (m/z): 290.2 [M+2].

(iv). Methyl 4-bromo-5-fluoro-1-(3-methoxy-3-oxopropanoyl)-2-methylindoline-7-carboxylate: To a solution of methyl 4-bromo-5-fluoro-2-methylindoline-7-carboxylate (1.10 g, 3.85 mmol) in anhydrous toluene (16 mL) was added methyl 3-chloro-3-oxo-propionate (2.10 g, 15.4 mmol). The mixture was stirred at 60° C. for 1 h. The mixture was cooled down to rt, diluted with EtOAc and extracted with NaHCO₃ saturated solution. The organic layer was further washed with water, dried on MgSO₄ and filtered. The solvent was evaporated, and the crude product was used in the next step without further purification. LCMS (m/z): 390.3 [M+2].

(v).Methyl 9-bromo-8-fluoro-6-hydroxy-2-methyl-4-oxo-1,2-dihydro-4H-pyrrolo[3,2,1-ij]quinoline-5-carboxylate: To a solution of methyl 4-bromo-5-fluoro-1-(3-methoxy-3-oxopropanoyl)-2-methylindoline-7-carboxylate (1.50 g, 3.85 mmol) in acetonitrile (35 mL) was added cesium carbonate (3.77 g, 11.6 mmol). The mixture was stirred at 60° C. for 1 h. The reaction mixture was quenched with water and acidified with 1N HCl solution to adjust the pH to 2-3. EtOAc was added, and the phases were separated. The aqueous layer was extracted with EtOAc, and the organic layer was dried on Na₂SO₄, filtered and concentrated. The crude product was used in the next step without further purification. LCMS (m/z): 358.3 [M+2].

(vi). Methyl 9-bromo-8-fluoro-2-methyl-4-oxo-6-(((trifluoromethyl)sulfonyl)oxy)-1,2-dihydro-4H-pyrrolo[3,2,1-ij]quinoline-5-carboxylate: To a solution of methyl 9-bromo-8-fluoro-6-hydroxy-2-methyl-4-oxo-1,2-dihydro-4H-pyrrolo[3,2,1-ij]quinoline-5-carboxylate (0.67 g, 1.88 mmol) in anhydrous DCM (12 mL) was added triethylamine (0.57 g, 0.79 mL, 5.64 mmol) and triflic anhydride (1.59 g, 0.95 mL, 5.64 mmol) at 0° C. The reaction was completed after 15 minutes. Evaporation of the solvent afforded the product as a dark brown oil. LCMS (m/z): 490.1 [M+2].

(vii). Methyl 9-bromo-8-fluoro-2-methyl-4-oxo-1,2-dihydro-4H-pyrrolo[3,2,1-ij]quinoline-5-carboxylate: Crude methyl 9-bromo-8-fluoro-2-methyl-4-oxo-6-(((trifluoromethyl)sulfonyl)oxy)-1,2-dihydro-4H-pyrrolo[3,2,1-ij]quinoline-5-carboxylate (918 mg, 1.88 mmol) was dissolved in DMF (10 mL). DPPP (233 mg, 0.56 mmol), palladium(II) acetate (63.3 mg, 0.28 mmol) and triethylsilane (547 mg, 0.75 mL, 4.70 mmol) were added sequentially under N₂ purging. The reaction mixture was stirred at 80° C. for 1 h. The crude mixture was diluted with EtOAc and extracted with water. The organic layer was washed with water, dried on Na₂SO₄ and filtered. The solvent was evaporated, and the crude residue was purified by silica gel chromatography (0-100% EtOAc/Hexane) to afford a yellow solid as the desired product (456 mg, 73% yield). LCMS (m/z): 342.2 [M+2].

(viii). Methyl 9-((R)-3-(1-((tert-butoxycarbonyl)amino)cyclopropyl)pyrrolidin-1-yl)-8-fluoro-2-methyl-4-oxo-1,2-dihydro-4H-pyrrolo[3,2,1-ij]quinoline-5-carboxylate: A microwave vial was charged with methyl 9-bromo-8-fluoro-2-methyl-4-oxo-1,2-dihydro-4H-pyrrolo[3,2,1-ij]quinoline-5-carboxylate (280 mg, 0.82 mmol) and toluene (3 mL). tert-Butyl (S)-(1-(pyrrolidin-3-yl)cyclopropyl)carbamate (279 mg, 1.24 mmol), RuPhos (19.2 mg, 0.04 mmol), RuPhos Pd G3 (34.4 mg, 0.04 mmol) and cesium carbonate (805 mg, 2.47 mmol) were added. The mixture was heated to 100° C. for 1 h. The crude mixture was filtered through a disposable filter funnel and volatiles were evaporated under reduced pressure. Silica gel chromatography (0-100% EtOAc/heptane) provided the diastereoisomeric mixture (200 mg) as a yellow solid. The diastereoisomeric mixture was separated by SFC (Column: X5 4.6×100 mm, CO₂/MeOH=90/10, flow rate 5ml/min) to afford the desired diastereoisomer (relative stereochemistry not determined, 70 mg, 17% yield). LCMS (m/z): 450.5 [M+1].

(ix). 9-((R)-3-(1-((tert-butoxycarbonyl)amino)cyclopropyl)pyrrolidin-1-yl)-8-fluoro-2-methyl-4-oxo-1,2-dihydro-4H-pyrrolo[3,2,1-ij]quinoline-5-carboxylic acid: Methyl 9-((R)-3-(1-((tert-butoxycarbonyl)amino)cyclopropyl)pyrrolidin-1-yl)-8-fluoro-2-methyl-4-oxo-1,2-dihydro-4H-pyrrolo[3,2,1-ij]quinoline-5-carboxylate (70.0 mg, 0.14 mmol) was dissolved in MeOH (3 mL) and water (1 mL). A 1M solution of lithium hydroxide (0.14 mL, 0.14 mmol) was added at rt. After stirring for 3 h the mixture was diluted with water and treated with 1 M HCl. The resulting mixture was extracted with EtOAc, and the organic extract was washed with brine, dried over MgSO₄, filtered and concentrated under reduced pressure. The product was used directly without further purification. LCMS (m/z): 472.5 [M+1].

(x). 9-((R)-3-(1-aminocyclopropyl)pyrrolidin-1-yl)-8-fluoro-2-methyl-4-oxo-1,2-dihydro-4H-pyrrolo[3,2,1-ij]quinoline-5-carboxylic acid hydrochloride salt: Crude 9-((R)-3-(1-((tert-butoxycarbonyl)amino)cyclopropyl)pyrrolidin-1-yl)-8-fluoro-2-methyl-4-oxo-1,2-dihydro-4H-pyrrolo[3,2,1-ij]quinoline-5-carboxylic acid (67.0 mg, 0.14 mmol) was dissolved in DCM (3 mL) and HCl 4N in dioxane (1 mL) was added. The mixture was stirred at rt for 3 h. The solvent was evaporated, and the crude residue was dissolved in a minimal amount of MeOH. The addition of acetonitrile afforded a suspension which was collected by filtration, affording the desired product (29.0 mg, 49% yield). ¹H NMR (400 MHz, Methanol-d₄) δ 8.59 (s, 1H), 7.35 (d, J=14.3 Hz, 1H), 5.07-5.01 (m, 1H), 3.96-3.85 (m, 4H), 3.68 (dt, J=9.8, 5.0 Hz, 1H), 3.49-3.42 (m, 1H), 2.66-2.61 (m, 1H), 2.13 (dt, J=11.7, 5.9 Hz, 1H), 1.79-1.66 (m, 1H), 1.61 (d, J=6.4 Hz, 3H), 1.04-0.97 (m, 4H). LCMS: tR=1.81 min, m/z=372.5 [M+1] (10 minute run, method A).

Using the procedures described for Example 9.1 the following compounds were prepared:

| | | | HPLC, | |
|---|---|---|---|---|
| Ex | | | LCMS | LCMS |
| # | Structure | Chemical Name | t_{R} (min) | [M + 1] |
| 9.2¹ | 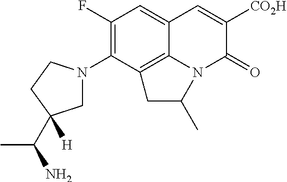 [SMILES: CC1Cc2c(N3CC[C@@H]([C@H](C)N)C3)c(F)cc3cc(C(=O)O)c(=O)n1c23] | 9-((R)-3-((S)-1- aminoethyl)pyrrolidin-1-yl)-8- fluoro-2-methyl-4-oxo-1,2-dihydro- 4H-pyrrolo[3,2,1-ij]quinoline-5- (carboxylic acid (TFA salt) | 1.81 | 360.4 |
| 9.3¹ | 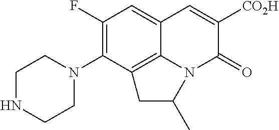 [SMILES: CC1Cc2c(N3CCNCC3)c(F)cc3cc(C(=O)O)c(=O)n1c23] | 8-fluoro-2-methyl-4-oxo-9- (piperazin-1-yl)-1,2-dihydro-4H- pyrrolo[3,2,1-ij]quinoline-5- carboxylic acid (TFA salt) | 0.92 | 332.4 |

¹Isomers mixture, SFC purification not performed at step viii.

HPLC method: Characterized by high performance liquid chromatography (HPLC) on a Waters ACQUITY H class UPLC system with 0.55 mL/min flow rate; column BEH-C18, 1.7 um, 2.1×50 mm from Waters, column temperature: ambiant; gradient (solvent A is 2 mM Ammonium Acetate and 0.1% Formic Acid in Water, solvent B is 0.1% Formic Acid in acetonitrile) : 5% solvent B hold for 0.4 min, 5-40% solvent B over 0.6 min, 40-60% solvent B over 1.2 min, 60-100% solvent B over 2.3 min then 100% solvent B over 3 min; compounds were detected by ultraviolet light (UV) absorption at 236 nm.

### Example 10

### (S)-10-((R)-3-(1-aminocyclopropyl)pyrrolidin-1-yl)-9-fluoro-3-methyl-5-oxo-2,3-dihydro-1H,5H-pyrido[1,2,3-de]quinoxaline-6-carboxylic acid hydrochloride salt

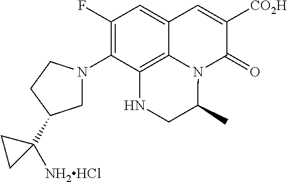

The title compound was prepared in accordance with the following scheme:

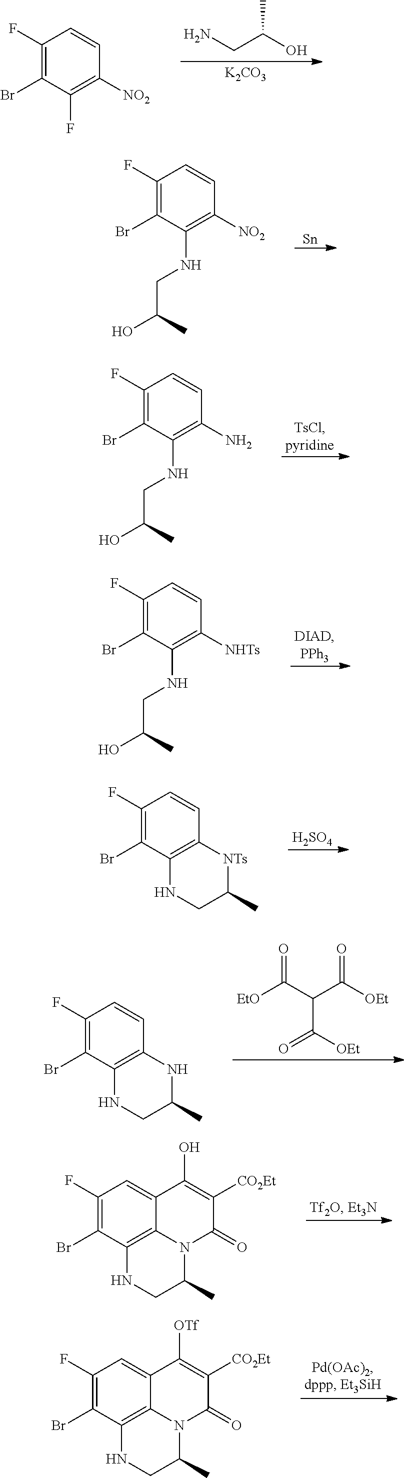

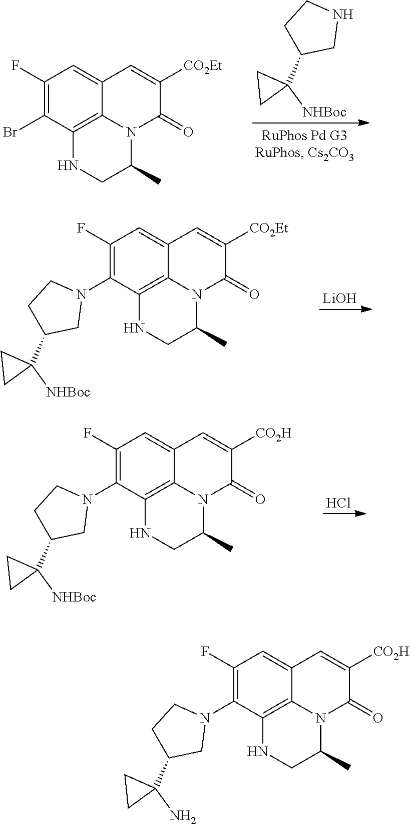

(i). (R)-1-((2-bromo-3-fluoro-6-nitrophenyl)amino)propan-2-ol: To a solution of (S)-1-aminopropan-2-ol hydrochloride (12.50 g, 112.6 mmol) in DMF (200 mL) at 0° C. was added K₂CO₃ (17.32 g, 125.3 mmol) and the resulting mixture was stirred for 10 minutes. 2-Bromo-1,3-difluoro-4-nitrobenzene (26.80 g, 112.6 mmol) was added and the mixture was stirred at 60° C. for 6 h. After completion the mixture was poured into ice cold water and extracted with EtOAc. The combined organic layer was washed with brine, dried over Na₂SO₄ and concentrated under vaccum to afford (R)-1-((2-bromo-3-fluoro-6-nitrophenyl)amino)propan-2-ol (27.00 g, 82% yield) as a yellow solid which was directly used in the next step without any further purification. ¹H NMR (400 MHz, Chloroform-d) δ 8.07 (td, J=10.4, 9.3, 7.2 Hz, 1H), 7.07 (s, 1H), 6.68 (dd, J=9.4, 7.1 Hz, 1H), 4.16 (q, J=7.1 Hz, 1H), 3.53 (ddd, J=13.1, 6.1, 3.4 Hz, 1H), 3.36-3.19 (m, 1H),1.38 (d, J=6.3 Hz, 3H). LCMS (m/z): 295.5 [M+2].

(ii). (R)-1-((6-amino-2-bromo-3-fluorophenyl)amino)propan-2-ol: (R)-1-((2-bromo-3-fluoro-6-nitrophenyl)amino)propan-2-ol (27.00 g, 92.15 mmol) was dissolved in THF (80 mL) and cooled to 0° C. Tin (38.05 g, 322.5 mmol) and 4N aqueous HCl (83.00 mL, 737.2 mmol) were added and the reaction mixture was refluxed for 2 h. After completion, the reaction mixture was cooled to rt and filtered through a celite pad. Water and EtOAc were added to the filtrate, and the phases were separated. The organic layer was washed with brine, dried over sodium sulfate and concentrated under vacuum to afford (R)-1-((6-amino-2-bromo-3-fluorophenyl)amino)propan-2-ol (24.00 g, 99% yield) as a colourless gum which was directly used in the next step without any further purification. ¹H NMR (400 MHz, Chloroform-d) δ 6.87-6.67 (m, 2H), 4.17 (q, J=7.1 Hz, 1H), 3.18 (dd, J=13.0, 2.9 Hz, 2H), 2.90 (dd, J=13.0, 8.7 Hz, 1H), 1.30 (dd, J=9.0, 6.7 Hz, 3H). LCMS (m/z): 265.5 [M+2].

(iii). (R)-N-(3-bromo-4-fluoro-2-((2-hydroxypropyl)amino)phenyl)-4-methylbenzenesulfonamide: (R)-1-((6-amino-2-bromo-3-fluorophenyl)amino)propan-2-ol (24.00 g, 91.25 mmol) was dissolved in pyridine (240 mL) followed by cooling to 0° C. p-TsCl (26.00 g, 136.9 mmol) was added and the reaction mixture was stirred at rt overnight. The reaction mixture was concentrated under vacuum, diluted with water and extracted with EtOAc. The combined organic layer was washed with aqueous NaHCO₃ saturated solution, brine, was dried over sodium sulfate and concentrated under vacuum to afford a crude residue which was purified by silica gel chromatography (45% EtOAc in hexane), affording the desired product (20.00 g, 52% yield) as a white solid. ¹H NMR (400 MHz, Chloroform-d) δ 8.49 (s, 1H), 7.80-7.69 (m, 2H), 7.48-7.38 (m,1H), 7.41-7.25 (m, 2H), 6.80 (dd, J=9.0, 7.8 Hz, 1H), 3.98 (ddt, J=11.6, 7.7, 3.9 Hz, 1H), 3.00 (dd, J=13.4, 2.7 Hz, 1H), 2.62 (dd, J=13.5, 8.5 Hz, 1H), 2.55-2.45 (m, 1H), 2.43 (s, 3H), 1.49 (s, 1H),1.25 (d, J=6.3 Hz, 3H). LCMS (m/z): 419.5 [M+2].

(iv). (S)-5-bromo-6-fluoro-2-methyl-1-tosyl-1,2,3,4-tetrahydroquinoxaline: (R)-N-(3-bromo-4-fluoro-2-((2-hydroxypropyl)amino)phenyl)-4-methylbenzenesulfonamide (20.00 g, 47.73 mmol) was dissolved in THF (150 mL) at 0° C. Triphenylphosphine (18.85 g 71.94 mmol) was added followed by the dropwise addition of DIAD (12.59 g, 62.35 mmol). The reaction mixture was stirred for 6 h at rt. The reaction mixture was diluted with water and extracted with EtOAc. The combined organic layer was washed with aqueous NaHCO₃ saturated solution, brine, dried over sodium sulfate and concentrated under vacuum to afford a crude residue which was purified by silica gel chromatography (15% EtOAc in hexane) affording the desired product (15.00 g, 78% yield) as a white solid. ¹H NMR (400 MHz, Chloroform-d) δ 7.69 (dd, J=9.0, 5.6 Hz, 1H), 7.44 (d, J=8.1 Hz, 2H), 7.25 (d, J=7.9 Hz, 2H), 6.56 (t, J=8.6 Hz, 1H), 4.55-4.52 (m, 2H), 3.02 (dd, J=11.5, 4.8 Hz, 1H), 2.74-2.66 (m, 1H), 2.43 (s, 3H), 1.13 (d, J=6.9 Hz, 3H). LCMS (m/z): 401.5 [M+2].

(v). (S)-5-bromo-6-fluoro-2-methyl-1,2,3,4-tetrahydroquinoxaline: (S)-5-bromo-6-fluoro-2-methyl-1-tosyl-1,2,3,4-tetrahydroquinoxaline (15.00 g, 37.59 mmol) was dissolved in DCM (150 mL) and cooled at 0° C. Concentrated H₂SO₄ (15.00 mL, 188.0 mmol) was added dropwise and the reaction mixture was stirred at rt for 2 h. The mixture was poured into ice cold water and extracted with EtOAc. The combined organic layer was washed with aqueous NaHCO₃ saturated solution, brine, dried over Na₂SO₄ and concentrated under vaccum to afford (S)-5-bromo-6-fluoro-2-methyl-1,2,3,4-tetrahydroquinoxaline (7.500 g, 91% yield) as a colorless gum. ¹H NMR (400 MHz, Chloroform-d) δ 6.45-6.33 (m, 2H), 4.42-4.38 (m, 1H), 3.47 (ddt, J=8.6, 6.3, 3.2 Hz, 2H), 3.13 (dd, J=11.4, 8.7 Hz, 1H), 1.30 (d, J=6.3 Hz, 3H). LCMS (m/z): 247.5 [M+2].

(vi). Ethyl (S)-10-bromo-9-fluoro-7-hydroxy-3-methyl-5-oxo-2,3-dihydro-1H,5H-pyrido[1,2,3-de]quinoxaline-6-carboxylate: To (S)-5-bromo-6-fluoro-2-methyl-1,2,3,4-tetrahydroquinoxaline (1.70 g, 6.94 mmol) in DMF (17 mL) was added triethylmethane tricarboxylate (8.05 g, 34.7 mmol) and the reaction was heated to 180° C. for 6 h. The reaction mixture was poured into ice cold water and extracted with EtOAc. The combined organic layer was washed with water, brine, dried over sodium sulfate and concentrated under vacuum to afford a crude residue which was purified using basic alumina chromatography (15% Methanolic ammonia in DCM), affording the desired product (0.95 g, 35% yield) as pale orange solid. LCMS (m/z): 387.5 [M+2].

(vii). Ethyl (S)-10-bromo-9-fluoro-3-methyl-5-oxo-7-(((trifluoromethyl)sulfonyl)oxy)-2,3-dihydro-1H,5H-pyrido[1,2,3-de]quinoxaline-6-carboxylate: To ethyl (S)-10-bromo-9-fluoro-7-hydroxy-3-methyl-5-oxo-2,3-dihydro-1H,5H-pyrido[1,2,3-de]quinoxaline-6-carboxylate (0.95 g, 2.47 mmol) in DCM (20 mL) was added Et₃N (2.24 g, 3.07 mL, 22.20 mmol) followed by cooling to -78° C. Trifluoromethanesulfonic anhydride (3.14 g, 1.87 mL, 11.10 mmol) was added dropwise and the reaction mixture was stirred at -78° C. for 30 minutes. The reaction mixture was concentrated under reduced pressure to afford the desired product (1.00 g) which was directly used in the next step without any further purification. LCMS (m/z): 519.2 [M+2].

(viii). Ethyl (S)-10-bromo-9-fluoro-3-methyl-5-oxo-2,3-dihydro-1H,5H-pyrido[1,2,3-de]quinoxaline-6-carboxylate: Ethyl (S)-10-bromo-9-fluoro-3-methyl-5-oxo-7-(((trifluoromethyl)sulfonyl)oxy)-2,3-dihydro-1H,5H-pyrido[1,2,3-de]quinoxaline-6-carboxylate (1.00 g, 1.93 mmol) was dissolved in dry DMF (5 mL) and degassed with nitrogen for 5 minutes. DPPP (0.24 g, 0.58 mmol), Pd(II)OAc (65.0 mg, 0.29 mmol) were added and the reaction mixture was degassed with nitrogen for another 5 minutes. The reaction mixture was cooled to 0° C., triethylsilane (0.67 g, 0. 9 2 m L , 5.80 mmol) was added and the reaction mixture was stirred at 65° C. for 4 h . The reaction mixture was quenched with ice cold water and extracted with EtOAc. The organic layer was washed with cold water, brine, dried over sodium sulfate and concentrated under vacuum to afford a crude residue which was purified by silica gel chromatography (20-30% EtOAc in Hexane) affording the desired product (0.40 g, 43% yield, 2 steps). ¹H NMR (400 MHz, Chloroform-d) δ 8.32 (d, J=12.1 Hz, 1H), 6.85 (d, J=7.9 Hz, 1H), 5.38-5.36 (m, 1H), 4.96-4.94 (m, 1H), 4.59-4.41 (m, 2H), 3.53 (q, J=13.7, 13.1 Hz, 2H), 1.41 (dd, J=15.4, 6.6 Hz, 6H). LCMS (m/z): 371.3 [M+2].

(ix). Ethyl (S)-10-((R)-3-(1-((tert-butoxycarbonyl)amino)cyclopropyl)pyrrolidin-1-yl)-9-fluoro-3-methyl-5-oxo-2,3-dihydro-1H,5H-pyrido[1,2,3-de]quinoxaline-6-carboxylate: Ethyl (S)-10-bromo-9-fluoro-3-methyl-5-oxo-2,3-dihydro-1H,5H-pyrido[1,2,3-de]quinoxaline-6-carboxylate (0.200 g, 0.542 mmol), tert-butyl (R)-(1-(pyrrolidin-3-yl)cyclopropyl)carbamate (0.183 g, 0.813 mmol) and Cs₂CO₃ (0.529 g, 1.626 mmol) were suspended in toluene (8 mL) and degassed with nitrogen for 5 minutes. RuPhos (0.025 g, 0.054 mmol) and RuPhosPdG₃ (0.045 g, 0.054 mmol) were added and the reaction mixture was heated to 90° C. for 6 h . The reaction mixture was poured into water and extracted with EtOAc. The combined organic layer was washed with brine, dried over sodium sulfate and concentrated under vacuum to afford a crude residue which was purified by reverse phase chromatography (60-70% MeOH in water) affording the desired product (0.060 g, 23% yield) as yellow solid. LCMS (m/z): 515.6 [M+1].

(x). (S)-10-((R)-3-(1-((tert-butoxycarbonyl)amino)cyclopropyl)pyrrolidin-1-yl)-9-fluoro-3-methyl-5-oxo-2,3-dihydro-1H,5H-pyrido[1,2,3-de]quinoxaline-6-carboxylic acid: Ethyl (S)-10-((R)-3-(1-((tert-butoxycarbonyl)amino)cyclopropyl)pyrrolidin-1-yl)-9-fluoro-3-methyl-5-oxo-2,3-dihydro-1H,5H-pyrido[1,2,3-de]quinoxaline-6-carboxylate (0.06 g, 0.12 mmol) was dissolved in a mixture of MeOH:H₂O (3:1, 8 mL). LiOH (1M in water, 0.35 mL, 0.35 mmol) was added and the reaction mixture was stirred at rt for 3 h. The reaction mixture was quenched with cold water, acidified with diluted HCl to pH 4 and extracted with ethyl acetate. The organic layer was concentrated under vacuum to afford the desired product (45.0 mg, 79% yield) as a yellow solid. LCMS (m/z): 487.5 [M+1].

(xi). (S)-10-((R)-3-(1-aminocyclopropyl)pyrrolidin-1-yl)-9-fluoro-3-methyl-5-oxo-2,3-dihydro-1H,5H-pyrido[1,2,3-de]quinoxaline-6-carboxylic acid hydrochloride salt: (S)-10-((R)-3-(1-((tert-butoxycarbonyl)amino)cyclopropyl)pyrrolidin-1-yl)-9-fluoro-3-methyl-5-oxo-2,3-dihydro-1H,5H-pyrido[1,2,3-de]quinoxaline-6-carboxylic acid (45.0 mg, 0.09 mmol) was dissolved in DCM (5 mL) and HCl 4N in dioxane (0.6 mL) was added. The mixture was stirred at rt for 2 h. The solvent was evaporated, and the crude residue was triturated with EtOAc, affording the desired product (20.0 mg, 57% yield) as a yellow solid. ¹H NMR (400 MHz, Methanol-d₄) δ 8.81 (s, 1H), 7.06 (d, J=12.1 Hz, 1H), 5.31 (s, 1H), 3.57 (t, J=9.4 Hz, 2H), 3.32-3.29 (m, 2H), 3.20 (d, J=8.1 Hz, 1H), 2.86-2.77 (m, 1H), 2.24 (d, J=9.0 Hz, 1H), 1.85-1.75 (m, 1H), 1.44 (d, J=6.6 Hz, 3H),1.10-0.98 (m, 4H). LCMS: t_{R}=1.38 min, m/z=387.4 [M+1] (3 minutes run, method B).

### Example 11

### (S)-10-((R)-3-(1-aminocyclopropyl)pyrrolidin-1-yl)-9-fluoro-3-methyl-5-oxo-2,3-dihydro-5H-[1,4]thiazino[2,3,4-ij]quinoline-6-carboxylic acid hydrochloride salt

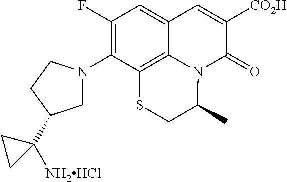

The title compound was prepared in accordance with the following scheme:

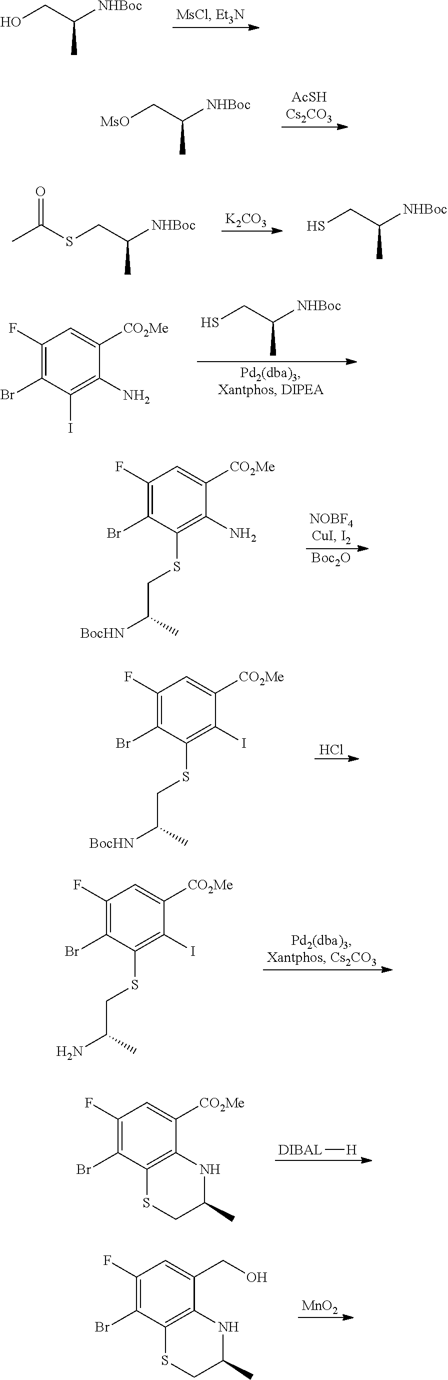

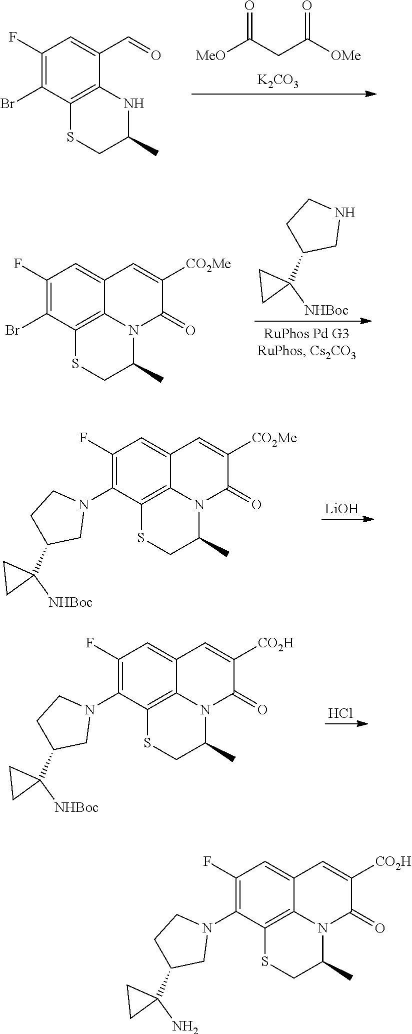

(i). (S)-2-((tert-butoxycarbonyl)amino)propyl methanesulfonate: To a solution of tert-butyl (S)-(1-hydroxypropan-2-yl)carbamate (20.0 g, 114.1 mmol) in DCM (100 mL) was added Et₃N (18.48 g, 25.38 mL, 182.6 mmol) and the resulting mixture was cooled to 0° C. Mesyl chloride (16.99 g, 12.00 mL, 148.3 mmol) was added dropwise and the mixture was stirred for 1 h. After completion the mixture was poured into ice cold water and extracted with DCM. The combined organic layer was washed with a saturated NaHCO₃ solution, dried over Na₂SO₄ and concentrated under vaccum to afford (S)-2-((tert-butoxycarbonyl)amino)propyl methanesulfonate (28.00 g, 97% yield) as a white solid which was directly used in the next step without any further purification. ¹H NMR (400 MHz, Chloroform-d) δ 4.71 (s, 1H), 4.28 (s, 1H), 4.21 (dd, J=10.0, 4.3 Hz, 1H), 4.07-3.98 (m, 1H), 3.09 (s, 3H), 1.50 (s, 9H), 1.29 (d, J=6.9 Hz, 3H). LCMS (m/z): 254.31 [M+1].

(ii). (S)-S-(2-((tert-butoxycarbonyl)amino)propyl) ethanethioate: To a solution of (S)-2-((tert-butoxycarbonyl)amino)propyl methanesulfonate carbamate (10.0 g, 39.5 mmol) in DMF (100 mL) was added at −78° C. thioacetic acid (3.61 g, 47.4 mmol) and Cs₂CO₃ (8.38 g, 25.7 mmol). The resulting mixture was slowly warmed to rt and stirred for 16 h. The reaction mixture was poured into ice cold water and extracted with EtOAc. The combined organic layer was washed with water, brine, dried over Na₂SO₄ and concentrated under vaccum to afford (S)-S-(2-((tert-butoxycarbonyl)amino)propyl) ethanethioate (7.18 g, 78% yield) as a colorless gum which was directly used in the next step without any further purification. ¹H NMR (400 MHz, Chloroform-d) δ 4.59 (s, 1H), 3.92 (s, 1H), 3.09 (q, J=7.1, 6.6 Hz, 2H), 2.30 (s, 3H), 1.50 (s, 9H), 1.23 (d, J=6.7 Hz, 3H). LCMS (m/z): 234.33 [M+1].

(iii). tert-Butyl (S)-(1-mercaptopropan-2-yl)carbamate: To a solution of (S)-S-(2-((tert-butoxycarbonyl)amino)propyl) ethanethioate (1.54 g, 6.57 mmol) in MeOH:water (1:1, 20 mL) was added K₂CO₃ (1.78 g, 12.9 mmol) and the resulting mixture was stirred at rt for 45 minutes. After completion the mixture was poured into water and extracted with EtOAc. The combined organic layer was washed with water, brine, dried over Na₂SO₄ and concentrated under vacuum to afford tert-butyl (S)-(1-mercaptopropan-2-yl)carbamate (1.00 g, 82% yield) as a colorless gum which was directly used in the next step without any further purification. LCMS (m/z): 192.29 [M+1].

(iv). Methyl (S)-2-amino-4-bromo-3-((2-((tert-butoxycarbonyl)amino)propyl)thio)-5-fluorobenzoate: Methyl 2-amino-4-bromo-5-fluoro-3-iodobenzoate (1.00 g, 2.67 mmol), tert-butyl (S)-(1-mercaptopropan-2-yl)carbamate (1.02 g, 5.34 mmol) and DIPEA (1.52 g, 2.04 mL, 11.8 mmol) were dissolved in toluene (20 mL) and degassed with nitrogen for 5 minutes. XantPhos (247 m g, 0.43 mmol) and Pd₂dba₃ (195 m g, 0.21 mmol) were added and the reaction mixture was heated to 80° C. for 6 h. The reaction mixture was quenched with water and extracted with EtOAc. The organic layer was washed with water, brine, dried over sodium sulfate and concentrated under vacuum. The crude residue was purified by silica gel chromatography (10-15% EtOAc in Hexane), affording the desired product (0.80 g, 68% yield) as a white solid. LCMS (m/z): 439.32 [M+2].

(v). Methyl (S)-3-((2-aminopropyl)thio)-4-bromo-5-fluoro-2-iodobenzoate: Methyl (S)-2-amino-4-bromo-3-((2-((tert-butoxycarbonyl)amino)propyl)thio)-5-fluorobenzoate (1.00 g, 2.29 mmol) in DCM (10 mL) was added to a stirring solution of NOBF₄ (0.53 g, 4.58 mmol) in DCM (10 mL) at −50° C. The reaction mixture was stirred at 0° C. for 1 h. After complete formation of the diazonium salt the reaction mixture was concentrated under vacuum and the crude residue was dissolved into MeCN (10 mL) and cooled to −30° C. CuI (0.44 g, 2.29 mmol) and iodine (0.29 g, 1.14 mmol) were added into the diazonium solution and the mixture was stirred at 0° C. for 1 h. The reaction mixture was quenched with water and extracted with EtOAc. The combined organic layer was washed with saturated aqueous Na₂S₂O₃, brine, dried over sodium sulfate and concentrated under vacuum to afford the desired product (0.520 g) which was directly used in the next step without any further purification. LCMS (m/z): 450.09 [M+2].

(vi). Methyl (S)-4-bromo-3-((2-((tert-butoxycarbonyl)amino)propyl)thio)-5-fluoro-2-iodobenzoate: Methyl (S)-3-((2-aminopropyl)thio)-4-bromo-5-fluoro-2-iodobenzoate (0.50 g, 1.11 mmol) was dissolved in DCM (10 mL). Et₃N (0.23 g, 0.31 mL, 2.22 mmol) and (Boc)₂O (0.27 g, 1.22 mmol) were added to the reaction mixture followed by stirring for 3 h. The mixture was poured into water and extracted with EtOAc. The combined organic layer was washed with water, brine, dried over sodium sulfate and concentrated under vacuum to afford a crude residue which was purified by silica gel chromatography (5-10% EtOAc in Hexane), providing the desired product (0.35 g, 57% yield) as a white solid. ¹H NMR (400 MHz, Chloroform-d) δ 7.32 (s, 1H), 4.80 (s, 1H), 3.99 (s, 3H), 3.17-3.13 (m, 3H), 1.46-1.37 (m, 12H). LCMS (m/z): 550.21 [M+2].

(vii). Methyl (S)-3-((2-aminopropyl)thio)-4-bromo-5-fluoro-2-iodobenzoate hydrochloride: Methyl (S)-4-bromo-3-((2-((tert-butoxycarbonyl)amino)propyl)thio)-5-fluoro-2-iodobenzoate (0.350 g, 0.638 mmol) was dissolved in DCM (5 mL) and cooled to 0° C. HCl in dioxane (4N, 3.5 mL) was added and the reaction mixture was stirred at rt for 2 h. The reaction mixture was concentrated under vacuum, triturated with n-pentane to afford the desired product (0.230 g, 80% yield) as a white solid. ¹H NMR (400 MHz, Methanol-d₄) δ 7.53 (d, J=8.1 Hz, 1H), 3.96 (s, 3H), 3.42 (dt, J=13.3, 6.6 Hz, 1H), 3.26 (dd, J=13.6, 5.5 Hz, 1H), 3.11 (dd, J=13.6, 8.0 Hz, 1H), 1.51 (d, J=6.5 Hz, 3H). LCMS (m/z): 450.09 [M+2].

(viii). Methyl (S)-8-bromo-7-fluoro-3-methyl-3,4-dihydro-2H-benzo[b][1,4]thiazine-5-carboxylate: To methyl (S)-3-((2-aminopropyl)thio)-4-bromo-5-fluoro-2-iodobenzoate hydrochloride (0.360 g, 0.743 mmol) in dioxane (15mL) was added Cs₂CO₃ (0.727 g, 1.425 mmol) and the suspension was degassed with nitrogen for 5 minutes. XantPhos (0.064 g, 0.111 mmol) and Pd₂dba₃ (0.034 g, 0.037 mmol) were added and the reaction mixture was heated to 90° C. for 16 h. The reaction mixture was quenched with water and extracted with EtOAc. The combined organic layer was washed with water, brine, dried over sodium sulfate and concentrated under vacuum. The crude residue was purified by silica gel chromatography (5-10% EtOAc in Hexane) affording the desired product (0.120 g, 50% yield) as a yellow solid. ¹H NMR (400 MHz, Chloroform-d) δ 8.18 (s, 1H), 7.49 (d, J=9.3 Hz, 1H), 3.90 (s, 3H), 3.85-3.78 (m, 1H), 3.01 (dd, J=12.4, 2.7 Hz, 1H), 2.82 (dd, J=12.4, 8.3 Hz, 1H), 1.43 (d, J=6.4 Hz, 3H). LCMS (m/z): 322.18 [M+2].

(ix). (S)-(8-bromo-7-fluoro-3-methyl-3,4-dihydro-2H-benzo[b][1,4]thiazin-5-yl)methanol: Methyl (S)-8-bromo-7-fluoro-3-methyl-3,4-dihydro-2H-benzo[b][1,4]thiazine-5-carboxylate (0.140 g, 0.437 mmol) was dissolved in toluene (10 mL) and cooled to -78° C. DIBAL (1M in toluene, 0.875 mL, 0.875 mmol) was added to the stirring mixture and the temperature was allowed to reach rt over 2 h. After completion of the reaction the mixture was quenched with EtOAc (1 mL) and slowly poured on to a slurry of wet sodium sulfate. The reaction mixture was filtered through a celite pad and concentrated under vacuum to afford the desired product (0.125 g, 90% yield) which was directly used in the next step without any further purification. ¹H NMR (400 MHz, Chloroform-d) δ 7.22 (d, J=7.9 Hz, 1H), 6.72 (d, J=8.3 Hz, 1H), 4.62 (s, 2H), 3.72-3.68 (m, 1H), 3.01 (dd, J=12.4, 2.6 Hz, 1H), 2.83 (dd, J=12.4, 8.3 Hz, 1H), 1.40 (d, J=6.3 Hz, 3H). LCMS (m/z): 294.17 [M+2].

(x). (S)-8-bromo-7-fluoro-3-methyl-3,4-dihydro-2H-benzo[b][1,4]thiazine-5-carbaldehyde: (S)-(8-bromo-7-fluoro-3-methyl-3,4-dihydro-2H-benzo[b][1,4]thiazin-5-yl)methanol (0.125 g, 0.428 mmol) was dissolved in DIVIF (5 mL). MnO₂ (0.558 g, 6.420 mmol) was added at 0° C. to the reaction mixture, followed by stirring at rt for 2 h. After completion, the reaction mixture was diluted with EtOAc and filtrated through a celite pad. The filtrate was quenched with water and extracted with EtOAc. The combined organic layer was washed with water, brine, dried over sodium sulfate and concentrated under vacuum to afford the desired product (0.120 g, 99% yield) as a yellow solid which was directly used in the next step without any further purification. ¹H NMR (400 MHz, Chloroform-d) δ 9.75 (s, 1H), 8.77 (s, 1H), 7.05 (d, J=8.0 Hz, 1H), 3.88 (s, 1H), 3.07-2.98 (m, 1H), 2.82 (dd, J=12.7, 7.9 Hz, 1H), 1.45 (d, J=6.4 Hz, 3H). LCMS (m/z): 292.15 [M+2].

(xi). Methyl (S)-10-bromo-9-fluoro-3-methyl-5-oxo-2,3-dihydro-5H-[1,4]thiazino[2,3,4-ij]quinoline-6-carboxylate: (S)-8-bromo-7-fluoro-3-methyl-3,4-dihydro-2H-benzo[b][1,4]thiazine-5-carbaldehyde (0.120 g, 0.413 mmol) was dissolved in toluene (5 mL). K₂CO₃ (0.028 g, 0.206 mmol), dimethyl malonate (0.082 g, 0.620 mmol), piperidine (0.06 mL) and acetic acid (0.06 mL) were added and the reaction mixture was stirred at 100° C. for 16 h. After completion, the reaction mixture was quenched with water and extracted with EtOAc. The combined organic layer was washed with water, brine, dried over sodium sulfate and concentrated under vacuum to afford a crude residue which was purified by trituration with diethyl ether, affording the desired product (0.080 g, 52% yield) as a yellow solid which was directly used in the next step without any further purification. ¹H NMR (400 MHz, Chloroform-d) δ 8.34 (s, 1H), 7.41 (d, 1H), 5.90 (dq, J=6.5, 3.3Hz, 1H), 4.01 (s, 3H), 3.28 (dd, J=13.6, 3.1 Hz, 1H), 3.14 (dd, J=13.6, 3.2 Hz, 1H), 1.47 (d, J=6.4 Hz, 3H). LCMS (m/z): 374.21 [M+2].

(xii). Methyl (S)-10-((R)-3-(1-((tert-butoxycarbonyl)amino)cyclopropyl)pyrrolidin-1-yl)-9-fluoro-3-methyl-5-oxo-2,3-dihydro-5H-[1,4]thiazino[2,3,4-ij]quinoline-6-carboxylate: Methyl (S)-10-bromo-9-fluoro-3-methyl-5-oxo-2,3-dihydro-5H-[1,4]thiazino[2,3,4-ij]quinoline-6-carboxylate (0.080 g, 0.215 mmol), tert-butyl (R)-(1-(pyrrolidin-3-yl)cyclopropyl)carbamate (0.072 g, 0.322 mmol) and Cs₂CO₃ (0.175 g, 0.537 mmol) were suspended in toluene (3 mL) and degassed with nitrogen for 5 minutes. RuPhos (0.015 g, 0.032 mmol) and RuPhosPdG₃ (0.026 g, 0.032 mmol) were added and the reaction mixture was heated to 80° C. for 6 h . The reaction mixture was poured into water and extracted with EtOAc. The combined organic layer was washed with brine, dried over sodium sulfate and concentrated under vacuum to afford a crude residue which was purified by reverse phase chromatography (60-70% MeOH in water), affording the desired product (0.100 g, 90% yield) as a yellow solid. LCMS (m/z): 518.6 [M+1].

(xiii). (S)-10-((R)-3-(1-((tert-butoxycarbonyl)amino)cyclopropyl)pyrrolidin-1-yl)-9-fluoro-3-methyl-5-oxo-2,3-dihydro-5H-[1,4]thiazino[2,3,4-ij]quinoline-6-carboxylic acid: Methyl (S)-10-((R)-3-(1-((tert-butoxycarbonyl)amino)cyclopropyl)pyrrolidin-1-yl)-9-fluoro-3-methyl-5-oxo-2,3-dihydro-5H-[1,4]thiazino[2,3,4-ij]quinoline-6-carboxylate (0.100 g, 0.193 mmol) was dissolved in a mixture of MeOH:H₂O (3:1, 12 mL). LiOH (1M in water) (0.580 mL, 0.580 mmol) was added and the reaction mixture was stirred at rt for 3 h. The reaction mixture was quenched with cold water, acidified with dilute HCl to pH 4 and extracted with ethyl acetate. The organic layer was concentrated under vacuum to afford the desired product (0.090 g, 92% yield) as a yellow solid. LCMS (m/z): 504.47 [M+1].

(xiv). (S)-10-((R)-3-(1-aminocyclopropyl)pyrrolidin-1-yl)-9-fluoro-3-methyl-5-oxo-2,3-dihydro-5H-[1,4]thiazino[2,3,4-ij]quinoline-6-carboxylic acid hydrochloric acid salt: (S)-10-((R)-3-(1-((tert-butoxycarbonyl)amino)cyclopropyl)pyrrolidin-1-yl)-9-fluoro-3-methyl-5-oxo-2,3-dihydro-5H-[1,4]thiazino[2,3,4-ij]quinoline-6-carboxylic acid (90.0 mg, 0.18 mmol) was dissolved in DCM (2 mL) and HCl 4N in dioxane (1.00 mL) was added. The mixture was stirred at rt for 2 h. The solvent was evaporated, and the crude residue was triturated with EtOAc, affording the desired product (20.0 mg, 20% yield) as a yellow solid. ¹H NMR (400 MHz, MeOD) δ 8.87 (s, 1H), 7.57 (d, J=11.7 Hz, 1H), 5.85 (s, 1H), 3.57-3.20 (m, 6H), 2.86-2.77 (m, 1H), 2.25 (s, 1H), 1.84 (d, J=12.1 Hz, 1H), 1.48 (s, J=6.5 Hz, 3H), 1.12-0.98 (m, 4H). LCMS: t_{R}=1.38 min, m/z=404.6 [M+1] (3 minutes run, method B).

### Example 12.1

### (S)-10-((R)-3-(1-aminocyclopropyl)pyrrolidin-1-yl)-8-fluoro-3-methyl-5-oxo-2,3-dihydro-5H-[1,4]oxazino[2,3,4-ij]quinoline-6-carboxylic acid trifluoroacetate salt

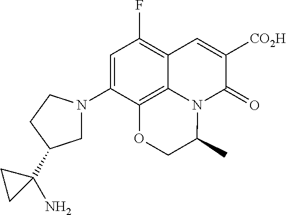 [SMILES: C[C@H]1COc2c(N3CC[C@@H](C4(N)CC4)C3)cc(F)c3cc(C(=O)O)c(=O)n1c23]

The title compound was prepared in accordance with the following scheme:

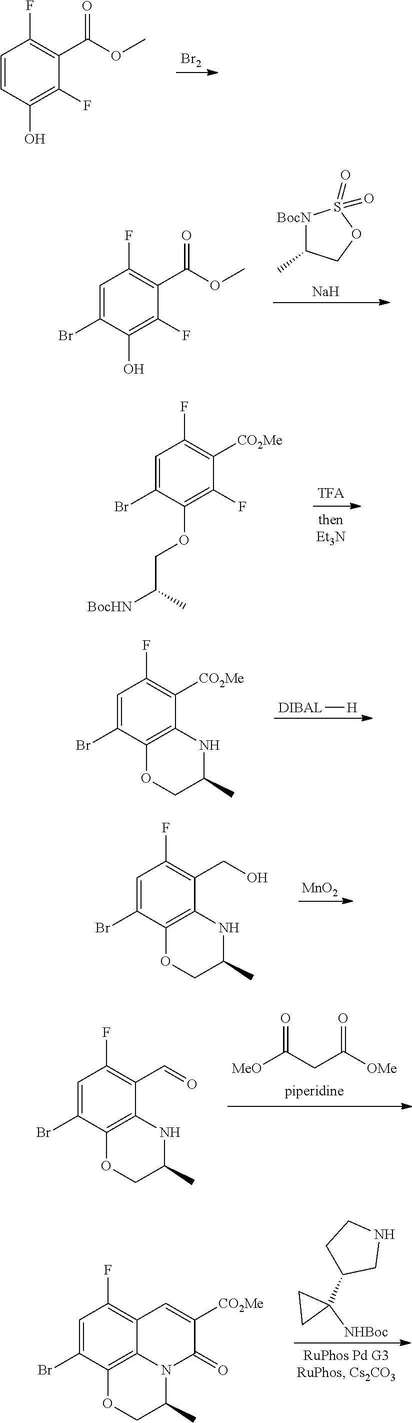

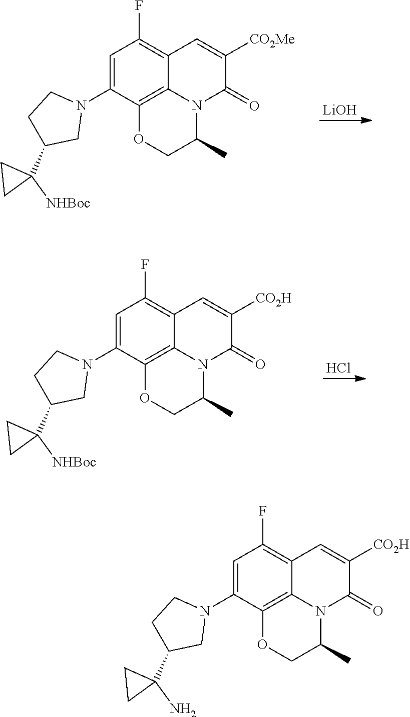 [SMILES: COC(=O)c1cc2c(F)cc(N3CC[C@@H](C4(NC(=O)OC(C)(C)C)CC4)C3)c3c2n(c1=O)[C@@H](C)CO3 ; "LiOH" ; C[C@H]1COc2c(N3CC[C@@H](C4(NC(=O)OC(C)(C)C)CC4)C3)cc(F)c3cc(C(=O)O)c(=O)n1c23 ; Cl ; C[C@H]1COc2c(N3CC[C@@H](C4(N)CC4)C3)cc(F)c3cc(C(=O)O)c(=O)n1c23]

(i). Methyl 4-bromo-2,6-difluoro-3-hydroxybenzoate: To 2,6-difluoro-3-benzoic acid methyl ester (750 mg, 3.99 mmol) in AcOH (20 mL) was added sodium acetate (360 mg, 4.39 mmol) and Br₂ (956 mg, 0.31 mL, 5.98 mmol). The mixture was stirred at rt for 4 h. After completion, a saturated solution of sodium thiosulfate was added followed by water and EtOAc. The phases were separated, and the organic layer was washed with water, brine, dried over Na₂SO₄, filtered and concentrated. The crude residue was purified by silica gel chromatography using 100% heptane to 50% EtOAc in heptane, affording the product (915 mg, 86% yield) as a white solid. ¹H NMR (400 MHz, Chloroform-d) δ 7.16 (dd, J=8.8, 2.3 Hz, 1H), 5.45 (br s, 1H), 3.96 (s, 3H).

(ii).Methyl (S)-4-bromo-3-(2-((tert-butoxycarbonyl)amino)propoxy)-2,6-difluorobenzoate: To a solution of methyl 4-bromo-2,6-difluoro-3-hydroxybenzoate (910 mg, 3.41 mmol) in DMF (10 mL) was added sodium hydride (150 mg, 3.75 mmol) and the resulting mixture was stirred at rt for 5 minutes. tert-Butyl (S)-4-methyl-1,2,3-oxathiazolidine-3-carboxylate 2,2-dioxide (889 mg, 3.75 mmol) was added and the mixture was stirred at rt for 2 h. EtOAc was added, followed by saturated aqueous NH₄Cl. The phase were separated and the aqueous layer was extracted with EtOAc. The combined organic layer was washed with water and brine. The organic phase was dried on Na₂SO₄, filtered, concentrated. The crude residue was purified by silica gel chromatography using 100% heptane to 40% EtOAc in heptane, affording the product (1.39 g, 96% yield) as a white solid. ¹H NMR (400 MHz, Chloroform-d) δ 7.18 (dd, J=8.8, 2.2 Hz, 1H), 4.05-3.98 (m, 3H), 3.94 (s, 3H), 1.45 (s, 9H), 1.36 (d, J=6.6 Hz, 3H).

(iii). Methyl (S)-8-bromo-6-fluoro-3-methyl-3,4-dihydro-2H-benzo[b][1,4]oxazine-5-carboxylate: TFA (5 mL) was added to a mixture of methyl (S)-4-bromo-3-(2-((tert-butoxycarbonyl)amino)propoxy)-2,6-difluorobenzoate (1.39 g, 3.27 mmol) in DCM (5 mL) at rt. After 15 minutes, the residue was partitioned between DCM and saturated aqueous NaHCO₃. The layers were separated and the aqueous layer was extracted again with DCM. The combined organic extract was washed with brine, dried over Na₂SO₄ and concentated under reduced pressure. To the crude product in DMSO (5 mL) was added triethylamine (1.65 g, 2.28 mL, 16.3 mmol) followed by stirring at 50° C. for 30 minutes. After completion, the reaction mixture was quenched with water and extracted with EtOAc. The combined organic layer was washed with water, brine, dried over sodium sulfate and concentrated under vacuum to afford the desired product (892 mg, 90% yield) as a yellow solid. LCMS (m/z): 304.3 [M].

(iv). (S)-(8-bromo-6-fluoro-3-methyl-3,4-dihydro-2H-benzo[b][1,4]oxazin-5-yl)methanol: Methyl (S)-8-bromo-6-fluoro-3-methyl-3,4-dihydro-2H-benzo[b][1,4]oxazine-5-carboxylate (890 mg, 2.93 mmol) was dissolved in toluene (15 mL) and cooled to −78° C. DIBAL (1M in toluene, 10.24 mL, 10.24 mmol) was added to the stirring mixture and the temperature was allowed to reach rt over lh. After completion of the mixture was quenched with Rochelle salt solution and extracted with EtOAc. The combined organic layer was washed with brine, dried over sodium sulfate and concentrated under vacuum. The crude residue was purified by silica gel chromatography using 100% heptane to 50% EtOAc in heptane, affording the product (655 mg, 81% yield) as a white solid. LCMS (m/z): 276.2 [M].

(v). (S)-8-Bromo-6-fluoro-3-methyl-3,4-dihydro-2H-benzo[b][1,4]oxazine-5-carbaldehyde: (S)-(8-bromo-6-fluoro-3-methyl-3,4-dihydro-2H-benzo[b][1,4]oxazin-5-yl)methanol (448 mg, 1.64 mmol) was dissolved in DMF (10 mL). MnO₂ (825 mg, 9.49 mmol) was added, followed by stirring at rt for 4 h. More MnO₂ (825 mg, 9.49 mmol) was added, and the reaction was stirred overnight. The mixture was diluted with EtOAc and filtered through a celite pad. The filtrate was quenched with water and extracted with EtOAc. The combined organic layer was washed with water, brine, dried over sodium sulfate and concentrated under vacuum. The crude residue was purified by silica gel chromatography using 100% heptane to 30% EtOAc in heptane, affording the product (448 mg, 69% yield) as a yellow solid. LCMS (m/z): 276.2 [M+2].

(vi). Methyl (S)-10-bromo-8-fluoro-3-methyl-5-oxo-2,3-dihydro-5H-[1,4]oxazino[2,3,4-ij]quinoline-6-carboxylate: (S)-8-Bromo-6-fluoro-3-methyl-3,4-dihydro-2H-benzo[b][1,4]oxazine-5-carbaldehyde (0.45 g, 1.64 mmol) was dissolved in toluene (15 mL). Dimethyl malonate (1.08 g, 0.94 mL, 8.17 mmol), piperidine (0.42 g, 0.48 mL, 4.90 mmol) and acetic acid (0.29 g, 0.28 mL, 4.90 mmol) were added and the reaction mixture was stirred at 110° C. for 4 h. After completion, the reaction mixture was concentrated under vacuum to afford a crude residue which was purified by silica gel chromatography using 100% heptane to 100% EtOAc, affording the desired product (0.54 g, 93% yield) as a yellow solid. LCMS (m/z): 358.3 [M+2].

(vii). Methyl (S)-10-((R)-3-(1-((tert-butoxycarbonyl)amino)cyclopropyl)pyrrolidin-1-yl)-8-fluoro-3-methyl-5-oxo-2,3-dihydro-5H-[1,4]oxazino[2,3,4-ij]quinoline-6-carboxylate: Methyl (S)-10-bromo-8-fluoro-3-methyl-5-oxo-2,3-dihydro-5H-[1,4]oxazino[2,3,4-ij]quinoline-6-carboxylate (250 mg, 0.70 mmol), tert-butyl (R)-(1-(pyrrolidin-3-yl)cyclopropyl)carbamate (238 mg, 1.05 mmol) and Cs₂CO₃ (686 mg, 2.11 mmol) were suspended in toluene (35 mL) and degassed with nitrogen for 5 minutes. RuPhos (66.0 mg, 0.14 mmol) and RuPhosPdG₃ (117 mg, 0.14 mmol) were added and the reaction mixture was heated to 90° C. for 6 h . The reaction mixture was filtered on a disposable funnel and concentrated under vacuum. The crude residue was purified by silica gel chromatography using 100% heptane to 100% EtOAc, affording the desired product (112 mg, 32% yield) as a yellow solid. LCMS (m/z): 502.6 [M+1].

(viii). (S)-10-((R)-3-(1-((tert-butoxycarbonyl)amino)cyclopropyl)pyrrolidin-1-yl)-8-fluoro-3-methyl-5-oxo-2,3-dihydro-5H-[1,4]oxazino[2,3,4-ij]quinoline-6-carboxylic acid: Methyl (S)-10-((R)-3-(1-((tert-butoxycarbonyl)amino)cyclopropyl)pyrrolidin-1-yl)-8-fluoro-3-methyl-5-oxo-2,3-dihydro-5H-[1,4]oxazino[2,3,4-ij]quinoline-6-carboxylate (0.112 g, 0.223 mmol) was dissolved in a mixture of MeOH:H₂O (3:1, 6 mL). LiOH (0.075 g, 1.786 mmol) was added and the reaction mixture was stirred at rt overnight. The reaction mixture was quenched with water, acidified with 1M HCl and extracted with ethyl acetate. The organic layer was dried over Na₂SO₄, filtered and concentrated under vacuum to afford the desired product as yellow solid. LCMS (m/z): 488.6 [M+1].

(ix). (S)-10-((R)-3-(1-aminocyclopropyl)pyrrolidin-1-yl)-8-fluoro-3-methyl-5-oxo-2,3-dihydro-5H-[1,4]oxazino[2,3,4-ij]quinoline-6-carboxylic acid trifluoroacetate salt: (S)-10-((R)-3-(1-((tert-butoxycarbonyl)amino)cyclopropyl)pyrrolidin-1-yl)-8-fluoro-3-methyl-5-oxo-2,3-dihydro-5H-[1,4]oxazino[2,3,4-ij]quinoline-6-carboxylic acid (105 mg, 0.22 mmol) was dissolved in DCM (5 mL) and HCl 4N in dioxane (2.5 mL) was added. The mixture was stirred at rt for 2 h. The solvent was evaporated, and the crude residue was purified on reverse phase HPLC with a TFA buffer, affording the desired product (46.0 mg, 40% yield) as a yellow solid. ¹H NMR (400 MHz, Methanol-d₄) δ 8.64 (s, 1H), 6.59 (d, J=12.1 Hz, 1H), 5.07 (q, J=6.4 Hz, 1H), 4.44 (d, J=11.3 Hz, 1H), 4.01-3.87 (m, 2H), 3.76 (dd, J=26.1, 7.3 Hz, 2H), 3.55-3.50 (m, 1H), 2.68-2.63 (m, 1H), 2.18-2.09 (m, 1H), 1.85-1.66 (m, 1H), 1.43 (d, J=6.6 Hz, 3H), 1.00 (d, J=16.5 Hz, 4H). LCMS: tR=1.73 min, m/z=388.5 [M+1] (10 minute run, method A).

Using the procedures described for Example 12.1 the following compound were prepared:

| | | | HPLC | |
|---|---|---|---|---|
| Ex | | | method, | LCMS |
| # | Structure | Chemical Name | t_{R} (min) | [M + 1] |
| 12.2 | 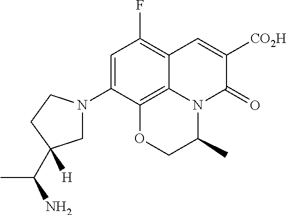 [SMILES: C[C@H](N)[C@@H]1CCN(c2cc(F)c3cc(C(=O)O)c(=O)n4c3c2OC[C@@H]4C)C1] | (S)-10-((R)-3-((S)-1- aminoethyl)pyrrolidin-1-yl)-8-fluoro-3- methyl-5-oxo-2,3-dihydro-5H- [1,4]oxazino[2,3,4-ij]quinoline-6-carboxylic acid (TFA salt) | A, 1.62 | 376.5 |
| 12.3¹ | 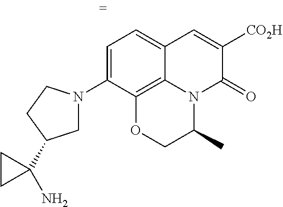 [SMILES: C[C@H]1COc2c(N3CC[C@@H](C4(N)CC4)C3)ccc3cc(C(=O)O)c(=O)n1c23] | (S)-10-((R)-3-(1- aminocyclopropyl)pyrrolidin-1-yl)-3- methyl-5-oxo-2,3-dihydro-5H- [1,4]oxazino[2,3,4-ij]quinoline-6-carboxylic acid (HCl salt) | B, 1.32 | 370.5 |
| 12.4¹ | 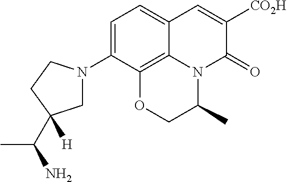 [SMILES: C[C@H](N)[C@@H]1CCN(c2ccc3cc(C(=O)O)c(=O)n4c3c2OC[C@@H]4C)C1] | (S)-10-((R)-3-((S)-1- aminoethyl)pyrrolidin-1-yl)-3-methyl-5- oxo-2,3-dihydro-5H-[1,4]oxazino[2,3,4- ij]quinoline-6-carboxylic acid (HCl salt) | B, 1.35 | 358.4 |
| 12.5¹ | 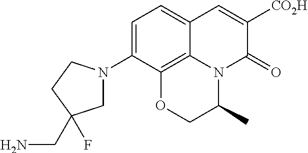 [SMILES: C[C@H]1COc2c(N3CCC(F)(CN)C3)ccc3cc(C(=O)O)c(=O)n1c23] | (3S)-10-(3-(aminomethyl)-3- fluoropyrrolidin-1-yl)-3-methyl-5-oxo-2,3- dihydro-5H-[1,4]oxazino[2,3,4-ij]quinoline- 6-carboxylic acid (HCl salt) | B, 1.31 | 362.4 |
| 12.6¹ | 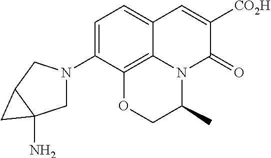 [SMILES: C[C@H]1COc2c(N3CC4CC4(N)C3)ccc3cc(C(=O)O)c(=O)n1c23] | (3S)-10-(1-amino-3- azabicyclo[3.1.0]hexan-3-yl)-3-methyl-5- oxo-2,3-dihydro-5H-[1,4]oxazino[2,3,4- ij]quinoline-6-carboxylic acid (HCl salt) | B, 1.32 | 342.4 |

¹Start at step ii, use 4-bromo-2-fluoro-3-hydroxybenzoate as starting material

HPLC method A: Characterized by high performance liquid chromatography (HPLC) on a Waters ACQUITY UPLC system with 1.2 mL/min flow rate; column Kinetex-C18, 2.6 um, 2.1×50 mm from Phenomenex, column temperature: 50° C.; gradient: 2-88% MeCN in water with 0.1% TFA over a 9.29 min period (unless indicated otherwise); compounds were detected by ultraviolet light (UV) absorption at 220nm.

HPLC method B: Characterized by high performance liquid chromatography (HPLC) on a Waters ACQUITY H class UPLC system with 0.55 mL/min flow rate; column BEH-C18, 1.7 um, 2.1×50 mm from Waters, column temperature: ambiant; gradient (solvent A is 2 mM Ammonium Acetate and 0.1% Formic Acid in Water, solvent B is 0.1% Formic Acid in acetonitrile): 5% solvent B hold for 0.4 min, 5-40% solvent B over 0.6 min, 40-60% solvent B over 1.2 min, 60-100% solvent B over 2.3 min then 100% solvent B over 3 min; compounds were detected by ultraviolet light (UV) absorption at 236 nm.

### Example 13.1

### (S)-10-((R)-3-(1-aminocyclopropyl)pyrrolidin-1-yl)-8,9-difluoro-3-methyl-5-oxo-2,3-dihydro-5H-[1,4]oxazino[2,3,4-ij]quinoline-6-carboxylic acid hydrochloride salt

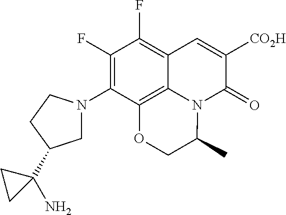 [SMILES: C[C@H]1COc2c(N3CC[C@@H](C4(N)CC4)C3)c(F)c(F)c3cc(C(=O)O)c(=O)n1c23]

The title compound was prepared in accordance with the following scheme:

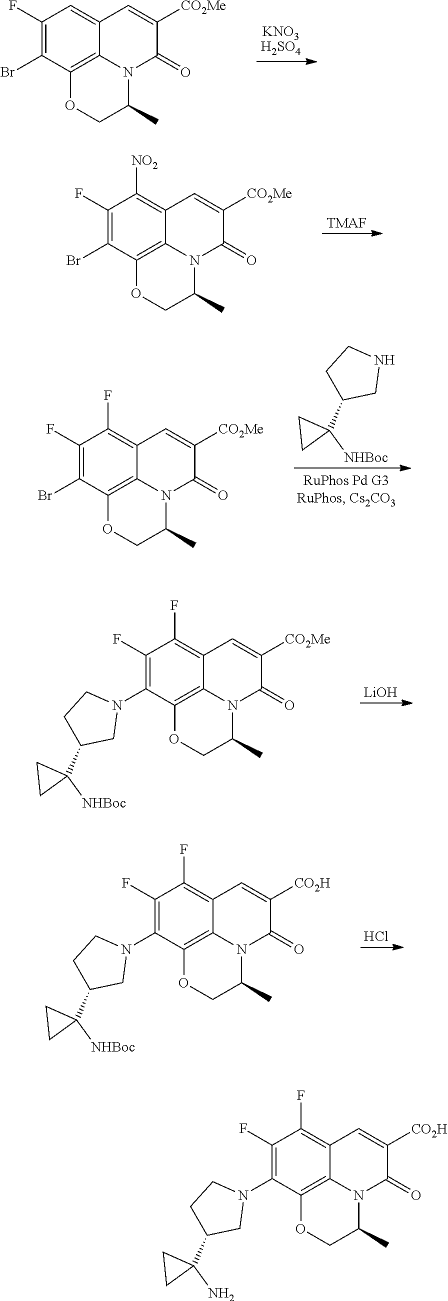

(i). Methyl (S)-10-bromo-9-fluoro-3-methyl-8-nitro-5-oxo-2,3-dihydro-5H-[1,4]oxazino[2,3,4-ij]quinoline-6-carboxylate: Methyl (S)-10-bromo-9-fluoro-3-methyl-5-oxo-2,3-dihydro-5H-[1,4]oxazino[2,3,4-ij]quinoline-6-carboxylate (3.50 g, 9.85 mmol) was dissolved in concentrated H₂SO₄ (35 mL) at 0° C. KNO₃ (1.04 g, 10.4 mmol) was added and the reaction mixture was stirred at rt for 1 h. The reaction mixture was poured into ice and extracted with EtOAc. The combined organic layer was washed with saturated NaHCO₃, brine, dried over sodium sulfate and concentrated under vacuum to afford the desired product (3.40 g, 86% yield) as a yellow solid which was directly used in the next step without any further purification. LCMS (m/z): 403.2 [M+2].

(ii).Methyl (S)-10-bromo-8,9-difluoro-3-methyl-5-oxo-2,3-dihydro-5H-[1,4]oxazino[2,3,4-ij]quinoline-6-carboxylate: Methyl (S)-10-bromo-9-fluoro-3-methyl-8-nitro-5-oxo-2,3-dihydro-5H-[1,4]oxazino[2,3,4-ij]quinoline-6-carboxylate (3.40 g, 8.47 mmol) was dissolved in DMF (15 mL). Tetramethylammonium fluoride (1.18 g, 12.7 mmol) was added slowly at 0° C. and the mixture was stirred at rt for 16 h. The reaction mixture was poured into ice cold water and extracted with EtOAc. The combined organic layer was washed with water, brine, dried over sodium sulfate and concentrated under vacuum to afford the desired product (1.93 g, 61% yield) which was directly used in the next step without any further purification. ¹H NMR (400 MHz, Chloroform-d) δ 8.68 (s, 1H), 5.19 (d, J=7.0 Hz, 1H), 4.60 (dd, J=11.4, 1.2 Hz, 1H), 4.34-4.12 (m, 1H), 4.02 (s, 3H), 1.46 (d, J=6.6 Hz, 3H). LCMS (m/z): 376.2 [M+2].

(iii). Methyl (S)-10-((R)-3-(1-((tert-butoxycarbonyl)amino)cyclopropyl)pyrrolidin-1-yl)-8,9-difluoro-3-methyl-5-oxo-2,3-dihydro-5H-[1,4]oxazino[2,3,4-ij]quinoline-6-carboxylate: Methyl (S)-10-bromo-8,9-difluoro-3-methyl-5-oxo-2,3-dihydro-5H-[1,4]oxazino[2,3,4-ij]quinoline-6-carboxylate (0.250 g, 0.668 mmol), tert-butyl (R)-(1-(pyrrolidin-3-yl)cyclopropyl)carbamate (0.302 g, 1.330 mmol) and Cs₂CO₃ (0.544 g, 1.670 mmol) were suspended in toluene (10 mL) and degassed with nitrogen for 5 minutes. Ruphos (0.044 g, 0.100 mmol) and PdG₃RuPhos (0.083 g, 0.100 mmol) were added and the reaction mixture was heated at 90° C. for 10 h. The reaction mixture was filtered through celite pad and washed with excess EtOAc. The organic layer was concentrated under vacuum to afford a crude residue which was purified by reverse phase chromatography (60-70% MeOH in water) affording the desired product (0.190 g, 54% yield) as a pale yellow solid. LCMS (m/z): 520.6 [M+1].

(iv). (S)-10-((R)-3-(1-((tert-butoxycarbonyl)amino)cyclopropyl)pyrrolidin-1-yl)-8,9-difluoro-3-methyl-5-oxo-2,3-dihydro-5H-[1,4]oxazino[2,3,4-ij]quinoline-6-carboxylic acid: Methyl (S)-10-((R)-3-(1-((tert-butoxycarbonyl)amino)cyclopropyl)pyrrolidin-1-yl)-8,9-difluoro-3-methyl-5-oxo-2,3-dihydro-5H-[1,4]oxazino[2,3,4-ij]quinoline-6-carboxylate (0.19 g, 0.37 mmol) was added to a mixture of MeOH:H₂O (3:1, 5 mL). LiOH.H₂O (1M in water, 1.10 mL, 0.11 mmol) was added and the reaction mixture was stirred at rt for 3 h. The reaction mixture was quenched with cold water, acidified with diluted HCl to pH 4 and extracted with ethyl acetate. The organic layer was dried over sodium sulfate and concentrated under vacuum to afford the desired product (175 mg, 94% yield) as a yellow solid which was directly used in the next step without any further purification. ¹H NMR (400 MHz, Chloroform-d) δ 14.26 (s, 1H), 5.12 (d, J=7.0 Hz, 1H), 5.03 (d, J=1.3 Hz, 1H), 4.41 (d, J=11.3 Hz, 1H), 4.00-3.75 (m, 5H), 2.27-2.23 (m, 1H), 2.03-2.01 (m, 1H), 1.55-1.52 (m, 1H), 1.50-1.39 (m, 9H), 1.41-1.35 (d, J=1.1 Hz, 3H), 1.28 (s, 2H), 0.93-0.87 (m, 1H), 0.82-0.79 (m, 1H). LCMS (m/z): 506.5 [M+1].

(vi). (S)-10-((R)-3-(1-aminocyclopropyl)pyrrolidin-1-yl)-8,9-difluoro-3-methyl-5-oxo-2,3-dihydro-5H-[1,4]oxazino[2,3,4-ij]quinoline-6-carboxylic acid hydrochloride: (S)-10-((R)-3-(1-((tert-butoxycarbonyl)amino)cyclopropyl)pyrrolidin-1-yl)-8,9-difluoro-3-methyl-5-oxo-2,3-dihydro-5H-[1,4]oxazino[2,3,4-ij]quinoline-6-carboxylic acid (0.175 g, 0.349 mmol) was dissolved in DCM (3 mL). HCl-Dioxane (4M, 2 mL) was added and the reaction mixture was stirred at rt for 2 h. The reaction mixture was concentrated under vacuum to afford a crude residue which was triturated with EtOAc, affording the desired product (0.137 g, 95% yield) as a yellow solid. ¹H NMR (400 MHz, MeOD) δ 8.83 (s, 1H), 5.14 (d, J=6.7 Hz, 1H), 4.54 (d, J=11.4 Hz, 1H), 4.07 (d, J=11.5 Hz, 2H), 3.97-3.79 (m, 3H), 2.70-2.61 (m, 1H), 2.20-2.12 (m, 1H), 1.76 (q, J=10.4 Hz, 1H), 1.47 (d, J=6.7 Hz, 3H), 1.08-0.99 (m, 3H). LCMS: tR=1.38 min, m/z=406.3 [M+1] (10 minute run, method B).

Using the procedures described for Example 13.1 the following compound were prepared:

| | | | HPLC, | |
|---|---|---|---|---|
| Ex | | | t_{R} | LCMS |
| # | Structure | Chemical Name | (min) | [M + 1] |
| 13.2 | 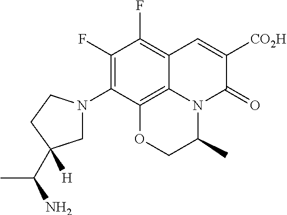 [SMILES: C[C@H](N)[C@@H]1CCN(c2c(F)c(F)c3cc(C(=O)O)c(=O)n4c3c2OC[C@@H]4C)C1] | (S)-10-((R)-3-((S)-1- aminoethyl)pyrrolidin-1-yl)-8,9-difluoro- 3-methyl-5-oxo-2,3-dihydro-5H- [1,4]oxazino[2,3,4-ij]quinoline-6- carboxylic acid (TFA salt) | 1.38 | 394.4 |
| 13.3 | 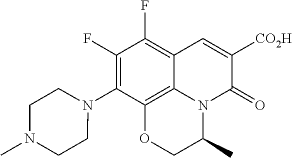 [SMILES: C[C@H]1COc2c(N3CCN(C)CC3)c(F)c(F)c3cc(C(=O)O)c(=O)n1c23] | (S)-10-((R)-3-(1- aminocyclopropyl)pyrrolidin-1-yl)-3- methyl-5-oxo-2,3-dihydro-5H- [1,4]oxazino[2,3,4-ij]quinoline-6- carboxylic acid | 1.31 | 380.5 |
| 13.4¹ | 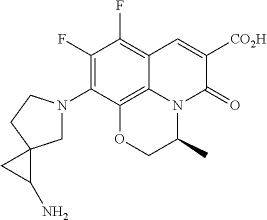 [SMILES: C[C@H]1COc2c(N3CCC4(CC4N)C3)c(F)c(F)c3cc(C(=O)O)c(=O)n1c23] | (3S)-10-(1-amino-5-azaspiro[2.4]heptan- 5-yl)-8,9-difluoro-3-methyl-5-oxo-2,3- dihydro-5H-[1,4]oxazino[2,3,4- ij]quinoline-6-carboxylic acid (TFA salt) | 1.36 | 392.5 |
| 13.5 | 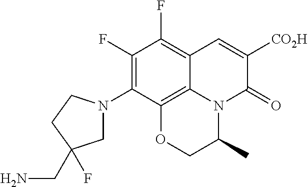 [SMILES: C[C@H]1COc2c(N3CCC(F)(CN)C3)c(F)c(F)c3cc(C(=O)O)c(=O)n1c23] | (3S)-10-(3-(aminomethyl)-3- fluoropyrrolidin-1-yl)-8,9-difluoro-3- methyl-5-oxo-2,3-dihydro-5H- [1,4]oxazino[2,3,4-ij]quinoline-6- carboxylic acid (HCl salt) | 1.34 | 398.5 |
| 13.6¹ | 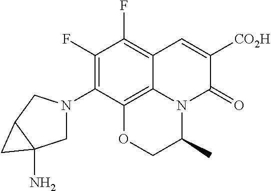 [SMILES: C[C@H]1COc2c(N3CC4CC4(N)C3)c(F)c(F)c3cc(C(=O)O)c(=O)n1c23] | (3S)-10-(1-amino-3- azabicyclo[3.1.0]hexan-3-yl)-8,9-difluoro- 3-methyl-5-oxo-2,3-dihydro-5H- [1,4]oxazino[2,3,4-ij]quinoline-6- carboxylic acid (TFA salt) | 1.32 | 378.7 |
| 13.7¹ | 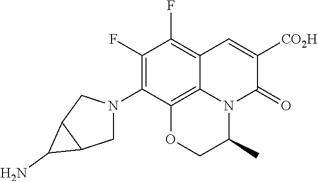 [SMILES: C[C@H]1COc2c(N3CC4C(N)C4C3)c(F)c(F)c3cc(C(=O)O)c(=O)n1c23] | (3S)-10-(6-amino-3- azabicyclo[3.1.0]hexan-3-yl)-8,9-difluoro- 3-methyl-5-oxo-2,3-dihydro-5H- [1,4]oxazino[2,3,4-ij]quinoline-6- carboxylic acid (TFA salt) | 1.33 | 378.48 |
| 13.8 | 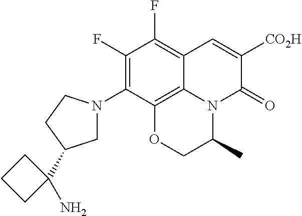 [SMILES: C[C@H]1COc2c(N3CC[C@@H](C4(N)CCC4)C3)c(F)c(F)c3cc(C(=O)O)c(=O)n1c23] | (S)-10-((R)-3-(1- aminocyclobutyl)pyrrolidin-1-yl)-8,9- difluoro-3-methyl-5-oxo-2,3-dihydro-5H- [1,4]oxazino[2,3,4-ij]quinoline-6- carboxylic acid (HCl salt) | 1.40 | 420.3 |

¹Single diastereoisomer, absolute stereochemistry unknown.

HPLC method : Characterized by high performance liquid chromatography (HPLC) on a Waters ACQUITY H class UPLC system with 0.55 mL/min flow rate; column BEH-C18, 1.7 um, 2.1×50 mm from Waters, column temperature: ambiant; gradient (solvent A is 2 mM Ammonium Acetate and 0.1% Formic Acid in Water, solvent B is 0.1% Formic Acid in acetonitrile) : 5% solvent B hold for 0.4 min, 5-40% solvent B over 0.6 min, 40-60% solvent B over 1.2 min, 60-100% solvent B over 2.3 min then 100% solvent B over 3 min; compounds were detected by ultraviolet light (UV) absorption at 236 nm.

### Example 14

### (S)-8-amino-10-((R)-3-(1-aminocyclopropyl)pyrrolidin-1-yl)-9-fluoro-3-methyl-5-oxo-2,3-dihydro-5H-[1,4]oxazino[2,3,4-ij]quinoline-6-carboxylic acid hydrochloride salt

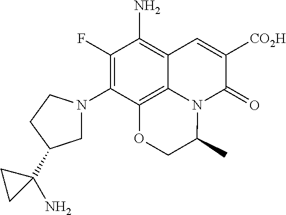 [SMILES: C[C@H]1COc2c(N3CC[C@@H](C4(N)CC4)C3)c(F)c(N)c3cc(C(=O)O)c(=O)n1c23]

The title compound was prepared in accordance with the following scheme:

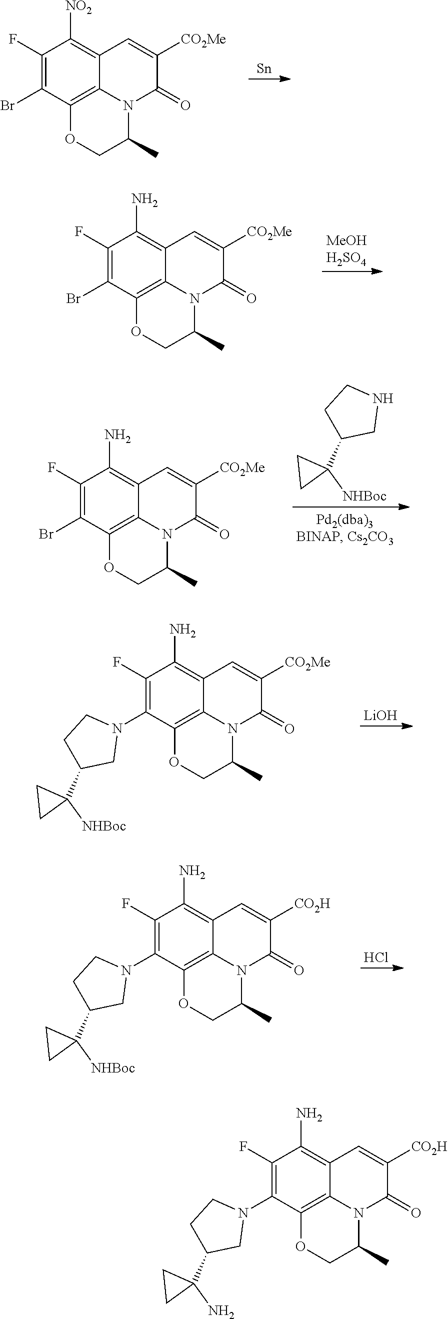

(i). Methyl (S)-8-amino-10-bromo-9-fluoro-3-methyl-5-oxo-2,3-dihydro-5H-[1,4]oxazino[2,3,4-ij]quinoline-6-carboxylate: Methyl (S)-10-bromo-9-fluoro-3-methyl-8-nitro-5-oxo-2,3-dihydro-5H-[1,4]oxazino[2,3,4-ij]quinoline-6-carboxylate (0.12 g, 0.30 mmol) was dissolved in THF (5 mL) and cooled to 0° C. Tin (36.0 mg, 0.30 mmol) and 4M HCl (0.5 ml) were added and the reaction mixture was heated to 45° C. for 30 minutes. The reaction mixture was quenched with cold water, acidified with diluted HCl to pH 4 and extracted with ethyl acetate. The organic layer was dried over sodium sulfate and concentrated under vacuum to afford the desired product (0.11 g, 98% yield) as a beige solid which was directly used in the next step without any further purification. LCMS (m/z): 359.1 [M+2].

(ii).Methyl (S)-8-amino-10-bromo-9-fluoro-3-methyl-5-oxo-2,3-dihydro-5H-[1,4]oxazino[2,3,4-ij]quinoline-6-carboxylate: (S)-8-amino-10-bromo-9-fluoro-3-methyl-5-oxo-2,3-dihydro-5H-[1,4]oxazino[2,3,4-ij]quinoline-6-carboxylate (0.11 g, 0.31 mmol) was dissolved in methanol (12 mL) and cooled to 0° C. H₂SO₄ (3 drops) was added and the reaction mixture was heated to reflux for 6 h. The reaction mixture was quenched with cold water and extracted with ethyl acetate. The organic layer was dried over sodium sulfate and concentrated under vacuum to afford the desired product (0.10 g, 87% yield) as a beige solid which was directly used in the next step without any further purification. LCMS (m/z): 373.3 [M+2].

(iii). Methyl (S)-8-amino-10-((R)-3-(1-((tert-butoxycarbonyl)amino)cyclopropyl) pyrrolidin-1-yl)-9-fluoro-3-methyl-5-oxo-2,3-dihydro-5H-[1,4]oxazino[2,3,4-ij]quinoline-6-carboxylate: Methyl (S)-8-amino-10-bromo-9-fluoro-3-methyl-5-oxo-2,3-dihydro-5H-[1,4]oxazino[2,3,4-ij]quinoline-6-carboxylate (0.080 g, 0.215 mmol), tert-butyl (R)-(1-(pyrrolidin-3-yl)cyclopropyl)carbamate (0.058 g, 0.258 mmol) and Cs₂CO₃ (0.175 g, 0.539 mmol) were suspended in toluene (10 mL) and degassed with nitrogen for 5 minutes. BINAP (0.020 g, 0.032 mmol) and Pd₂dba₃ (0.029 g, 0.032 mmol) were added and the reaction mixture was heated to 110° C. for 10 h. The reaction mixture was filtered through a celite pad and washed with an excess of EtOAc. The organic layer was concentrated under vacuum to afford a crude residue which was purified by Prep TLC using 80% EtOAc in hexane, providing the desired product (0.06 g, 54% yield) as a yellow gum. LCMS (m/z): 517.5 [M+1].

(iv). (S)-8-amino-10-((R)-3-(1-((tert-butoxycarbonyl)amino)cyclopropyl)pyrrolidin-1-yl)-9-fluoro-3-methyl-5-oxo-2,3-dihydro-5H-[1,4]oxazino[2,3,4-ij]quinoline-6-carboxylic acid: Methyl (S)-8-amino-10-((R)-3-(1-((tert-butoxycarbonyl)amino)cyclopropyl) pyrrolidin-1-yl)-9-fluoro-3-methyl-5-oxo-2,3-dihydro-5H-[1,4]oxazino[2,3,4-ij]quinoline-6-carboxylate (0.06 g, 0.12 mmol) was added into a mixture of MeOH:H₂O (3:1, 5 mL). LiOH.H₂O (1M in water, 0.34 mL, 0.35 mmol) was added and the reaction mixture was stirred at rt for 2 h. The reaction mixture was quenched with cold water, acidified with diluted HCl to pH 4 and extracted with ethyl acetate. The organic layer was dried over sodium sulfate and concentrated under vacuum to afford the desired product (0.05 g, 86% yield) as a beige solid which was directly used in the next step without any further purification. LCMS (m/z): 503.5 [M+1].

(v). (S)-8-amino-10-((R)-3-(1-aminocyclopropyl)pyrrolidin-1-yl)-9-fluoro-3-methyl-5-oxo-2,3-dihydro-5H-[1,4]oxazino[2,3,4-ij]quinoline-6-carboxylic acid: (S)-8-amino-10-((R)-3-(1-((tert-butoxycarbonyl)amino)cyclopropyl)pyrrolidin-1-yl)-9-fluoro-3-methyl-5-oxo-2,3-dihydro-5H-[1,4]oxazino[2,3,4-ij]quinoline-6-carboxylic acid (0.05 g, 0.09 mmol) was dissolved in DCM (2 mL). HCl-Dioxane (4M, 1 mL) was added and the reaction mixture was stirred at rt for 1 h. The reaction mixture was concentrated under vacuum to afford a crude residue which was purified by trituration with EtOAc, affording the desired product (12.0 mg, 30% yield) as a yellow solid. ¹H NMR (400 MHz, MeOD) δ 8.92 (s, 1H), 5.08 (d, J=6.4 Hz, 1H), 5.04-5.00 (m, 1H), 4.41 (d, J=10.6 Hz, 1H), 4.03-4.00 (m, 1H), 3.95-3.92 (m, 1H), 3.77-3.74 (m, 2H), 2.36-2.34 (m, 1H), 2.07-2.04 (m, 1H), 1.77-1.75 (m, 1H), 1.42 (d, J=6.6 Hz, 3H), 0.81-75 (m, 4H). LCMS: tR=1.34 min, m/z=403.4 [M+1] (10 minute run, method B).

### Example 15

### (S)-8-amino-10-((R)-3-(1-aminocyclopropyl)pyrrolidin-1-yl)-9-methoxy-3-methyl-5-oxo-2,3-dihydro-5H-[1,4]oxazino[2,3,4-ij]quinoline-6-carboxylic acid formate salt

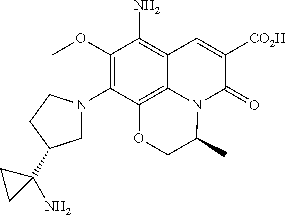 [SMILES: COc1c(N2CC[C@@H](C3(N)CC3)C2)c2c3c(cc(C(=O)O)c(=O)n3[C@@H](C)CO2)c1N]

The title compound was prepared in accordance with the following scheme:

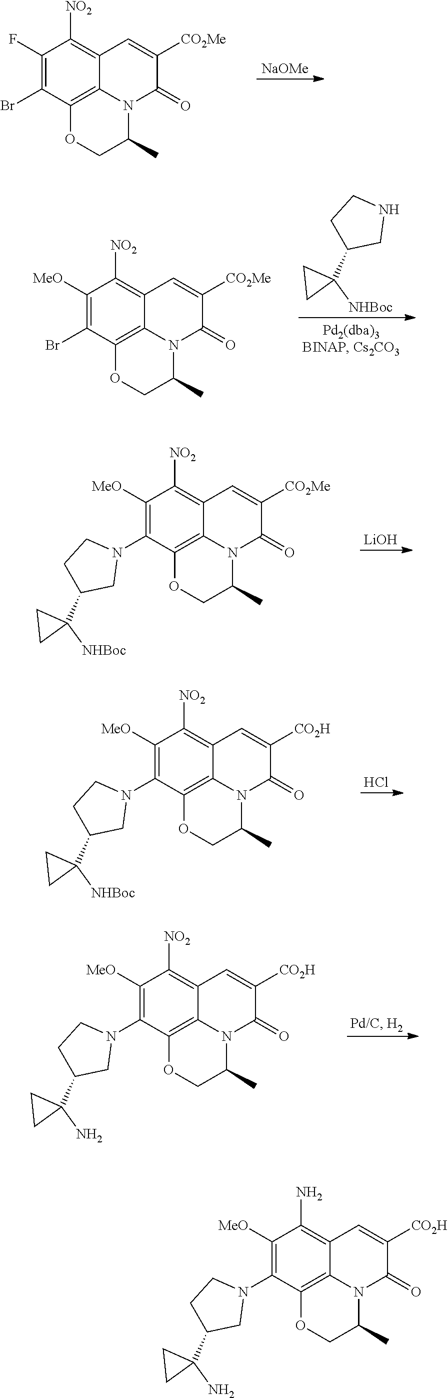

(i). Methyl (S)-10-bromo-9-methoxy-3-methyl-8-nitro-5-oxo-2,3-dihydro-5H-[1,4]oxazino[2,3,4-ij]quinoline-6-carboxylate: Methyl (S)-10-bromo-9-fluoro-3-methyl-8-nitro-5-oxo-2,3-dihydro-5H-[1,4]oxazino[2,3,4-ij]quinoline-6-carboxylate (0.20 g, 0.50 mmol) was dissolved in methanol (4 mL) and cooled to 0° C. NaOMe (0.03 g, 055 mmol) was added at 0° C. and the reaction mixture was stirred at rt for 16 h. The reaction mixture was concentrated under vacuum to afford a crude residue which was diluted with EtOAc and washed with water. The organic layer was dried over sodium sulfate and concentrated under vacuum to afford the desired product (0.18 g, 86% yield) as a beige solid which was directly used in the next step without any further purification. LCMS (m/z): 415.17 [M+2].

(ii).Methyl (S)-10-((R)-3-(1-((tert-butoxycarbonyl)amino)cyclopropyl)pyrrolidin-1-yl)-9-methoxy-3-methyl-8-nitro-5-oxo-2,3-dihydro-5H-[1,4]oxazino[2,3,4-ij]quinoline-6-carboxylate: Methyl (S)-10-bromo-9-methoxy-3-methyl-8-nitro-5-oxo-2,3-dihydro-5H-[1,4]oxazino[2,3,4-ij]quinoline-6-carboxylate (0.178 g, 0.430 mmol), tert-butyl (R)-(1-(pyrrolidin-3-yl)cyclopropyl)carbamate (0.116 g, 0.517 mmol) and Cs₂CO₃ (0.351 g, 1.077 mmol) were suspended in toluene (10 mL) and degassed with nitrogen for 5 minutes. BINAP (0.040 g, 0.064 mmol) and Pd₂dba₃ (0.059 g, 0.064 mmol) were added and the reaction mixture was heated to 110° C. for 16 h. The reaction mixture was filtered through a celite pad and washed with an excess of EtOAc. The organic layer was concentrated under vacuum to afford a crude residue which was purified by silica gel chromatography using 100% heptane to 100% EtOAc as eluent, affording the desired product (0.110 g, 46% yield) as a pale yellow gum. LCMS (m/z): 559.5 [M+1].

(iii). (S)-10-((R)-3-(1-((tert-butoxycarbonyl)amino)cyclopropyl)pyrrolidin-1-yl)-9-methoxy-3-methyl-8-nitro-5-oxo-2,3-dihydro-5H-[1,4]oxazino[2,3,4-ij]quinoline-6-carboxylic acid: Methyl (S)-10-((R)-3-(1-((tert-butoxycarbonyl)amino)cyclopropyl)pyrrolidin-1-yl)-9-methoxy-3-methyl-8-nitro-5-oxo-2,3-dihydro-5H-[1,4]oxazino[2,3,4-ij]quinoline-6-carboxylate (0.110 g, 0.197 mmol) was added to a mixture of MeOH:H₂O (3:1, 5 mL). LiOH.H₂O (1M in water, 0.59 mL, 0.591 mmol) was added and the reaction mixture was stirred at rt for 3 h. The reaction mixture was quenched with water, acidified with diluted HCl to pH 4 and extracted with ethyl acetate. The organic layer was dried over sodium sulfate and concentrated under vacuum to afford the desired product (0.100 g, 93% yield) as a beige solid which was directly used in the next step without any further purification. LCMS (m/z): 545.6 [M+1].

(iv). (S)-10-((R)-3-(1-aminocyclopropyl)pyrrolidin-1-yl)-9-methoxy-3-methyl-8-nitro-5-oxo-2,3-dihydro-5H-[1,4]oxazino[2,3,4-ij]quinoline-6-carboxylic acid hydrochloride: (S)-10-((R)-3-(1-((tert-butoxycarbonyl)amino)cyclopropyl)pyrrolidin-1-yl)-9-methoxy-3-methyl-8-nitro-5-oxo-2,3-dihydro-5H-[1,4]oxazino[2,3,4-ij]quinoline-6-carboxylic acid (0.10 g, 0.18 mmol) was dissolved in DCM (2 mL). HCl-Dioxane (4M, 1 mL) was added and the reaction mixture was stirred at rt for 1 h. The reaction mixture was concentrated under vacuum to afford a crude residue which was triturated with EtOAc, providing the desired product (0.04 g, 49% yield) as a yellow solid. ¹H NMR (400 MHz, MeOD) δ 7.36 (s, 1H), 5.21 (s, 1H), 4.61 (d, J=11.6, 1H),4.22-4.19 (m, 1H), 4.03-3.99 (m, 1H), 3.89 (s, 3H), 3.73 (d, J=8.4 Hz, 2H), 3.61-3.57 (m, 1H), 2.57-2.55 (m, 1H), 2.30 (dq, J=13.4, 7.0 Hz, 1H), 1.96 (dt, J=12.8 , 6.6 Hz, 1H), 1.49 (d, J=5.9 Hz, 3H), 1.04 (br s, 4H). LCMS (m/z): 445.5 [M+1].

(v). (S)-8-amino-10-((R)-3-(1-aminocyclopropyl)pyrrolidin-1-yl)-9-methoxy-3-methyl-5-oxo-2,3-dihydro-5H-[1,4]oxazino[2,3,4-ij]quinoline-6-carboxylic acid formate salt: (S)-10-((R)-3-(1-aminocyclopropyl)pyrrolidin-1-yl)-9-methoxy-3-methyl-8-nitro-5-oxo-2,3-dihydro-5H-[1,4]oxazino[2,3,4-ij]quinoline-6-carboxylic acid hydrochloride (0.22 g, 0.05 mmol) was dissolved in methanol/ammonia solution (2M, 4 mL). 10% Pd/C (50% in water, 0.03 g, 0.14 mmol) was added and the reaction mixture was stirred under 1 atmosphere hydrogen pressure for 5h at rt. The reaction mixture was filtered through a celite pad and washed with excess MeOH. The organic layer was concentrated under vacuum to afford a crude residue which was purified by reverse phase Prep HPLC, providing the desired product (0.01 g, 50% yield) as a pale yellow solid. ¹H NMR (400 MHz, MeOD) δ 8.98 (s, 1H), 5.10 (d, J=7.5 Hz, 1H), 4.41 (d, J=11.3 Hz, 1H), 3.98 (dd, J=25.5, 9.7 Hz, 2H), 3.79 (d, J=9.5 Hz, 1H), 3.8-3.63 (m, 5H), 2.44-2.42 (m, 1H), 2.11-2.09 (m, 1H), 1.77 (q, J=10.5 Hz, 1H), 1.43 (d, J=6.7 Hz, 3H), 0.86-0.82 (m, 4H). LCMS: t_{R}=1.31 min, m/z=415.5 [M+1] (10 minute run, method B).

### Example 16

### (S)-10-((R)-3-(1-aminocyclopropyl)pyrrolidin-1-yl)-9-methoxy-3-methyl-5-oxo-2,3-dihydro-5H-[1,4]oxazino[2,3,4-ij]liquinoline-6-carboxylic acid formate salt

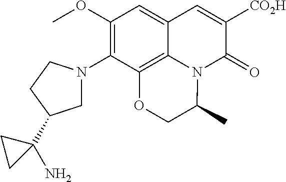 [SMILES: COc1cc2cc(C(=O)O)c(=O)n3c2c(c1N1CC[C@@H](C2(N)CC2)C1)OC[C@@H]3C]

The title compound was prepared in accordance with the following scheme:

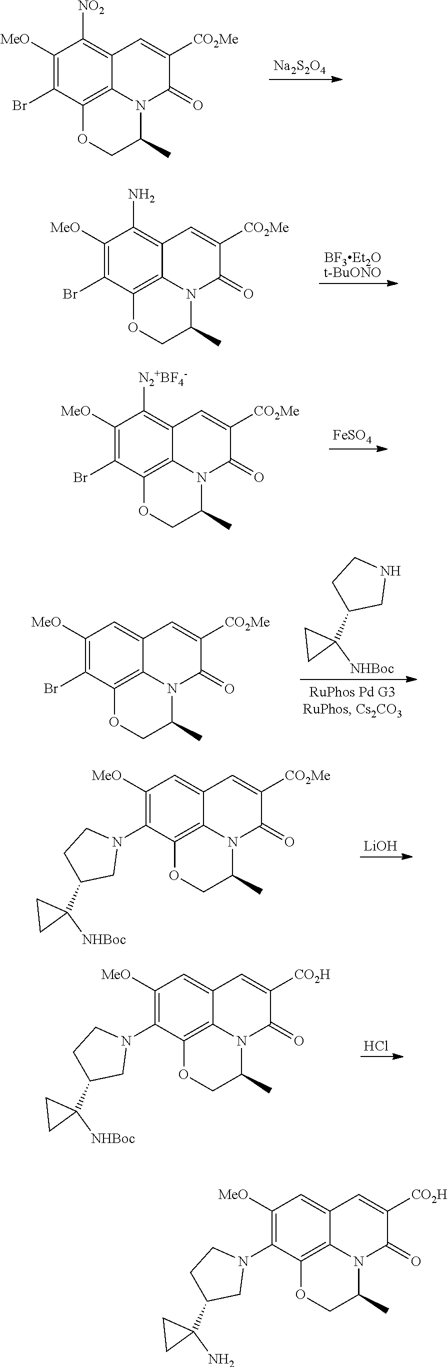

(i). Methyl (S)-8-amino-10-bromo-9-methoxy-3-methyl-5-oxo-2,3-dihydro-5H-[1,4]oxazino[2,3,4-ij]quinoline-6-carboxylate: Methyl (S)-10-bromo-9-methoxy-3-methyl-8-nitro-5-oxo-2,3-dihydro-5H-[1,4]oxazino[2,3,4-ij]quinoline-6-carboxylate (0.200 g, 0.484 mmol) was dissolved in ethanol (9 mL) and water (0.4 mL). The reaction mixture was heated to reflux and sodium dithionate (0.421 g, 2.421 mmol) was added. The reaction mixture was stirred for another 4 h. After completion the reaction mixture was concentrated under vacuum, the residue was dissolved in EtOAc and extracted with water. The combined organic layer was washed with water, brine, dried over sodium sulfate and concentrated under vacuum to afford the desired product (0.160 g, 86% yield) as a beige solid. ¹H NMR (400 MHz, Chloroform-d) δ 8.64 (s, 1H), 5.18 (d, J=6.7 Hz, 1H), 4.49 (d, J=11.3 Hz, 1H), 4.37-4.34 (m, 1H), 4.01(s, 3H), 3.98 (s, 3H), 1.45 (d, J=6.7 Hz, 3H). LCMS (m/z): 415.5 [M+2].

(ii). (S)-10-bromo-9-methoxy-6-(methoxycarbonyl)-3-methyl-5-oxo-2,3-dihydro-5H-[1,4]oxazino[2,3,4-ij]quinoline-8-diazonium tetrafluoroborate: BF₃.Et₂O (0.088 g, 0.626 mmol) was diluted in DCM (5 mL) at -10° C. Methyl (S)-8-amino-10-bromo-9-methoxy-3-methyl-5-oxo-2,3-dihydro-5H-[1,4]oxazino[2,3,4-ij]quinoline-6-carboxylate (0.160 g, 0.417 mmol) in DCM (5 mL) was added dropwise and the reaction mixture was stirred at −10° C. for 5 minutes. t-Butyl nitrite (0.051 g, 0.501 mmol) was added and the reaction mixture was stirred at −10° C. for 30 minutes and then at 0° C. for 20 minutes. After completion of the reaction the DCM was decanted and the residue triturated with n-Pentane and diethyl ether, affording the desired product (0.200 g, 93% yield).

(iii). Methyl (S)-10-bromo-9-methoxy-3-methyl-5-oxo-2,3-dihydro-5H-[1,4]oxazino[2,3,4-ij]quinoline-6-carboxylate: (S)-10-bromo-9-methoxy-6-(methoxycarbonyl)-3-methyl-5-oxo-2,3-dihydro-5H-[1,4]oxazino[2,3,4-ij]quinoline-8-diazonium tetrafluoroborate (0.200 g, 0.415 mmol) was dissolved in DMF (10 mL). FeSO₄.7H₂O (0.138 g, 0.498 mmol) was added at 0° C. and the reaction mixture was stirred at rt for 30 minutes. The reaction mixture was quenched with water and extracted with EtOAc. The combined organic layer was washed with brine, dried over sodium sulfate and concentrated under vacuum to afford a crude residue which was purified by silica gel chromatography (20-30% EtOAc in Hexane) affording the desired product (0.070 g, 52% yield). ¹H NMR (400 MHz, Chloroform-d) δ 8.48 (s, 1H), 6.80 (s, 1H), 5.21 (s, 1H), 4.60 (d, J=11.6 Hz, 1H), 4.03-3.98 (m, 7H), 1.47 (d, J=6.6 Hz, 3H). LCMS (m/z): 370.5 [M+2].

(iv). Methyl (S)-10-((R)-3-(1-((tert-butoxycarbonyl)amino)cyclopropyl)pyrrolidin-1-yl)-3-methyl-5-oxo-2,3-dihydro-5H-[1,4]oxazino[2,3,4-ij]quinoline-6-carboxylate: Methyl (S)-10-bromo-9-methoxy-3-methyl-5-oxo-2,3-dihydro-5H-[1,4]oxazino[2,3,4-ij]quinoline-6-carboxylate (0.070 g, 0.190 mmol), tert-butyl (R)-(1-(pyrrolidin-3-yl)cyclopropyl)carbamate (0.086 g, 0.380 mmol) and Cs₂CO₃ (0.186 g, 0.570 mmol) were suspended in toluene (10 mL) and degassed with nitrogen for 5 minutes. RuPhos (0.013 g, 0.028 mmol) and RuPhosPdG₃ (0.024 g, 0.028 mmol) were added and the reaction mixture was heated to 95° C. for 5 h. The reaction mixture was filtered through a celite pad and washed with excess EtOAc. The filtrate was concentrated under vacuum to give a crude residue which was purified by Prep TLC (60% EtOAc in Hexane), affording the desired product (0.050 g, 51% yield). LCMS (m/z): 514.59 [M+1].

(v). (S)-10-((R)-3-(1-((tert-butoxycarbonyl)amino)cyclopropyl)pyrrolidin-1-yl)-9-methoxy-3-methyl-5-oxo-2,3-dihydro-5H-[1,4]oxazino[2,3,4-ij]quinoline-6-carboxylic acid: Methyl (S)-10-((R)-3-(1-((tert-butoxycarbonyl)amino)cyclopropyl)pyrrolidin-1-yl)-3-methyl-5-oxo-2,3-dihydro-5H-[1,4]oxazino[2,3,4-ij]quinoline-6-carboxylate (0.050 g, 0.097 mmol) was dissolved in a mixture of MeOH:H₂O (3:1, 3.0 mL). LiOH.H₂O (1M in water, 0.292 mL, 0.292 mmol) was added and the reaction mixture was stirred at rt for 3 h. The reaction mixture was quenched with cold water, acidified with diluted HCl to pH 4 and extracted with EtOAc. The combined organic layer was washed with brine, dried over sodium sulfate and concentrated under vacuum to afford the desired product (0.050 g) which was directly used in the next step without any further purification. ¹H NMR (400 MHz, Chloroform-d) δ 14.78 (s, 1H), δ 8.78 (s, 1H), 6.78 (s, 1H), 5.17 (s, 1H), 5.01 (s, 1H), 4.45 (d, J=11.7 Hz, 1H), 4.07 (d, J=9.9 Hz, 1H), 3.90 (s, 3H), 3.8-3.60 (m, 3H), 2.11-1.80 (m, 3H), 1.48 (s, 9H), 1.30 (d, 3H). LCMS (m/z): 500.56 [M+1].

(vi). (S)-10-((R)-3-(1-aminocyclopropyl)pyrrolidin-1-yl)-9-methoxy-3-methyl-5-oxo-2,3-dihydro-5H-[1,4]oxazino[2,3,4-ij]quinoline-6-carboxylic acid formate salt: (S)-10-((R)-3-(1-((tert-butoxycarbonyl)amino)cyclopropyl)pyrrolidin-1-yl)-9-methoxy-3-methyl-5-oxo-2,3-dihydro-5H-[1,4]oxazino[2,3,4-ij]quinoline-6-carboxylic acid (0.050 g, 0.100 mmol) was dissolved in DCM (2 mL) and cooled to 0° C. HCl-Dioxane (4M, 0.600 mL) was added and the reaction mixture was stirred at rt for 2 h. The reaction mixture was concentrated under vacuum to afford a crude residue which was purified by reverse phase prep HPLC, affording the desired product (0.005 g, 11% yield) as a yellow solid. ¹H NMR (400 MHz, Methanol-d₄) δ 8.44 (s, 1H), 7.03 (s, 1H), 5.10 (s, 1H), 4.51 (d, J=11.3 Hz, 1H), 4.10 (d, J=11.1 Hz, 1H), 3.92 (s, 3H), 3.78-3.58 (m, 4H), 2.19 (d, J=7.9 Hz, 2H), 1.93 (s, 1H), 1.40 (d, J=6.6 Hz, 3H), 0.96-0.86 (m, 4H). LCMS: t_{R}=1.34 min, m/z=400.5 [M+1] (10 minute run, method B).

### Example 17

### (S)-10-((R)-3-(1-aminocyclopropyl)pyrrolidin-1-yl)-9-cyano-3-methyl-5-oxo-2,3-dihydro-5H-[1,4]oxazino[2,3,4-ij]quinoline-6-carboxylic acid trifluoroacetate salt

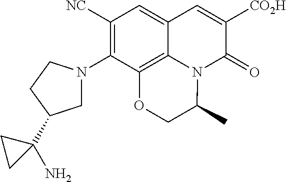 [SMILES: C[C@H]1COc2c(N3CC[C@@H](C4(N)CC4)C3)c(C#N)cc3cc(C(=O)O)c(=O)n1c23]

The title compound was prepared in accordance with the following scheme:

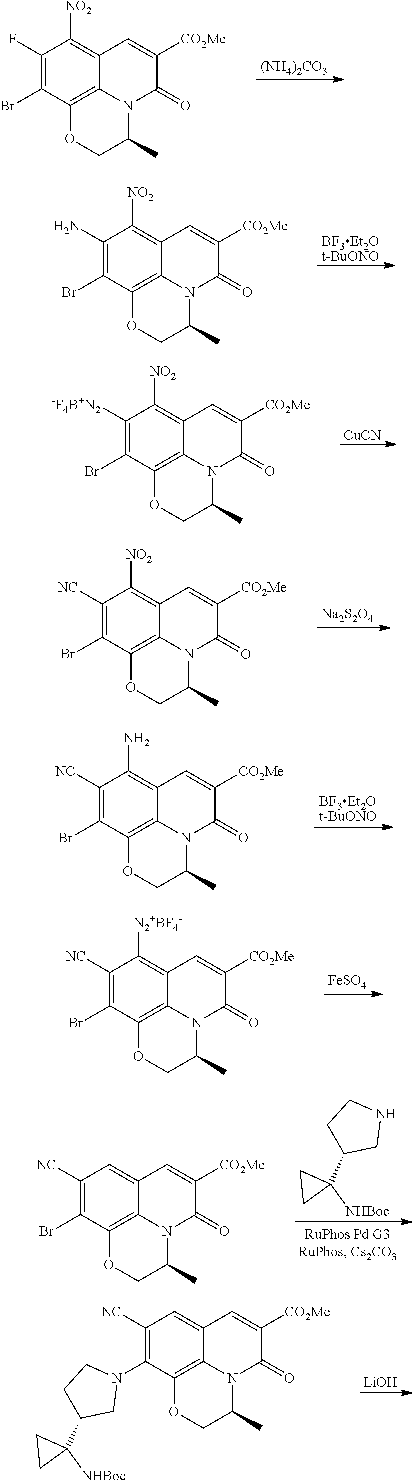

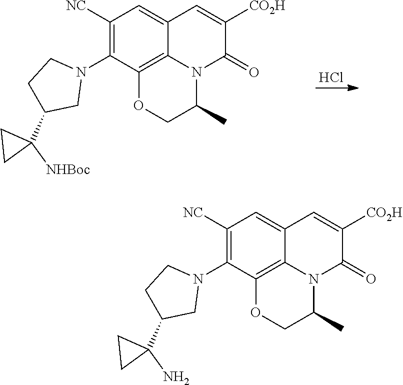 [SMILES: C[C@H]1COc2c(N3CC[C@@H](C4(NC(=O)OC(C)(C)C)CC4)C3)c(C#N)cc3cc(C(=O)O)c(=O)n1c23 ; Cl ; C[C@H]1COc2c(N3CC[C@@H](C4(N)CC4)C3)c(C#N)cc3cc(C(=O)O)c(=O)n1c23]

(i). Methyl (S)-9-amino-10-bromo-3-methyl-8-nitro-5-oxo-2,3-dihydro-5H-[1,4]oxazino[2,3,4-ij]quinoline-6-carboxylate: Methyl (S)-10-bromo-9-fluoro-3-methyl-8-nitro-5-oxo-2,3-dihydro-5H-[1,4]oxazino[2,3,4-ij]quinoline-6-carboxylate (0.600 g, 1.500 mmol) was dissolved in DMF (15 mL). Ammonium carbonate (1.440 g, 15.04 mmol) was added and the reaction mixture was stirred at 90° C. for 5 h. After completion, the reaction mixture was poured on ice cold water, the yellow precipitate was filtered and dried under vacuum to afford the desired product (0.550 g, 93% yield). LCMS (m/z): 400.21 [M+2].

(ii). (S)-10-bromo-6-(methoxycarbonyl)-3-methyl-8-nitro-5-oxo-2,3-dihydro-5H-[1,4]oxazino[2,3,4-ij]quinoline-9-diazonium tetrafluoroborate: BF₃.Et₂O (0.40 g, 2.83 mmol) was dissolved in DCM (15 mL) at -10° C. Methyl (S)-9-amino-10-bromo-3-methyl-8-nitro-5-oxo-2,3-dihydro-5H-[1,4]oxazino[2,3,4-ij]quinoline-6-carboxylate (0.75 g, 1.88 mmol) in DCM (15 mL) was added and the reaction mixture was stirred at −10° C. for 5 minutes. t-Butyl nitrite (0.23 g, 1.80 mmol) was added and the reaction mixture was stirred at −10° C. for 30 minutes and 0° C. for 20 minutes. After completion of the reaction, the DCM was decanted and the residue was triturated with n-pentane and diethyl ether to afford the desired product (0.98 g, 93% yield).

(iii). Methyl-(S)-10-bromo-9-cyano-3-methyl-8-nitro-5-oxo-2,3 hydro-5H-[1,4]oxazino[2,3,4-ij]quinoline-6-carboxylate: (S)-10-bromo-6-(methoxycarbonyl)-3-methyl-8-nitro-5-oxo-2,3-dihydro-5H-[1,4]oxazino[2,3,4-ij]quinoline-9-diazonium tetrafluoroborate (0.98 g, 1.99 mmol) and CuCN (0.18 g, 3.97 mmol) were dissolved in MeCN (20 mL) and the reaction mixture was stirred at 50° C. for 10 minutes. The reaction mixture was quenched with water and extracted with EtOAc. The combined organic layer was washed with brine, dried over sodium sulfate and concentrated under vacuum to afford a crude residue which was purified by silica gel chromatography (20-30% EtOAc in Hexane), affording the desired product (0.44 g, 54% yield). ¹H NMR (400 MHz, Chloroform-d) δ 8.52 (s, 1H), 5.29-5.34 (m, 1H), 4.73 (dd, J=11.5, 1.0 Hz, 1H), 4.34-4.26 (m, 1H), 4.02 (s, 3H), 1.50 (d, J=6.7 Hz, 3H). LCMS (m/z): 410.3 [M+2].

(iv). Methyl-(S)-8-amino-10-bromo-9-cyano-3-methyl-5-oxo-2,3-hydro-5H-[1,4]oxazino[2,3,4-ij]quinoline-6-carboxylate: Methyl-(S)-10-bromo-9-cyano-3-methyl-8-nitro-5-oxo-2,3-dihydro-5H-[1,4]oxazino[2,3,4-ij]quinoline-6-carboxylate (0.40 g, 0.98 mmol) was dissolved in EtOH (20 mL) and the reaction mixture was heated at 90° C. Na₂S₂O₄ (0.51 g, 2.94 mmol) in water (2 mL) was added dropwise and the reaction mixture was stirred at 90° C. for 10 minutes. The reaction mixture was concentrated under vacuum, diluted with water and extracted with EtOAc. The combined organic layer was washed with brine, dried over sodium sulfate and concentrated under vacuum to afford a crude residue which was triturated with n-pentane, affording the desired product (0.15 g, 36% yield). ¹H NMR (400 MHz, DMSO-d₆) δ 8.93 (s, 1H), 6.99 (s, 2H), 4.92 (d, J=7.1 Hz, 1H), 4.45 (d, J=11.3 Hz, 1H), 4.07 (dd, J=11.5, 2.5 Hz, 1H), 3.83 (s, 3H), 1.26 (d, J=6.6 Hz, 3H). LCMS (m/z): 380.2 [M+2].

(v). (S)-10-bromo-9-cyano-6-(methoxycarbonyl)-3-methyl-5-oxo-2,3-dihydro-5H-[1,4]oxazino[2,3,4-ij]quinoline-8-diazonium tetrafluoroborate: BF₃.Et₂O (0.084 g, 0.592 mmol) was dissolved in DCM (3 mL) at −10° C. Methyl-(S)-8-amino-10-bromo-9-cyano-3-methyl-5-oxo-2,3-dihydro-5H-[1,4]oxazino[2,3,4-ij]quinoline-6-carboxylate (0.150 g, 0.396 mmol) in DCM (2 mL) was added and the reaction mixture was stirred at −10° C. for 5 minutes. t-Butyl nitrite (0.062 g, 0.592 mmol) was added and the reaction mixture was stirred at −10° C. for 30 minutes and then at 0° C. for 20 minutes. After completion of the reaction the DCM was decanted, the residue triturated with n-pentane and diethyl ether, affording the desired product (0.110 g, 90% yield).

(vi). Methyl-(S)-10-bromo-9-cyano-3-methyl-5-oxo-2,3-dihydro-5H-[1,4]oxazino[2,3,4-ij]quinoline-6-carboxylate: (S)-10-bromo-9-cyano-6-(methoxycarbonyl)-3-methyl-5-oxo-2,3-dihydro-5H-[1,4]oxazino[2,3,4-ij]quinoline-8-diazonium tetrafluoroborate (0.188 g, 0.394 mmol), FeSO₄.7H₂O (0.131 g, 0.473 mmol) were dissolved in DMF (2 mL) at 0° C. and stirred at rt for 10 minutes. The reaction mixture was quenched with water and extracted with EtOAc. The combined organic layer was washed with brine, dried over sodium sulfate and concentrated under vacuum to afford a crude residue which was purified by silica gel chromatography (20-30% EtOAc in Hexane), affording the desired product (0.050 g, 35% yield). ¹H NMR (400 MHz, Chloroform-d) δ 8.46 (s, 1H), 7.69 (s, 1H), 5.22 (d, J=6.7 Hz, 1H), 4.65 (d, J=11.6 Hz, 1H), 4.23 (d, J=10.8 Hz, 1H), 4.00 (s, 3H), 1.49 (d, J=6.7 Hz, 3H). LCMS (m/z): 365.2 [M+2].

(vii). Methyl (S)-10-((R)-3-(1-((tert-butoxycarbonyl)amino)cyclopropyl)pyrrolidin-1-yl)-9-cyano-3-methyl-5-oxo-2,3-dihydro-5H-[1,4]oxazino[2,3,4-ij]quinoline-6-carboxylate: Methyl-(S)-10-bromo-9-cyano-3-methyl-5-oxo-2,3-dihydro-5H-[1,4]oxazino [2,3,4-ij]quinoline-6-carboxylate (0.050 g, 0.140 mmol), tert-butyl (R)-(1-(pyrrolidin-3-yl)cyclopropyl)carbamate (0.062 g, 0.226 mmol) and Cs₂CO₃ (0.111 g, 0.342 mmol) were suspended in toluene (5 mL) and degassed with nitrogen for 5 minutes. RuPhos (0.010 g, 0.020 mmol) and RuPhosPdG₃ (0.016 g, 0.020 mmol) were added and the reaction mixture was heated at 100° C. for 2 h. The reaction mixture was filtered through a celite pad and washed with excess EtOAc. The filtrate was concentrated under vacuum to afford a crude residue which was purified by Prep TLC with 60% EtOAc/40% hexane, affording the desired product (0.050 g, 58% yield). LCMS (m/z): 509.3 [M+1].

(viii). (S)-10-((R)-3-(1-((tert-butoxycarbonyl)amino)cyclopropyl)pyrrolidin-1-yl)-9-cyano-3-methyl-5-oxo-2,3-dihydro-5H-[1,4]oxazino[2,3,4-ij]quinoline-6-carboxylic acid: Methyl (S)-10-((R)-3-(1-((tert-butoxycarbonyl)amino)cyclopropyl)pyrrolidin-1-yl)-9-cyano-3-methyl-5-oxo-2,3-dihydro-5H-[1,4]oxazino[2,3,4-ij]quinoline-6-carboxylate (0.050 g, 0.098 mmol) was dissolved into a mixture of MeOH:H₂O (3:1, 3 mL). LiOH.H₂O (1M in water, 0.300 mL, 0.300 mmol) was added and the reaction mixture was stirred at rt for 3 h. The reaction mixture was quenched with cold water, acidified with diluted HCl to pH 4 and extracted with EtOAc. The combined organic layer was dried over sodium sulfate and concentrated under vacuum to afford the desired product (0.040 g) which was directly used in the next step without any further purification. LCMS (m/z): 495.22 [M+1].

(ix). (S)-10-((R)-3-(1-aminocyclopropyl)pyrrolidin-1-yl)-9-cyano-3-methyl-5-oxo-2,3-dihydro-5H-[1,4]oxazino[2,3,4-ij]quinoline-6-carboxylic acid trifluoroacetate salt: (S)-10-((R)-3-(1-((tert-butoxycarbonyl)amino)cyclopropyl)pyrrolidin-1-yl)-9-cyano-3-methyl-5-oxo-2,3-dihydro-5H-[1,4]oxazino[2,3,4-ij]quinoline-6-carboxylic acid (0.040 g, 0.081 mmol) was dissolved in DCM (3 mL) and cooled to 0° C. TFA (0.5 mL) was added and the reaction mixture was stirred at rt for 3 h. The reaction mixture was concentrated under vacuum to afford a crude residue which was purified by trituration with DCM and diethyl ether, affording the desired product (0.032 g, 76% yield) as a yellow solid. ¹H NMR (400 MHz, Methanol-d₄) δ 8.79 (s, 1H), 7.94 (s, 1H), 5.15 (d, J=7.3 Hz, 1H), 4.58 (d, J=11.3 Hz, 1H), 4.26-4.07 (m, 2H), 3.95-3.84 (m, 3H), 2.70-2.68 (m, 1H), 2.21-2.05 (m, 1H), 1.82-1.77 (m, 1H), 1.47 (d, J=6.6 Hz, 3H), 1.04-0.93 (m, 4H). LCMS: t_{R}=1.33 min, m/z=395.58 [M+1] (10 minute run, method B).

### Example 18

### (S)-10-((R)-3-(1-aminocyclopropyl)pyrrolidin-1-yl)-9-chloro-3-methyl-5-oxo-2,3-dihydro-5H-[1,4]oxazino[2,3,4-ij]quinoline-6-carboxylic acid hydrochloride salt

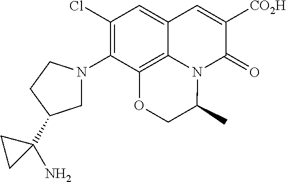 [SMILES: C[C@H]1COc2c(N3CC[C@@H](C4(N)CC4)C3)c(Cl)cc3cc(C(=O)O)c(=O)n1c23]

The title compound was prepared in accordance with the following scheme:

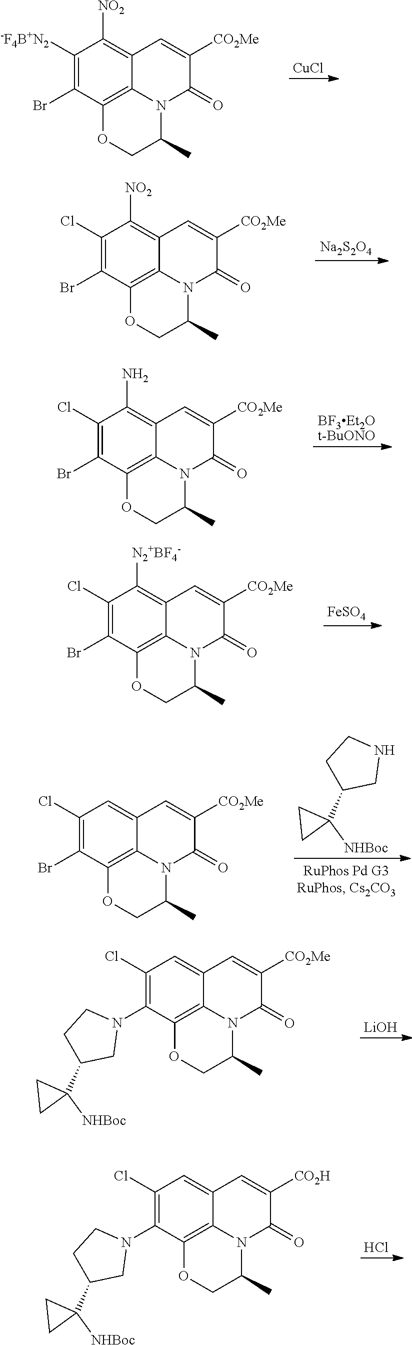

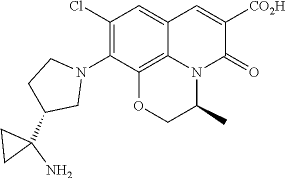 [SMILES: C[C@H]1COc2c(N3CC[C@@H](C4(N)CC4)C3)c(Cl)cc3cc(C(=O)O)c(=O)n1c23]

(i). Methyl (S)-10-bromo-9-chloro-3-methyl-8-nitro-5-oxo-2,3-dihydro-5H-[1,4]oxazino[2,3,4-ij]quinoline-6-carboxylate: (S)-10-bromo-6-(methoxycarbonyl)-3-methyl-8-nitro-5-oxo-2,3-dihydro-5H-[1,4]oxazino[2,3,4-ij]quinoline-9-diazonium tetrafluoroborate (0.450 g, 0.912 mmol), CuCl (0.108 g, 1.095 mmol) were dissolved in MeCN (10 mL) and the reaction mixture was stirred at 70° C. for 10 minutes. The reaction mixture was quenched with water and extracted with EtOAc. The organic layer was washed with brine, dried over sodium sulfate and concentrated under vacuum to afford a crude residue which was purified by silica gel chromatography (20-30% EtOAc in hexane), affording the desired product (0.280 g, 59% yield). ¹H NMR (400 MHz, DMSO-d₆) δ 8.81 (s, 1H), 4.92 (d, J=7.1 Hz, 1H), 4.46 (d, J=11.3 Hz, 1H), 4.07 (d, J=11.0 Hz, 1H), 4.00 (s, 3H), 1.49 (d, 3H). LCMS (m/z): 418.6 [M+2].

(ii).Methyl (S)-8-amino-10-bromo-9-chloro-3-methyl-5-oxo-2,3-dihydro-5H-[1,4]oxazino[2,3,4-ij]quinoline-6-carboxylate: Methyl (S)-10-bromo-9-chloro-3-methyl-8-nitro-5-oxo-2,3-dihydro-5H-[1,4]oxazino[2,3,4-ij]quinoline-6-carboxylate (0.20 g, 0.52 mmol) was dissolved in EtOH (15 mL) and the mixture was heated at 90° C. Na2S2O4 (0.41 g, 2.35 mmol) in water (2 mL) was added and the reaction mixture was stirred at 90° C. for 1 h. The reaction mixture was concentrated under vacuum, diluted with water and extracted with EtOAc. The combined organic layer was washed with brine, dried over sodium sulfate and concentrated under vacuum, affording the desired product (0.18 g, 69% yield). ¹H NMR (400 MHz, Chloroform-d) δ 8.28 (s, 1H), 5.25 (s, 1H), 4.68 (d, J=11.5 Hz, 1H), 4.28 (d, J=11.6 Hz, 1H), 4.00 (s, 3H), 1.29 (d, 3H). LCMS (m/z): 388.61 [M+1].

(iii). (S)-10-bromo-9-chloro-6-(methoxycarbonyl)-3-methyl-5-oxo-2,3-dihydro-5H-[1,4]oxazino[2,3,4-ij]quinoline-8-diazonium tetrafluoroborate: BF₃.Et₂O (0.09 mL, 0.70 mmol) was diluted in DCM (5 mL) at −15° C. Methyl (S)-8-amino-10-bromo-9-chloro-3-methyl-5-oxo-2,3-dihydro-5H-[1,4]oxazino[2,3,4-ij]quinoline-6-carboxylate (0.18 g, 0.46 mmol) in DCM (5 mL) was added and the reaction mixture was stirred at −15° C. for 5 minutes. t-Butyl nitrite (0.07 mL, 0.56 mmol) was added and the reaction mixture was stirred at −15° C. for 30 minutes and at 0° C. for 20 minutes. After completion of the reaction, the DCM was decanted, the residue was triturated with n-pentane and diethyl ether, affording the desired product (0.180 g).

(iv). Methyl (S)-10-bromo-9-chloro-3-methyl-5-oxo-2,3-dihydro-5H-[1,4]oxazino[2,3,4-ij]quinoline-6-carboxylate: (S)-10-bromo-9-chloro-6-(methoxycarbonyl)-3-methyl-5-oxo-2,3-dihydro-5H-[1,4]oxazino[2,3,4-ij]quinoline-8-diazonium tetrafluoroborate (0.180 g, 0.463 mmol), FeSO₄.7H_{2O (}0.154 g, 0.556 mmol) were dissolved in DMF (5 mL) at 0° C. and the mixture was stirred at rt for 1 h. The reaction mixture was quenched with water and extracted with EtOAc. The organic layer was washed with brine, dried over sodium sulfate and concentrated under vacuum to afford a crude residue which was purified by silica gel chromatography (20-30% EtOAc in hexane), affording the desired product (0.050 g, 29% yield). LCMS (m/z): 374.6 [M+2].

(v).Methyl (S)-10-((R)-3-(1-((tert-butoxycarbonyl)amino)cyclopropyl)pyrrolidin-1-yl)-9-chloro-3-methyl-5-oxo-2,3-dihydro-5H-[1,4]oxazino[2,3,4-ij]quinoline-6-carboxylate: Methyl (S)-10-bromo-9-chloro-3-methyl-5-oxo-2,3-dihydro-5H-[1,4]oxazino[2,3,4-ij]quinoline-6-carboxylate (0.040 g, 0.103 mmol), tert-butyl (R)-(1-(pyrrolidin-3-yl)cyclopropyl)carbamate (0.036 g, 0.161 mmol) and Cs₂CO₃ (0.104 g, 0.322 mmol) were suspended in toluene (4 mL) and degassed with nitrogen for 5 minutes. RuPhos (0.008 g, 0.016 mmol) and RuPhosPdG₃ (0.0136 g, 0.016 mmol) were added and the reaction mixture was heated at 90° C. for 1 h. The reaction mixture was filtered through a celite pad and washed with excess EtOAc. The organic layer was concentrated under vacuum to afford a crude residue which was purified by Prep TLC affording the desired product (0.020 g, 29% yield). LCMS (m/z):519.1 [M+1].

(vi). (S)-10-((R)-3-(1-((tert-butoxycarbonyl)amino)cyclopropyl)pyrrolidin-1-yl)-9-chloro-3-methyl-5-oxo-2,3-dihydro-5H-[1,4]oxazino[2,3,4-ij]quinoline-6-carboxylic acid: Methyl (S)-10-((R)-3-(1-((tert-butoxycarbonyl)amino)cyclopropyl)pyrrolidin-1-yl)-9-chloro-3-methyl-5-oxo-2,3-dihydro-5H-[1,4]oxazino[2,3,4-ij]quinoline-6-carboxylate (0.015 g, 0.028 mmol) was dissolved into a mixture of MeOH:H₂O (3:1, 1.3 mL). LiOH.H₂O (1M in water, 0.086 mL, 0.086 mmol) was added and the reaction mixture was stirred at rt for 1.5 h. The reaction mixture was quenched with cold water, acidified with diluted HCl to pH 4 and extracted with EtOAc. The organic layer was washed with brine, dried over sodium sulfate and concentrated under vacuum to afford the desired product (0.017 g, 87% yield) which was directly used in the next step without any further purification. LCMS (m/z): 504.98 [M+1].

(vii). (S)-10-((R)-3-(1-aminocyclopropyl)pyrrolidin-1-yl)-9-chloro-3-methyl-5-oxo-2,3-dihydro-5H-[1,4]oxazino[2,3,4-ij]quinoline-6-carboxylic acid) hydrochloride salt: (S)-10-((R)-3-(1-((tert-butoxycarbonyl)amino)cyclopropyl)pyrrolidin-1-yl)-9-chloro-3-methyl-5-oxo-2,3-dihydro-5H-[1,4]oxazino[2,3,4-ij]quinoline-6-carboxylic acid (0.040 g, 0.030 mmol) was dissolved in DCM (2 mL) and cooled to 0° C. HCl in dioxane (4M, 0.150 mL) was added and the reaction mixture was stirred at rt for 1 h. The reaction mixture was concentrated under vacuum to afford a crude residue which was purified by trituration with ethyl acetate, affording the desired product (0.008 g, 59% yield). ¹H NMR (400 MHz, Methanol-d₄) δ 1H NMR (400 MHz, Methanol-d4) δ 8.84 (s, 1H), 7.72 (s, 1H), 5.18 (d, J=6.5 Hz, 1H), 4.63 (d, J=11.6 Hz, 1H), 4.22-4.19 (m, 1H), 3.67 (d, J=8.3 Hz, 1H), 3.62-3.58 (m, 2H), 3.51-3.42 (m, 1H), 2.68-2.59 (m, 1H), 2.25 (s, 1H), 1.90 (dd, J=12.6, 8.0 Hz, 1H), 1.47 (d, J=6.7 Hz, 3H), 1.07-0.97 (m, 4H). LCMS: t_{R}=1.37 min, m/z=404.6 [M+1] (10 minute run, method B).

### Example 19.1

### (S)-10-((R)-3-(1-aminocyclopropyl)pyrrolidin-1-yl)-8-chloro-9-fluoro-3-methyl-5-oxo-2,3-dihydro-5H-[1,4]oxazino[2,3,4-ij]quinoline-6-carboxylic acid formate salt

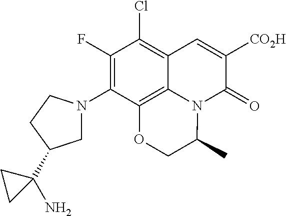 [SMILES: C[C@H]1COc2c(N3CC[C@@H](C4(N)CC4)C3)c(F)c(Cl)c3cc(C(=O)O)c(=O)n1c23]

The title compound was prepared in accordance with the following scheme:

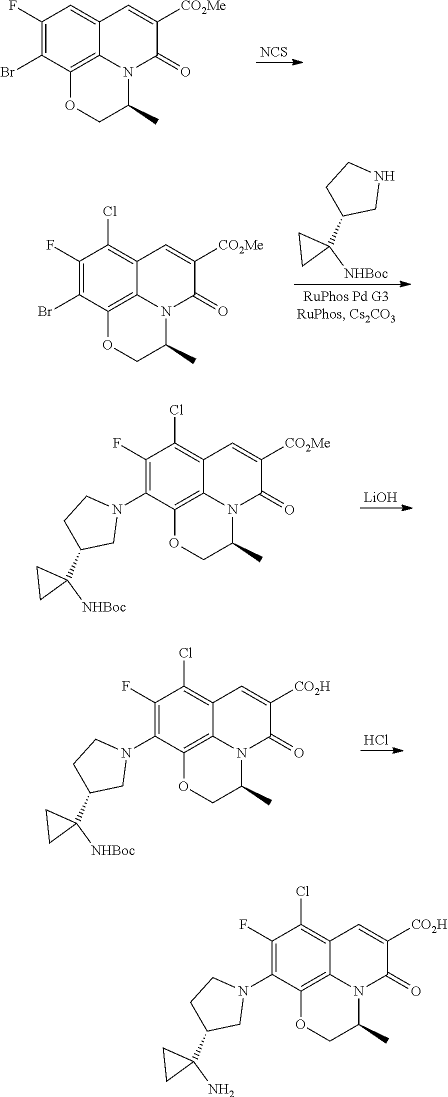

(i). Methyl (S)-10-bromo-8-chloro-9-fluoro-3-methyl-5-oxo-2,3-dihydro-5H-[1,4] oxaz ino[2,3,4-ij]quinoline-6-carboxylate: Methyl (S)-10-bromo-9-fluoro-3-methyl-5-oxo-2,3-dihydro-5H-[1,4]oxazino[2,3,4-ij]quinoline-6-carboxylate (0.20 g, 0.56 mmol) was dissolved in DMF (5 mL). N-Chlorosuccinamide (0.22 g, 1.68 mmol) was added and the resulting suspension was stirred at rt for 24 h. The reaction mixture was diluted with cold water and extracted with Et0Ab The combined organic layer was washed with water, brine, dried over sodium sulfate and concentrated under vacuum to afford the desired product (0.18 g, 81% yield) as a yellow solid. ¹H NMR (400 MHz, MeOD) δ 8.81 (s, 1H), 5.25-5.21 (m, 1H), 4.63 (d, J=11.2 Hz, 1H), 4.23-4.20 (m, 1H), 4.01 (s, 3H), 1.47 (d, J=6.8 Hz, 3H). LCMS (m/z): 392.6 [M+1].

(ii).Methyl (S)-10-((R)-3-(1-((tert-butoxycarbonyl)amino)cyclopropyl)pyrrolidin-1-yl)-8-chloro-9-fluoro-3-methyl-5-oxo-2,3-dihydro-5H-[1,4]oxazino[2,3,4-ij]quinoline-6-carboxylate: Methyl (S)-10-bromo-8-chloro-9-fluoro-3-methyl-5-oxo-2,3-dihydro-5H-[1,4]oxazino[2,3,4-ij]quinoline-6-carboxylate (0.150 g, 0.384 mmol) and cesium carbonate (0.312 g, 0.960 mmol) in toluene (5 mL) were degased with nitrogen for 10 minutes. tert-Butyl (R)-(1-(pyrrolidin-3-yl)cyclopropyl)carbamate (0.130 g, 0.576 mmol), RuPhos (0.027 g, 0.058 mmol) and RuPhos Pd G₃ (0.032 g, 0.038 mmol) were added and the reaction mixture was heated at 90° C. for 8 h. The crude reaction mixture was filtered through a celite pad and washed with excess of EtOAc. The filtrate was concentrated under vacuum to afford a crude residue which was purified by flash chromatography (30-35% EtOAc in Hexane), providing the desired product (0.140 g, 56% yield) as a yellow solid. ¹H NMR (400 MHz, Chloroform-d) δ 8.78 (s, 1H), 7.26 (d, J=11.6 Hz, 1H), 5.19-5.17 (m, 1H), 4.42 (dd, J=11.9, 6.7 Hz, 2H), 3.98 (s, 3H), 3.68-3.66 (m, 1H), 3.45-3.41 (m, 2H), 2.47-2.24 (m, 2H), 2.14-1.97 (m, 1H), 1.63 (s, 9H), 1.49-1.45 (m, 5H), 0.90-0.81 (m, 2H). LCMS (m/z): 537.1 [M+1].

(iii). (S)-10-((R)-3-(1-((tert-butoxycarbonyl)amino)cyclopropyl)pyrrolidin-1-yl)-8-chloro-9-fluoro-3-methyl-5-oxo-2,3-dihydro-5H-[1,4]oxazino[2,3,4-ij]quinoline-6-carboxylic acid: Methyl (S)-10-((R)-3-(1-((tert-butoxycarbonyl)amino)cyclopropyl)pyrrolidin-1-yl)-8-chloro-9-fluoro-3-methyl-5-oxo-2,3-dihydro-5H-[1,4]oxazino[2,3,4-ij]quinoline-6-carboxylate (0.09 g, 0.17 mmol) was dissolved in MeOH (2 mL) and water (0.5 mL). 1M Lithium hydroxide in water (0.50 mL, 0.50 mmol) was added and the reaction mixture was stirred at rt for 2 h. The reaction mixture was diluted with water, acidified to pH 4 with 1 N HCl and extracted with EtOAc. The combined organic layer was dried over sodium sulfate and concentrated under vacuum to afford the desired product (0.07 g, 80% yield) as a yellow solid which was directly used in the next step without any further purification. LCMS (m/z): 522.9 [M+1].

(iv). (S)-10-((R)-3-(1-aminocyclopropyl)pyrrolidin-1-yl)-3-ethyl-9-fluoro-5-oxo-2,3-dihydro-5H-[1,4]oxazino[2,3,4-ij]quinoline-6-carboxylic acid formate salt: (S)-10-((R)-3-(1-((tert-butoxycarbonyl)amino)cyclopropyl)pyrrolidin-1-yl)-8-chloro-9-fluoro-3-methyl-5-oxo-2,3-dihydro-5H-[1,4]oxazino[2,3,4-ij]quinoline-6-carboxylic acid (70.0 mg, 0.13 mmol) was dissolved in DCM (1 mL) and cooled to 0° C. HCl in dioxane (4M, 2 mL) was added and the reaction mixture was stirred at rt for 2 h. After completion, the reaction mixture was concentrated under vacuum to afford a crude residue which was purified by reverse phase Prep HPLC, providing the desired product (20.0 mg, 35% yield) as a yellow solid. ¹H NMR (400 MHz, MeOD) δ 8.66 (s, 1H), 8.56 (s, 1H), 5.15-5.11 (m, 1H), 4.53 (d, J=11.4 Hz, 1H), 4.09 (d, J=11.1 Hz, 1H), 3.96 (d, J=9.0 Hz, 1H), 3.76-3.70 (m, 3H), 2.47-2.43 (m, 1H), 2.12-2.09 (m, 1H), 1.78-1.75 (m, 1H), 1.40-1.31 (m, 3H), 0.91-0.88 (m, 4H). LCMS: t_{R}=1.38 min, m/z=422.37 [M+1] (10 minute run, method B). Using the procedures described for Example 19.1 the following compound were prepared:

| | | | HPLC, | |
|---|---|---|---|---|
| Ex | | | t_{R} | LCMS |
| # | Structure | Chemical Name | (min) | [M + 1] |
| 19.2 | 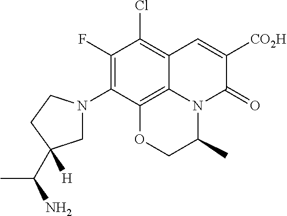 [SMILES: C[C@H](N)[C@@H]1CCN(c2c(F)c(Cl)c3cc(C(=O)O)c(=O)n4c3c2OC[C@@H]4C)C1] | (S)-10-((R)-3-((S)-1-aminoethyl)pyrrolidin-1- yl)-8-chloro-9-fluoro-3-methyl-5-oxo-2,3- dihydro-5H-[1,4]oxazino[2,3,4-ij]quinoline-6- carboxylic acid (formate salt) | 1.38 | 410.5 |
| 19.3¹ | 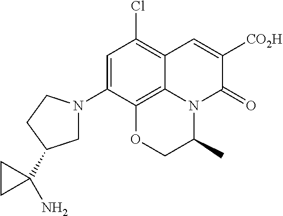 [SMILES: C[C@H]1COc2c(N3CC[C@@H](C4(N)CC4)C3)cc(Cl)c3cc(C(=O)O)c(=O)n1c23] | (S)-10-((R)-3-(1-aminocyclopropyl)pyrrolidin- 1-yl)-8-chloro-3-methyl-5-oxo-2,3-dihydro- 5H-[1,4]oxazino[2,3,4-ij]quinoline-6- carboxylic acid (HCl salt) | 1.39 | 404.5 |

¹In step 1, use methyl (S)-10-bromo-3-methy1-5-oxo-2,3-dihydro-5H-[1,4]oxazino[2,3,4-ij]quinoline-6-carboxylate as starting material.

HPLC method: Characterized by high performance liquid chromatography (HPLC) on a Waters ACQUITY H class UPLC system with 0.55 mL/min flow rate; column BEH-C18, 1.7 um, 2.1×50 mm from Waters, column temperature: ambiant; gradient (solvent A is 2 mM Ammonium Acetate and 0.1% Formic Acid in Water, solvent B is 0.1% Formic Acid in acetonitrile) : 5% solvent B hold for 0.4 min, 5-40% solvent B over 0.6 min, 40-60% solvent B over 1.2 min, 60-100% solvent B over 2.3 min then 100% solvent B over 3 min; compounds were detected by ultraviolet light (UV) absorption at 236 nm.

### Example 20

### (S)-8-amino-10-((R)-3-(1-aminocyclopropyl)pyrrolidin-1-yl)-3-methyl-5-oxo-2,3-dihydro-5H-[1,4]oxazino[2,3,4-ij]quinoline-6-carboxylic acid

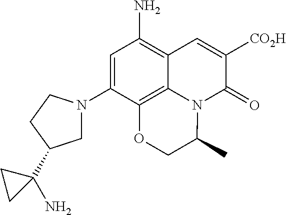 [SMILES: C[C@H]1COc2c(N3CC[C@@H](C4(N)CC4)C3)cc(N)c3cc(C(=O)O)c(=O)n1c23]

The title compound was prepared in accordance with the following scheme:

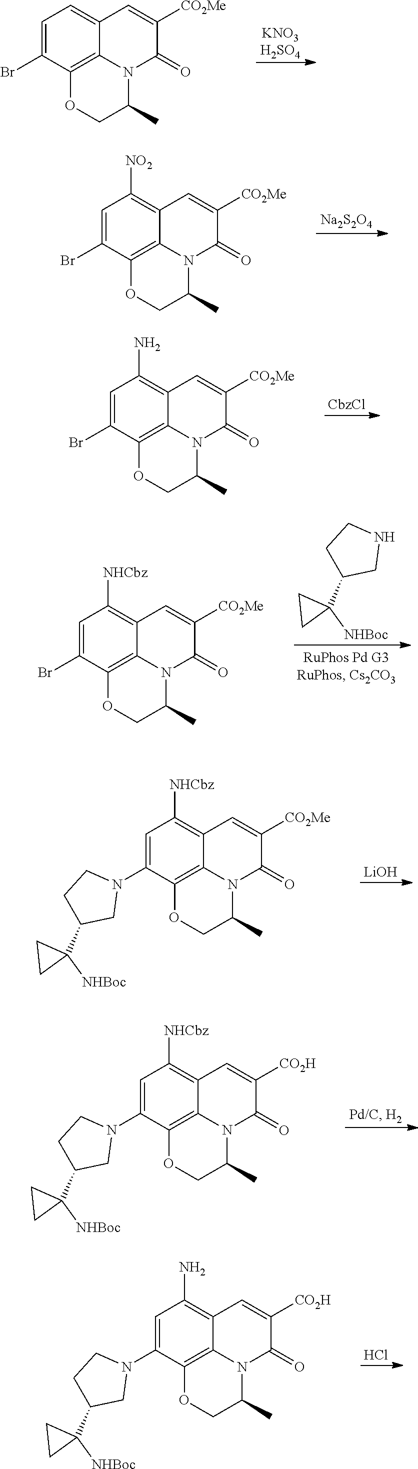

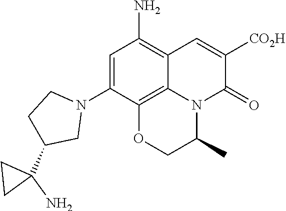 [SMILES: C[C@H]1COc2c(N3CC[C@@H](C4(N)CC4)C3)cc(N)c3cc(C(=O)O)c(=O)n1c23]

(i). Methyl (S)-10-bromo-3-methyl-8-nitro-5-oxo-2,3-dihydro-5H-[1,4]oxazino[2,3,4-ij]quinoline-6-carboxylate: Methyl (S)-10-bromo-3-methyl-5-oxo-2,3-dihydro-5H-[1,4]oxazino[2,3,4-ij]quinoline-6-carboxylate (300 mg, 0.89 mmol) was dissolved in concentrated H₂SO₄ (3 mL) at 0° C. KNO₃ (94.0 mg, 0.93 mmol) was added slowly and the reaction mixture was stirred at rt for 1 h. The reaction mixture was poured into crushed ice and extracted with EtOAc. The combined organic layer was washed with saturated NaHCO₃ solution, dried over sodium sulfate and concentrated under vacuum to afford the desired product (300 mg, 88% yield) as a yellow solid. ¹H NMR (400 MHz, Chloroform-d) δ 9.25 (d, J=3.4 Hz, 1H), 8.30 (s, 1H), 5.37-5.30 (m, 1H), 4.69 (d, J=11.4 Hz, 1H), 4.29 (dd, J=11.5, 2.6 Hz, 1H), 4.02 (s, 3H), 1.48 (d, J=6.7 Hz, 3H). LCMS (m/z): 383.15 [M+2].

(ii).Methyl (S)-8-amino-10-bromo-3-methyl-5-oxo-2,3-dihydro-5H-[1,4]oxazino[2,3,4-ij]quinoline-6-carboxylate: Methyl (S)-10-bromo-3-methyl-8-nitro-5-oxo-2,3-dihydro-5H-[1,4]oxazino[2,3,4-ij]quinoline-6-carboxylate (0.300 g, 0.783 mmol) was dissolved in ethanol (15 mL) and water (0.3 mL). Sodium dithionite (0.681 g, 3.916 mmol) was added and the reaction mixture was stirred at 60° C. for 16 h. The reaction mixture was poured into cooled water and extracted with EtOAc. The combined organic layer was washed with brine, dried over sodium sulfate and concentrated under vacuum to afford the desired product (0.220 g, 79% yield) which was directly used in the next step without any further purification. ¹H NMR (400 MHz, DMSO-d₆) δ 8.71 (s, 1H), 6.70 (s, 1H), 6.12 (s, 2H), 4.91 (d, J=7.1 Hz, 1H), 4.41 (d, J=11.2 Hz, 1H), 4.10-3.96 (m, 1H), 3.82 (s, 3H), 1.24 (d, J=6.9 Hz, 3H). LCMS (m/z): 353.17 [M+2].

(iii). Methyl (S)-8-(((benzyloxy)carbonyl)amino)-10-bromo-3-methyl-5-oxo-2,3-dihydro-5H-[1,4]oxazino[2,3,4-ij]quinoline-6-carboxylate: Methyl (S)-8-amino-10-bromo-3-methyl-5-oxo-2,3-dihydro-5H-[1,4]oxazino[2,3,4-ij]quinoline-6-carboxylate (0.220 g, 0.623 mmol) and benzyl chloroformate (0.321 g, 1.869 mmol) were dissolved in toluene (15 mL) and stirred at 80° C. for 16 h. After completion the mixture was poured into ice cold water and extracted with EtOAc. The combined organic layer was washed with a saturated NaHCO₃ solution, dried over sodium sulfate and concentrated under vacuum to afford a crude residue which was purified by silica gel column chromatography (15-20% EtOAc in Hexane), affording the desired product (0.220 g, 72% yield) as a yellow solid. ¹H NMR (400 MHz, Chloroform-d) δ 8.59 (s, 1H), 7.76 (s, 1H), 7.48-7.36 (m, 4H), 6.93 (s, 1H), 5.28 (s, 2H), 5.21 (d, J=6.9 Hz, 1H), 4.55 (d, J=11.3 Hz, 1H), 4.28-4.13 (m, 1H), 3.98 (s, 3H), 1.56-1.43 (m, 3H). LCMS (m/z): 487.31 [M+2].

(iv). Methyl (S)-8-(((benzyloxy)carbonyl)amino)-10-((R)-3-(1-((tert-butoxycarbonyl)amino)cyclopropyl)pyrrolidin-1-yl)-3-methyl-5-oxo-2,3-dihydro-5H-[1,4]oxazino[2,3,4-ij]quinoline-6-carboxylate: Methyl (S)-8-(((benzyloxy)carbonyl)amino)-10-bromo-3-methyl-5-oxo-2,3-dihydro-5H-[1,4]oxazino[2,3,4-ij]quinoline-6-carboxylate (0.220 g, 0.450 mmol), tert-butyl (R)-(1-(pyrrolidin-3-yl)cyclopropyl)carbamate (0.239 g, 0.901 mmol) and Cs₂CO₃ (0.367 g, 1.127 mmol) were suspended in toluene (20 mL) and degassed with nitrogen for 5 minutes. RuPhos (0.031 g, 0.067 mmol) and RuPhosPdG₃ (0.056 g, 0.067 mmol) were added and the reaction mixture was heated to 80° C. for 6 h. The reaction mixture was poured into water and extracted with EtOAc. The combined organic layer was washed with brine, dried over sodium sulfate and concentrated under vacuum to afford a crude residue which was purified by reverse phase chromatography (60-70% MeOH in water), providing the desired product (0.120 g, 42% yield) as yellow solid. LCMS (m/z): 632.71 [M+1].

(v). (S)-8-(((benzyloxy)carbonyl)amino)-10-((R)-3-(1-((tert-butoxycarbonyl) amino)cyclopropyl)pyrrolidin-1-yl)-3-methyl-5-oxo-2,3-dihydro-5H-[1,4]oxazino[2,3,4-ij]quinoline-6-carboxylic acid: Methyl (S)-8-(((benzyloxy)carbonyl)amino)-10-((R)-3-(1-((tert-butoxycarbonyl)amino)cyclopropyl)pyrrolidin-1-yl)-3-methyl-5-oxo-2,3-dihydro-5H-[1,4]oxazino[2,3,4-ij]quinoline-6-carboxylate (0.120 g, 0.189 mmol) was dissolved into a mixture of MeOH:H₂O (3:1, 12 mL). LiOH.H₂O (1M in water, 0.569 mL, 0.569 mmol) was added and the reaction mixture was stirred at rt for 3 h. The reaction mixture was quenched with water, acidified with diluted HCl to pH 4 and extracted with ethyl acetate. The combined organic layer was dried over sodium sulfate and concentrated under vacuum to afford the desired product (0.110 g, 94% yield) as ayellow solid which was directly used in the next step without any further purification. LCMS (m/z): 618.69 [M+1].

(vi). (S)-8-amino-10-((R)-3-(1-((tert-butoxycarbonyl)amino)cyclopropyl)pyrrolidin-1-yl)-3-methyl-5-oxo-2,3-dihydro-5H-[1,4]oxazino[2,3,4-ij]quinoline-6-carboxylic acid: (S)-8-(((benzyloxy)carbonyl)amino)-10-((R)-3-(1-((tert-butoxycarbonyl)amino)cyclopropyl)pyrrolidin-1-yl)-3-methyl-5-oxo-2,3-dihydro-5H-[1,4]oxazino[2,3,4-ij]quinoline-6-carboxylic acid (0.110 g, 0.177 mmol) was dissolved in 2M methanolic ammonia (10mL). 10% Pd/C (50% in water, 0.177 g) was added and the reaction mixture was stirred under 1 atmosphere of hydrogen at rt for 4 h. After completion, the reaction mixture was filtered through a celite pad and concentrated under vacuum to afford the desired product (0.080 g, 93% yield) which was directly used in the next step without any further purification. LCMS (m/z): 484.55 [M+1].

(vii). (S)-8-amino-10-((R)-3-(1-aminocyclopropyl)pyrrolidin-1-yl)-3-methyl-5-oxo-2,3-dihydro-5H-[1,4]oxazino[2,3,4-ij]quinoline-6-carboxylic acid: (S)-8-amino-10-((R)-3-(1-((tert-butoxycarbonyl)amino)cyclopropyl)pyrrolidin-1-yl)-3-methyl-5-oxo-2,3-dihydro-5H-[1,4]oxazino[2,3,4-ij]quinoline-6-carboxylic acid (0.080 g, 0.165 mmol) was dissolved in DCM (2 mL) and cooled to 0° C. HCl-Dioxane (4M, 0.8 mL) was added and the reaction mixture was stirred at rt for 2 h. The reaction mixture was concentrated under vacuum to afford a crude residue which was purified by reverse phase Prep HPLC affording the desired product (0.014 g, 24% yield) as a yellow solid. ¹H NMR (400 MHz, MeOD) δ 8.84 (s, 1H), 6.08 (s, 1H), 5.06 (d, J=6.9 Hz, 1H), 4.36 (d, J=11.2 Hz, 1H), 3.87-3.57 (m, 5H), 2.31-2.29 (m, 1H), 2.07-2.05 (m, 1H), 1.78 (d, J=9.9 Hz, 1H), 1.43 (d, J=6.7 Hz, 3H), 0.72 (d, J=7.9 Hz, 4H). LCMS: t_{R}=1.36 min, m/z=384.44 [M+1] (10 minute run, method B).

### Example 21.1

### (S)-10-((R)-3-(1-aminocyclopropyl)pyrrolidin-1-yl)-8-cyano-9-fluoro-3-methyl-5-oxo-2,3-dihydro-5H-[1,4]oxazino[2,3,4-ij]quinoline-6-carboxylic acid trifluoroacetate salt

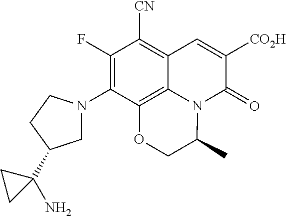 [SMILES: C[C@H]1COc2c(N3CC[C@@H](C4(N)CC4)C3)c(F)c(C#N)c3cc(C(=O)O)c(=O)n1c23]

The title compound was prepared in accordance with the following scheme:

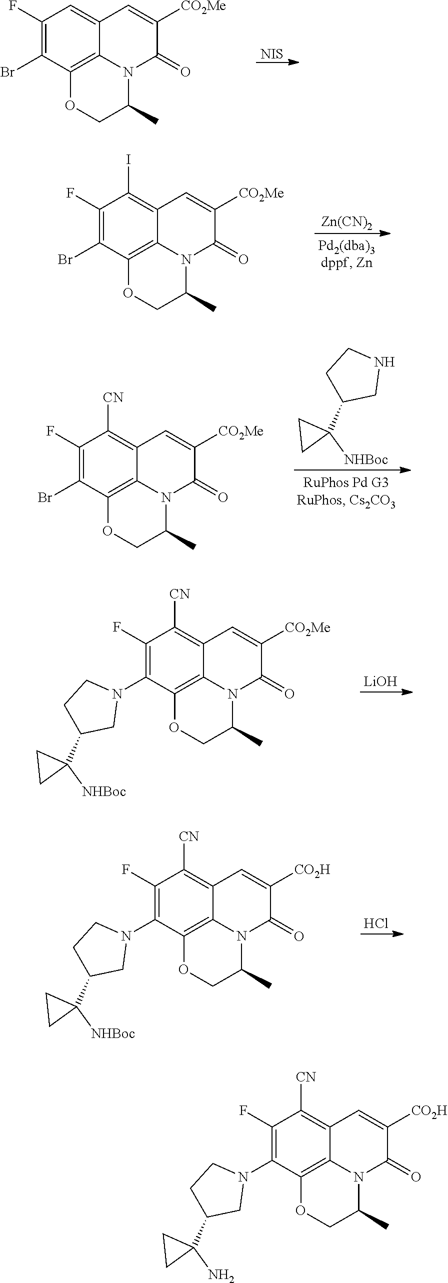

(i). Methyl (S)-10-bromo-9-fluoro-8-iodo-3-methyl-5-oxo-2,3-dihydro-5H-[1,4]oxazino[2,3,4-ij]quinoline-6-carboxylate: Methyl (S)-10-bromo-9-fluoro-3-methyl-5-oxo-2,3-dihydro-5H-[1,4]oxazino[2,3,4-ij]quinoline-6-carboxylate (1.50 g, 4.21 mmol) was dissolved in DCM (7 mL) and NIS (1.90 g, 8.46 mmol) was added. The reaction mixture was cooled to 0° C., and H₂SO₄ (7 mL) was slowly added followed by stirring at rt for 1 h. The reaction mixture was diluted with ice cold water and extracted with EtOAc. The combined organic layer was washed with brine, dried over sodium sulfate and concentrated under vacuum to afford the desired product (1.50 g, 74% yield) which was directly used in the next step without any further purification. ¹H NMR (400 MHz, Chloroform-d) δ 8.73 (s, 1H), 5.30-5.19 (m, 1H), 4.63 (dd, J=11.4, 1.2 Hz, 1H), 4.23 (dd, J=11.4, 2.5 Hz, 1H), 4.03 (s, 3H), 1.46 (d, J=6.7 Hz, 3H). LCMS (m/z): 484.2 [M+2].

(ii).Methyl (S)-10-bromo-8-cyano-9-fluoro-3-methyl-5-oxo-2,3-dihydro-5H-[1,4]oxazino[2,3,4-ij]quinoline-6-carboxylate: Methyl (S)-10-bromo-9-fluoro-8-iodo-3-methyl-5-oxo-2,3-dihydro-5H-[1,4]oxazino[2,3,4-ij]quinoline-6-carboxylate (200 mg, 0.41 mmol), Zinc cyanide (100 mg, 0.82 mmol), and Zn dust (6.00 mg, 0.10 mmol) were dissolved in DMA (5 mL) and degassed with nitrogen for 5 minutes. 1,1′-Bis(diphenylphosphanyl)ferrocene (18.0 mg, 0.03 mmol), Pd₂(dba)₃ (15.0 mg, 0.02 mmol) were added and the reaction mixture was heated with microwave irradiation at 130° C. for 2 h. The reaction mixture was quenched with cold water and extracted with EtOAc. The organic layer was washed with brine, dried over sodium sulfate and concentrated under vacuum to afford a crude residue which was purified by silica gel chromatography (10-20% EtOAc in Hexane) affording the desired product (60.0 mg, 38% yield). ¹H NMR (400 MHz, Chloroform-d) δ 8.65 (s, 1H), 5.27 (d, J=6.2 Hz, 1H), 4.72 (d, J=11.6 Hz, 1H), 4.31 (dd, J=11.5, 2.5 Hz, 1H), 4.03 (s, 3H), 1.47 (d, J=6.8 Hz, 3H). LCMS (m/z): 383.33 [M+2].

(iii). Methyl (S)-10-((R)-3-(1-((tert-butoxycarbonyl)amino)cyclopropyl)pyrrolidin-1-yl)-8-cyano-9-fluoro-3-methyl-5-oxo-2,3-dihydro-5H-[1,4]oxazino[2,3,4-ij]quinoline-6-carboxylate: Methyl (S)-10-bromo-8-cyano-9-fluoro-3-methyl-5-oxo-2,3-dihydro-5H-[1,4]oxazino[2,3,4-ij]quinoline-6-carboxylate (0.060 g, 0.150 mmol), tert-butyl (R)-(1-(pyrrolidin-3-yl)cyclopropyl)carbamate (0.046 g, 0.204 mmol) and Cs₂CO₃ (0.153 g, 0.470 mmol) were suspended in toluene (5 mL) and degassed with nitrogen for 5 minutes. RuPhos (0.011 g, 0.023 mmol) and RuPhosPdG₃ (0.020 g, 0.023 mmol) were added and the reaction mixture was heated to 80° C. for 4 h. The reaction mixture was filtered through a celite pad and washed with excess EtOAc. The organic layer was concentrated under vacuum to afford a crude residue which was purified by preparative TLC (100% EtOAc) affording the desired product (0.050 g, 60% yield). LCMS (m/z): 527.56 [M+1].

(iv). (S)-10-((R)-3-(1-((tert-butoxycarbonyl)amino)cyclopropyl)pyrrolidin-1-yl)-8-cyano-9-fluoro-3-methyl-5-oxo-2,3-dihydro-5H-[1,4]oxazino[2,3,4-ij]quinoline-6-carboxylic acid: Methyl (S)-10-((R)-3-(1-((tert-butoxycarbonyl)amino)cyclopropyl)pyrrolidin-1-yl)-8-cyano-9-fluoro-3-methyl-5-oxo-2,3-dihydro-5H-[1,4]oxazino[2,3,4-ij]quinoline-6-carboxylate (0.050 g, 0.090 mmol) was added into a mixture of MeOH:H₂O (3:1, 4 mL). LiOH.H₂O (1M in water, 0.3 mL, 0.280 mmol) was added and the reaction mixture was stirred at rt for 3 h. The reaction mixture was quenched with cold water, acidified with diluted HCl to pH 4 and extracted with EtOAc. The organic layer was washed with brine, dried over sodium sulfate and concentrated under vacuum to afford the desired product (0.045 g) which was directly used in the next step without any further purification. LCMS (m/z): 513.66 [M+1].

(v). (S)-10-((R)-3-(1-aminocyclopropyl)pyrrolidin-1-yl)-8-cyano-9-fluoro-3-methyl-5-oxo-2,3-dihydro-5H-[1,4]oxazino[2,3,4-ij]quinoline-6-carboxylic acid trifluoroacetate salt: (S)-10-((R)-3-(1-((tert-butoxycarbonyl)amino)cyclopropyl)pyrrolidin-1-yl)-8-cyano-9-fluoro-3-methyl-5-oxo-2,3-dihydro-5H-[1,4]oxazino[2,3,4-ij]quinoline-6-carboxylic acid (0.050 g, 0.080 mmol) was dissolved in DCM (2 mL) and cooled to 0° C. TFA (0.4 mL) was added and the reaction mixture was stirred at rt for 1 h. The reaction mixture was concentrated under vacuum to afford a crude residue which was triturated with diethyl ether, affording the desired product (0.032 g, 88% yield). ¹H Wit (400 MHz, Methanol-d₄) δ 8.78 (s, 1H), 5.23-5.21 (m, 1H), 4.66 (d, J=11.1 Hz, 1H), 4.22 (d, J=11.3 Hz, 1H), 4.07-4.05 (m, 1H), 3.83-3.81 (m, 3H), 2.66-2.64 (m, 1H), 2.17-2.15 (m, 1H), 1.76-1.74 (m, 1H), 1.47 (d, J=6.7 Hz, 3H), 1.04 (d, J=15.6 Hz, 4H). LCMS: t_{R}=1.37 min, m/z=413.49 [M+1] (10 minute run, method B).

Using the procedures described for Example 21.1 the following compound were prepared:

| | | | HPLC, | |
|---|---|---|---|---|
| Ex | | | t_{R} | LCMS |
| # | Structure | Chemical Name | (min) | [M + 1] |
| 21.2¹ | 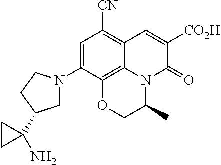 [SMILES: C[C@H]1COc2c(N3CC[C@@H](C4(N)CC4)C3)cc(C#N)c3cc(C(=O)O)c(=O)n1c23] | (S)-10-((R)-3- (1-aminocyclopropyl)pyrrolidin- 1-yl)-8-cyano-3-methyl-5-oxo- 2,3-dihydro-5H- [1,4]oxazino[2,3,4-ij]quinoline- 6-carboxylic acid (TFA salt) | 1.38 | 395.33 |

¹In step 1, use methyl (S)-10-bromo-3-methy1-5-oxo-2,3-dihydro-5H-[1,4]oxazino[2,3,4-ij]quinoline-6-carboxylate as starting material.

HPLC method: Characterized by high performance liquid chromatography (HPLC) on a Waters ACQUITY H class UPLC system with 0.55 mL/min flow rate; column BEH-C18, 1.7 um, 2.1×50 mm from Waters, column temperature: ambiant; gradient (solvent A is 2 mM Ammonium Acetate and 0.1% Formic Acid in Water, solvent B is 0.1% Formic Acid in acetonitrile): 5% solvent B hold for 0.4 min, 5-40% solvent B over 0.6 min, 40-60% solvent B over 1.2 min, 60-100% solvent B over 2.3 min then 100% solvent B over 3 min; compounds were detected by ultraviolet light (UV) absorption at 236 nm.

### Example 22

### (S)-10-((R)-3-(1-aminocyclopropyl)pyrrolidin-1-yl)-9-fluoro-3,8-dimethyl-5-oxo-2,3-dihydro-5H-[1,4]oxazino[2,3,4-ij]quinoline-6-carboxylic acid

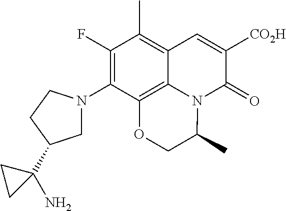 [SMILES: Cc1c(F)c(N2CC[C@@H](C3(N)CC3)C2)c2c3c1cc(C(=O)O)c(=O)n3[C@@H](C)CO2]

The title compound was prepared in accordance with the following scheme:

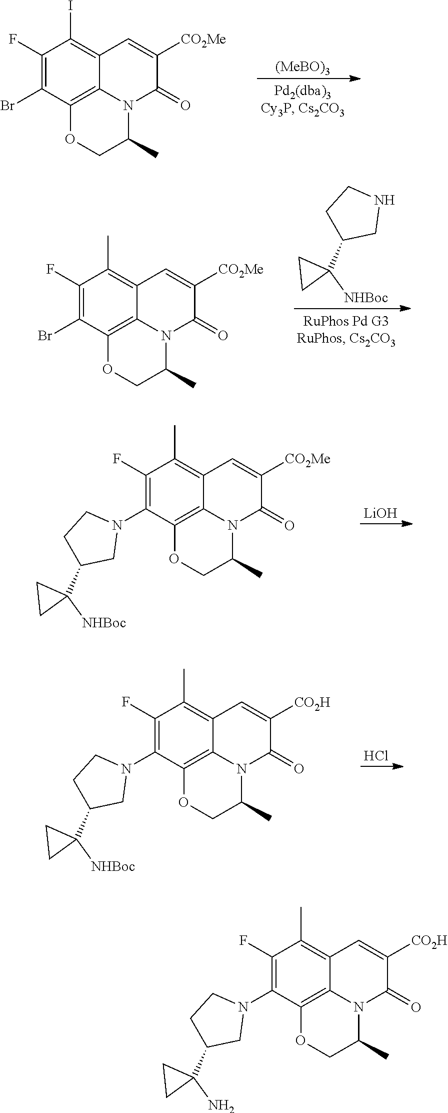

(i).Methyl (S)-10-((R)-3-(1-((tert-butoxycarbonyl)amino)cyclopropyl)pyrrolidin-1-yl)-8-chloro-9-fluoro-3-methyl-5-oxo-2,3-dihydro-5H-[1,4]oxazino[2,3,4-ij]quinoline-6-carboxylic acid: A suspension of methyl (S)-10-bromo-9-fluoro-8-iodo-3-methyl-5-oxo-2,3-dihydro-5H-[1,4]oxazino[2,3,4-ij]quinoline-6-carboxylate (0.250 g, 0.518 mmol) and cesium carbonate (0.507 g, 1.556 mmol) in toluene (10 mL) were degassed with nitrogen for 10 minutes. Trimethylboroxime (0.025 g, 0.245 mmol), tricyclohexyl phospine (0.021 g, 0.077 mmol) and Pd₂dba₃ (0.023 g, 0.025 mmol) were added and the reaction mixture was heated to 70° C. for 9 h. The reaction mixture was filtered through a celite pad and volatiles were evaporated under reduced pressure. The residue was purified by silica gel chromatography (20-50% EtOAc/hexane) affording the desired product as a yellow solid. ¹H NMR (400 MHz, Chloroform-d) δ 8.67 (s, 1H), 5.34-5.24 (m, 1H), 4.61 (dd, J=16.4, 11.4 Hz, 1H), 4.26-4.16 (m, 1H), 4.06 (s, 3H), 2.55 (s, 3H), 1.46 (d, J=6.7 Hz, 3H). LCMS (m/z): 372.17 [M+2].

(ii).Methyl (S)-10-((R)-3-(1-((tert-butoxycarbonyl)amino)cyclopropyl)pyrrolidin-1-yl)-9-fluoro-3,8-dimethyl-5-oxo-2,3-dihydro-5H-[1,4]oxazino[2,3,4-ij]quinoline-6-carboxylate: Methyl (S)-10-((R)-3-(1-((tert-butoxycarbonyl)amino)cyclopropyl)pyrrolidin-1-yl)-8-chloro-9-fluoro-3-methyl-5-oxo-2,3-dihydro-5H-[1,4]oxazino[2,3,4-ij]quinoline-6-carboxylic acid (0.137 g, 0.370 mmol), tert-butyl (R)-(1-(pyrrolidin-3-yl)cyclopropyl)carbamate (0.280 g, 1.243 mmol) and Cs₂CO₃ (0.607 g, 1.863 mmol) were suspended in toluene (10 mL) and degassed with nitrogen for 5 minutes. RuPhos (0.043 g, 0.093 mmol) and RuPhosPdG₃ (0.077 g, 0.093 mmol) were added and the reaction mixture was heated to 70° C. for 8 h. The reaction mixture was poured into water and extracted with EtOAc. The combined organic layer was washed with brine, dried over sodium sulfate and concentrated under vacuum to afford a crude residue which was purified by reverse phase chromatography (60-70% MeOH in water), providing the desired product (0.118 g, 62% yield) as a yellow solid. ¹H NMR (400 MHz, Chloroform-d) δ 8.67 (s, 1H), 5.34-5.24 (m, 1H), 4.37-4.22 (m, 2H), 4.25-4.11 (m, 1H), 4.06 (s, 3H), 3.92-3.86 (m, 4H), 2.53 (s, 3H), 2.30-2.18 (m, 2H), 2.20-2.02 (m, 1H), 1.51-1.38 (m, 9H), 1.42-1.30 (m, 5H), 1.02 (dd, J=6.6, 3.0 Hz, 2H). LCMS (m/z): 516.6 [M+1].

(iii). ((S)-10-((R)-3-(1-((tert-butoxycarbonyl)amino)cyclopropyl)pyrrolidin-1-yl)-9-fluoro-3,8-dimethyl-5-oxo-2,3-dihydro-5H-[1,4]oxazino[2,3,4-ij]quinoline-6-carboxylic acid: Methyl (S)-10-((R)-3-(1-((tert-butoxycarbonyl)amino)cyclopropyl)pyrrolidin-1-yl)-9-fluoro-3,8-dimethyl-5-oxo-2,3-dihydro-5H-[1,4]oxazino[2,3,4-ij]quinoline-6-carboxylate (0.118 g, 0.373 mmol) was dissolved into a mixture of MeOH:H₂O (4:1, 2.5 mL). LiOH.H₂O (1M in water, 0.500 mL, 1.119 mmol) was added and the reaction mixture was stirred at rt for 2 h. The reaction mixture was quenched with water, acidified with diluted HCl to pH 4 and extracted with ethyl acetate. The combined organic layer was dried over sodium sulfate and concentrated under vacuum to afford the desired product as yellow solid which was directly used in the next step without any further purification. LCMS (m/z): 502.6 [M+1].

(iv). (S)-10-((R)-3-(1-aminocyclopropyl)pyrrolidin-1-yl)-9-fluoro-3,8-dimethyl-5-oxo-2,3-dihydro-5H-[1,4]oxazino[2,3,4-ij]quinoline-6-carboxylic acid: ((S)-10-((R)-3-(1-((tert-butoxycarbonyl)amino)cyclopropyl)pyrrolidin-1-yl)-9-fluoro-3,8-dimethyl-5-oxo-2,3-dihydro-5H-[1,4]oxazino[2,3,4-ij]quinoline-6-carboxylic acid was dissolved in DCM (1 mL) and cooled to 0° C. HCl in dioxane (4M, 2 mL) was added and the reaction mixture was allowed to stir at rt for 2 h. The reaction mixture was concentrated under vacuum to afford a crude residue which was purified by reverse phase Prep HPLC, affording the desired product (0.045 g, 33% yield) as a yellow solid. ¹H NMR (400 MHz, MeOD) δ δ 6 8.94 (s, 1H), 5.23-5.15 (m, 1H), 4.49-4.41 (m, 1H), 4.10-4.00 (m, 2H), 3.94-3.84 (m, 1H), 3.76-3.65 (m, 2H), 2.48 (s, 3H), 2.07-2.02 (m, 2H), 1.84-1.75 (m, 1H), 1.49 (d, J=6.6 Hz, 3H), 0.72-0.62 (m, 2H), 0.58-0.56 (m, 2H). LCMS: t_{R}=1.40 min, m/z=402.4 [M+1] (10 minute run, method B).

### Example 23

### (S)-10-((R)-3-(1-aminocyclopropyl)pyrrolidin-1-yl)-8-ethyl-9-fluoro-3-methyl-5-oxo-2,3-dihydro-5H-[1,4]oxazino[2,3,4-ij]quinoline-6-carboxylic acid

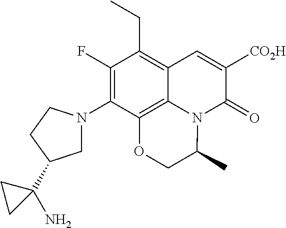 [SMILES: CCc1c(F)c(N2CC[C@@H](C3(N)CC3)C2)c2c3c1cc(C(=O)O)c(=O)n3[C@@H](C)CO2]

The title compound was prepared in accordance with the following scheme:

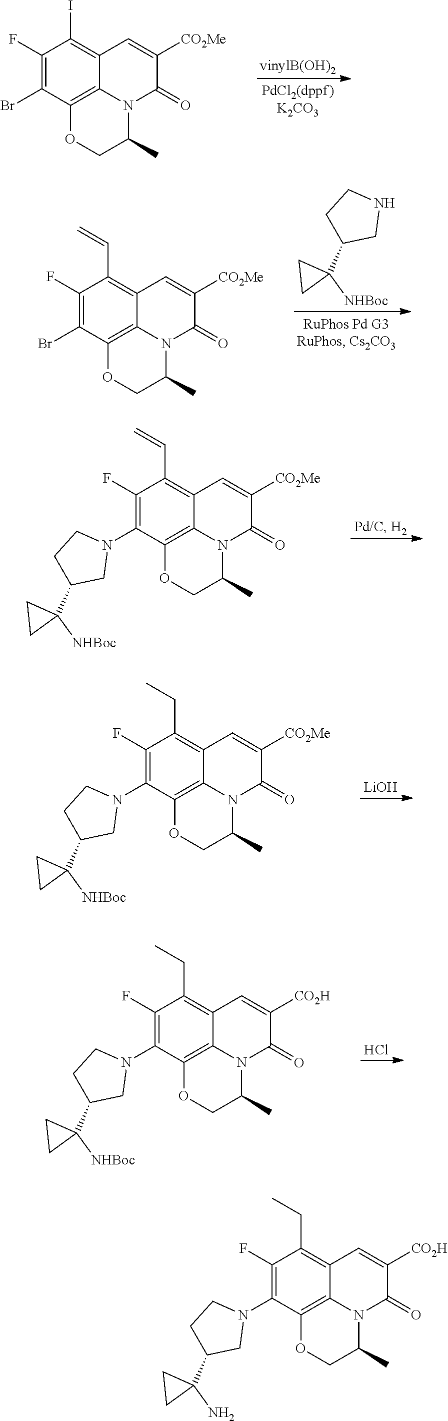

(i). Methyl (S)-10-bromo-9-fluoro-3-methyl-5-oxo-8-vinyl-2,3-dihydro-5H-[1,4]oxazino[2,3,4-ij]quinoline-6-carboxylate: Methyl (S)-10-bromo-9-fluoro-8-iodo-3-methyl-5-oxo-2,3-dihydro-5H-[1,4]oxazino[2,3,4-ij]quinoline-6-carboxylate (1.20 g, 2.48 mmol) was dissolved in DMF (25 mL). K₂CO₃ (1.03 g, 7.46 mmol) and vinylboronic acid (4.62 g, 2.99 mmol) were added and the suspension was degassed with nitrogen for 5 minutes. PdCl₂(dppf) DCM complex (0.11 g, 0.12 mmol) was added and reaction was heated at 70° C. for 16 h. The reaction mixture was diluted with water and extracted with EtOAc. The combined organic layer was washed with brine, dried over sodium sulfate and concentrated under vacuum to afford a crude residue which was purified by silica gel column chromatography (0-30% EtOAc in Hexane), providing the desired product (0.70 g, 74% yield). ¹H NMR (400 MHz, Chloroform-d) δ 8.79 (s, 1H), 6.90 (dd, J=17.7, 11.6 Hz, 1H), 5.91-5.76 (m, 2H), 5.30-5.21 (m, 1H), 4.61 (dd, J=11.5, 1.3 Hz, 1H), 4.22 (dd, J=11.4, 2.5 Hz, 1H), 4.01 (s, 3H), 1.47 (d, J=6.7 Hz, 3H). LCMS (m/z): 384.2 [M+2].

(ii).Methyl (S)-10-((R)-3-(1-((tert-butoxycarbonyl)amino)cyclopropyl)pyrrolidin-1-yl)-9-fluoro-3-methyl-5-oxo-8-vinyl-2,3-dihydro-5H-[1,4]oxazino[2,3,4-ij]quinoline-6-carboxylate: Methyl (S)-10-bromo-9-fluoro-3-methyl-5-oxo-8-vinyl-2,3-dihydro-5H-[1,4]oxazino[2,3,4-ij]quinoline-6-carboxylate (0.500 g, 1.308 mmol), tert-butyl (R)-(1-(pyrrolidin-3-yl)cyclopropyl)carbamate (0.384 g, 1.701 mmol) and Cs₂CO₃ (1.3 g, 3.926 mmol) were suspended in toluene (15 mL) and degassed with nitrogen for 15 minutes. RuPhos (0.092 g, 0.196 mmol) and RuPhosPdG₃ (0.164 g, 0.196 mmol) were added and the reaction mixture was heated at 75° C. for 2 h. The reaction mixture was filtered through a celite pad and washed with excess EtOAc. The filtrate was concentrated under vacuum to afford a crude residue which was purified by Prep TLC (70% EtOAc in Hexane) affording the desired product (0.670 g, 97% yield). LCMS (m/z): 528.6 [M+1].

(iii). Methyl (S)-10-((R)-3-(2-((tert-butoxycarbonyl)amino)propan-2-yl)pyrrolidin-1-yl)-8-ethyl-9-fluoro-3-methyl-5-oxo-2,3-dihydro-5H-[1,4]oxazino[2,3,4-ij]quinoline-6-carboxylate: Methyl (S)-10-((R)-3-(1-((tert-butoxycarbonyl)amino)cyclopropyl)pyrrolidin-1-yl)-9-fluoro-3-methyl-5-oxo-8-vinyl-2,3-dihydro-5H-[1,4]oxazino[2,3,4-ij]quinoline-6-carboxylate (0.060 g, 0.011 mmol) was dissolved in methanolic ammonia (2M, 6 mL). 10% Pd/C (50% in water, 0.020 g) was added and the reaction mixture was stirred under an atmosphere of hydrogen (1 atm) for 1 h at rt. The reaction mixture was filtered through a celite pad, washed with excess methanol and concentrated under vacuum to afford the desired product along with the corresponding amide as impurity. The product was directly used in the next step without any further purification. LCMS (m/z): 530.61 [M+1].

(iv). (S)-10-((R)-3-(1-((tert-butoxycarbonyl)amino)cyclopropyl)pyrrolidin-1-yl)-8-ethyl-9-fluoro-3-methyl-5-oxo-2,3-dihydro-5H-[1,4]oxazino[2,3,4-ij]quinoline-6-carboxylic acid: Methyl (S)-10-((R)-3-(2-((tert-butoxycarbonyl)amino)propan-2-yl)pyrrolidin-1-yl)-8-ethyl-9-fluoro-3-methyl-5-oxo-2,3-dihydro-5H-[1,4]oxazino[2,3,4-ij]quinoline-6-carboxylate (0.052 g, 0.098 mmol) was added into a mixture of MeOH:H₂O (3:1, 4 mL). LiOH.H₂O (1M in water, 0.294 mL, 0.294 mmol) was added and the reaction mixture was stirred at rt for 2 h. The reaction mixture was poured into ice cold water, acidified with diluted HCl to pH 4 and extracted with EtOAc. The combined organic layer was dried over sodium sulfate and concentrated under vacuum to afford the desired product which was directly used in the next step without any further purification. LCMS (m/z): 516.6 [M+1].

(v). (S)-10-((R)-3-(1-aminocyclopropyl)pyrrolidin-1-yl)-8-ethyl-9-fluoro-3-methyl-5-oxo-2,3-dihydro-5H-[1,4]oxazino[2,3,4-ij]quinoline-6-carboxylic acid: (S)-10-((R)-3-(1-((tert-butoxycarbonyl)amino)cyclopropyl)pyrrolidin-1-yl)-8-ethyl-9-fluoro-3-methyl-5-oxo-2,3-dihydro-5H-[1,4]oxazino[2,3,4-ij]quinoline-6-carboxylic acid (0.050 g, 0.097 mmol) was dissolved in DCM (5 mL) and cooled to 0° C. HCl in dioxane (4M, 0.5 mL) was added and the reaction mixture was stirred at rt for 3 h. The reaction mixture was concentrated under vacuum to afford a crude residue which was purified by reverse phase Prep HPLC affording the desired product (0.007 g, 17% yield).1H NMR (400 MHz, Methanol-d4) δ 8.96 (s, 1H), 5.16 (s, 1H), 4.52 (d, J=11.2 Hz, 1H), 4.06 (d, J=11.5 Hz, 1H), 3.96 (s, 1H), 3.70 (d, J=11.1 Hz, 3H), 2.99 (d, J=7.9 Hz, 2H), 2.47-2.44 (m, 1H), 2.13-2.11 (m, 1H), 1.79-1.77 (m, 1H), 1.42 (d, J=6.5 Hz, 3H), 1.27 (q, J=11.1, 7.7 Hz, 3H), 0.89 (br s, 4H). LCMS: t_{R}=1.39 min, m/z=416.6 [M+1] (10 minute run, method B).

### Pharmaceutical Activity

The activity of a compound according to the present invention can be assessed by the following in vitro methods.

### Assessment of Antibactieral Activity In Vitro

Bacterial isolates were cultivated from −80° C. frozen stocks by overnight passages at 35° C. in ambient air on Mueller-Hinton agar plates (MHA, Becton Dickinson, Franklin Lakes, NB) with the exception of S. pneumoniae which was grown overnight at 35° C. in the presence of 5% CO₂ on blood agar plates (tryptic soy agar with 5% sheep blood (Thermo Scientific, Waltham, Mass.) . The following quality control and wild type strains were obtained from the American Type Culture Collection (ATCC; Rockville, Md.) and are coded in the Novartis strain collection as indicated: E. coli ATCC 25922 (NB27001), E. faecalis ATCC 29212 (NB04001), S. aureus ATCC 49951 (NB01006) and S. pneumoniae ATCC 25922 (NB07001). S. aureus NB01006-AVR005, derived from S. aureus ATCC 49951 by selection on ciprofloxacin-containing Mueller Hinton agar, carries mutations resulting in amino acid substitutions in gyrA (S84L), grlA (580F) and grlB (E471K). S. aureus NB01080 is a fluoroquinolone-resistant clinical isolate with amino acid substitution in gyrA (S84L, E88G) and parC (580F, E84K). P. aeruginosa NB52019, obtained from Queen's University (Kingston, Ontario, Canada), is the wild-type PAO1 strain.

Minimal Inhibitory Concentrations (MIC) were determined by the broth microdilution method in accordance with Clinical and Laboratories Institute (CLSI) guidelines. In brief, fresh bacterial overnight cultures were re-suspended in sterile saline, adjusted to a 0.5 McFarland turbidity standard and then diluted to yield a final inoculum of approximately 5×10⁵ colony-forming units (CFU)/mL. All bacterial suspensions were prepared in cation adjusted Mueller-Hinton Broth (CAMHB; Becton Dickinson, Franklin Lakes, N.J.;) with the exception of S. pneumoniae which was prepared in CAMHB supplemented with 5% lysed horse blood (Hardy Diagnostics, Santa Maria, Calif.). Two-fold serial dilutions of compounds were prepared in 100% dimethyl sulfoxide (DMSO) at 100-fold the highest final assay concentration; the resulting dilution series of compounds were diluted 1:10 with sterile water. Ten μL of the drug dilution series in 10% DMSO was transferred to microtiter wells and 90 μL of bacterial suspension was inoculated into the wells. All inoculated microdilution trays were incubated in ambient air at 35° C. for 20 hours. Following incubation, assay plates were read in a microtiter plate reader at 600 nm and visually inspected to confirm the MIC endpoint well with the OD value. The lowest concentration of the compound that prevented visible growth was recorded as the MIC (in μg/mL). Performance of the assay was monitored by testing ciprofloxacin or moxifloxacin against laboratory quality control strains in accordance with guidelines of the CLSI.

TABLE 2
Antibacterial activity (μg/mL) of compounds of the invention:
C ≥ 32; 32 > B ≥ 1; 1 > A

| | | NB01006- | | | | | |
|---|---|---|---|---|---|---|---|
| Example # | NB01006 | AVR005 | NB01080 | NB04001 | NB07001 | NB27001 | NB52019 |
| 1.1 | A | B | A | A | A | B | C |
| 1.2 | B | C | C | B | B | B | B |
| 1.3 | A | B | B | B | B | B | C |
| 1.4 | B | C | C | C | C | B | C |
| 1.5 | A | B | A | A | A | B | B |
| 1.6 | A | C | B | B | B | B | C |
| 1.7 | A | B | A | A | A | C | — |
| 1.8 | A | B | A | A | A | C | C |
| 1.9 | B | C | C | C | B | C | C |
| 1.10 | B | C | B | B | A | C | C |
| 1.11 | B | C | B | B | A | B | C |
| 1.12 | A | B | B | B | A | B | C |
| 1.13 | B | C | C | B | B | B | C |
| 1.14 | A | B | B | B | B | C | C |
| 1.15 | A | B | B | B | A | B | C |
| 1.16 | A | B | B | B | A | B | C |
| 1.17 | C | C | C | C | C | C | C |
| 1.18 | A | C | C | B | B | C | C |
| 1.19 | B | C | B | B | A | C | C |
| 1.20 | B | C | B | B | B | C | C |
| 1.21 | B | C | C | C | B | C | C |
| 1.22 | B | C | B | B | B | C | C |
| 1.23 | C | C | C | C | B | B | C |
| 1.24 | A | B | B | A | A | B | C |
| 1.25 | A | C | A | B | B | C | C |
| 1.26 | B | C | B | B | B | B | C |
| 1.27 | B | C | B | B | A | B | C |
| 1.28 | B | C | C | B | A | C | C |
| 1.29 | C | C | C | C | C | C | C |
| 1.30 | B | C | C | B | B | C | C |
| 1.31 | A | C | B | B | A | B | C |
| 1.32 | B | C | C | B | B | C | C |
| 1.33 | B | C | B | B | B | B | C |
| 1.34 | B | C | C | C | C | C | C |
| 1.35 | B | C | B | B | B | B | C |
| 1.36 | A | B | A | A | A | B | C |
| 1.37 | C | C | C | C | C | C | C |
| 1.38 | A | C | B | B | B | A | B |
| 1.39 | C | C | C | C | C | C | C |
| 1.40 | B | B | B | B | A | B | C |
| 1.41 | A | B | B | B | A | B | C |
| 1.42 | A | B | B | B | A | A | B |
| 1.43 | A | B | B | A | A | B | C |
| 1.44 | A | C | C | B | B | B | C |
| 1.45 | A | B | A | A | A | B | C |
| 1.46 | A | B | A | A | A | B | C |
| 1.47 | A | B | B | B | A | B | C |
| 1.48 | A | B | B | A | A | C | C |
| 1.49 | A | B | B | A | A | B | B |
| 1.50 | A | B | B | B | A | C | C |
| 1.51 | C | C | C | C | C | C | C |
| 1.52 | A | B | A | A | A | A | C |
| 1.53 | A | B | A | A | A | B | C |
| 1.54 | B | C | C | C | C | C | C |
| 1.55 | B | C | B | B | B | B | C |
| 1.56 | A | B | A | A | A | A | B |
| 1.57 | C | C | C | C | C | B | C |
| 1.58 | A | B | B | B | B | B | C |
| 1.59 | B | C | B | C | B | B | C |
| 1.60 | B | C | C | B | B | B | B |
| 1.61 | C | C | C | C | C | C | C |
| 1.62 | B | C | C | C | C | C | C |
| 1.63 | C | C | C | C | C | B | C |
| 1.64 | B | C | C | B | B | C | C |
| 1.65 | B | C | C | C | B | B | C |
| 1.66 | B | C | C | C | B | B | C |
| 1.67 | C | C | C | C | C | C | C |
| 1.68 | C | C | C | C | C | C | C |
| 1.69 | B | C | C | C | B | C | C |
| 1.70 | B | C | C | C | B | C | C |
| 1.71 | C | C | C | C | C | C | C |
| 1.72 | B | C | C | C | C | C | C |
| 1.73 | B | C | C | C | C | B | C |
| 1.74 | A | B | B | B | A | B | C |
| 1.75 | A | B | B | B | A | B | C |
| 1.76 | C | C | C | C | C | C | C |
| 1.77 | B | C | C | C | B | C | C |
| 1.78 | B | C | C | C | B | B | C |
| 1.79 | B | C | C | C | C | C | C |
| 2 | C | C | C | C | C | C | C |
| 3 | C | C | C | C | C | C | C |
| 4 | A | B | B | B | A | B | C |
| 5 | A | A | A | A | A | B | B |
| 6.1 | A | A | A | A | A | B | C |
| 6.2 | B | B | A | B | B | B | C |
| 7.1 | — | B | B | — | A | B | B |
| 7.2 | — | B | C | — | B | B | B |
| 7.3 | — | C | C | — | C | C | C |
| 7.4 | — | C | C | — | C | C | C |
| 7.5 | — | C | C | — | C | C | C |
| 7.6 | — | C | B | — | C | C | C |
| 7.7 | — | C | C | — | C | C | C |
| 7.8 | — | C | C | — | C | C | C |
| 7.9 | A | A | B | A | A | A | B |
| 7.10 | A | A | B | B | A | A | B |
| 8 | A | A | A | A | A | B | B |
| 9.1 | A | B | A | A | A | B | C |
| 9.2 | A | C | B | A | A | B | C |
| 9.3 | C | C | C | C | C | B | C |
| 10 | B | C | B | B | B | B | C |
| 11 | A | B | A | A | A | B | C |
| 12.1 | A | A | A | A | A | A | B |
| 12.2 | A | B | B | A | A | B | B |
| 12.3 | A | B | A | A | A | A | C |
| 12.4 | A | B | B | A | A | B | B |
| 12.5 | B | C | B | B | B | B | C |
| 12.6 | B | C | B | B | B | B | C |
| 13.1 | A | A | A | A | A | A | B |
| 13.2 | A | A | A | A | A | A | B |
| 13.3 | B | C | C | C | C | B | C |
| 13.4 | A | B | B | A | A | A | B |
| 13.5 | A | B | B | A | A | B | C |
| 13.6 | A | B | A | A | A | B | C |
| 13.7 | A | B | B | A | A | A | B |
| 13.8 | A | A | A | A | A | B | B |
| 14 | A | B | B | A | A | B | C |
| 15 | C | C | C | C | B | C | C |
| 16 | C | C | C | C | B | C | C |
| 17 | A | B | B | A | A | C | C |
| 18 | B | C | B | B | A | C | C |
| 19.1 | A | A | A | A | A | B | C |
| 19.2 | A | A | A | A | A | A | B |
| 19.3 | A | A | A | A | A | B | C |
| 20 | A | B | B | B | A | B | C |
| 21.1 | A | A | A | A | A | B | C |
| 21.2 | A | B | B | B | A | B | C |
| 22 | A | B | A | A | A | B | C |
| 23 | B | C | B | B | B | C | C |
| CIP | A | C | C | A | A | A | A |
| MOX | A | B | C | A | A | A | B |

Note:
Ciprofloxacin (CIP) and moxifloxacin (MOX) were included as control agents. The MIC values reported in the table for the controls are mode values from at least 35 assays.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A compound of formula (I): 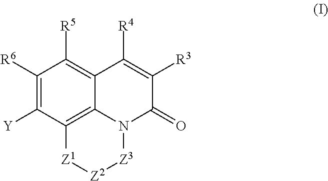
wherein:
Z¹ is selected from the group consisting of O, S, NR¹, and C(R¹)₂;
Z² is selected from C(R¹)₂, O, —C(R¹)₂—C(R¹)₂—, and a bond connecting Z¹ to Z³, provided that when Z² is O, Z¹ is C(R¹)₂;
Z³ is C(R¹)₂;
wherein R¹ is independently selected at each occurrence from H and C₁-C₃ alkyl that is optionally substituted with up to three groups selected from halo, hydroxyl, C₁-C₃-alkoxy, and CN;
R³ is selected from the group consisting of H, -L¹-OR², -L¹-CN, -L¹-N(R²)₂, -L¹-COOR², -L¹-CON(R²)₂, -L¹-N(R²)C(O)R², -L¹-N(R²)C(O)OR, -L¹-SO₂R, -L¹-N(R²)-SO₂-R, and -L¹-SO₂-N(R²)₂; wherein each L¹ is a bond, or a C₁-C₄ straight or branched chain alkylene linker;
each R is independently C₁-C₄ alkyl optionally substituted with one to three groups selected from halogen, —OH, alkoxy, CN, —NH₂, —NH(C₁-C₄ alkyl), —N(C₁-C₄ alkyl)₂, —SO₂(C₁-C₄ alkyl), and oxo;
each R² is independently H or C₁-C₄ alkyl optionally substituted with up to three groups selected from halogen, —OH, alkoxy, CN, —NR¹²R¹³, —SO₂R and oxo;
or two R² on the same nitrogen can be taken together to form a 4-6 membered heterocyclic ring optionally containing an additional heteroatom selected from N, O and S as a ring member and optionally substituted with up to three groups selected from halogen, —OH, C₁-C₄ alkyl, C₁-C₄ alkoxy, CN, —NR¹²R¹³, and oxo;
R⁴ is selected from the group consisting of H, halo, C₁-C₆ alkyl, C₁-C₄ haloalkyl, -L²-OR², -L²-CN, -L²-N(R²)₂, and -L²-NR²C(O)—R²;
each L² is independently selected from a bond and a divalent straight chain or branched C₁-C₆ alkyl;
R⁵ is selected from the group consisting of H, halo, amino, CN, C₁-C₄ alkyl, C₁-C₄ alkoxy, and C₁-C₄ haloalkyl;
R⁶ is selected from the group consisting of H, halo, CN, C₁-C₄ alkyl, C₁-C₄ alkoxy, and C₁-C₄ haloalkyl;
Y is a group of the formula —NR^{7A}R^{7B},
wherein R^{7A} is selected from the group consisting of H, —C(O)R², —C(O)OR², and C₁-C₆ alkyl optionally substituted with up to two groups independently selected from halogen, —OH, C₁-C₄ haloalkyl, C₁-C₄ alkoxy, oxo, ═N—OR², —N(R²)₂, C₃-C₇ cycloalkyl, —COOR², —C(O)N(R²)₂, —NR²C(O)R², —NR²C(O)OR, and a 4-6 membered heteroaryl or heterocyclyl group that contains up to two heteroatoms selected from N, O and S as ring members and is optionally substituted with up to two groups selected from hydroxy, amino, halogen, C₁-C₄ alkyl, C₁-C₄ haloalkyl, and C₁-C₄ alkoxy;
R^{7B} is -L³-Q³ or C₁-C₆ alkyl optionally substituted with up to two groups independently selected from halogen, —OH, C₁-C₄ haloalkyl, C₁-C₄ alkoxy, oxo, —N(R²)₂, C₃-C₇ cycloalkyl, —COOR², —C(O)N(R²)₂, —NR²C(O)R², —NR²C(O)OR, and a 4-6 membered heteroaryl or heterocyclyl group that contains up to two heteroatoms selected from N, O and S as ring members and is optionally substituted with up to two groups selected from hydroxy, amino, halogen, C₁-C₄ alkyl, C₁-C₄ haloalkyl, and C₁-C₄ alkoxy,
wherein L³ is a bond or a straight or branched chain C₁-C₆ alkyl linker, and Q³ is selected from pyridinyl and a 4-7 membered heterocyclyl containing one or two heteroatoms selected from N, O and S as ring members, and wherein Q³ is optionally substituted with up to three groups selected from halogen, CN, —OH, C₁-C₄ alkyl, C₁-C₄ haloalkyl, C₁-C₄ alkoxy, oxo,=N—OR², —N(R²)₂, —COOR², —C(O)N(R²)₂, —NR²C(O)R², —NR²C(O)OR;
or R^{7A} and R^{7B} together with the nitrogen atom to which they are attached form a 4- to 7-membered monocyclic group optionally including one additional heteroatom selected from N, O and S as a ring member, or a 6-10 membered bicyclic heterocyclic group optionally including one or two additional heteroatoms selected from N, O and S as ring members, wherein the monocyclic or bicyclic heterocyclic group formed by R^{7A} and R^{7B} together with the nitrogen atom to which they are attached is optionally substituted by up to four groups selected from halogen, —CN, hydroxy, phenyl, oxo, —OR⁹, —N(R⁹)₂, —COOR⁹, —C(O)N(R⁹)₂, C₁-C₄ alkyl, ═C(R⁸)₂, C₁-C₄ haloalkyl, C₁-C₄ alkoxy, oxo, C₃-C₆ cycloalkyl, and a 4-6 membered heteroaryl or heterocyclyl group that contains up to two heteroatoms selected from N, O and S as ring members,
wherein the C₁-C₄ alkyl, C₃-C₆ cycloalkyl, phenyl, and 4-6 membered heteroaryl or heterocyclyl are each optionally substituted by up to three groups independently selected from halogen, —CN, hydroxy, oxo, —OR¹⁰, ═N—OR¹⁰, —N(R¹⁰)₂, —COOR¹⁰, —N(R¹⁰)—C(O)—O—(C₁-C₄ alkyl), —C(O)N(R¹⁰)₂, C₁-C₄ alkyl, C₁-C₄ haloalkyl, and C₁-C₄ alkoxy;
R⁸ is selected independently at each occurrence from the group consisting of H, halo, CN, C₁-C₄ alkoxy, C₁-C₄ haloalkyl, and C₁-C₄ alkyl optionally substituted with hydroxy or amino;
R⁹ and R¹⁰ are each independently selected from H and C₁-C₄ alkyl optionally substituted with up to three groups selected from halogen, —OH, C₁-C₄ alkoxy, CN, —NR¹²R¹³, —SO₂R and oxo;
or two R⁹ or two R¹⁰ on the same nitrogen can be taken together to form a 4-6 membered heterocyclic ring optionally containing an additional heteroatom selected from N, O and S as a ring member and optionally substituted with up to three groups selected from halogen, —OH, C₁-C₄ alkyl, C₁-C₄ alkoxy, CN, —NR¹²R¹³, and oxo;
each R¹¹ is independently hydrogen or C₁-C₄ alkyl optionally substituted with one or two groups selected from halogen, —OH, C₁-C₄ alkoxy, CN, —NH₂, —NH(C₁-C₄ alkyl), —N(C₁-C₄ alkyl)₂, —SO₂(C₁-C₄ alkyl), and oxo;
each R¹² and R¹³ is independently hydrogen or C₁-C₄ alkyl optionally substituted with one or two groups selected from halogen, —OH, C₁-C₄ alkoxy, CN, —NH₂, —NH(C₁-C₄ alkyl), —N(C₁-C₄ alkyl)₂, —SO₂(C₁-C₄ alkyl), and oxo;
or each R¹² and R¹³ together with the nitrogen atom to which they are both attached can form a 4- to 6-membered heterocyclyl optionally including an additional heteroatom selected from N, O and S as a ring member and optionally substituted by one to three substituents selected from OH, halogen, oxo, ═N—OR¹¹, C₁-C₆ alkyl optionally substituted by one to three halogen atoms or NH₂, C₁-C₆ alkoxy optionally substituted by one or more OH or C₁-C₆ alkoxy groups, and —C(O)O—C₁-C₆ alkyl;
or a pharmaceutically acceptable salt thereof.

2. The compound of claim 1, wherein R³ is H or COOR².

3. The compound of claim 1, or a pharmaceutically acceptable salt thereof, wherein R⁵ is H or halogen.

4. The compound of claim 1, wherein R⁶ is H or F; or a pharmaceutically acceptable salt thereof.

5. The compound of claim 1, wherein each R¹ is independently selected from H and methyl; or a pharmaceutically acceptable salt thereof.

6. The compound of claim 1, wherein R³ is H; or a pharmaceutically acceptable salt thereof.

7. The compound of claim 1, wherein R³ is —COOH; or a pharmaceutically acceptable salt thereof.

8. The compound of claim 1, wherein R⁴ is H; or a pharmaceutically acceptable salt thereof.

9. The compound of claim 1, wherein R⁴ is —CH₂—N(R²)₂; or a pharmaceutically acceptable salt thereof.

10. The compound of claim 1, which is of the formula (II): 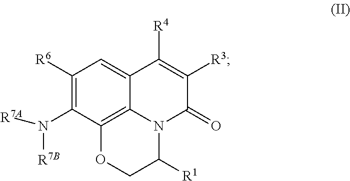
or a pharmaceutically acceptable salt thereof.

11. The compound according to claim 10, wherein R^{7A} is H; or a pharmaceutically acceptable salt thereof.

12. The compound according to claim 10, wherein R^{7A} and R^{7B} together with the nitrogen atom to which they are attached form a 4- to 7-membered monocyclic heterocyclic group optionally including one additional heteroatom selected from N, O and S as a ring member,
or a 6-10 membered bicyclic heterocyclic group optionally including one or two additional heteroatoms selected from N, O and S as ring members,
wherein the monocyclic or bicyclic heterocyclic group formed by R^{7A} and R^{7B} together with the nitrogen atom to which they are attached is optionally substituted by up to three groups selected from halogen, —CN, hydroxy, phenyl, oxo, —OR⁹, —N(R⁹)₂, —COOR⁹, —C(O)N(R⁹)₂, C₁-C₄ alkyl, C₁-C₄ haloalkyl, C₁-C₄ alkoxy, oxo, C₃-C₆ cycloalkyl, and a 4-6 membered heteroaryl or heterocyclyl group that contains up to two heteroatoms selected from N, O and S as ring members,
wherein the C₁-C₄ alkyl, C₃-C₆ cycloalkyl, phenyl, and 4-6 membered heteroaryl or heterocyclyl are each optionally substituted by up to three groups independently selected from halogen, —CN, hydroxy, oxo, -OR¹⁰, ═N—OR¹⁰, —N(R¹⁰)_{2, —COOR}¹⁰, —C(O)N(R¹⁰)₂, C₁-C₄ alkyl, C₁-C₄ haloalkyl, and C₁-C₄ alkoxy;
or a pharmaceutically acceptable salt thereof.

13. The compound according to claim 1, or a pharmaceutically acceptable salt thereof, wherein the compound has formula (IV): 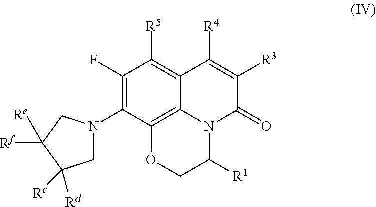
wherein,
R¹ is independently at each occurrence hydrogen or methyl;
R³ is hydrogen, halo , C₁₋₂ alkyl, or C₁₋₂ haloalkyl;
R⁴ is H or —CH₂NH₂,
R⁵ is H, Me or halo;
R^{c} and R^{f} are independently selected from hydrogen and halo, or R^{c} and R^{f} taken together with the atoms to which they are attached form a cyclopropyl ring;
R^{d} and R^{e} are each independently selected from the group consisting of H, —NH₂, —CH₂NH₂, —CH₂NHCH₃, OH, CH₂OH,

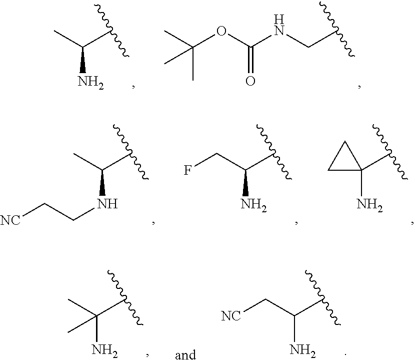

14. The compound according to claim 1, or a pharmaceutically acceptable salt thereof,
wherein:
R³ is hydrogen, C₁₋₂ alkyl, C₁₋₂ haloalkyl, CN, —C(O)OH, C(O)—O—(C₁-C₄ alkyl) or —S(O)₂—(C₁-C₄ alkyl);
each R¹ is independently H or methyl;
Y is selected from the group consisting of:

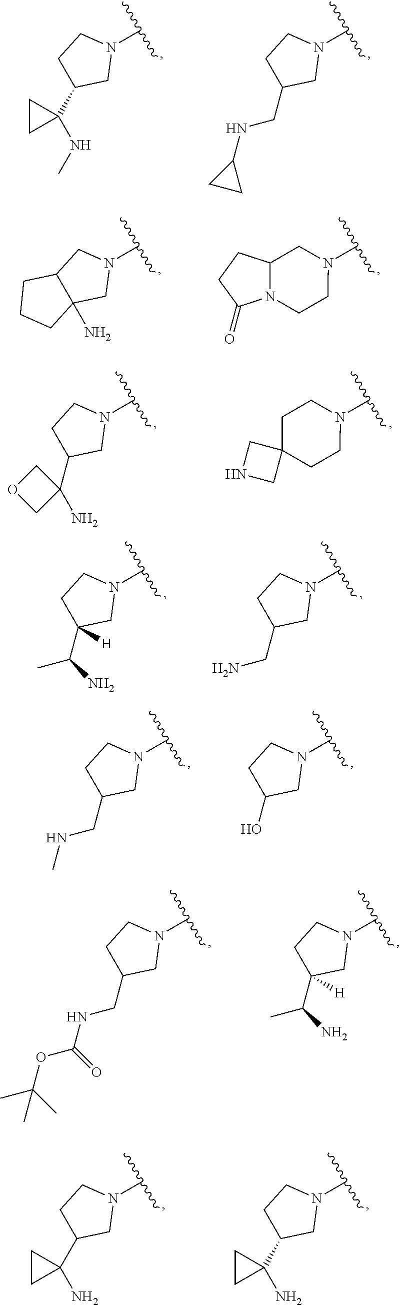

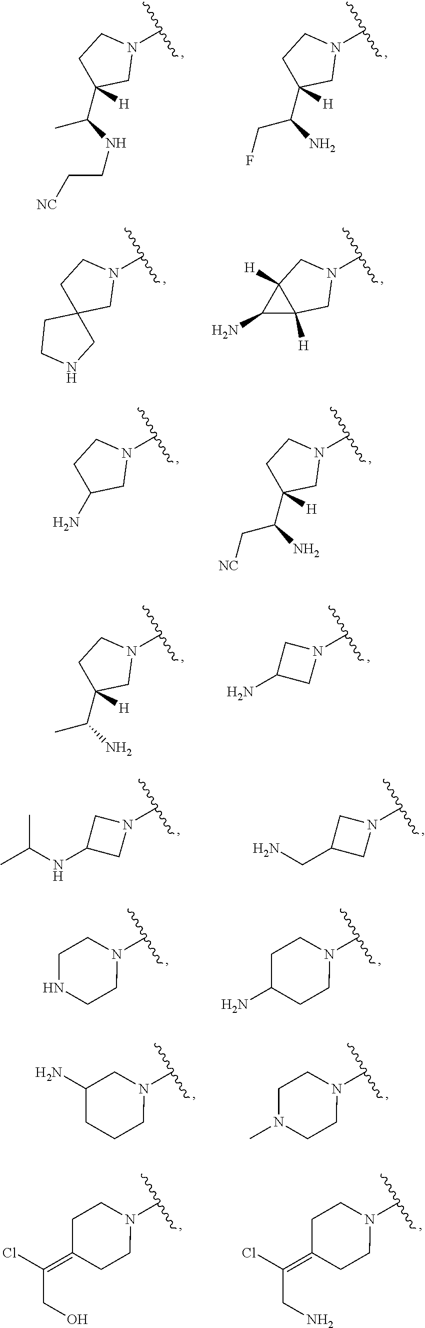

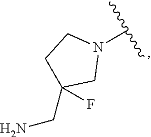 [SMILES: *N1CCC(F)(CN)C1]

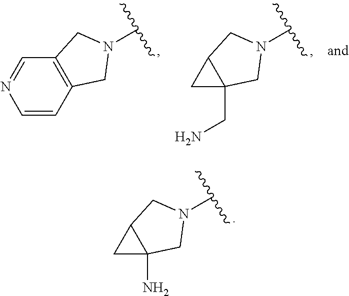 [SMILES: *N1Cc2ccncc2C1 ; *N1CC2CC2(CN)C1 ; *N1CC2CC2(N)C1]

15. A compound of formula (VI): 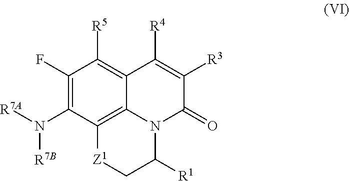
wherein,
R¹ is H, methyl, CH₂F, CH₂OH, or CH₂OMe;
R³ is hydrogen or —COOR²;
R² is H or C₁-C₄ alkyl;
R⁴ is hydrogen or —CH₂NH₂;
Z¹ is O or CH₂;
R⁵ is hydrogen, Me or halo; and
R^{7A} and R^{7B} together with the nitrogen atom to which they are attached form a 5- to 6-membered monocyclic heterocyclic group optionally including one additional heteroatom selected from N, O and S as a ring member, or a 6-10 membered bicyclic heterocyclic group optionally including one additional heteroatom selected from N, O and S as a ring member,
wherein the monocyclic or bicyclic heterocyclic group formed by R^{7A} and R^{7B} together with the nitrogen atom to which they are attached is optionally substituted by up to four groups selected from halogen, —CN, hydroxy, phenyl, oxo, —OR⁹, —N(R⁹)₂, —COOR⁹, —C(O)N(R⁹)₂, C₁-C₄ alkyl, C₁-C₄ haloalkyl, C₃-C₆ cycloalkyl, and a 4-6 membered heteroaryl or heterocyclyl group that contains up to two heteroatoms selected from N, O and S as ring members,
wherein the C₁-C₄ alkyl, C₃-C₆ cycloalkyl, phenyl, and 4-6 membered heteroaryl or heterocyclyl are each optionally substituted by up to three groups independently selected from halogen, —CN, hydroxy, oxo, —OR¹⁰, —N(R¹⁰)₂, —COOR¹⁰, —N(R¹⁰)—C(O)—O—(C₁-C₄ alkyl), —C(O)N(R¹⁰)₂, C₁-C₄ alkyl, C₁-C₄ haloalkyl, and C₁-C₄ alkoxy;
or a pharmaceutically acceptable salt thereof.

16. The compound of claim 15, wherein the group represented by —NR^{7A}R^{7B} is selected from: 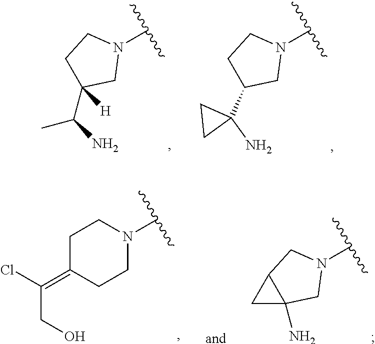
or a pharmaceutically acceptable salt thereof.

17. A pharmaceutical composition, comprising:
the compound according to claim 1, and a
pharmaceutically acceptable carrier, adjuvant or vehicle.

18. The pharmaceutical composition according to claim 17, further comprising an additional therapeutic agent with antibacterial activity.

19. A method for treating a subject having a bacterial infection, comprising:
administering to the subject in need thereof an antibacterially effective amount of the compound according to claim 1.

20. The method of claim 19, wherein the bacterial infection is an infection comprising at least one bacterium selected from the group consisting of Pseudomonas aeruginosa and other Pseudomonas species, Stenotrophomonas maltophilia, Burkholderia cepacia and other Burkholderia species, Acinetobacter baumannii and other Acinetobacter species, Achromobacter xylosoxidans, Alcaligenes denitrificans and other Achromobacteraceae, Citrobacter freundii and other Citrobacter species, Campylobacter jejuni, Klebsiella pneumoniae, Klebsiella oxytoca and other Klebsiella species, Enterobacter cloacae, Enterobacter aerogenes and other Enterobacter species, Escherichia coli, Salmonella enterica and other Salmonella species, Yersinia pestis, Proteus vulgaris and other Proteus species, Serratia marscens and other Serratia species, Morganella morganii and other members of the Enterobacteriaceae family, Neisseria meningitidis, Haemophilus influenzae, Moraxella cattharallis, Bacteroides fragilis, Bacteroides thetaiotaomicron and other Bacteriodes species, Pasteurella multicoda and other Pasteurella species, Fransicella tularensis, Shigella dysenteriae and other Shigella species, Vibrio cholera and other Vibrio species, Bordetella pertussis and other Bordetella species, Helicobactor pylori and other Helicobacter species, Legionella pneumophila and Campylobactor jejuni, Staphylococcus aureus, Staphylococcus epidermidis and other Staphylococcus species, Enterococcus faecalis, Enterococcus faecium and other Enterococcus species, Streptococcus pneumoniae, Streptococcus pyogenes, Streptococcus agalactiae and other Streptococcus species, Bacillus anthracis and other Bacillus species, Peptostreptococcus magnus and other Peptostreptococcus species, Clostridium difficile and other Clostridium species, Listeria monocytogenes and other Listeria species, and Corynebacterium diptheriae and other Corynebacterium species.
